(19) United States
(12) Patent Application Publication
(10) Pub. No.: US 2009/0221428 A1
(43) Pub. Date: Sep. 3, 2009

(54) Methods of Genome-Wide Location Analysis in Stem Cells

(76) Inventors: Richard A. Young, Weston, MA (US); Tong Ihn Lee, Cambridge, MA (US); Matthew Guenther, Cambridge, MA (US); Laurie A. Boyer, Westborough, MA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/065,076

(22) PCT Filed: Sep. 7, 2006
(86) PCT No.: PCT/US06/34962

§ 371 Date: Nov. 3, 2008

Related U.S. Application Data
(60) Provisional application No. 60/715,363, filed on Sep. 7, 2005.
(60) Provisional application No. 60/790,209, filed on Apr. 7, 2006.

(51) Int. Cl. C40B 20/04; C12Q 1/68; C07H 1/00; C40B 50/14

(52) U.S. Cl. 506/4; 435/6; 536/55.3; 506/30

(57) ABSTRACT

The invention relates to improved methods of identifying the genomic regions to which a protein of interest binds, and in particular, to methods that apply to stem cells such as but not limited to; embryonic stem cells and adult stem cells. The invention also provides methods of identifying agents which modulate differentiation of stem cells. The invention also provides methods of defining the differentiation potential of a cell and of designing array oligonucleotides.

### CROSS-REFERENCE TO RELATED APPLICATIONS

This application claims the benefit of the filing date of U.S. Application No. 60/715,363, filed Sep. 7, 2005, entitled “METHODS OF GENOME-WIDE LOCATION ANALYSIS,” and U.S. Application No. 60/790,209, filed Apr. 7, 2006, entitled “GENOME-WIDE LOCATION ANALYSIS OF POLYCOMB AND RNA POLYMERASE II IN HUMAN EMBRYONIC STEM CELLS.” The entire teachings of the referenced application is herein incorporated by reference.

### STATEMENT REGARDING FEDERALLY-SPONSORED RESEARCH OR DEVELOPMENT

The invention described herein was supported, in whole or in part, by NHGRI grant HG002668 and NIH grant GM069400. The United States government has certain rights in the invention.

### BACKGROUND OF THE INVENTION

Genome-wide analysis methods have been used to determine how tagged transcriptional regulators encoded in Saccharomyces cerevisae are associated with the genome in living yeast cells and to model the transcriptional regulatory circuitry of these cells. These methods have also been used in human cell lines cells to identify target genes for several transcriptional regulators. A need remains, however, for developing methods that allow the identification of binding sites on the genome at higher resolutions and that allow the identification of changes in the DNA-binding properties of proteins in stem cells and in differentiating cells. The present invention provides these and other methods.

### SUMMARY OF THE INVENTION

One aspect of the invention provides a method for identifying regions of a genome to which a protein of interest binds, the method comprising the steps of: (a) producing a mixture comprising DNA fragments to which the protein of interest is bound; (b) isolating one or more DNA fragments to which the protein of interest is bound from the mixture produced in step (a); and (c) identifying regions of the genome which are complementary to the DNA fragments isolated in step (b), thereby identifying regions of the genome to which the protein of interest binds. In one embodiment, the mixture in step (a) is from a stem cell. In some embodiments, the stem cell is an embryonic stem cell. In certain aspects, the stem cell is an adult stem cell. In one embstem cells derived from mammals such as mice, rodents, rats, rabbits, cows, pigs, humans, primates, horses, or non-human primates. In some embodiments, the stem cells are from a non-human mammal. In some embodiments, the stem cell maintains its potential to differentiate into one or more of endoderm, mesoderm, and ectoderm tissues.

Some embodiments of the methods for identifying regions of a genome to which a protein of interest binds comprise, between steps (b) and (c), the step of generating a probe from the one or more of the isolated DNA fragments. In certain embodiments, step (c) comprises combining the probe with one or more sets of distinct oligonucleotide features bound to a surface of a solid support, wherein the distinct oligonucleotide features are each complementary to a region of the genome, under conditions in which specific hybridization between the probe and the oligonucleotide features can occur, and detecting said hybridization, wherein hybridization between the labeled probe and a oligonucleotide feature relative to a suitable control indicates that the protein of interest is bound to the region of the genome to which the sequence of the oligonucleotide feature is complementary. In some embodiments, each set of distinct oligonucleotide features is complementary to a regulatory region of a gene for regulating transcription of the gene. Regulatory regions may include sequences upstream and downstream of the transcriptional start site of the gene. In certain aspects, however, the regulatory region does not include a sequence downstream of the transcription start site. In some embodiments, the regulatory regions comprise the regions to which a RNA polymerase binds. In certain embodiments, the regulatory region of the gene comprises from about 8 kb upstream to about 2 kb downstream of a transcriptional start site. In other embodiments, it comprises from at least about 5, 6, 7, 8, 9, 10, 11, 12, 13, 14, 15, 20 or 25 kb upstream of the transcription start site to at least about 50, 100, 200, 300, 400, 500, 600, 800, 1000, 1200, 1500, 2000, 2500, 3000, 4000 or 5000 bp downstream of the transcriptional start site.

In some embodiments, the distinct oligonucleotide features are complementary to adjacent regions in the genome that are spaced anywhere from about 10 bp to about 5000 bp of each other. In certain embodiments, the oligonucleotide features comprise DNA or RNA or modified forms thereof, such as PNA or LNA molecules. In certain embodiments, the oligonucleotide features bound to a surface of a solid support include sequences representative of regulatory regions distributed across at least a portion of a genome. In certain embodiments, the oligonucleotide features are complementary to at least 50, 60, 70, 80, 90, or 95% of the regulatory regions in the genome.

In certain embodiments, regulatory regions are promoter regions. In certain embodiments, the solid support is a planar substrate. In other embodiments, the planar substrate is glass. In certain embodiments, the sets of distinct oligonucleotide features bound to a solid surface comprise an array. In certain embodiments, the array is a tiled array.

The invention also provides a method for identifying the differentiation potential of a test cell. One such method comprises (i) identifying regions of the genome of the test cell to which a protein binds, according to the methods described herein, (ii) comparing the regions identified in (i) to one or more reference sets of bound regions, each reference set of bound regions indicative of a differentiation potential of a cell; wherein a match between the regions identified in (i) and one of the reference set of bound regions indicates that the test cell has the differentiation potential corresponding to the reference set of bound regions.

In certain aspects, the method comprises identifying regions of the genome to which a plurality of proteins bind, and the reference set of bound regions comprises regions of the genome to which the plurality of proteins bind. As defined herein, a “plurality” refers to at least 2, at least 3, at least 5, at least 10, or at least 20. The reference set of bound regions can be in the form of a graphical image or can be derived from data in a database (e.g., a relational database including data relating to regions of the genome to which the protein binds).

In certain embodiments, the protein is Oct4, Sox2 or Nanog. In certain aspects, the method comprises determining regions where at least two of Oct4, Sox2 and Nanog binds, or determining regions where the three proteins bind. In certain aspects, the method comprises identifying regions that are occupied by at least two or all three of: Oct4, Sox2 and Nanog. In certain aspects, the method comprises identifying regions that are occupied by RNA polymerase II or one of the components of the RNA polymerase holoenzyme, or by Polycomb Repression Complex 2 (PRC2), or one of its components. In one embodiment, the component is SUZ12.

In one embodiment, the stem cell is an embryonic stem cell. In one embodiment, the embryonic stem cell is a human embryonic stem cell, such as human embryonic stem cell H9. In one embodiment, the cell maintains the potential to differentiate into at least one of endoderm, mesoderm, and ectoderm tissues. In one embodiment, the cell maintains the potential to differentiate into endoderm, mesoderm, and ectoderm tissues. In another embodiment, the cell maintains the potential to differentiate into a neuronal cell type, such as a neuronal cell type is selected from motor neurons, sensory neurons, dopaminergic neurons, cholinergic neurons, interneurons, serotonergic neurons, peptidergic neurons, astrocytes, and oligodendrocytes. In one embodiment, the stem cell is an adult stem cell, such as an adult stem cell selected from mesenchymal stem cells, neural stem cells, neural crest stem cells, hematopoietic stem cells, and pancreatic stem cells. In one embodiment, the stem cell is isolated from an embryo fertilized in vitro. In one embodiment, the stem cell is isolated from bone marrow or from umbilical cord blood.

The invention also provides a method for identifying an agent or condition that promotes the differentiation of a stem cell to a differentiated cell type, or for identifying loss of stem cell potential (even irrespective of a cell type to differentiate into), comprising (a) contacting a stem cell with the agent; (b) identifying regions of the genome of the agent-exposed stem cell in (b) to which a protein binds according to the methods described herein, and comparing the regions to regions of the genome in a reference stem cell to which the protein binds, wherein the genome occupancy of the protein identifies the differentiation of the stem cell to a differentiated cell type when it differs from the genome occupancy for the reference stem cell and the agent or condition is identified as an agent or condition which promotes the differentiation of the stem cell. One aspect of the invention provides a method for identifying an agent that promotes the differentiation of a stem cell, comprising (a) providing a culture comprising the stem cell; (b) contacting said culture with the agent; (c) identifying regions of the genome of the stem cell to which a protein binds according to the method of claim 1; and (d) comparing the regions identified in (c) to one or more reference sets of bound regions indicative of a differentiating cell, wherein a match between the regions identified in (c) and one of the reference sets of bound regions indicates that the agent promotes the differentiation of the stem cell. The method may further comprise identifying an agent that promotes differentiation to a particular cell type (e.g., such as a neuronal cell type, ectodermal cell type, mesodermal cell type or further differentiated cell type) by comparing the regions of the genome of the agent-exposed stem cell to which the protein binds to regions of the genome of a reference differentiated cell to which the protein binds. In certain aspects, the condition includes conditions under which the stem cell is cultured and/or stored. In certain aspects, the binding patterns of a plurality of proteins is determined.

The invention further provides a method for identifying an agent or condition that promotes the differentiation of a stem cell to a particular differentiated cell type, comprising (a) contacting the stem cell with the agent; (b) identifying regions of the genome of the stem cell to which a protein binds, according to the methods described herein; (c) comparing the regions identified in (b) to one or more reference set of bound regions representative of a particular differentiated cell type to determine if they match, wherein a match in (c) indicates that the agent promotes differentiation of the stem cell to the particular differentiated cell type.

The invention also provides a method for identifying an agent that promotes the differentiation of a stem cell towards a given lineage, comprising (a) providing a culture comprising the stem cell; (b) contacting said culture with the agent; (c) identifying regions of the genome of the stem cell to which a protein binds according to the method of claim 1; and (d) comparing the regions identified in (c) to one or more reference sets of bound regions indicative of cell differentiating towards the given lineage, wherein a match between the regions identified in (c) and one of the reference sets of bound regions indicates that the agent promotes the differentiation of the stem cell towards the given lineage. In certain embodiments, the protein is Oct4, Sox2 or Nanog. In certain embodiments, the protein is RNA polymerase II or SUZ12 or a PRC1 or PRC2 subunit.

In certain aspects, the binding pattern of a plurality of proteins is determined wherein the plurality includes at least one, two or three of Oct4, Sox2, Nanog, or a PRC1 or PRC2 subunit. In certain embodiments, the agent is selected from nucleic acids, peptides, polypeptides, small organic molecules, aptamers, antibodies, affibodies, ribozymes, antisense oligonucleotides, and RNAi constructs.

In one aspect, a target gene “associated with” a target region is within less than about 20 kb of the transcriptional start site, within less than about 10 kb, or within less than about 8 kb. In another aspect, deletion or mutation of the target region alters transcription levels of the target gene. In certain aspects, a target region may include nontranslated regions within a transcribed region of a gene. In some embodiments, the method further includes determining the differentiation state of the cell. For example, in one aspect, the method comprises validating that a cell which has been cultured or stored comprises the same target regions bound by the protein as a reference cell (e.g., such as a reference stem cell) thereby determining whether the cell being cultured or stored comprises the same characteristics (e.g., stem cell potential) as the reference cell. In one embodiment, the target gene encodes (a) a transcriptional regulator; (b) a DNA binding protein; (c) a member of a chromatin remodeling complex; (d) a DNA replication or repair protein, or (e) a combination thereof. In one embodiment, the first protein, the second protein, or both, are transcriptional regulators. In one embodiment, the target gene encodes a member of the Polycomb Repressive Complex 1 or 2.

The invention further provides a method for identifying regions of a genome to which a first protein and a second protein bind. One such method comprises the steps of: (i) identifying the regions in the genome to which the first protein binds; (ii) identifying the regions in the genome to which the second protein binds; (iii) comparing the regions identified in steps (i) and (ii) to identify regions of the genome to which both proteins bind, wherein the steps (i) and (ii) are performed according to the methods described herein.

In one embodiment, step (ii) comprises identifying the regions in the genome to which the first protein binds in a stem cell. In one embodiment, the stem cell is an embryonic stem cell. In certain embodiments, the stem cell is derived from mice, rats, rabbits, cows, pigs, humans, or non-human primates. In one embodiment, the stem cell maintains the potential to differentiate into endoderm, mesoderm, and ectoderm tissues. In one embodiment, step (i) comprises producing a mixture comprising DNA fragments from a stem cell to which the first protein is bound in a cell which does not express the second protein. In another embodiment, step (ii) comprises producing a mixture comprising DNA fragments to which the second protein is bound in a stem cell which does not express the first protein. In another embodiment, step (i) comprises producing a mixture comprising DNA fragments to which the first protein is bound in a cell which expresses the second protein; and (ii) comprises producing a mixture comprising DNA fragments to which the second protein is bound in a cell which expresses the first protein.

The invention further provides a method of obtaining a set of nucleotide array probes tiling the promoter region of a gene, the method comprising: (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected: (a) have one or more desirable hybridization properties; and (b) tile the promoter region with an average density of about from 100 to about 1000 bp.

In another embodiment, the transcriptional start site for the gene is selected from a group of transcriptional start sites, each resulting in a transcript, wherein the start site selected results in the longest transcript. In another embodiment, the window of genomic DNA sequence spans from about 8 kb upstream of the start site to about 2 kb downstream. In another embodiment, each probe in the subset has a length of between 50 and 70 nucleotides. In another embodiment, the desirable properties for the array probes are selected from (a) a GC nucleotide content of between 30 and 100 percent; (b) low self-binding; (c) low complexity; and (d) high uniqueness. In another embodiment, the array probes in the subgroup tile the promoter region with a density of about from 200 bp to about 300 bp. In another embodiment, the method comprises the step of selecting additional preliminary probes for the set of nucleotide array probes, wherein the additional preliminary probes: (a) have less desirable hybridization properties than the preliminary probes selected in step (iv); and (b) are flanked by two nucleotide array probes, wherein the distance between the two flanking probes along the genome is greater than the average distance between adjacent nucleotide array probes of step (iv).

In another embodiment, the distance along the genome between the two flanking probes along the genome is greater than the average distance between adjacent nucleotide array probes of step (iv) by at least 100 bp. In another embodiment, the method comprises (v) synthesizing a plurality of nucleic acid probes, each probe having the nucleotide sequence of a nucleotide array probe. In another embodiment, the method comprises (vi) immobilizing the plurality of probes on a glass surface. In another embodiment, the gene is a mammalian gene. In one embodiment, the gene is a human gene. In one embodiment, the method further comprises synthesizing at least one of the nucleotide array probes and optionally immobilizing it on a microarray.

The invention further provides a method of obtaining a set of nucleotide array probes tiling the promoter region of a set of genes from a genome or a portion thereof, the method comprising identifying, for each gene, a set of nucleotide array probes tiling the promoter region of the gene according to the methods provided herein.

The invention further provides a computer readable medium having computer readable program codes embodied therein for obtaining a set of nucleotide array probes tiling the promoter region of a gene, the computer readable medium program codes performing functions comprising: (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected (a) have one or more desirable hybridization properties; and (b) tile the promoter region with an average density of about from 100 to about 1000 bp.

The invention further provides a system for obtaining a set of nucleotide array probes tiling the promoter region of a gene, the system comprising: a computer; a computer readable medium, operatively coupled to the computer, the computer readable medium program codes performing functions comprising: (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected (a) have one or more desirable hybridization properties; and (b) tile the promoter region with an average density of about from 100 to about 1000 bp.

### BRIEF DESCRIPTION OF THE DRAWINGS

FIGS. 1A-1B show genome-wide ChIP-Chip in human embryonic stem cells. (A) DNA segments bound by transcriptional regulators were identified using chromatin-immunoprecipitation (ChIP) and identified with DNA microarrays containing 60-mer oligonucleotide probes covering the region from −8 kb to +2 kb for 17,917 annotated transcription start sites for human genes. ES cell growth and quality control, ChIP protocol, DNA microarray probe design and data analysis methods are described in detail in Experimental Procedures. (B) Examples of Oct4 bound regions. Plots display unprocessed ChIP-enrichment ratios for all probes within a genomic region. Genes are shown to scale below plots (exons and introns are represented by thick vertical and horizontal lines, respectively), and the genomic region represented is indicated beneath the plot. The transcription start site and transcript direction are denoted by arrows.

FIGS. 2A-2C show Oct4, Sox2 and Nanog target genes in human ES cells. (A) Venn diagram representing the overlap of Oct4, Sox2, and Nanog promoter bound regions. (B) Representative examples of protein-coding genes co-occupied by Oct4, Sox21 and Nanog. Plots display unprocessed ChIP enrichment ratios for all probes within a genomic region. Genes are shown to scale relative to their chromosomal position. Exons and introns are represented by thick vertical and horizontal lines, respectively. The start and direction of transcription are denoted by arrows. Green, red, and purple lines represent Nanog, Sox2, and Oct4 bound regions, respectively. (C) Oct4, Sox2, and Nanog bind in close proximity. The distances between the midpoint of bound regions for pairs of transcription factors was calculated for the 353 regions bound by all three transcription factors. Negative and positive values indicate whether the first factor is upstream or downstream of the second factor in relation to the gene. The frequency of different distances between the bound regions is plotted as a histogram.

FIGS. 3A-3B show expression of Oct4, Sox2, and Nanog co-occupied genes. (A) Affymetrix expression data for ES cells was compared to a compendium of expression data from 158 experiments representing 79 other differentiated tissues and cell types. Ratios were generated by comparing gene expression in ES cells to the median level of gene expression across all datasets for each individual gene. Genes were ordered by relative expression in ES cells and the results were clustered by expression experiment using hierarchical clustering. Each gene is represented as a separate row and individual expression experiments are in separate columns. Red indicates higher expression in ES cells relative to differentiated cells. Green indicates lower expression in ES cells relative to differentiated cells. Examples of bound genes that are at the top and bottom of the rank order list are shown. (B) Relative levels of gene expression in H9 ES cells compared to differentiated cells were generated and converted to log 2 ratios. The distribution of these fold changes was calculated to derive a profile for different sets of genes. Data are shown for the distribution of expression changes between H9 ES cells and differentiated tissues for transcription factor genes that are not occupied by Oct4, Sox2 or Nanog (solid black line) and transcription factor genes occupied by all three (dotted line). The change in relative expression is indicated on the x axis and the numbers of genes in each bin are indicated on the y axes (left axis for unoccupied genes, right axis for occupied genes). The shift in distribution of expression changes for genes occupied by Oct4, Sox2 and Nanog is significant (p-value <0.001 using a two-sampled Kolmogorov-Smirnov test), consistent with the model that Oct4, Sox2 and Nanog are contributing to the regulation of these genes.

FIGS. 4A-4B show transcriptional regulatory motifs in human ES cells. (A) Shown here is an example of feed-forward transcriptional regulatory circuitry in human ES cells. Regulators are represented by blue circles; gene promoters are represented by red rectangles. Binding of a regulator to a promoter is indicated by a solid arrow. Genes encoding regulators are linked to their respective regulators by dashed arrows. (B) The interconnected autoregulatory loop formed by Oct4, Sox2, and Nanog.

FIG. 5 shows core transcriptional regulatory network in human ES cells. A model for the core transcriptional regulatory network was constructed by identifying Oct4, Sox2, and Nanog target genes that encode transcription factors and chromatin regulators, and integrating knowledge of the functions of these downstream regulators based on comparison to multiple expression datasets and to the literature. A subset of active and inactive genes co-occupied by the three factors in human ES cells is shown here. Regulators are represented by blue circles; gene promoters are represented by red rectangles; grey boxes represent putative downstream target genes. Positive regulation was assumed if the target gene was expressed whereas negative regulation was assumed if the target gene was not transcribed.

FIGS. 6A-6B show the Distribution of transcription factor binding sites and transcriptional regulatory elements relative to transcription start sites. (A) Distribution of transcription factor binding sites from TRANSFAC from −8 kb to +3 kb around the transcription start site. (B) Distribution of functional regulatory elements from the TRRD (database of transcriptional regulatory regions, http://www.bionet.nsc.ru/trrd/34/) from −8 kb to +3 kb around the transcription start site.

FIG. 7 shows Oct4, Sox2, and Nanog Co-occupy Each of Their Promoters. Plots display unprocessed ChIP enrichment ratios for all probes within a genomic region. Genes are shown to scale relative to their chromosomal position. Exons and introns are represented by thick vertical and horizontal lines, respectively. The start and direction of transcription are denoted by arrows. Green, red, and purple lines represent Nanog, Sox2, and Oct4 bound regions, respectively.

FIG. 8 shows Immunohistochemical Analysis of Pluripotency Markers. Human ES cells were analyzed by immunohistochemistry for the characteristic pluripotency markers Oct4 and SSEA-3. For reference, nuclei were stained with DAPI. Our analysis indicated that >>80% of the colonies were positive for Oct4 and SSEA-3. Alkaline phosphatase activity was also strongly detected in hES cells.

FIG. 9 shows that H9 Cells Maintain Differentiation Potential in Teratoma Assay. Teratomas were analyzed for the presence of markers for ectoderm (Tuj1), mesoderm (MF20) and endoderm (AFP) For reference, nuclei are stained with DAPI. Antibody reactivity was detected for derivatives of all three germ layers confirming that the human embryonic stem cells used in our analysis have maintained differentiation potential.

FIGS. 10A-10B show Control Chromatin Immunoprecipitations. (A) Oct4, Sox2, and Nanog targets were not enriched using preimmune sera in human ES cells. ChIP was carried out using rabbit or goat IgG to assess antibody specificity. Labeled IP material and control DNA were hybridized to self-printed promoter arrays. Background subtracted normalized log 2 intensities are plotted. Red lines represent enrichment/exclusion p-values of <10-3. Example shown is for the goat IgG control experiment. (B) Potential antibody cross-reactivity with other family members was assessed by performing ChIP experiments in HepG2 cells. Data were analyzed as above. Example shown for Oct4 (sc-9081) in HepG2.

FIG. 11 shows Oct4 and Sox2 Binding to UTF1 and FGF4. Plots display unprocessed ChIP enrichment ratios for all probes within a genomic region. Genes are shown to scale relative to their chromosomal position. Exons and introns are represented by thick vertical and horizontal lines, respectively. The start and direction of transcription are denoted by arrows. Green, red, and purple lines represent Nanog, Sox2, and Oct4 bound regions, respectively.

FIG. 12 shows the Distribution of Oct4, Sox2, and Nanog Bound Regions Relative to Transcription Start Sites. Histogram of the distance between transcription factor bound regions and the nearest transcription start site. Green, red, and purple lines represent Nanog, Sox2, and Oct4 bound regions, respectively. A distance of 0 refers to bound regions that overlap a transcription start site.

FIGS. 13A-13E show genome-wide ChIP-Chip in human embryonic stem cells. (A) DNA segments bound by the initiation form of RNA polymerase II or Suz12 were isolated using chromatin-immunoprecipitation (ChIP) and identified with DNA microarrays containing over 4.6 million unique 60-mer oligonucleotide probes spanning the entire non-repeat portion of the human genome. (B) Examples of RNA polymerase II ChIP signals from genome-wide ChIP-Chip. The plots show unprocessed enrichment ratios (blue) for all probes within a genomic region (ChIP vs. whole genomic DNA). Chromosomal positions are from NCBI build 35 of the human genome. Genes are shown to scale below plots (exons are represented by vertical bars). The start and direction of transcription are noted by arrows. (C) Examples of Suz12 ChIP signals from genome-wide ChIP-Chip. The plots show unprocessed enrichment ratios (green) for all probes within a genomic region (ChIP vs. whole genomic DNA). Chromosomal positions, genes and notations are as described in b. (D) Chart showing percentage of all annotated genes bound by RNA polymerase II (blue), Suz12 (green), both (yellow) or neither (grey). (E) Distribution of the distance between bound probes and the closest transcription start sites from RefSeq, Ensembl, MGC, UCSC Known Genes and H-Inv databases for Suz12 (green line) and RNA polymerase II (blue line). The number of bound probes is given as the percentage of total probes and is calculated for 400 bp intervals from the start site. The null-distribution of the distance between all probes and the closest transcription is shown as a black line.

FIGS. 14A-14C show Suz12 is associated with Eed, histone H3K27me3 modification and transcriptional repression in ES cells. (A) Venn diagram showing the overlap of genes bound by Suz12 at high-confidence, genes bound by Eed at high-confidence and genes trimethylated at H3K27 at high-confidence. The data are from promoter microarrays that contain probes tiling −8 kb and +2 kb around transcription start. 72% of the genes bound by Suz12 at high-confidence are also bound by Eed at high-confidence; others are bound by Eed at lower confidence. (B) Suz12 (top), Eed (middle) and H3K27me3 (bottom) occupancy at NeuroD1. The plots show unprocessed enrichment ratios for all probes within this genomic region (Suz12 ChIP vs. whole genomic DNA, Eed ChIP vs whole genomic DNA and H3K27me3 ChIP vs. total H3 ChIP). Chromosomal positions are from NCBI build 35 of the human genome. NeuroD1 is shown to scale below plots (exons are represented by vertical bars). The start and direction of transcription are noted by arrows. (C) Relative expression levels of 604 genes occupied by PRC2 and trimethylated at H3K27 in ES cells. Comparisons were made across 4 ES cell lines and 79 differentiated cell types. Each row corresponds to a single gene that is bound by Suz12, associated with Eed and H3K27me3 and for which Affymetrix expression data is available. Each column corresponds to a single expression microarray. ES cells are in the following order; H1, H9, HSF6, HSF1. For each gene, expression is shown relative to the average expression level of that gene across all samples, with shades of red indicating higher than average expression and green lower than average expression according to the scale on the right. Cell types are grouped by tissue or organ function and genes are ranked according the significance of their relative level of gene expression in ES cells.

FIGS. 15-15B show cellular functions of genes occupied by Suz12. (A) Genes bound by Suz12 or RNA polymerase II were compared to biological process gene ontology categories; highly represented categories are shown. Ontology terms are shown on the y-axis; p-values for the significance of enrichment are graphed along the x-axis (Suz12 in green, RNA polymerase II in blue). (B) Selected examples of developmental transcription factor families bound by Suz12. Suz12 is represented by the green oval; individual transcription factors are represented by circles and grouped by family as indicated. Examples of transcription factors with defined roles in development are labeled. Transcription factor families include homeobox protein (HOX), basic helix-loop-helix domain containing, class B (BHLHB), Hox co-factors (MEIS/EVX), distal-less homeobox (DLX), Forkhead box (FOX), NEUROD, GATA binding protein (GATA), runt related transcription factor (RUNX), paired box and paired-like (PAX), LIM homeobox (LHX), sine oculis homeobox homolog (SIX), NK transcription factor related (NKX), SRY box (SOX), POU domain containing, classes 3 and 4 (POU), early B-cell factor (EBF), atonal homolog (ATOH), hairy and enhancer of split protein (HES), myogenic basic domain (MYO), T-box (TBX), caudal type homeobox (CDX), and iroquois homeobox protein (IRX).

FIGS. 16A-16C show Suz12 occupies large portions of genes encoding transcription factors with roles in development. (A) The fraction of Suz12 target genes associated with different sizes of binding domains. Genes are grouped into four categories according to their function: Signaling, Adhesion/migration, Transcription and Other. (B) Examples of Suz12 (green) and RNA polymerase II (blue) binding at the genes encoding developmental regulators TBX5 and PAX6. The plots show unprocessed enrichment ratios for all probes within a genomic region (ChIP vs. whole genomic DNA). Genes are shown to scale below plots (exons are represented by vertical bars). The start and direction of transcription are noted by arrows. (C) Binding profiles of Suz12 (green) and RNA polymerase II (blue) across ˜500 kb regions encompassing Hox clusters A-D. Unprocessed enrichment ratios for all probes within a genomic region are shown (ChIP vs. whole genomic DNA). Approximate Hox cluster region sizes are indicated within black bars.

FIGS. 17A-17B show Suz12 binding is associated with highly conserved regions. (A) Suz12 occupancy (green) and conserved elements are shown at NKX2-2 and adjacent genomic regions. The plots show unprocessed enrichment ratios for all probes within this genomic region (Suz12 ChIP vs. whole genomic DNA). Conserved elements (red) with LoD scores >160 derived from the PhastCons program (Siepel et al., 2005) are shown to scale above the plot. Genes are shown to scale below plots (exons are represented by vertical bars). A higher resolution view is also shown below. (B) Enrichment of conserved non-coding elements within Suz12 (green) and RNA polymerase II (blue) bound regions. The maximum non-exonic PhastCons conservation score was determined for each bound region. For comparison, the same parameter was determined using a randomized set of genomic regions with the same size distribution. The graph displays the ratio of the number of bound regions with that score versus the number of randomized genomic regions with that score.

FIGS. 18A-18F show referential activation of PRC2 target genes during ES cell differentiation. (A) Fold enrichment in the number of genes induced or repressed during ES cell differentiation. The change in gene expression is given as the log(2) transformed ratio of the signals in differentiated H1 cells versus pluripotent H1 cells and is binned into 6 groups. The upper limit of each bin is indicated on the x-axis. The two lines show genes transcriptionally inactive in ES cells (absence of RNA polymerase II) and bound by Suz12 (green) and genes transcriptionally inactive in ES cells and repressed by other means (blue). In both cases, fold enrichment is calculated against the total population of genes and normalized for the number of genes present in each group. (B) Expression changes of genes encoding developmental regulators during ES cell differentiation. Expression ratio (differentiated/pluripotent) is represented by color, with shades of red indicating upregulation and shades of green downregulation according to the scale shown above. Genes are ordered according to change in gene expression, with genes exhibiting higher expression in pluripotent ES cells to the left and genes exhibiting higher expression in differentiated cells to the right. Genes bound by Suz12 in undifferentiated ES cells are indicated by blue lines in the lower panel. (C) Fold enrichment in the number of genes induced or repressed in Suz12-deficient mouse cells. The change in gene expression is given as the log(2) transformed ratio of the signals in Suz12-deficient cells versus wild-type ES cells. The two lines show genes transcriptionally inactive in human ES cells (absence of RNA polymerase II) and bound by Suz12 (green) and genes transcriptionally inactive in human ES cells and repressed by other means (blue). In both cases, fold enrichment is calculated against the total population of genes. (D) Gene expression ratios (log base 2) of Suz12 target genes in differentiated human H1 ES cells relative to pluripotent H1 ES cells (x-axis) and in Suz12-deficient mouse cells relative to wild-type mouse ES cells (y-axis). Upper right quadrant: genes upregulated during human ES cell differentiation and in Suz12-deficient mouse cells; lower right: genes upregulated during ES cell differentiation and downregulated in Suz12-deficient cells; lower left: genes downregulated during ES cell differentiation and in Suz12-deficient cells; upper left: genes downregulated during ES cell differentiation and upregulated in Suz12-deficient cells. (E) Suz12 binding profiles across the gene encoding muscle regulator MYOD1 in H9 human ES cells (green) and primary human skeletal myotubes (grey). The plots show unprocessed enrichment ratios for all probes within a genomic region (ChIP vs. whole genomic DNA). Genes are shown to scale below plots (exons are represented by vertical bars). The start and direction of transcription are noted by arrows. (F) Suz12 binding profiles across the gene encoding LHX9 in H9 human ES cells (green) and primary human skeletal myotubes (grey). The plots show unprocessed enrichment ratios for all probes within a genomic region (ChIP vs. whole genomic DNA). Genes are shown to scale below plots (exons are represented by vertical bars). The start and direction of transcription are noted by arrows.

FIGS. 19A-19B show Suz12 is localized to genes also bound by ES cell transcriptional regulators. (A) Transcriptional regulatory network model of developmental regulators governed by Oct4, Sox2, Nanog, RNA polymerase II and Suz12 in human ES cells. The ES cell transcription factors each bound to approximately one-third of the PRC2-occupied, developmental genes. Developmental regulators were selected based on gene ontology. Regulators are represented by dark blue circles; RNA polymerase II is represented by a light blue circles; Suz12 is represented by a green circle; gene promoters for developmental regulators are represented by small red circles. (B) Suz12 occupies a set of repressed developmental regulators also bound by Oct4, Sox2 and Nanog in human ES cells. Genes annotated as bound by Oct4, Sox2 and Nanog previously and identified as active or repressed based on expression data (Boyer et al., 2005) were tested to see if they were bound by Suz12 or RNA polymerase II. Ten of eleven previously identified active genes were found to be bound by RNA polymerase II at known promoters while eleven of twelve previously identified repressed genes were bound by Suz12. Regulators are represented by dark blue circles; RNA polymerase II by a light blue circle and Suz12 by a green circle. Gene promoters are represented by red rectangles.

FIG. 20 shows a functional block diagram of general purpose computer system 2000 for performing the functions of the software provided by the invention.

### DETAILED DESCRIPTION OF THE INVENTION

### I. Overview

The invention provides, in part, methods of identifying regions of a genome to which a protein of interest binds in a cell, such as a stem cell or a progenitor cell. One aspect of the invention provides a method of identifying regions of a genome to which a protein of interest binds in a stem cell, or in a cell suspected of being a stem cell. Another aspect of the invention provides methods of identifying the differentiation potential of a test, such as a stem cell, by determining the regions of the genome of the test cell to which a protein binds and comparing it to the genome occupancy of the protein in stem cells having know differentiation potentials. In certain aspects, the method comprises determining regions where Oct4, Sox2, Nanog, RNA polymerase II or one of its subunits, or where PRC2, or one of its components, binds on a genome.

Another aspect of the invention provides a method for identifying an agent or condition that promotes the differentiation of a stem cell or that maintains a stem cell in an undifferentiated state. A related aspect provides methods of identifying an agent or condition that promotes the differentiation of a stem cell to a particular differentiated cell type. In preferred embodiments, these methods include ChIP-CHIP analysis of DNA binding proteins, such as Oct4, Sox2, Suz12 or Nanog.

Another aspect of the invention provides a method of identifying a transcriptional feedforward regulatory loops between transcription factors in a cell, preferably a stem cell. Another aspect provides methods for identifying regions of a genome to which a first protein and a second protein bind.

The invention further provides methods of obtaining a set of nucleotide array probes tiling the promoter region of a gene, which may be useful for the automated generation of probes to spot on an array. Probes include probes that tile the regulatory regions of genes where DNA-binding proteins are expected to bind. Related aspects provide a method of obtaining a set of nucleotide array probes tiling the promoter region of a set of genes from a genome or a portion thereof.

Another aspect of the invention provides a computer readable medium having computer readable program codes embodied therein, and related computer systems, for obtaining a set of nucleotide array probes tiling the promoter region of a gene, as well as systems for obtaining a set of nucleotide array probes tiling the promoter region of a gene.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

“Recombinant” when used with-reference, e.g., to a nucleic acid, cell, virus, plasmid, vector, or the like, indicates that these have been modified by the introduction of an exogenous, non-native nucleic acid or the alteration of a native nucleic acid, or have been derived from a recombinant nucleic acid, cell, virus, plasmid, or vector. Recombinant protein refers to a protein derived from a recombinant nucleic acid, virus, plasmid, vector, or the like.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “microarray” refers to an array of distinct polynucleotides or oligonucleotides synthesized on a substrate, such as paper, nylon or other type of membrane, filter, chip, glass slide, or any other suitable solid support.

A probe that is “labeled” is detectable, either directly or indirectly, by spectroscopic, photochemical, biochemical, immunochemical, isotopic, or chemical means. For example, useful labels include ³²P, ³³P, ³⁵S, ¹⁴C, ³H, ¹²⁵I, stable isotopes, fluorescent dyes and fluorettes (Rozinov and Nolan (1998) Chem. Biol 5:713-728; Molecular Probes, Inc. (2003) Catalogue, Molecular Probes, Eugene Oreg.), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs radiant energy of one wavelength and emits radiant energy of a second, longer wavelength.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe. The probes are preferably directly labeled as with isotopes, chromophores, fluorophores, chromogens, or indirectly labeled such as with biotin to which a streptavidin complex or avidin complex can later bind.

A “nucleic acid probe” is a nucleic acid capable of binding to a target nucleic acid of complementary sequence, usually through complementary base pairing, e.g., through hydrogen bond formation. A probe may include natural, e.g., A, G, C, or T, or modified bases, e.g., 7-deazaguanosine, inosine, etc. The bases in a probe can be joined by a linkage other than a phosphodiester bond. Probes can be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

“Polymerase chain reaction” (PCR) refers, e.g., to a procedure or product where a specific region or segment of a nucleic acid is amplified, and where the segment is bracketed by primers used by RNA polymerase (Bernard and Wittwer (2002). Clin. Chem. 48: 1178-1185; Joyce (2002) Methods Mol. Biol. 193:83-92; Ong and Irvine (2002) Hematol. 7:59-67).

A “promoter” is a nucleic acid sequence that directs transcription of a nucleic acid. A promoter includes nucleic acid sequences near the start site of transcription, e.g., a TATA box, see, e.g., Butler and Kadonaga (2002) Genes Dev. 16:2583-2592; Georgel (2002) Biochem. Cell Biol. 80:295-300. A promoter also optionally includes distal enhancer or repressor elements, which can be located as much as several thousand base pairs on either side from the start site of transcription. A “constitutive” promoter is a promoter that is active under most environmental and developmental conditions, while an “inducible”, promoter is a promoter is active or activated under, e.g., specific environmental or developmental conditions.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics; and antibody mimetics. As a therapeutic, a small molecule may be more permeable to cells, less susceptible to degradation, and less apt to elicit an immune response than large molecules. Small molecule toxins are described, see, e.g., U.S. Pat. No. 6,326,482 issued to Stewart, et al.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins) or polysaccharides (starches, or polysugars), as well as other chemical entities that contain repeating units of like chemical structure.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “oligonucleotide” as used herein denotes single stranded nucleotide multimers of from about 10 to 100 nucleotides and up to 200 nucleotides in length.

The term “functionalization” as used herein relates to modification of a solid substrate to provide a plurality of functional groups on the substrate surface. By a “functionalized surface” is meant a substrate surface that has been modified so that a plurality of functional groups are present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Sensitivity is a term used to refer to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

“Differentiation” in the present context means the formation of cells expressing markers known to be associated with cells that are more specialized and closer to becoming terminally differentiated cells incapable of further division or differentiation. The pathway along which cells progress from a less committed cell, to a cell that is increasingly committed to a particular cell type, and eventually to a terminally differentiated cell is referred to as progressive differentiation or progressive commitment. [0120] Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that the term “differentiation” or “differentiated” refers to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the invention contemplates the use of stem cells isolated from any tissue source.

The term “tissue” refers to a group or layer of similarly specialized cells which together perform certain special functions

As used herein, “neuronal cell” or “cell of the nervous system” include both neurons and glial cells.

### III. Methods of Identifying Chromosome Regions

One aspect of the invention provides methods for identifying a region of a genome of a cell to which a protein of interest binds. One aspect provides a method of identifying the regions of nuclear DNA to which a DNA-binding protein is bound in a cell. One specific aspect of the invention provides a method for identifying at least one region of a genome to which a protein of interest binds, the method comprising the steps of: producing a mixture comprising DNA fragments to which the protein of interest is bound; (b) isolating one or more DNA fragments to which the protein of interest is bound from the mixture produced in step (a); and (c) identifying regions of the genome which are complementary to the DNA fragments isolated in step (b), thereby identifying at least one (one or more) region of the genome to which the protein of interest is bound. In some embodiments, the method further comprises generating a probe from the one or more of the isolated DNA fragments, such as between steps (b) and (c). In one embodiment, the probe comprises a nucleic acid, which may comprise a detectable label. In a preferred embodiment, the cell is a stem cell.

In one embodiment of the methods described herein, the protein of interest is covalently crosslinked to the genomic DNA prior to fragmenting the genomic DNA. There are a variety of methods which can be used to link a DNA-binding to genomic DNA. In one embodiment of the methods described herein, the crosslinking is formaldehyde crosslinking (Solomon, M. J. and Varshavsky, A., Proc. Natl. Sci. USA 82:6470-6474; Orlando, V., TIBS, 25:99-104). UV light may also be used (Pashev et al. Trends Biochem Sci. 1991; 16(9):323-6; Zhang L et al. Biochem Biophys Res Commun. 2004; 322(3):705-11).

In one embodiment of the methods described herein where the protein of interest is covalently crosslinked to the genomic DNA prior to fragmenting the genomic DNA of the cell, separating the DNA fragment from the protein of interest comprises the step of reversing the crosslink. In a specific embodiment, it comprises the steps of (i) isolating a DNA fragment to which the protein of interest is bound from the mixture produced in (a); and (ii) separating (1) the DNA fragment from (2) the protein of interest. In a specific embodiment, separating the DNA fragment from the protein of interest to which it is bound comprises the steps of removing the crosslink between the DNA fragment and the protein of interest and removing the protein of interest from the DNA fragment. This may be accomplished, for example, by degrading the protein of interest. In one embodiment, a protease such as proteinase K is used to degrade the protein of interest.

Suitable non-limiting methods for purifying the DNA fragment include column chromatography (U.S. Pat. No. 5,707,812), the use of hydroxylated silica polymers (U.S. Pat. No. 5,693,785), rehydrated silica gel (U.S. Pat. No. 4,923,978), boronated silicates (U.S. Pat. No. 5,674,997), modified glass fiber membranes (U.S. Pat. Nos. 5,650,506; 5,438,127), fluorinated adsorbents (U.S. Pat. No. 5,625,054; U.S. Pat. No. 5,438,129), diatomaceous earth (U.S. Pat. No. 5,075,430), dialysis (U.S. Pat. No. 4,921,952), gel polymers (U.S. Pat. No. 5,106,966) and the use of chaotropic compounds with DNA-binding reagents (U.S. Pat. No. 5,234,809). Commercially available DNA isolation and purification kits are also available from several sources including Stratagene (CLEARCUT Miniprep Kit), and Life Technologies (GLASSMAX DNA Isolation Systems).

In some embodiments of the methods described herein, the genomic DNA is fragmented mechanically, such as by hydrodynamic shearing or sonication. Mechanical fragmentation can occur by any method known in the art, including shearing of DNA by passing it through the narrow capillary or orifice (Oefner et al., 1996, Nucleic Acids Res.; 24(20):3879-86; Thorstenson et al., 1998, Genome Res.; 8(8):848-55), sonicating the DNA, such as by ultrasound (Bankier, 1993, Methods Mol Biol.; 23:47-50, or grinding in cell homogenizers (Rodriguez L V. Arch Biochem Biophys. 1980; 200(1): 116-29). Mechanical fragmentation usually results in double strand breaks within the DNA molecule. Sonication may also be performed with a tip sonicator, such as a multi-tip sonicator, or more preferably using acoustic soundwaves. A Microplate Sonicator® (Misonix Inc.) may be used to partially fragment the DNA. Such a device is described in U.S. Patent Publication No. 2002/0068872. Another acoustic-based system that may be used to fragment DNA is described in U.S. Pat. No. 6,719,449, manufactured by Covaris Inc. U.S. Pat. No. 6,235,501 describes a mechanical method of producing high molecular weight DNA fragments by application of rapidly oscillating reciprocal mechanical energy to cells in the presence of a liquid medium in a closed container, which may be used to mechanically fragment the DNA.

Genomic sequences may be amplified prior to or after a fragmentation step. In one embodiment, an amplification step is used which does not substantially reduce the complexity of the initial source of nucleic acids, e.g., genomic DNA is obtained without a pre-selection step or genomic DNA which has been enriched by selecting for fragments which bind to a protein of interest, and amplification employs a random set of primers or primers whose complements occur at a desired frequency throughout the genome or whose complements are engineered to be included in a plurality (e.g., all) genomic fragments obtained from a sample (e.g., such as linkers ligated to the ends of genomic fragments).

However, in other embodiments, amplification can be performed which enriches for certain types of sequences, e.g., sequences which contains a consensus binding site for a protein of interest.

Methods for amplifying nucleic sequences can vary. In one aspect, nucleic acids are amplified using an isothermal amplification technique. In another aspect, nucleic acids are amplified using a strand displacement technique, such as multiple strand displacement.

In a further aspect, the nucleic acid is amplified using random primers, degenerate primers and/or primers which bind to a constant sequence ligated to ends of genomic fragments in a sample.

In certain aspects, amplified isolated DNA fragments are labeled, e.g., labeled probes are generated from the fragments by labeling an amplification product of the fragments using methods known in the art.

In a preferred embodiment, the chromatin fragments bound by the protein of interest (e.g. a transcriptional regulator or a histone) are isolated using chromatin immunoprecipitation (ChIP). Briefly, this technique involves the use of a specific antibody to immunoprecipitate chromatin complexes comprising the corresponding antigen i.e. the protein of interest, and examination of the nucleotide sequences present in the immunoprecipitate. Immunoprecipitation of a particular sequence by the antibody is indicative of interaction of the antigen with that sequence. See, for example, O'Neill et al., in Methods in Enzymology, Vol. 274, Academic Press, San Diego, 1999, pp. 189-197; Kuo et al. (1999) Method 19:425-433; and Ausubel et al., supra, Chapter 21. Accordingly, in one embodiment, the DNA fragment bound by the protein of interest is identified using an antibody which binds to the protein of interest.

In one embodiment, the chromatin immunoprecipitation technique is applied as follows in the context of a histone. Cells which express the histone are treated with an agent that crosslinks the histone to chromatin, such as with formaldehyde treatment or ultraviolet irradiation. Subsequent to crosslinking, cellular nucleic acid is isolated, fragmented and incubated in the presence of an antibody directed against the histone. Antibody-antigen complexes are precipitated, crosslinks are reversed (for example, formaldehyde-induced DNA-protein crosslinks can be reversed by heating) so that the sequence content of the immunoprecipitated DNA is tested for the presence of one or more specific sequences. The antibody may bind directly to an epitope on the histone or it may bind to an affinity tag on the histone, such as a myc tag recognized by an anti-Myc antibody (Santa Cruz Biotechnology, sc-764). A non-antibody agent with affinity for the transcriptional regulator, or for a tag fused to it, may be used in place of the antibody. For example, if the histone comprises a six-histidine tag, complexes may be isolated by affinity chromatography to nickel-containing sepharose. Additional variations on ChIP methods may be found in Kurdistani et al. Methods. 2003; 31(1):90-5; O'Neill et al. Methods. 2003, 31(1):76-82; Spencer et al., Methods. 2003; 31(1):67-75; and Orlando et al. Methods 11: 205-214 (1997).

In one embodiment of the methods described herein, DNA fragments from a control immunoprecipitation reaction are used in place of the isolated chromatin as a control. For example, an antibody that does not react with a histone being tested may be used in a chromatin IP procedure to isolate control chromatin, which can then be compared to the chromatin isolated using an antibody that binds to the histone. In preferred embodiments, the antibody that does not bind to the histone being tested also does not react with other histone or other DNA-binding proteins.

The identification of genomic regions from the isolated DNA fragments may be achieved by generating DNA or RNA probes from the fragment (such as by using the isolated DNA fragments as templates for DNA or RNA synthesis), and hybridizing them to a DNA microarray, such as a DNA microarray comprising immobilized nucleic acids complementary to regions of the genome. In one embodiment, the probes are labeled to facilitate their detection. The probes may be labeled during their synthesis, such as by synthesizing them in the presence of labeled nucleotides, or they may be labeled subsequent to their synthesis. In other embodiments, detection agents may be used to label the DNA/RNA probes once they have hybridized to a DNA microarray. Such detection agents include antibodies, antibody fragments, and dendrimers among others.

In one embodiment, labeled probes are generated by using the DNA fragments as templates for DNA or RNA synthesis by polymerases using techniques well known in the art, such as using the polymerase chain reaction. DNA synthesis may be primed using random primers. Random priming is described in U.S. Pat. Nos. 5,106,727 and 5,043,272. In some embodiments, the labeled probes are generated using ligation-mediated polymerase chain reaction (LM-PCR). LM-PCR is described, for example, in U.S. Application No. 2003/0143599. Other methods for DNA labeling include direct labeling, 77 RNA polymerase amplification, aminoallyl labeling and hapten-antibody enzymatic labeling. In one embodiment, the labeled probes comprise a fluorescent molecule, such as Cy3 or Cy5 dyes. In another embodiment, the labeled probes comprise semiconducting nanocrystals, also known as quantum dots. Quantum dots are described in U.S. Publication Nos. 2003/0087239 and 2002/0028457, and in international PCT publication No. WO01/61040.

Extension products that are produced as described above are typically labeled in the present methods. As such, the reagents employed in the subject primer extension reactions typically include a labeling reagent, where the labeling reagent may be the primer or a labeled nucleotide, which may be labeled with a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., dCTP. Fluorescent moieties which may be used to tag nucleotides for producing labeled nucleic acids include, but are not limited to: fluorescein, the cyanine dyes, such as Cy3, Cy5, Alexa 555, Bodipy 630/650, and the like. Other labels may also be employed as are known in the art.

When control probes are used, the control probes may be labeled with the same label or different labels as the experimental probes, depending on the actual assay protocol employed. For example, where each set of probes is to be contacted with different but identical arrays, each set of probes may carry the same label. Alternatively, where both sets are to be simultaneously contacted with a single array of immobilized oligonucleotide features, the sets may be differentially labeled.

In some embodiments, the nucleic acid probes are not labeled. For example, in certain embodiments, binding events on the surface of a substrate (such as an oligonucleotide microarray) may be detected by means other than by detection of a labeled nucleic acids, such as by change in conformation of a conformationally labeled immobilized oligonucleotide, detection of electrical signals caused by binding events on the substrate surface, etc.

In one embodiment, identifying a region of the genome of the cell which is complementary to the isolated DNA fragments comprises combining the probe(s) with one or more sets of distinct oligonucleotide features bound to a surface of a solid support under conditions such that nucleic acid hybridization to the surface immobilized features can occur, wherein the distinct oligonucleotide features are each complementary to a region of the genome, under conditions in which specific hybridization between the probe and the oligonucleotide features can occur, and detecting said hybridization, wherein hybridization between the probe and the oligonucleotide features relative to a suitable control indicates that the protein of interest is bound to the region of the genome to which the sequence of the oligonucleotide features is complementary. “Specific hybridization” refers to hybridization occurring under stringent conditions.

The experimental and control probes can be contacted to the surface immobilized features either simultaneously or serially. In many embodiments the compositions are contacted with the plurality of surface immobilized features, e.g., the array of distinct oligonucleotides of different sequence, simultaneously. Depending on how the collections or populations are labeled, the collections or populations may be contacted with the same array or different arrays, where, when the collections or populations are contacted with different arrays, the different arrays are substantially, if not completely, identical to each other in terms of feature content and organization.

An oligonucleotide bound to a surface of a solid support refers to an oligonucleotide or mimetic thereof, e.g., PNA or LNA molecule, that is immobilized on a surface of a solid substrate in a feature or spot, where the substrate can have a variety of configurations, e.g., a sheet, bead, or other structure. In certain embodiments, the collections of features of oligonucleotides employed herein are present on a surface of the same planar support, e.g., in the form of an array.

Arrays refer to an ordered array presented for binding to nucleic acids and the like, and include microarrays. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids. An “array,” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain. Exemplary arrays are described in U.S. Patent Pub No. 2004/0191813.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

The number of nucleic acid features of an array may vary, where the number of features present on the surface of the array may be at least 2, 5, or 10 or more such as at least 20 and including at least 50, where the number may be as high as about 100, as about 500, as about 1000, as about 5000, as about 10000 or higher. In representative embodiments, the subject arrays have a density ranging from about 100 to about 100,000 features/cm, such as from about 500 to about 20,000 features/cm², including from about 1000 to about 20,000 features/cm. In representative embodiments, the density of single-stranded nucleic acids within a given feature is selected to optimize efficiency of the RNA polymerase. In certain of these representative embodiments, the density of the single-stranded nucleic acids may range from about 10-3 to about 1 pmol/mm², such as from about 10-2 to about 0.1 pmol/mm², including from about 5×10⁻² to about 0.1 pmol/mm².

In certain aspects, even at high density (e.g., at least about 10,000 features/cm², at least about 50,000 features/cm², or at least about 100,000 features/cm², there are interfeature areas between the majority of features, substantially free of oligonucleotides.

Additionally, the sequence of nucleotides in a given feature may vary based on a particular synthesis reaction. For example, while the majority of oligonucleotides in a feature may be 60 mer, some may be less than 60 mer but otherwise comprise subsequences of the 60 mer sequence. However, in one aspect, at least about 75%, at least about 80%, at least about 90%, at least about 95% of the oligonucleotides of a feature comprise identical sequences (e.g., sequences of identical base composition and length).

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces.

In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

In one embodiment, an array is synthesized using a method as described in U.S. Ser. No. 10/813,467, the entirety of which is incorporated by reference herein.

In some embodiments, previously identified regions from a particular chromosomal region of interest are used as array elements. Such regions are becoming available as a result of rapid progress of the worldwide initiative in genomics. In certain embodiments, the array can include features made up of surface immobilized oligonucleotides which “tile” a particular region (which have been identified in a previous assay), by which is meant that the features correspond to region of interest as well as genomic sequences found at defined intervals on either side of the particular region of interest, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled arrays tailored to the initially identified region to further assay the region at a higher resolution, e.g., in an iterative protocol. Accordingly, the subject methods include at least two iterations, where the first iteration of the subject methods identifies a region of interest, and the one or more subsequent iterations assay the region with sets of tiled surface immobilized features, e.g., of increasing or alternate resolution.

In some embodiments, adjacent tiled oligonucleotide features may be spaced at about at least 10 bp, 25 bp, 50 bp, 100 bp, 150 bp, 200 bp, 300 bp, 500 bp, 750 bp, 1 kb, 2 kb, 3 kb, 4 kb, 5 kb, 6 kb, 7 kb, 8 kb, 9 kb, 10 kb or 20 kb relative to their positions in the genome. In other embodiments, adjacent tiled oligonucleotide features may be spaced at about at most 10 bp, 25 bp, 50 bp, 100 bp, 150 bp, 200 bp, 300 bp, 500 bp, 750 bp, 1 kb, 2 kb, 3 kb, 4 kb, 5 kb, 6 kb, 7 kb, 8 kb, 9 kb, 10 kb or 20 kb relative to their positions in the genome.

In one embodiment, the oligonucleotide features comprise a nucleic acid having a length ranging from about 10 to about 200 nt including from about 10 or about 20 nt to about 100 nt, where in many embodiments the immobilized nucleic acids range in length from about 50 to about 90 nt or about 50 to about 80 nt, such as from about 50 to about 70 nt. In a preferred embodiment, the nucleic acid has a length of about 60 nucleotides.

In one embodiment, the oligonucleotide features bound to a surface of a solid support includes sequences representative of locations distributed across at least a portion of a genome. In one embodiment, the oligonucleotide features have target complements spaced (uniformly or non-uniformly) throughout the genome. In one aspect, a probe set comprises probe sequences representing 47 different loci, one on each p and q arm of the 23 human chromosomes plus one locus on the Y-chromosome. In another aspect, the probe set comprises probe sequences which include repetitive sequences (e.g., such as Alu sequences, centromeric sequences, telomere sequences, LINE sequences, SINE sequences and the like). In one embodiment, the oligonucleotide features bound to a surface of a solid support samples the portion of the genome at least about every 20, 10, 5, 4, 3, 2, 1, or 0.5 kb. In one embodiment, the portion of the genome comprises at regulatory regions for at least 20%, 30%, 40%, 50%, 60%, 70%, 80%, 90%, 95% of the total genes in the genome. In one embodiment, the portion of the genome comprises at least 1 Mb, 2 Mb, 3 Mb, 4 Mb, 5 Mb, 10 Mb, 15 Mb, 25 Mb, 50 Mb, 100 Mb, 200 Mb, 500 Mb, 1000 Mb, 2000 Mb or 3000 Mb of genomic sequence.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

In certain embodiments of particular interest, in situ prepared arrays are employed. In situ prepared oligonucleotide arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further described in copending application Ser. No. 10/449,838, the disclosure of which is herein incorporated by reference. Other features of in situ prepared arrays that make such array formats of particular interest in certain embodiments of the present invention include, but are not limited to: feature density, oligonucleotide density within each feature, feature uniformity, low intra-feature background, low inter-feature background, e.g., due to hydrophobic interfeature regions, fidelity of oligonucleotide features making up the individual features, array/feature reproducibility, and the like. The above benefits of in situ produced arrays assist in maintaining adequate sensitivity while operating under stringency conditions required to accommodate highly complex samples.

Generally, nucleic acid hybridizations between the probes and the arrays comprise the following major steps: (1) provision of array of surface immobilized nucleic acids or features; (2) optionally pre-hybridization treatment to increase accessibility of features, and to reduce nonspecific binding; (3) hybridization of the nucleic acid probes to the features on the solid surface, typically under high-stringency conditions; (4) post-hybridization washes to remove probes not bound in the hybridization; and (5) detection of the hybridized probes. The reagents used in each of these steps and their conditions for use vary depending on the particular application.

As indicated previously, hybridization is carried out under suitable hybridization conditions, which may vary in stringency as desired. In certain embodiments, highly-stringent hybridization conditions may be employed. The term “highly-stringent hybridization conditions” as used herein refers to conditions that are compatible to produce nucleic acid binding complexes on an array surface between complementary binding members, i.e., between immobilized features and complementary solution phase nucleic acids in a sample. Representative high-stringency assay conditions that may be employed in these embodiments are provided above.

The hybridization step may include agitation of the immobilized features and the sample of solution phase nucleic acids, where the agitation may be accomplished using any convenient protocol, e.g., shaking, rotating, spinning, and the like. Following hybridization, the surface of immobilized nucleic acids is typically washed to remove unbound nucleic acids. Washing may be performed using any convenient washing protocol, where the washing conditions are typically stringent, as described above.

Following hybridization and washing, as described above, the hybridization of the probes to the array is then detected using standard techniques so that the surface of immobilized features, e.g., array, is read. Reading of the resultant hybridized array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable devices and methods are described in U.S. patent application Ser. No. 09/846,125 “Reading Multi-Featured Arrays” by Dorsel et al.; and U.S. Pat. No. 6,406,849, which references are incorporated herein by reference.

Arrays, however, may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere).

In the case of indirect labeling, subsequent treatment of the array with the appropriate reagents may be employed to enable reading of the array. Some methods of detection, such as surface plasmon resonance, do not require any labeling of the nucleic acids, and are suitable for some embodiments. In some embodiments, detecting the hybridization between the labeled/unlabeled probes and the nucleic acids complimentary to the genome is facilitated by contacting the complexes between the labeled or unlabeled probe and the nucleic acid on the array with a detection agent, wherein the amount of detection agent that binds to the complex is indicative of the level of hybridization. In one embodiment, the detection agent comprises an antibody or fragment thereof. In another embodiment, the detection agent comprises a dendrimer. The use of dendrimers for the detection microarray hybridization has been described in U.S. Pat. Pub. Nos. 2002/0051981 and 2002/0072060, hereby incorporated by reference in their entirety. In another embodiment, the detection agent binds to a double stranded nucleic acid selected from the group consisting of a DNA-DNA, DNA-RNA or RNA-RNA double stranded-nucleic acids.

Results from the reading or evaluating may be raw results (such as fluorescence intensity readings for each feature in one or more color channels) or may be processed results, such as obtained by subtracting a background measurement, or by rejecting a reading for a feature which is below a predetermined threshold and/or forming conclusions based on the pattern read from the array (such as whether or not a particular feature sequence may have been present in the sample, or whether or not a pattern indicates a particular condition of an organism from which the sample came).

DNA microarray and methods of analyzing data from microarrays are well-described in the alt, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

In certain embodiments of the methods described herein, one or more steps are performed in different locations. In one embodiment, the fragments to which the protein of interest binds are isolated in a first location, while hybridization of the probes to an array is performed in a second location. An optional step of synthesizing probes from the fragments may be performed at either location. When two locations are used, method comprises, in some embodiments, the transport of DNA fragments or probes generated therefrom from the first location to the second location. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. In one embodiment, two locations that are remote relative to each other are at least 1, 2, 3, 4, 5, 10, 20, 50, 100, 200, 500, 1000, 2000 or 5000 km apart. In another embodiment, the two location are in different countries, where one of the two countries is the United States.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the methods comprises one or more data transmission steps between the locations. In one embodiment, the data transmission step occurs via an electronic communication link, such as the internet. In one embodiment, the data transmission step from the first to the second location comprises experimental parameter data, wherein the experimental parameter data comprises data selected from: (a) the phylogenetic species of the genome; (b) clinical data from the organism from which the genome was derived; and (c) a microarray to which the labeled probes are to be hybridized.

In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from identifying regions of a genome is transmitted from the second location to the first location.

### IV. Protein of Interest

The protein of interest used in the methods described herein may be native to the cell, or it may be a recombinant protein. By native it is meant that the protein of interest occurs naturally in the cell. In some embodiments, the protein of interest is from a species which is different from that of the genome. In some embodiments, a recombinant protein of interest may have missense mutations, truncations, or inserted sequences such as entire domains, relative to naturally-occurring forms of the proteins. A tagged protein of interest may be used in some embodiments, especially when the tag facilitates its immunoprecipitation.

In certain embodiments of the invention, the protein of interest comprises specific transcription factors, coactivators, repressors, corepressors or complexes thereof. Transcription factors bind to specific cognate DNA elements such as promoters, enhancers and silencer elements, and are responsible for regulating gene expression. Transcription factors may be activators of transcription, repressors of transcription or both, depending on the cellular context. Transcription factors may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes.

Antibodies directed to any transcriptional coactivator or corepressor may also be used according to the invention. Examples of specific coactivators include CBP, CTIIA, and SRA, while specific examples of corepressors include the mSin3 proteins, MITR, and LEUNIG. Furthermore, the genes regulated by proteins associated with transcriptional complexes, such as the histone acetylases (HATs) and histone deacetylases (HDACs), may also de determined using the methods described herein. Histone Deacetylases are described, for example, in Johnstone, R. W., “Histone-Deacetylase Inhibitors: Novel Drugs for the Treatment of Cancer”, Nature Reviews, Volume I, pp. 287-299, (2002) and PCT Publication Nos. 00/10583, 01/18045, 01/42437 and 02/08273. U.S. Patent Application No. 2005/0159470 describes members of the three classes of histone acetylases in Tables 1-3.

In other embodiments of the methods described herein, the protein of interest is a DNA-binding protein, such as a basal transcription factor or a component of the basal transcription machinery. Exemplary components of the basal transcription machinery include RNA polymerases, including polI, polII and polIII, TBP, NTF-1 and Sp1 and any other component of TFIID, including, for example, the TAFs (e.g. TAF250, TAF150, TAF135, TAF95, TAF80, TAF55, TAF31, TAF28, and TAF20), or any other component of a polymerase holoenzyme. In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20).

In some embodiments, the protein of interest is a histone. Histones are small, positively charged proteins that are rich in basic amino acids (positively charged at physiological pH), which contact the phosphate groups (negatively charged at physiological pH) of DNA. There are five main classes of histones H1, H2A, H2B, H3, and H4, any of which may be used in the invention. Four pairs of each of H2A, H2B, H3 and H4 together form a disk-shaped octomeric protein core, around which DNA (about 140 base pairs) is wound to form a nucleosome. In one embodiment, the protein of interest is a modified histone, such as one that is methylated or acetylated, either singly or multiply modified.

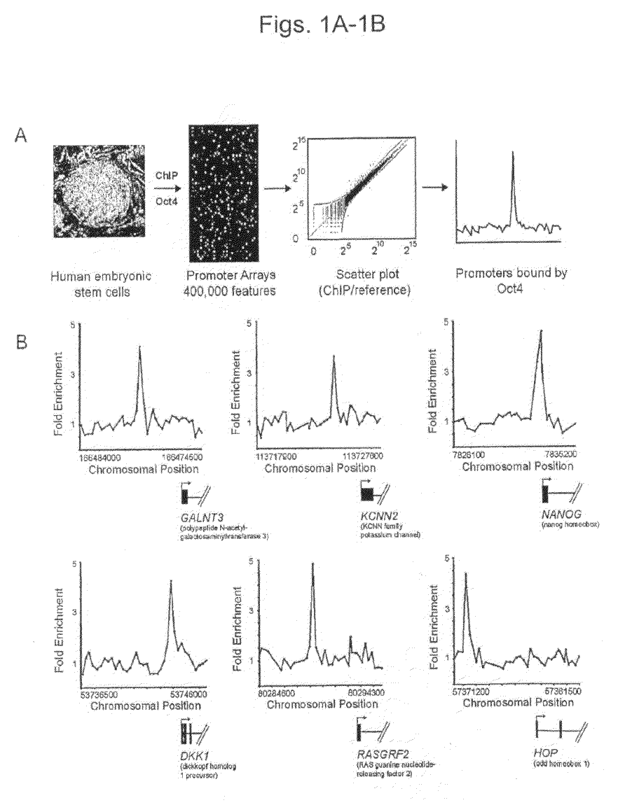

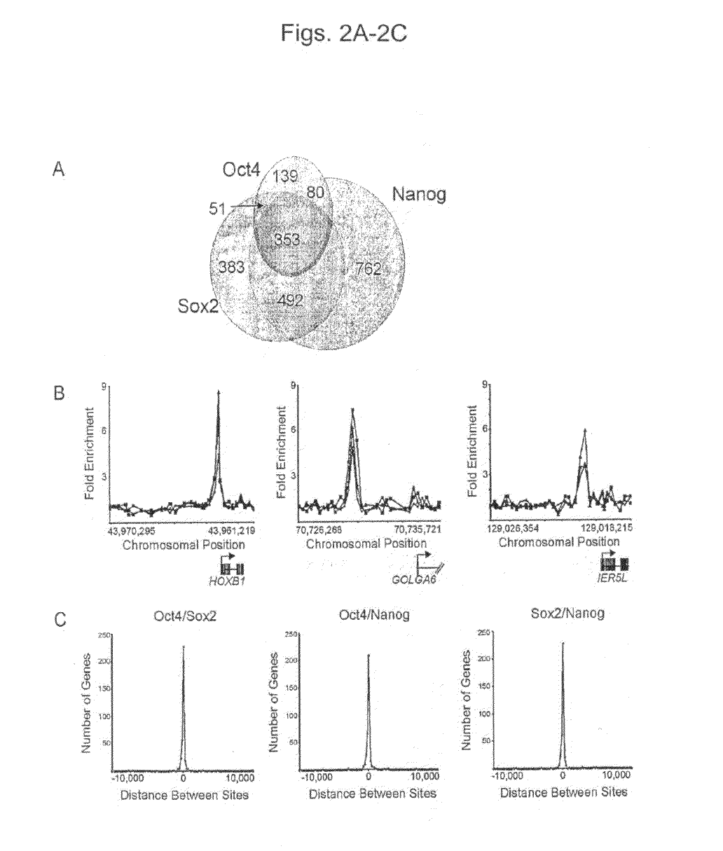

In one embodiment, the protein of interest is a PRC2 subunit. Subunits of the PRC2 complex are described, for example, in Kuzmichev et al. Proc Natl Acad Sci USA. 2005; 102(6):1859-64 and Guitton et al, Int. J. Dev. Biol. 49: 707-716 (2005) and in particular in FIG. 1 of Guitton. In one embodiment, the PRC2 subunit is SUZ12, EED or EZH2. In one embodiment, the protein of interest is a PRC1 subunit, such as BMI1.

The methods described herein may be applied to protein of interest that has been causally implicated in a disease. Examples of diseases and transcriptional regulators which cause them may be found in the scientific and medical literature by one skilled in the art, including in Medical Genetics, L. V. Jorde et al., Elsevier Science 2003, and Principles of Internal Medicine, 15th edition, ed by Braunwald et al., McGraw-Hill, 2001; American Medical Association Complete Medical Encyclopedia (Random House, Incorporated, 2003); and The Mosby Medical Encyclopedia, ed by Glanze (Plume, 1991). In some embodiments, the disorder is characterized by impaired function of at least one of the following organs or tissues: brain, spinal cord, heart, arteries, esophagus, stomach, small intestine, large intestine, liver, pancreas, lungs, kidney, urinary tract, ovaries, breasts, uterus, testis, penis, colon, prostate, bone, scalp, muscle, cartilage, thyroid gland, adrenal gland, pituitary, bone marrow, blood, thymus, spleen, lymph nodes, skin, eye, ear, nose, teeth or tongue.

### V. Identifying the Differentiation Potential of a Cell

One aspect of the invention provides methods of identifying the differentiation potential of a test cell. In one embodiments, the method comprises identifying regions in the genome of the test cell to which a protein binds, and comparing the set of regions bound by the protein to one or more reference set of bound regions, each reference set of bound regions being indicative of the differentiation potential of a type of cell. In one embodiment, the method for identifying the differentiation potential of a test cell comprises (i) identifying regions of the genome of the test cell to which a protein binds; (ii) comparing the regions identified in (i) to one or more reference set of bound regions, each reference set of bound regions indicative of a differentiation potential, wherein a match between the regions identified in (i) and one of the reference set of bound regions indicates that the test cell has the differentiation potential corresponding to the reference set of bound regions. In one embodiment, a match between the regions identified in (i) and one of the reference set of bound regions exists when both sets are at least 60, 65, 75, 80, 85, 90, 92, 95, 96, 97, 98, 99 or 100% identical.

The reference set may be indicative, for example, of the differentiation potential of totipotent stem cells, a pluripotent stem cell, or a stem cell limited to differentiating into defined lineages, such as into one of mesoderm, ectoderm and endoderm. One or more of the reference sets may also be indicative of a differentiated cell. The test cell is said to have the differentiation potential of the cell from the reference set if at least 75, 80, 85, 90, 92, 95, 96, 97, 98, 99 or 100% of the chromosome regions that are bound by the protein are present in the reference set. In some preferred embodiments, the reference set is indicative of the differentiation potential of a stem cell. In one embodiment, the chromosome regions are the genes, or their regulatory regions, to which the protein binds.

Reference sets representative of the differentiation potential of a particular cell may be generated by identifying the regions of the genome to which the protein binds in those particular cells. The methods described herein for performing ChIP-CHIP analysis are particularly useful. For example, a reference set representative of a hepatocyte progenitor cell may be generated by performing genome-wide location analysis using chromatin from hepatocyte progenitor cells (see for example those described in Corcelle et al. Exp Cell Res. 2006; 312(15):2826-36) and using a protein such as a histone or a transcriptional regulator to generate the set of regions bound by the protein. A match in binding regions between the test cell and the reference set of the hepatocyte progenitor cell indicates that the test cell has the differentiation potential of the hepatocyte progenitor cell. Indeed, in some embodiments, the method comprises generating a reference set of bound regions for one or more cells of known differentiation potential. In one embodiment, a match results when at least 50%, 60%, 70%, 80%, 90%, 95%, 96%, 97%, 98%, 99% or 100% of the genes bound by the protein are the same as those in the reference set.

In one embodiment, the protein whose binding regions are identified is a transcriptional regulator. In one embodiment, the protein is a DNA-binding protein, such as a basal transcription factor or a component of the basal transcription machinery. In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20). In some embodiments, the protein is a histone. In one embodiment, the protein is a PRC1 or PRC2 subunit such as SUZ12, EED or EZH2. In one embodiment, the protein is a PRC1 subunit, such as BMI1. In one embodiment, the protein is Oct4, Sox2 or Nanog. In another embodiment the protein is RNA polymerase II. In one embodiment the protein is a component of the Polycomb Repression Complex 2 (PRC2) such as SUZ12.

In one embodiment, the test cell may, for example, a stem cell, an embryonic stem cell, a human embryonic stem cell, a cancer stem cell, an adult stem cell, a mammalian stem cell, a human stem cell, a non-human mammalian stem cell, a test cell that maintains the potential to differentiate into at least one tissue selected from of endoderm, mesoderm, and ectoderm tissue, a test cell that maintains the potential to differentiate into endoderm, mesoderm, and ectoderm tissues, a test cell that maintains the potential to differentiate into a neuronal cell type, mesenchymal stem cells, neural stem cells, neural crest stem cells, hematopoietic stem cells, and pancreatic stem cells. In one embodiment, the test cell is isolated from umbilical cord blood. In one embodiment, the stem cell is isolated from umbilical cord blood. In one embodiment, the stem cell is from mice, rats, rodents, rabbits, cows, pigs, primates, humans, non-human mammals or non-human primates.

In one embodiment, one or more of the reference set is indicative of the differentiation state or differentiation potential of a stem cell, an embryonic stem cell, a human embryonic stem cell, a cancer stem cell, an adult stem cell, a mammalian stem cell, a human stem cell, a non-human mammalian stem cell, a cell that maintains the potential to differentiate into at least one tissue selected from of endoderm, mesoderm, and ectoderm tissue, a cell that maintains the potential to differentiate into endoderm, mesoderm, and ectoderm tissues, a cell that maintains the potential to differentiate into a neuronal cell type, mesenchymal stem cells, neural stem cells, neural crest stem cells, hematopoietic stem cells, or pancreatic stem cells.

### V. Identification of Agents

One aspect of the invention provides methods of identifying an agent that promote the differentiation of a stem cell. One aspect of the invention provides a method for identifying an agent that promotes the differentiation of a stem cell, comprising (a) providing a culture comprising the stem cell; (b) contacting said culture with the agent; (c) identifying regions of the genome of the stem cell to which a protein binds according to one of the methods described; and (d) comparing the regions identified in (c) to one or more reference sets of bound regions indicative of a differentiating cell, wherein a match between the regions identified in (c) and one of the reference sets of bound regions indicates that the agent promotes the differentiation of the stem cell. In some embodiments, the method comprises generating a reference set of bound regions indicative of a differentiating cell.

Another aspect of the invention provides a method for identifying an agent that promotes the differentiation of a stem cell towards a given lineage, comprising (a) providing a culture comprising the stem cell; (b) contacting said culture with the agent; (c) identifying regions of the genome of the stem cell to which a protein binds according to one of the methods described; and (d) comparing the regions identified in (c) to one or more reference sets of bound regions indicative of cell differentiating towards the given lineage, wherein a match between the regions identified in (c) and one of the reference sets of bound regions indicates that the agent promotes the differentiation of the stem cell towards the given lineage. In some embodiments, the method comprises generating one or more reference sets of bound regions indicative of cell differentiating towards the given lineage. In one embodiment, the given lineage is a particular differentiated state, such as being a hepatocyte, cardiomyocyte or vascular cell, while in other it is a cell that is partially differentiated.

In one embodiment, the protein used a transcriptional regulator. In one embodiment, the protein is a DNA-binding protein, such as a basal transcription factor or a component of the basal transcription machinery. In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20). In some embodiments, the protein is a histone. In one embodiment, the protein is a PRC1 or PRC2 subunit such as SUZ12, EED or EZH2. In one embodiment, the protein is a PRC1 subunit, such as BMI1. In one embodiment, the protein is Oct4, Sox2 or Nanog. In another embodiment the protein is RNA polymerase II. In one embodiment the protein is a component of the Polycomb Repression Complex 2 (PRC2) such as SUZ12.

In one embodiment, the stem cell used in the methods of identifying an agent that promotes the differentiation of the stem cell is an embryonic stem cell, a human embryonic stem cell, a cancer stem cell, an adult stem cell, a mammalian stem cell, a human stem cell, a non-human mammalian stem cell, a stem cell that maintains the potential to differentiate into at least one tissue selected from of endoderm, mesoderm, and ectoderm tissue, a stem cell that maintains the potential to differentiate into endoderm, mesoderm, and ectoderm tissues, a stem cell that maintains the potential to differentiate into a neuronal cell type, a mesenchymal stem cell, a cancer stem cell, a neural stem cell, a neural crest stem cell, a hematopoietic stem cell, and a pancreatic stem cell. In one embodiment, the stem cell is isolated from umbilical cord blood. In one embodiment, the stem cell is from mice, rats, rodents, rabbits, cows, pigs, primates, humans, non-human mammals or non-human primates. In one embodiment, the stem cell is an human embryonic stem cell H9.

Conditions that may be used to induce the differentiation of a stem cell into a given lineage are known, and therefore reference sets of bound regions indicative of cell differentiating towards the given lineage, or simply of differentiating cells, may be generated using these cells as they differentiate. For example, Li X J et al. Methods Mol. Biol. 2006; 331:169-77 describes the in vitro differentiation of neural precursors from human embryonic stem cells; Wang L et al. Methods Mol Biol. 2006; 331:179-200 describes the derivation and characterization of hematopoietic cells from human embryonic stem cells; Carpenter et al., Methods Mol Biol. 2006; 331:153-67 describes the derivation and characterization of neuronal precursors and dopaminergic neurons from human embryonic stem cells in vitro; Baharvand et al. Int J Dev Biol. 2006; 50(7):645-52 describes the differentiation of human embryonic stem cells into hepatocytes in 2D and 3D culture systems in vitro; Lamba et al. Proc Natl Acad Sci USA. 2006 Aug. 22; 103(34): 12769-74 describes an efficient generation of retinal progenitor cells from human embryonic stem cells; Samadikuchaksaraei et al. Tissue Eng. 2006 April; 12(4):867-75 describes the derivation of distal airway epithelium from human embryonic stem cells; Ji et al. Tissue Eng. 2006 April; 12(4):665-79 describes the generation and differentiation of human embryonic stem cell-derived keratinocyte precursors; Olivier et al. Stem Cells. 2006 August; 24(8):1914-22 describes the differentiation of human embryonic stem cells into bipotent mesenchymal stem cells; Taylor et al. Nat Methods. 2006; 3(3): 179-81 describes the formation of human prostate tissue from embryonic stem cells; Iuchi et al. Proc Natl Acad Sci USA. 2006; 103(6):1792-7 describes the immortalized keratinocyte lines derived from human embryonic stem cells; Xiong et al. Stem Cells Dev. 2005; 14(6):671-5 describes the derivation of adipocytes from human embryonic stem cells; Schwartz et al. Stem Cells Dev. 2005; 14(6):643-55 describes defined conditions for development of functional hepatic cells from human embryonic stem cells; Gaur et al. J Thromb Haemost. 2006 February; 4(2):436-42 describes the megakaryocytes derived from human embryonic stem cells; Passier et al. Handb Exp Pharmacol. 2006; (174):101-22 describes cardiomyocytes derived from human embryonic stem cells; Muotri et al. Proc Natl Acad Sci USA. 2005; 102(51):18644-8 describes the development of functional human embryonic stem cell-derived neurons in mouse brain; Qiu et al, Exp Hematol. 2005; 33(12):1450-8 describes the differentiation of human embryonic stem cells into hematopoietic cells by coculture with human fetal liver cells; Tibbitts et al. Stem Cells Dev. 2006; 15(2):200-8 describes the generation of uniform adherent neural progenitor populations from rhesus embryonic stem cells; Zhang et al. Neuroreport. 2006; 17(10):981-6 describes the neural differentiation of embryonic stem cells induced by conditioned medium from neural stem cell; Lau et al. Neuroreport. 2006; 17(10):975-9 describes the rapid and efficient differentiation of dopaminergic neurons from mouse embryonic stem cells. Additional methods of inducing differentiation in ex vivo expanded stem cells are described in U.S. 2005/0220774.

The above differentiating condition may also be used to detect agents that block the differentiating of the stem cell, by culturing the cell in the conditions that promote differentiation, contacting the cell with the agent, and determining if the agent inhibits the differentiation of the cell. Such methods may be used to identify agents to promote the undifferentiated state of stem cells. In some embodiments of the methods described herein, the regions of the genome that are bound by the protein of interest are determined in both a stem cell that is contacted with an agent and in a stem cell that is not contacted with the agent, or that is contacted with a different amount of the agent. Such methods may be used to identify compounds that maintain the undifferentiated state of a stem cell, or that promote their differentiation towards a particular differentiated cell type.

In some embodiments of the methods described herein, the stem cell has been treated with an agent, such as compound or a drug, prior to the fragmenting of genomic DNA and preferably while the stem cell is alive. Some preferred agents include those which bind to and/or regulate the expression of transcriptional regulators, or which are suspected of doing so. In some embodiments, the regions of the genome that are bound by a given transcriptional regulator are determined both in a stem cell that is contacted with an agent and in a stem cell that is not contacted with the agent, or that is contacted with a different amount of the agent. Such methods may be used to identify compounds that alter the types of genes and/or the extent to which a transcriptional regulators controls transcription of genes or stem cell differentiation and maintenance of the undifferentiated state. Furthermore, such approaches may be used to screen for agents which alter the activity, DNA-binding specificity or expression of a transcriptional regulator in a stem cell.

In some embodiments of the methods described herein, the experimental agent or drug comprises a small molecule drug, an antisense nucleic acid, an antibody, a peptide, a ligand, a fatty acid, a hormone or a metabolite. Exemplary compounds that may be used as experimental agents (e.g., a single compound, a combination of two or more compounds, a library of compounds) include nucleic acids, peptides, polypeptides, peptidomimetics, antibodies, antisense oligonucleotides, RNAi constructs (including siRNAs), ribozymes, chemical compounds, and small organic molecules, such as those having a molecular weight of less that 2 kDa, and preferably less than 1 kDa. Compounds may be screened individually, in combination, or as a library of compounds.

The assays described herein may also be used to screen a library of compounds to test the activity of each library member on the DNA-binding properties of protein of interest. Library members may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (about 100 kDa to 10 kDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others. Library members may be contact arrays of cell populations singly or as groups/pools of two or more members.

### VI. Cells

The stem cell type from which the chromatin is obtained may be any stem cell type. In some preferred embodiments, the stem cell is a mammalian cell, such as a stem cell from a rodent, a primate or a human. In more preferred embodiments, the stem cell is an embryonic stem cells or an adult stem cell. In one embodiment, the stem cells may be from humans, or from a nonhuman mammal. In one embodiment, the stem cell is derived from mice, rats, rabbits, cows, pigs, humans, or non-human primates.

Methods of isolating human embryonic stem cells are well-know in the art. (See, for example, Mitalipova et al. Isolation and characterization of human embryonic stem cells. Methods Mol Biol. 2006; 331:55-76; Amit et al. Derivation and maintenance of human embryonic stem cells. Methods Mol Biol. 2006; 331:43-53; Maintenance of human embryonic stem cells in animal serum- and feeder layer-free culture conditions. Methods Mol Biol. 2006; 331:105-13; Dravid et al. Culture of human embryonic stem cells on human and mouse feeder cells; Methods Mol Biol. 2006; 331:91-104; McWhir et al. Routine culture and differentiation of human embryonic stem cells. Methods Mol Biol. 2006; 331:77-90; Turksen et al. Human embryonic stem cells: isolation, maintenance, and differentiation. Methods Mol Biol. 2006; 331:1-12.; Hoffman et al. Characterization and culture of human embryonic stem cells. Nat Biotechnol. 2005 June; 23(6):699-708; Oh et al. Methods for expansion of human embryonic stem cells; Stem Cells. 2005 May; 23(5):605-9; and references cited therein). Pluripotent embryonic stem cells are described in U.S. Pub No. 2004/0071672. Primate embryonic stem cells are described in U.S. Pat. No. 6,200,806. Human pluripotent adult stem cells are described in U.S. 20050260751. Isolated adult pluripotent stem cells are described in 20050014254. Adult stem cells fare described in 20050260748. Multipotent stem cells from peripheral tissues are described in 20030003574. Generation and differentiation of adult stem cell lines are described in 20060093586. Multipotent stem cells from peripheral tissues are described in 20020123143. The isolation and characterization of residual undifferentiated mouse embryonic stem cells from embryoid body cultures by fluorescence tracking is described in Ensenat-Waser et al. In Vitro Cell Dev Biol Anim. 2006 May-June; 42(5-6): 115-23. The derivation, maintenance, and characterization of rat embryonic stem cells in vitro is described in Schulze et al. Methods Mol Biol. 2006; 329:45-58.

The cell may be a naturally occurring cell or a cell that has been genetically modified by recombinant means (see for example Eiges R. et al, Genetic manipulation of human embryonic stem cells by transfection. Methods Mol Biol. 2006; 331:221-39) or by exposure to mutagens such as DNA modification agents or radiation. In some embodiments, the stem cell is from an organism afflicted by a disease. In some embodiments, the stem cell comprises a genetic mutation, somatic or germline, that contributes to the disease state. In one embodiment, the disease is cancer.

In one embodiment, fragmenting the genomic DNA comprises fragmenting the genomic DNA of a population of cells. In one embodiments of the methods described herein, the population of cells comprises less than 10⁸, 10⁷, 10⁶, 10⁵, 10⁴, 10³ or 10²: In some embodiments, the population of cells comprises less than 10⁸, 10⁷, 10⁶, 10⁵, 10⁴, 10³ or 10² cells which express the protein of interest, but also comprises cells which do not express the protein of interest. In one embodiment, the cell population is a population that has been isolated using fluorescent-activated cell-sorting (FACS).

In one embodiment of the methods described herein, the chromatin is from stem cells that have undergone minimum passaging in vitro, such as less than 20, 15, 10, 8, 6, 5, 4, 3 2 or less doublings in vitro. In one embodiment, the stem cell is a cancer stem cell. Isolation of cancer stem cells is described in U.S. Patent Pub Nos. 20030119080 titled “Strategies for the identification and isolation of cancer stem cells and non-cancerous stem cells” and 2002/0119565 titled “Isolation and use of solid tumor stem cells.”

In preferred embodiments of the methods described herein, the cell populations are contained within wells of multi-well plates to facilitate parallel handling of cells and reagents. In specific embodiments, the multi-well plate has 24, 48, 96 or 384 wells. Standard 96 well microtiter plates which are 86 mm by 129 mm, with 6 mm diameter wells on a 9 mm pitch, may be used for compatibility with current automated loading and robotic handling systems. The microplate is typically 20 mm by 30 mm, with cell locations that are 100-200 microns in dimension on a pitch of about 500 microns. Methods for making microplates are described in U.S. Pat. No. 6,103,479, incorporated by reference herein in its entirety.

Microplates may consist of coplanar layers of materials to which cells adhere, pattered with materials to which cells will not adhere, or etched 3-dimensional surfaces of similarly pattered materials. For the purpose of the following discussion, the terms “well” and “microwell” refer to a location in an array of any construction to which cells adhere and within which the cells are imaged. Microplates may also include fluid delivery channels in the spaces between the wells. The smaller format of a microplate increases the overall efficiency of the system by minimizing the quantities of the reagents, storage and handling during preparation and the overall movement required for the scanning operation. In addition, the whole area of the microplate can be imaged more efficiently. Multi-well test plates used for isotopic and non-isotopic assays are well known in the art and are exemplified, for example, by those described in U.S. Pat. Nos. 3,111,489; 3,540,856; 3,540,857; 3,540,858; 4,304,865; 4,948,442; and 5,047,215.

Microfluidic devices may also be used at any of the steps of the methods described herein. For example, Chung et al. (2004) Lab Chip; 4(2):141-7 describe a high efficiency DNA extraction microchip was designed to extract DNA from lysed cells using immobilized beads and shaking solution, which allows extraction of as little as 10³ cells. Guijt et al. (2003) Lab Chip; 3(1): 14 describes microfluidic devices with accurate temperature control, as might be used to cycle temperature during PCR amplification. Similarly, Liu et al. (2002) Electrophoresis; 23(10):1531-6 teaches a microfluidic device for performing PCR amplification using as little as 12 nL of sample. Cady et al. (2003) Biosens Bioelectron. 30; 19(1):59-66 describes a microfluidic device that may be used to purify DNA.

### VII. Nucleotide Array Probes

One aspect of the invention provides a method of obtaining a set of nucleotide array probes tiling the promoter region of one or more genes. In one embodiment, the method of obtaining a set of nucleotide array probes tiling the promoter region of a gene comprises (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected (a) have one or more desirable hybridization properties; and (b) tile the promoter region with an average density of about from 100 to about 1000 bp.

In one embodiment, selecting a transcriptional start site for the gene is done by selecting a well-characterized transcription start sites are used. These may be collected from any one of several databases, such as RefSeq, Ensembl, MGC, VEGA (www.vega.sanger.ac.uk) and Broad (www.broad.mit.edu). The first three are commonly used databases for gene annotation, the last two are manually annotated databases covering subsets of the human genome from the Sanger Institute and Broad Institute, respectively. In one embodiment, all transcription start sites that appear in at least one or two or more of these five databases are filtered. In one embodiment start sites separated by less than 500, 400, 300, 200 or 100 bp in any of the databases are considered identical for the filtering step. In cases where there were multiple start sites with different genomic coordinates, the start site that would result in the longest transcript may be selected in some embodiments.

The program ArrayOligoSelector (AOS, Bozdech et al., 2004) may be used to score 60-mers for every unmasked subsequence greater than 62 bp across all promoter regions. The scores for each oligo may be retained but not put through the built-in AOS selection process. In one embodiment, the collection of scored 60-mers is divided by promoter and sorted further by genomic position. In one embodiment, each set of 60-mers is then filtered based on the oligo scoring criteria. In one embodiment, one or more or all of the following criteria are used: (1) GC content, (2) self-binding, (3) complexity and (4) uniqueness. In one embodiment using stringent filter, the following ranges for each parameter are selected: GC content between 30 percent and 100 percent, self-binding score less than 100, complexity score less than or equal to 24, uniqueness greater than or equal to 40. Other parameters include GC content between 40 percent and 90 percent, self-binding score less than 80, complexity score less than or equal to 20, uniqueness greater than or equal to −50.

From a subset of 60-mers, oligos designed to cover the promoter region with an estimated density of one probe every about 400-200, 350-250, 320-270 or about 280 basepairs are selected. To achieve more uniform tiling, a simple method to find probes within a particular distance from each other may be applied. For example, a starting at the upstream end of the region, a first qualified probe may be selected, then the next qualified probe that is between a set range of distances away, 100 bp and 400 bp for example, may be selected. If there are multiple, eligible probes, one may chose the most distal probe within the upper range limit, e.g. 400 bp in this example. If there are no probes within this limit, one may continue scanning until the next nearest acceptable probe is located. The process may then repeated with the most recently selected probe until the end of the promoter region is reached.

In one embodiment, for regions that are not covered by high quality probes, one may return to the full set of scored 60-mers and filter using less stringent criteria to identify additional 60-mer probes. This may provide additional sets of 60-mers that may be used to fill gaps in the coverage. After this second pass, gaps in the coverage may be identified, and oligos that are properly spaced and that best fit the criteria, regardless of whether they passed the filter cutoffs, may be used. This iterative process gives a compromise between optimal probe quality and optimal probe spacing. In one embodiment, for each start site, a region at of less than 20, 15, 10, 8, 6 or 5 kb upstream and 5, 4, 3, 2, 1.5 or 1 kb downstream of the site is used for tiling.

In one embodiment, the transcriptional start site for the gene is selected from a group of transcriptional start sites, each resulting in a transcript, wherein the start site selected results in the longest transcript. In one embodiment, the window of genomic DNA sequence spans from about 8 kb upstream of the start site to about 2 kb downstream. In one embodiment, each probe in the subset has a length of between 50 and 70 nucleotides. In one embodiment, the desirable properties for the array probes are selected from (a) a GC nucleotide content of between 30 and 100 percent; (b) low self-binding; (c) low complexity; and (d) high uniqueness. In one embodiment, the array probes in the subgroup tile the promoter region with a density of about from 200 bp to about 300 bp. In one embodiment, the method further comprising the step of (v) selecting additional preliminary probes for the set of nucleotide array probes, wherein the additional preliminary probes: (a) have less desirable hybridization properties than the preliminary probes selected in step (iv); and (b) are flanked by two nucleotide array probes, wherein the distance between the two flanking probes along the genome is greater than the average distance between adjacent nucleotide array probes of step (iv). In one embodiment, the distance along the genome between the two flanking probes along the genome is greater than the average distance between adjacent nucleotide array probes of step (iv) by at least 100 bp. In one embodiment, the method comprises the step of (v) synthesizing a plurality of nucleic acid probes, each probe having the nucleotide sequence of a nucleotide array probe. In one embodiment, the method comprises the step of (vi) immobilizing the plurality of probes on a glass surface. In one embodiment, the gene is a mammalian gene. In one embodiment, the gene is a human gene. In one embodiment, the method comprises synthesizing at least one of the nucleotide array probes. In one embodiment, the method comprises immobilizing the nucleotide array probe on a microarray.

### VIII. Program Products/Systems

Another aspect of the invention provides a program product (i.e. software product) for use in a computer device that executes program instructions recorded in a computer-readable medium to perform one or more steps of the methods described herein for obtaining a set of nucleotide array probes tiling the promoter region of one or more genes.

On aspect of the invention provides a computer readable medium having computer readable program codes embodied therein for obtaining a set of nucleotide array probes tiling the promoter region of a gene or set of genes, the computer readable medium program codes performing one or more of the following functions: (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected (a) have one or more desirable hybridization properties; and (b) tile the promoter region with an average density of about from 100 to about 1000 bp.

Another related aspect of the invention provides kits comprising the program product or the computer readable medium, optionally with a computer system. On aspect of the invention provides a system for obtaining a set of nucleotide array probes tiling the promoter region of a gene, the system comprising: a computer; a computer readable medium, operatively coupled to the computer, the computer readable medium program codes performing one or more of the following functions: (i) selecting a transcriptional start site for the gene; (ii) identifying masked and unmasked subsequences within a contiguous window of genomic DNA sequence that comprises the transcriptional start site; (iii) for each unmasked subsequence greater than 20 nucleotides in length, generate a subset of preliminary probes, the preliminary probe having (a) a length of between 40 and 80 nucleotides; and (b) a sequence identical to a portion of the unmasked subsequence, and (iv) from the preliminary probes of step (iii), selecting a set of nucleotide array probes tiling the promoter region of the gene, wherein the array probes that are selected (a) have one or more desirable hybridization properties; and (b) file the promoter region with an average density of about from 100 to about 1000 bp.

In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on literature, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM).

Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein.

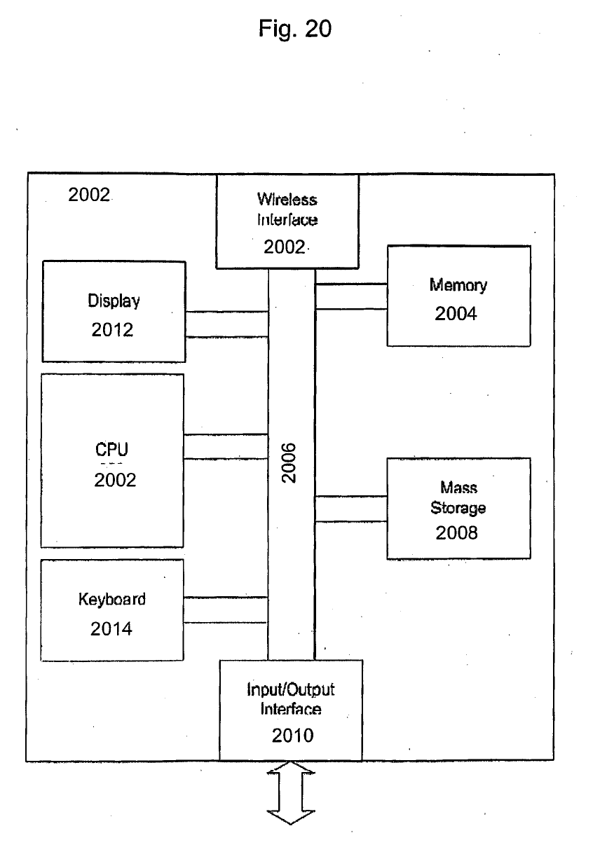

FIG. 20 shows a functional block diagram of general purpose computer system 2000 for performing the functions of the software according to an illustrative embodiment of the invention. The exemplary computer system 2000 includes a central processing unit (CPU) 2002, a memory 2004, and an interconnect bus 2006. The CPU 2002 may include a single microprocessor or a plurality of microprocessors for configuring computer system 2000 as a multi-processor system. The memory 2004 illustratively includes a main memory and a read only memory. The computer 2000 also includes the mass storage device 2008 having, for example, various disk drives, tape drives, etc. The main memory 2004 also includes dynamic random access memory (DRAM) and high-speed cache memory. In operation, the main memory 2004 stores at least portions of instructions and data for execution by the CPU 2002.

The mass storage 2008 may include one or more magnetic disk or tape drives or optical disk drives, for storing data and instructions for use by the CPU 2002. At least one component of the mass storage system 2008, preferably in the form of a disk drive or tape drive, stores one or more databases, such as databases containing of transcriptional start sites, genomic sequence, promoter regions, or other information.

The mass storage system 2008 may also include one or more drives for various portable media, such as a floppy disk, a compact disc read only memory (CD-ROM), or an integrated circuit non-volatile memory adapter (i.e. PC-MCIA adapter) to input and output data and code to and from the computer system 2000.

The computer system 2000 may also include one or more input/output interfaces for communications, shown by way of example, as interface 2010 for data communications via a network. The data interface 2010 may be a modem, an Ethernet card or any other suitable data communications device. To provide the functions of a computer 104 according to FIG. 1, the data interface 2010 may provide a relatively high-speed link to a network, such as an intranet, internet, or the Internet, either directly or through an another external interface. The communication link to the network may be, for example, optical, wired, or wireless (e.g., via satellite or cellular network). Alternatively, the computer system 2000 may include a mainframe or other type of host computer system capable of Web-based communications via the network.

The computer system 2000 also includes suitable input/output ports or use the interconnect bus 2006 for interconnection with a local display 2012 and keyboard 2014 or the like serving as a local user interface for programming and/or data retrieval purposes. Alternatively, server operations personnel may interact with the system 2000 for controlling and/or programming the system from remote terminal devices via the network.

The computer system 2000 may run a variety of application programs and stores associated data in a database of mass storage system 2008. One or more such applications may enable the receipt and delivery of messages to enable operation as a server, for implementing server functions relating to obtaining a set of nucleotide array probes tiling the promoter region of a gene or set of genes.

The components contained in the computer system 2000 are those typically found in general purpose computer systems used as servers, workstations, personal computers, network terminals, and the like. In fact, these components are intended to represent a broad category of such computer components that are well known in the art.

It will be apparent to those of ordinary skill in the art that methods involved in the present invention may be embodied in a computer program product that includes a computer usable and/or readable medium. For example, such a computer usable medium may consist of a read only memory device, such as a CD ROM disk or conventional ROM devices, or a random access memory, such as a hard drive device or a computer diskette, having a computer readable program code stored thereon.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, transcriptional regulators, cell types, antibodies, ChIP conditions, or data analysis methods, all without limitation, can be employed, without departing from the scope of the invention as claimed.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

Various publications, patents, and patent publications are cited throughout this application the contents of which are incorporated herein by reference in their entirety.

### Example 1

### Oct4 Promoter Occupancy in Human ES Cells

DNA sequences occupied by Oct4 in human H9 ES cells (NIH code WA09) were identified in a replicate set of experiments using chromatin-immunoprecipitation (ChIP) combined with DNA microarrays (FIG. 1A). For this purpose, DNA microarrays were designed that contain 60-mer oligonucleotide probes covering the region from −8 kb to +2 kb relative to the transcript start sites for 17,917 annotated human genes. Although some transcription factors are known to regulate genes from distances greater than 8 kb, applicants found that 98% of known binding sites for human transcription factors occur within 8 kb of target genes. The sites occupied by Oct4 were identified as peaks of ChIP-enriched DNA that span closely neighboring probes (FIG. 1B). Oct4 was found to be associated with 623 (3%) of the promoter regions for known protein-coding genes and 5 (3%) of the promoters for known miRNA genes in human ES cells.

Two lines of evidence suggested that this protein-DNA interaction dataset is of high quality. First, the genes occupied by Oct4 in our analysis included many previously identified or supposed target genes in mouse ES cells or genes whose transcripts are highly enriched in ES cells, including POU5F1/OCT4, SOX2, NANOG, LEFTY2/EBAF, CDX2, HAND1, DPPA4, GJA1/CONNEXIN43, FOXO1A, TDGF1 and ZIC3 (Abeyta et al., 2004; Brandenberger et al., 2004; Catena et al., 2004; Kuroda et al., 2005; Niwa, 2001; Okumura-Nakanishi et al., 2005; Rodda et al., 2005; Sato et al., 2003; Wei et al., 2005) (Table 3). Second, we have used improved protocols and DNA microarray technology in these experiments that should reduce false positive rates relative to those obtained in previous genome-scale experiments (Odom et al., 2004). By using this new technology with yeast transcription factors, where considerable prior knowledge of transcription factor binding sites has been established, we estimated that this platform has a false positive rate of <1% and a false negative rate of 20%.

### Example 2

### Oct4. Sox2, and Nanog Co-Occupy Many Target Genes

We next identified protein-coding and miRNA genes targeted by the stem cell regulators Sox2 and Nanog using location analysis. Sox2 and Nanog were found associated with 1271 (7%) and 1687 (9%), respectively, of the promoter regions for known protein-coding genes in human ES cells (Table 3). It was immediately evident that many of the target genes were shared by Oct4, Sox2, and Nanog (FIG. 2A). Examples of protein-coding genes that are co-occupied by the three regulators are shown in FIG. 2B. Control experiments showed that the set of promoters bound by the cell cycle transcription factor E2F4 in these human ES cells did not overlap substantially with those bound by the three stem cell regulators (Table 3). We found that Oct4, Sox2 and Nanog together occupy at least 353 genes in human ES cells.

Previous studies have shown that Sox2 and Oct4 can interact cooperatively to synergistically activate transcription of target genes in murine ES cells and that this activity is dependent upon the juxtaposition of Oct4 and Sox2 binding sites (Ambrosetti et al., 1997; Remenyi et al., 2004). Our results revealed that approximately half of the genes occupied by Oct4 were also bound by Sox2 in human ES cells (FIG. 2A; Table 3). It was surprising, however, to find that >90% of promoter regions bound by both Oct4 and Sox2 were also occupied by Nanog. Furthermore, we found that Oct4, Sox2, and Nanog binding sites occurred in close proximity at nearly all of the genes that they co-occupied FIG. 2C). These data suggest that Oct4, Sox2 and Nanog function together to regulate a significant proportion of their target genes in human ES cells.

A class of small non-coding RNAs known as microRNAs (miRNA) play vital roles in gene regulation and recent studies indicate that more than a third of mammalian protein-coding genes are conserved miRNA targets (Bartel, 2004; Lewis et al., 2005). ES cells lacking the machinery that processes miRNA transcripts are unable to differentiate (Kanellopoulou et al., 2005). Moreover, recent evidence indicates that microRNAs play an important role in organismal development through regulation of gene expression (Pasquinelli et al., 2005). Oct4, Sox2, and Nanog were found associated with 14 miRNA genes and co-occupied the promoters of at least two miRNA genes, mir-137 and mir-301 (Table 1).

TABLE 1
miRNA Loci Near Oct4, Sox2 and Nanog Bound Regions

| | Transcription Factor | | |
|---|---|---|---|
| miRNA | Oct4 | Sox2 | Nanog |
| mir-7-1 | | + | |
| mir-10a | + | | |
| mir-22 | | + | + |
| mir-32 | | + | + |
| mir-128a | | | + |
| mir-135b | | + | + |
| mir-137 | + | + | + |
| mir-196a-1 | | | + |
| mir-196b | + | | |
| mir-204 | | + | + |
| mir-205 | | + | + |
| mir-301 | + | + | + |
| mir-361 | | | + |
| mir-448 | + | | |

Proximal binding of Oct4, Sox2 and Nanog to miRNAs from the RFAM database. Transcription factors bound are indicated by a “+”.

Our results suggest that miRNA genes which have been implicated in developmental processes are likely regulated by Oct4, Sox2 and Nanog in human ES cells and are important components of the transcriptional regulatory circuitry in these cells.

### Example 3

### ES Cell Transcription Factors Occupy Active and Inactive Genes

Oct4 and Sox2 are known to be involved in both gene activation and repression in vivo (Botquin et al., 1998; Nishimoto et al., 1999; Yuan et al., 1995), so we sought to identify the transcriptional state of genes occupied by the stem cell regulators. To this end, the set of genes bound by Oct4, Sox2, and Nanog were compared to gene expression datasets generated from multiple ES cell lines (Abeyta et al., 2004; Brandenberger et al., 2004; Sato et al., 2003; Wei et al., 2005) to identify transcriptionally active and inactive genes (Table 3). The results showed that one or more of the stem cell transcription factors occupied 1303 actively transcribed genes and 957 inactive genes.

The importance of Oct4, Sox2, and Nanog for early development and ES cell identity led us to focus additional analyses on the set of 353 genes that are co-occupied by these regulators in human ES cells (Table S5). We first identified transcriptionally active genes. Transcripts were consistently detected in ES cells for approximately half of the genes co-bound by Oct4, Sox2, and Nanog. Among these active genes, several encoding transcription factors (e.g. POU5F1/OCT4, SOX2, NANOG, STAT3, ZIC3) and components of the Tgf-β (e.g. TDGF1, LEFTY2/EBAF) and Wnt (e.g. DKK1, FRAT2) signaling pathways were notable targets. Recent studies have shown that Tgf-β and Wnt signaling play a role in pluripotency and self-renewal in both mouse and human ES cells (James et al., 2005; Sato et al., 2004). These observations suggest that Oct4, Sox2, and Nanog promote pluripotency and self-renewal through positive regulation of their own genes and genes encoding components of these key signaling pathways.

Among transcriptionally inactive genes co-occupied by Oct4, Sox2, and Nanog, we noted a striking enrichment for transcription factor genes (p<10⁻¹⁸), many of which have been implicated in developmental processes. These included genes that specify transcription factors important for differentiation into extra-embryonic, endodermal, mesodermal, and ectodermal lineages (e.g. ESX1l, HOXB1, MEIS1, PAX6, LHX5, LBX1, MYF5, ONECUT1). Moreover, nearly half of the transcription factor genes that were bound by the three regulators and transcriptionally inactive encoded developmentally important homeodomain proteins (Table 2). These results demonstrate that Oct4, Sox2, and Nanog occupy a set of repressed genes that are key to developmental processes.

TABLE 2
Examples of Inactive Homeodomain Genes
Co-occupied by Oct4, Sox2, and Nanog

| Gene | Entrez | |
|---|---|---|
| Symbol | Gene ID | Gene Name |
| ATBF1 | 463 | AT-binding transcription factor 1 |
| DLX1 | 1745 | distal-less homeo box 1 |
| DLX4 | 1748 | distal-less homeo box 4 |
| DLX5 | 1749 | distal-less homeo box 5 |
| EN1 | 2019 | engrailed homolog 1 |
| ESX1L | 80712 | extraembryonic, spermatogenesis, homeobox |
| | | 1-like |
| GBX2 | 2637 | gastrulation brain homeobox 2 |
| GSC | 145258 | goosecoid |
| HOP | 84525 | homeodomain-only protein |
| HOXB1 | 3211 | homeo box B1 |
| HOXB3 | 3213 | homeo box B3 |
| HOXC4 | 3221 | homeo box C4 |
| IPF2 | 3651 | insulin promoter factor 2 |
| ISL1 | 3670 | ISL1 transcription factor, LIM/homeodomain |
| | | (islet-1) |
| LBX1 | 10660 | transcription factor similar to D. melanogaster |
| | | homeodomain protein lady bird late |
| LHX2 | 9355 | LIM homeobox 2 |
| LHX5 | 64211 | LIM homeobox 5 |
| MEIS1 | 4211 | myeloid ecotropic viral integration site 1 |
| | | homolog (mouse) |
| NKX2-2 | 4821 | NK2 transcription factor related, locus 2 |
| | | (Drosophila) |
| NKX2-3 | 159296 | NK2 transcription factor related, locus 3 |
| | | (Drosophila) |
| ONECUT1 | 3175 | one cut domain, family member 1 |
| OTP | 23440 | orthopedia homolog (Drosophila) |
| OTX1 | 5013 | orthodenticle homolog 1(Drosophila) |
| PAX6 | 5080 | paired box gene 6 |
| TITF1 | 7080 | thyroid transcription factor 1 |

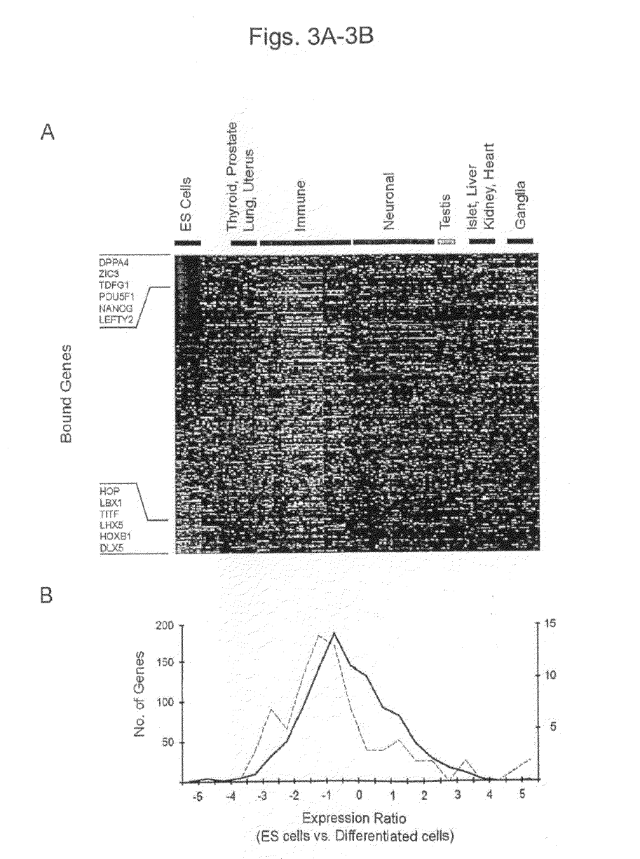

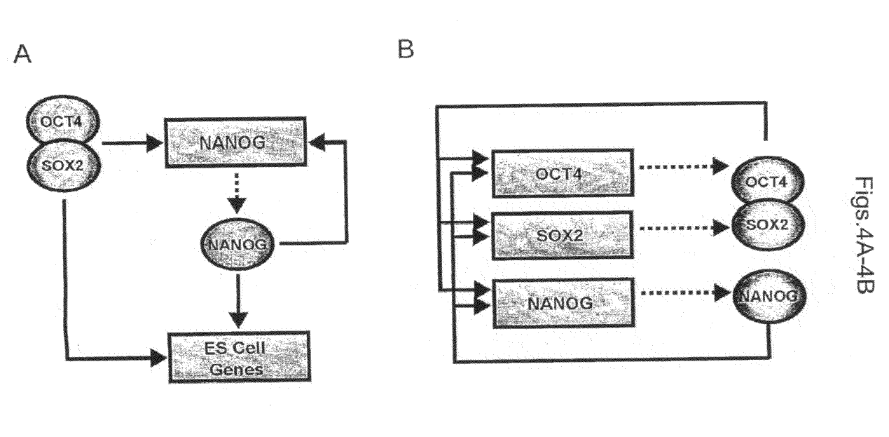

To determine which of the Oct4, Sox2, and Nanog bound genes were preferentially expressed in ES cells, we compared expression datasets (Abeyta et al., 2004; Sato et al., 2003) from ES cells and a compendium of differentiated tissues and cell types (Su et al., 2004) (FIG. 3). It was notable that DPPA4, TDGF1, OCT4, NANOG, and LEFTY2 were at the top of the rank order list of genes that are bound and preferentially expressed in ES cells (FIG. 3A). All five of these genes have been implicated in pluripotency (James et al., 2005; Mitsui et al., 2003; Chambers et al., 2003; Nichols et al., 1998; Bortvin et al., 2003). Moreover, several genes that encode developmentally important homeodomain proteins such as DLX5, HOXB1, LHX5, TITF1, LBX1, and HOP were at the bottom of this list, indicating that they are preferentially repressed in ES cells.

The observation that Oct4, Sox2, and Nanog bound to transcriptionally active genes that have roles in pluripotency and transcriptionally inactive genes that promote development suggests that these binding events are regulatory. Two additional lines of evidence indicated that many of the binding events identified in this study contribute to regulation of their target genes. First, some of the genes identified here (e.g., OCT4, SOX2, and NANOG) were previously shown to be regulated by Oct4 and Sox2 in mouse ES cells (Catena et al., 2004; Kuroda et al., 2005, Okumura-Nakanishi et al., 2005; Rodda et al., 2005). Second, we further explored the hypothesis that bound genes are regulated by these transcription factors by taking advantage of the fact that Oct4 and Nanog are expressed in ES cells but their expression is rapidly downregulated upon differentiation. We compared the expression of Oct4, Sox2 and Nanog occupied genes in human ES cells with expression patterns in 79 differentiated cell types (Su et al., 2004) and focused the analysis on transcription factor genes because these were the dominant functional class targeted by the ES cell regulators (FIG. 3B). We expected that for any set of genes that there would be a characteristic change in expression levels between ES cells and differentiated cells. If Oct4, Sox2 and Nanog do not regulate the genes they occupy, then these genes should have the same general expression profile as the control population. We found, however, a significant shift in the distribution of expression changes for genes occupied by Oct4, Sox2 and Nanog (p-value <0.001). Taken together, these data support the model that Oct4, Sox2 and Nanog functionally regulate the genes they occupy and suggest that loss of these regulators upon differentiation results in increased expression of genes necessary for development and reduced expression of a set of genes required for the maintenance of stem cell identity.

Our results suggest that Oct4, Sox2, and Nanog contribute to pluripotency and self-renewal by activating their own genes and genes encoding components of key signaling pathways and by repressing genes that are key to developmental processes. It is presently unclear how the three key regulators can activate some genes and repress others. It is likely that the activity of these key transcription factors is further controlled by additional cofactors, the precise levels of Oct4, Sox2, and Nanog, and by post-translational modifications.

### Example 4

### Core Transcriptional Regulatory Circuitry in ES Cells

In order to identify regulatory network motifs associated with Oct4, Sox2 and Nanog, we assumed that regulator binding to a gene implies regulatory control and used algorithms that were previously devised to discover such regulatory circuits in yeast (Lee et al., 2002). The simplest units of commonly used transcriptional regulatory network architecture, or network motifs, provide specific regulatory capacities such as positive and negative feedback loops to control the levels of their components (Lee et al., 2002; Milo et al., 2002; Shen-Orr et al., 2002).

Our data indicated that Oct4, Sox2 and Nanog form feedforward loops that involve at least 353 protein coding and 2 miRNA genes (FIG. 4A). Feedforward loop motifs contain a regulator that controls a second regulator, and have the additional feature that both regulators bind a common target gene. The feedforward loop has multiple regulatory capacities that may be especially useful for stem cells. When both regulators are positive, the feedforward loop can provide consistent activity that is relatively insensitive to transient changes in input (Mangan et al., 2003; Shen-Orr et al., 2002). If the regulators have positive and negative functions, the feedforward loop can act as a switch that enables a rapid response to inputs by providing a time-sensitive delay where the downstream regulator acts to counter the effects of the upstream regulator in a delayed fashion (Mangan and Alon, 2003; Mangan et al., 2003). In ES cells, both regulatory capacities could be useful for maintaining the pluripotent state while retaining the ability to react appropriately to differentiation signals. Previous studies have shown that feedforward loop architecture has been highly favored during the evolution of transcriptional regulatory networks in less complex eukaryotes (Lee et al., 2002; Ma et al., 2004; Milo et al., 2002; Resendis-Antonio et al., 2005; Shen-Orr et al., 2002). Our data suggest that feedforward regulation is an important feature of human ES cells as well.

Our results also showed that Oct4, Sox2, and Nanog together bound to the promoters of their own genes, forming interconnected autoregulatory loops (FIG. 4B). Transcriptional regulation of Oct4, Sox2, and Nanog by the Oct4-Sox2 complex was recently described in murine ES cells (Catena et al., 2004; Kuroda et al., 2005; Okumura-Nakanishi et al., 2005; Rodda et al., 2005). Our data indicate that this autoregulatory loop is conserved in human ES cells and more importantly, that Nanog is a component of the regulatory apparatus at these genes. Thus, it is likely that the expression and function of these three key stem cell factors are inextricably linked to one another. Autoregulation is thought to provide several advantages, including reduced response time to environmental stimuli and increased stability of gene expression (McAdams and Arkin, 1997; Rosenfeld et al., 2002; Shen-Orr et al., 2002; Thieffry et al., 1998).

The autoregulatory and feedforward circuitry described here may provide regulatory mechanisms by which stem cell identity can be robustly maintained, yet permit cells to respond appropriately to developmental cues. Modifying Oct4 and Nanog levels and function can change the developmental potential of murine ES cells (Avilion et al., 2003; Chambers et al., 2003; Mitsui et al., 2003; Nichols et al., 1998; Niwa et al., 2000) and this might be interpreted as being a consequence of perturbing independent regulatory pathways under the control of these two regulators. Our results argue that the levels and functions of these key stem cell regulators are tightly linked at both target genes and at their own promoters and thus provide an additional framework for interpreting the genetic studies. Changes in the relative stoichiometry of these factors would disturb the autoregulatory and feedforward circuitry, producing changes in global gene regulation and thus cell fate.

### Example 5

### Expanded Transcriptional Regulatory Circuitry

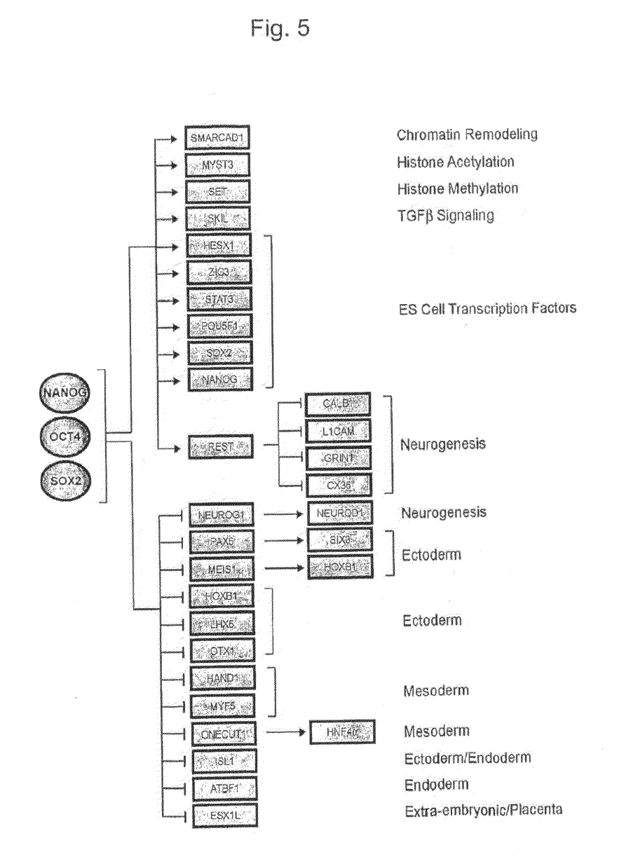

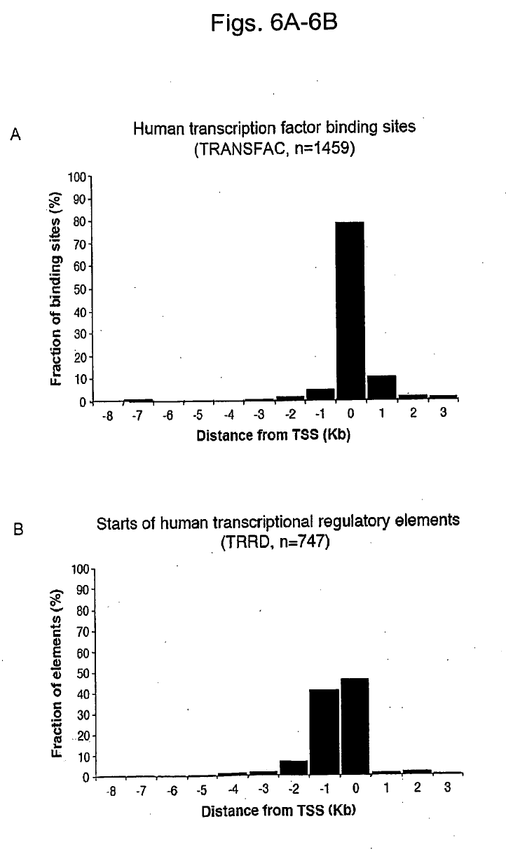

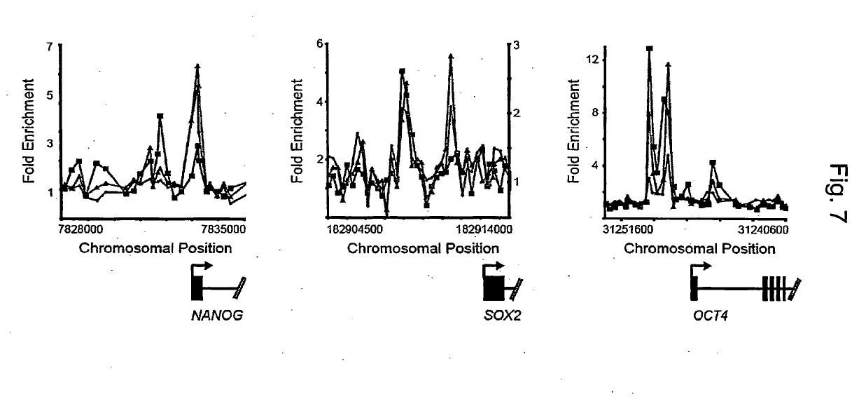

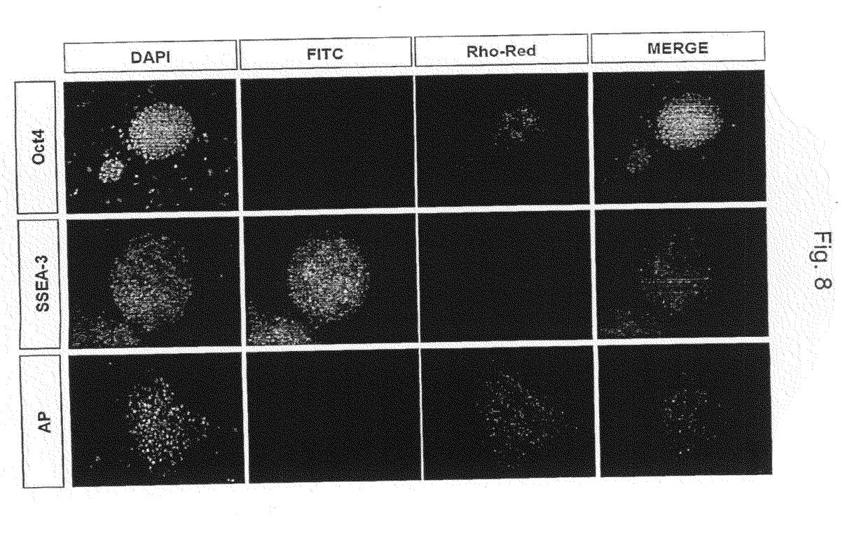

An initial model for ES cell transcriptional regulatory circuitry was constructed by identifying Oct4, Sox2, and Nanog target genes that encode transcription factors and chromatin regulators, and integrating knowledge of the functions of these downstream regulators in both human and mouse based on the available expression studies and literature (FIG. 5). The model includes a subset of active and a subset of repressed target genes based on the extensive expression characterization of the 353 co-bound genes as described earlier. The active targets include genes encoding components of chromatin remodeling and histone modifying complexes (e.g. SMARCAD1, MYST3, and SET), which may have general roles in transcriptional regulation and genes encoding transcription factors (e.g. REST, SKIL, HESX1, STAT3) which themselves are known to regulate specific genes. For instance, the REST protein has recently been shown to be present at high levels in ES cells and is required for the coordinate expression of a number of genes during neurogenesis, in part through repression of neuronal genes in the inappropriate cell type (Ballas et al., 2005). Previous studies have proposed that Nanog may function through the Tgf-β pathway in ES cells (Chambers, 2004). Our model suggests that this occurs through direct regulation of key components of this pathway (e.g. TDFG1, LEFTY2/EBAF) and through regulation of at least one transcription factor, SKIL, which controls the activity of downstream components of this pathway (SMAD2, SMAD4) (He et al., 2003). Our data also reveal that Oct4, Sox2, and Nanog co-occupy STAT3, a key regulator of self-renewal in mouse ES cells (Chambers, 2004), suggesting that Stat3 may also play a role in human ES cells.

The model described in FIG. 5 also depicts a subset of the genes bound by Oct4, Sox2, and Nanog that are inactive and that encode transcription factors that have key roles in differentiation and development. These include regulators with demonstrated roles in development of all embryonic lineages. This initial model for ES cell transcriptional regulatory circuitry is consistent with previous genetic studies in mice that suggest that Oct4 and Nanog maintain pluripotency through repression of differentiation programs (Chambers et al., 2003; Mitsui et al., 2003; Niwa et al., 2000). This model also provides a mechanistic framework for understanding bow this is accomplished through regulation of specific sets of genes that control cell fate specification.

Discovering how gene expression programs are controlled in living cells promises to improve our understanding of cell biology, development and human health. Identifying the target genes for key transcriptional regulators of human stem cells is a first critical step in the process of understanding these transcriptional regulatory networks and learning how they control cell identity. Mapping Oct4, Sox2, and Nanog to their binding sites within known promoters has revealed that these regulators collaborate to form regulatory circuitry in ES cells consisting of specialized autoregulatory and feedforward loops. Continued advances in our ability to culture and genetically manipulate human ES cells will allow us to test and manipulate this circuitry. Identification of the targets of additional transcription factors and chromatin regulators using the approaches described here should allow investigators to produce a more comprehensive map of transcriptional regulatory circuitry in these cells. Connecting signaling pathways to this circuit map may reveal how these pluripotent cells can be stimulated to differentiate into different cell types or how to reprogram differentiated cells back to a pluripotent state.

Experimental procedures were carried out as follows:

### Chromatin Immunoprecipitation Assays

Protocols describing all materials and methods can be downloaded from http://jura.wi.mit.edu/young/hESRegulation/.

Human embryonic stem cells were grown to a final count of 5×10⁷-1×10⁸ cells for each location analysis reaction. Cells were chemically crosslinked by the addition of one-tenth volume of fresh 11% formaldehyde solution for 15 minutes at room temperature. Cells were rinsed twice with 1×PBS and harvested using a silicon scraper and flash frozen in liquid nitrogen and stored at −80° C. prior to use. Cells were resuspended, lysed in lysis buffers and sonicated to solubilize and shear crosslinked DNA. Sonication conditions vary depending on cells, culture conditions, crosslinking and equipment. We used a Misonix Sonicator 3000 and sonicated at power 7 for 10×30 second pulses (90 second pause between pulses) at 4° C. while samples were immersed in an ice bath. The resulting whole cell extract was incubated overnight at 4° C. with 100 μl of Dynal Protein G magnetic beads that had been preincubated with 10 μg of the appropriate antibody. Beads were washed 5 times with RIPA buffer and 1 time with TE containing 50 mM NaCl. Bound complexes were eluted from the beads by heating at 65° C. with occasional vortexing and crosslinking was reversed by overnight incubation at 65° C. Whole cell extract DNA (reserved from the sonication step) was also treated for crosslink reversal. Immunoprecipitated DNA and whole cell extract DNA were then purified by treatment with RNAseA, proteinase K and multiple phenol:chloroformisoamyl alcohol extractions. Purified DNA was blunted and ligated to linker and amplified using a two-stage PCR protocol. Amplified DNA was labeled and purified using Invitrogen Bioprime random primer labeling kits (immunoenriched DNA was labeled with Cy5 fluorophore, whole cell extract DNA was labeled with Cy3 fluorophore). Labeled DNA was combined (5-6 μg each of immunoenriched and whole cell extract DNA) and hybridized to arrays in Agilent hybridization chambers for 40 hours at 40° C. Arrays were then washed and scanned.

### Control ChIPs

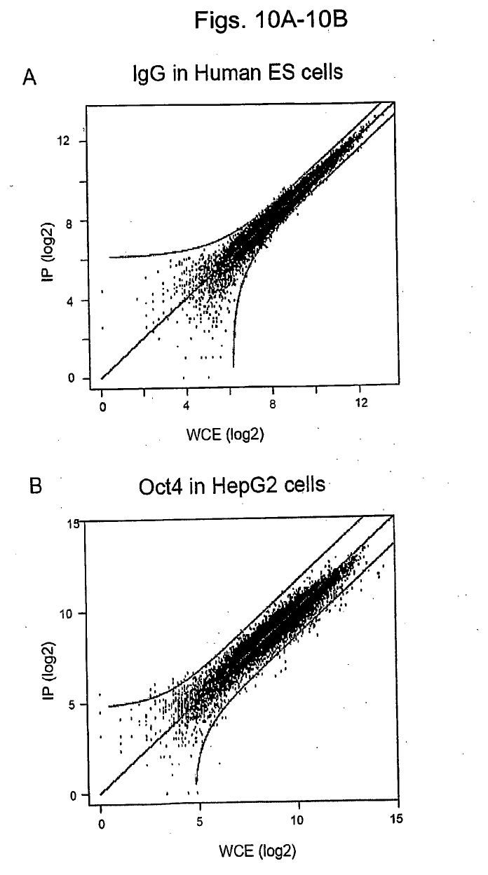

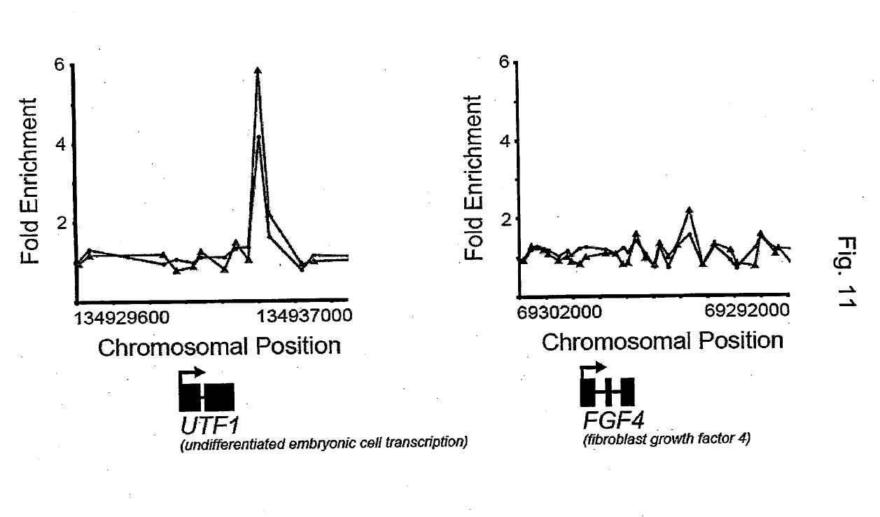

Location analysis experiments were performed with both rabbit and goat IgG in human ES cells as a negative control. We did not find any enrichment for sequences occupied by Oct4, Sox2 and Nanog (FIG. 10). Control ChIP experiments were also performed with E2F4. We did not observe any substantial overlap among the Oct4, Sox2, and Nanog targets and E2F4 target genes. Additional control experiments were performed to address the potential cross-reactivity of the antibodies to other family members FIG. 10). We carried out CHIP with Oct4 (sc-8628 Santa Cruz), Sox2 (AF2018 R&D Systems), and Nanog (AF1997 R&D Systems) antibodies in HepG2 cells that do not express these factors, but which express other POU and HMG domain proteins. This experiment did not yield any significantly enriched targets.

### Array Design

The following describes the design of the 10-slide promoter arrays that contain approximately 400,000 features used in this study. Arrays were produced by Agilent Technologies (www.agilent.com). All microarray data is available at ArrayExpress under the following accession designation E-WMIT-5.

### Selection of Regions and Design of Subsequences

To select well-characterized transcription start sites, we first collected the coordinates of all transcription start sites described in five different databases: RefSeq, Ensembl, MGC, VEGA (www.vega.sanger.ac.uk) and Broad (www.broad.mit.edu). The first three are commonly used databases for gene annotation, the last two are manually annotated databases covering subsets of the human genome from the Sanger Institute and Broad Institute, respectively. We then filtered for all transcription start sites that appeared in any two of these five databases (start sites separated by less than 500 bp in any of the databases were considered identical for this step). In cases where there were multiple start sites with different genomic coordinates, we selected the start site that would result in the longest transcript. A total of 18,002 start sites were selected. All sequences and coordinates are from the May 2004 build of the human genome (NCBI build 35), using the repeatmasked (-s) option which separates the genome into masked and unmasked subsequences. We used the program ArrayOligoSelector (AOS, Bozdech et al., 2004) to score 60-mers for every unmasked subsequence greater than 62 bp across all promoter regions. The scores for each oligo were retained but not put through the built-in AOS selection process.

The collection of scored 60-mers was divided by promoter and sorted by genomic position. Each set of 60-mers was then filtered based on the oligo scoring criteria. AOS uses a scoring system for four criteria: GC content, self-binding, complexity and uniqueness. For our most stringent filter, we selected the following ranges for each parameter: GC content between 30 percent and 100 percent, self-binding score less than 100, complexity score less than or equal to 24, uniqueness greater than or equal to −40.

From this subset of 60-mers, we selected oligos designed to cover the promoter region with an estimated density of one probe every 280 basepairs. To achieve more uniform tiling, we instituted a simple method to find probes within a particular distance from each other. Starting at the upstream end of the region, we selected the first qualified probe, then selected the next qualified probe that was between 150 bp and 280 bp away. If there were multiple, eligible probes, we chose the most distal probe within the 280 bp limit. If there were no probes within this limit, we continued scanning until we found the next acceptable probe. The process was then repeated with the most recently selected probe until we reached the end of the promoter region.

For regions that were not covered by high quality probes, we returned to the full set of scored 60-mers and filtered using less stringent criteria. This gave us an additional set of 60-mers that we then used to fill gaps in our coverage. After this second pass, we identified gaps in our coverage and added oligos that were properly spaced and best fit our criteria regardless of whether they passed the filter cutoffs. This iterative process gave us a compromise between optimal probe quality and optimal probe spacing. For each start site, we selected the region 8 kb upstream and 2 kb downstream of the site for tiling.

### Compiled Probes and Controls

The design process described was used to generate a set of 10 Agilent microarrays containing a total of 399,309 features designed for 18,002 transcription start sites representing 17,917 unique genes. Each array contains between 39,904 and 39,961 features. The probes are arranged such that array 1 begins with the first qualified transcription start site on the left arm of chromosome 1, array 2 picks up where array 1 ends, array 3 picks up where array 2 ends, and so on. There are some gaps in coverage that reflect our inability to identify high quality unique 60-mers: these tend to be unsequenced regions, highly repetitive regions that are not repeat masked (such as telomeres or gene families) and certain regions that are probably genome duplications. As an estimate of probe density, approximately 96% of all 60-mers are within 400 bp of another 60-mer; approximately 90% of all 60-mers are within 280 bp of another 60-mer.

We added several sets of control probes (2,043 total) to the array designs. On each array, there are 40 oligos designed against six Arabidopsis thaliana genes and printed in triplicate. These Arabidopsis oligos have been carried over from previous array designs and were intended for eventual use with spike-in controls. These oligos were BLASTed against the human genome and did not register any significant hits. An additional 543 Arabidopsis oligos were selected as negative controls based on their failure to show any significant BLAST hits against the human genome. Since E2F4 chromatin immunoprecipitations can be accomplished with a wide range of cell types and have provided a convenient positive control for ChIP-Chip experiments (for putative regulators where no prior knowledge of targets exist, for example), we added a total of 80 oligos representing four proximal promoter regions of genes that are known targets of the transcriptional regulator E2F4 (NM_{—}001211, NM_{—}002907, NM_{—}031423, NM_{—}001237). Each of the four promoters is represented by 20 different oligos that are evenly positioned across the region from 3 kb upstream to 2 kb downstream of the transcription start site. We also included a control probe set that provides a means to normalize intensities across multiple slides throughout the entire signal range. There are 384 oligos printed as intensity controls; based on test hybridizations, this set of oligos gives signal intensities that cover the entire dynamic range of the array. Twenty additional intensity controls, representing the entire range of intensities, were selected and printed fifteen times each for an additional 300 control features. We also incorporated 616 “gene desert” controls. To design these probes, we identified intergenic regions of 1 Mb or greater and designed probes in the middle of these regions. These are intended to identify genomic regions that are least likely to be bound by promoter-binding transcriptional regulators (by virtue of their extreme distance from any known gene). We have used these as normalization controls in situations where a factor binds to a large number of promoter regions. In addition to these 2,043 controls, there are 2,256 controls added by Agilent (standard) and a variable number of blank spots bringing the total number of features on each slide to 44,290.

| | Start | | End | | |
|---|---|---|---|---|---|
| Slide | Chr | Pos | Chr | Pos | Probes |
| 1 | 1 | 5575 | 1 | 224646230 | 39961 |
| 2 | 1 | 224694779 | 3 | 108726269 | 39909 |
| 3 | 3 | 109290599 | 5 | 147564193 | 39937 |
| 4 | 5 | 147665548 | 7 | 106280884 | 39935 |
| 5 | 7 | 106395416 | 10 | 15044190 | 39925 |
| 6 | 10 | 15119596 | 11 | 129697251 | 39905 |
| 7 | 11 | 129802259 | 14 | 94119500 | 39930 |
| 8 | 14 | 94140702 | 17 | 41335175 | 39938 |
| 9 | 17 | 41603407 | 20 | 30042900 | 39940 |
| 10 | 20 | 30054185 | Y | 57685547 | 39930 |

### Replicate Data Sets

Multiple batches of ES cells were cultured and each was tested for expression of pluripotency markers and the potential to differentiate into derivatives of the three embryonic lineages. Independent batches of ES cells were used to perform independent ChIP experiments as described above for each of the three transcription factors. Biological replicates were performed with the same Nanog (AF1997 R&D Systems) and Sox2 (AF2018 R&D Systems) antibody or two different antibodies against Oct4 (Sc-8628 Santa Cruz; AF1759 R&D Systems). ChIPs for each of the three different transcription factors were hybridized to independent Agilent array sets.

### Array Scan and Data Extraction

Slides were scanned using an Agilent DNA microarray scanner BA. PMT settings were set manually to normalize bulk signal in the Cy3 and Cy5 channel. For efficient batch processing of scans, we used GenePix (version 6.0) software. Scans were automatically aligned and then manually examined for abnormal features. Intensity data were then extracted in batch.

### Data Normalization and Analysis

GenePix software was used to obtain background-subtracted intensity values for each fluorophore for every feature on the array. To obtain set-normalized intensities, we first calculated, for each slide, the median intensities in each channel for a set of 1,420 control probes that are included on each array. We then calculated the average of these median intensities for the set of 10 slides. Intensities were then normalized such that the median intensity of each channel for an individual slide equaled the average of the median intensities of that channel across all slides.

Each slide contains a set of negative control spots that contain 60-mer sequences that do not cross-hybridize to human genomic DNA. We calculated the median intensity of these negative control spots in each channel and then subtracted this number from the set-normalized intensities of all other features.

To correct for different amounts of genomic and immunoprecipitated DNA hybridized to the chip, the set-normalized, negative control-subtracted median intensity value of the IP-enriched DNA channel was then divided by the median of the genomic DNA channel. This yielded a normalization factor that was applied to each intensity in the genomic DNA channel.

Next, we calculated the log of the ratio of intensity in the IP-enriched channel to intensity in the genomic DNA channel for each probe and used a whole chip error model (Hughes et al., 2000) to calculate confidence values for each spot on each array (single probe p-value). This error model functions by converting the intensity information in both channels to an X score which is dependent on both the absolute value of intensities and background noise in each channel. The X scores for an array are assumed to be normally distributed which allows for calculation of a p-value for the enrichment ratio seen at each feature.

### Identification of Bound Regions

To automatically determine bound regions in the datasets, we developed an algorithm to incorporate information from neighboring probes. For each 60-mer, we calculated the average X score of the 60-mer and its two immediate neighbors. If a feature was flagged as abnormal during scanning, we assumed it gave a neutral contribution to the average X score. Similarly, if an adjacent feature was beyond a reasonable distance from the probe (1000 bp), we assumed it gave a neutral contribution to the average X score. The distance threshold of 1000 bp was determined based on the maximum size of labeled DNA fragments hybridized to the array. Since the maximum fragment size was approximately 550 bp, we reasoned that probes separated by 1000 or more bp would not be able to contribute reliable information about a binding event halfway between them.

This set of averaged values gave us a new distribution that was subsequently used to calculate p-values of average X (probe set p-values). If the probe set p-value was less than 0.001, the three probes were marked as potentially bound.

As most probes were spaced within the resolution limit of chromatin immunoprecipitation, we next required that multiple probes in the probe set provide evidence of a binding event. Candidate bound probe sets were required to pass one of two additional filters: two of the three probes in a probe set must each have single probe p-values <0.005 or the center probe in the probe set has a single probe p-value <0.001 and one of the flanking probes has a single point p-value <0.1. These two filters cover situations where a binding event occurs midway between two probes and each weakly detects the event or where a binding event occurs very close to one probe and is very weakly detected by a neighboring probe. Individual probe sets that passed these criteria and were spaced closely together were collapsed into bound regions if the center probes of the probe sets were within 1000 bp of each other.

### Comparing Transcription Factor Bound Regions to Known Genes

The coordinates for the complete list of bound regions can be found in Tables S1, S3, S4, and S6 (see Index of Tables).

### Comparisons to Known Genes

The location of all bound regions were compared to a composite database of genes compiled from three databases: RefSeq (Pruitt et al., 2005), Mammalian Gene Collection (MGC) (Gerhard et al., 2004), and Ensembl (Hubbard et al., 2005). This database was generated by compiling genes with Entrez Gene IDs, and adding additional genes or transcription start sites from the above databases as necessary. Transcripts that overlapped multiple non-nested genes on the same strand were not used. By this method, 22,200 unique genes were identified. Genes lacking formal names are identified by their transcript ID number. All coordinate information was downloaded in January 2005 from the UCSC Genome Browser (NCBI build 35). The annotated gene lists are available for download from our website (web.wi.mit.edu/young/hESregulation/).

### Analysis of Error Rates in Location Analysis Experiments

Estimating a false positive and false negative rate is challenging as the estimates depend on perfect knowledge of a ground truth or confirmation by other experimental techniques that will each have their own bias. For the array platform used here, our experience with yeast provides an estimate of the error inherent in the platform. In this case, we selected a set of positives and negatives for the binding of Gcn4, a well-studied yeast transcription factor. The 84 positive genes were selected using three criteria: previous high confidence binding data (P≦0.001) (Harbison et al., 2004), the presence of a perfect or near perfect Gcn4 consensus binding site (TGASTCA) in the promoter region (−400 bp to +50 bp), and a greater then 2-fold change in steady state mRNA levels dependent on Gcn4 when shifted to amino acid starvation medium (Natarajan et al., 2001). The negative list of 222 genes was selected by weak binding (P≧0.1), absence of a motif near the presumed start site, and less then a 20% change in steady state mRNA levels in response to shift to amino acid starvation.

Using these positive and negative sets, we used ROC curve analysis (Statistics-ROC package for Perl) to evaluate a range of different IP/WCE ratio thresholds for false positive and false negative rates. Essentially, we examined a range of thresholds to denote “bound” and asked how many false positives and false negatives were detected at each threshold. Each gene was scored based on the maximum median-normalized IP/WCE ratio found in the region −250 to +50 bp from the UAS. With the optimal cutoff for minimizing false positives (a 3.5 fold ratio), the data suggest a false positive rate of less than 0.5% and a false negative rate of ˜20%. Thus, the oligo array platform is capable of generating extremely accurate, high quality data.

### Comparing Binding and Expression Data

### Processing Gene Expression Data

MPSS data: Three MPSS datasets were collected, two from a pool of the ES cell lines H1, H7 and H9 and one for HES-2 (Brandenberger et al., 2004; Wei et al., 2005). For each study, only MPSS tags detected at or over 4 transcripts per million (tpm) were used for further analysis. In addition, the data provided by Wei and colleagues (2005) allowed us to select only those tags that could be mapped to a single unique location in the human genome. For tags without a corresponding EntrezGene ID, IDs were assigned using the gene name or RNA accession numbers provided by the authors.

### Gene Expression Microarray Data:

Four Affymetrix HG-U133 gene expression datasets were collected for the cell lines H1, H9, HSF1 and HSF6 (Abeyta et al., 2004; Sato et al., 2003). EntrezGene IDs were assigned to the probe sets using Affymetrix annotation or using RNA accession numbers provided by the authors. For each probeset, we counted the number of “Present” calls in the three replicate array experiments performed for each cell line. Many genes are represented by more than one probeset and, to enable comparison to MPSS data, we then found the maximum number of P calls for each gene (defined by unique EntrezGene D). In each study, the cell lines were analyzed is triplicate. A gene was defined as detected if it was called “Present” in at least 2 of the 3 replicate experiments.

### Defining Expressed Genes Using Multiple Expression Datasets

In order for a gene to be defined as expressed, we required that the gene fit one of three criteria: detected in at least one MPSS experiment and at least one Affymetrix experiment, consistently detected across all three MPSS experiments or consistently detected across all four Affymetrix experiments. As described above, a gene was considered detected if present at 4 tpm or more by MPSS analysis or if two out of three Affymetrix replicates called the gene “Present”. These criteria allow us to capture the set of genes that were most consistently detected, including those genes where one experimental approach or the other is unable to detect expression due to technological limitations (for instance, genes detected by MPSS that are not included on the Affymetrix array).

Comparing Expression Patterns between ES cells and Differentiated Cells

We examined the relative expression levels of genes bound by Oct4, Sox2 and Nanog in ES cells compared to differentiated cell and tissue types. In order to compare ES cells with as many human cell and tissue types as possible, we combined the data from three studies, all performed using the Affymetrix HG-U133A platform: 3 replicates of H1 ES cells (Sato et al., 2003), 3 replicates each of H9, HSF1 and HSF6 ES cells (Abeyta et al., 2004) and 2 replicates of 79 other human cell and tissue types (Su et al., 2004). To generate a measurement of the expression changes between undifferentiated ES cells and differentiated cells, each dataset was scaled to 150 using GCOS (Affymetrix). Then, for each gene, ratios were generated from the median signal intensity of each gene across all experiments. EntrezGeneIDs were assigned to each probe-set and for genes with multiple probe-sets, the expression ratios averaged. This resulted in a final set of 12,968 unique genes. For each gene, the significance of relative overexpression in the 12 ES cell experiments versus the 158 non-ES cell experiments was identified using a Mann-Whitney U-test. This metric was used to order genes shown in FIG. 3A.

We further explored the hypothesis that bound genes are regulated by these transcription factors by taking advantage of the fact that Oct4 and Nanog are expressed in ES cells but their expression is rapidly downregulated upon differentiation. We compared the expression of Oct4, Sox2 and Nanog co-occupied genes in human ES cells with expression patterns in 79 differentiated cell types and focused the analysis on transcription factor genes because these were the dominant functional class targeted by the ES cell regulators (FIG. 3B). We expected that for any set of genes, there would be a characteristic change in expression levels between ES cells and differentiated cells. The distribution of fold change ratios (log base 2) was calculated for transcription factors bound by Oct4, Sox2 and Nanog and transcription factors not bound by any one of the three factors. If Oct4, Sox2 and Nanog do not regulate the genes they occupy, then these genes should have the same general expression profile as the control population. We found, however, a significant shift in the distribution of expression changes for genes occupied by Oct4, Sox2 and Nanog (p-value <0.001 using a two-sampled Kolmogorov-Smirnov test). The results for the H9 cell line are shown in FIG. 3B. Similar results were obtained when using any other ES cell line or when using the average of all four ES expression datasets.

Any factor-dependent effects on the profile could impact a combination of different characteristics, including the proportion of genes showing expression changes, the magnitude of changes or even whether the expression change is generally positive or negative. In general, these binding dependent effects on the profiles of sets of expression changes should be subtle. Many other factors are potentially contributing to the overall regulation of target genes and biologically relevant levels of gene expression changes may not be robust.

### Gene Ontology Classification

Gene Ontology datasets were downloaded from the NCBI and gene ontology websites in February 2005. P-values were calculated using RefSeq genes that are both represented on the promoter array set and that have an associated ontology.

### Oct4, Sox2 and Nanog Binding to the Oct4 Promoter Proximal Region

The oligo selection algorithm used for probe design has stringent criteria to assure the selection of unique and appropriately spaced probes covering each promoter of interest. However, this can result in an inability to find probes for some regions. In one case, the promoter region for Oct4 is poorly tiled on this set of arrays. As this promoter is one of the key targets in this study, we hybridized Oct4, Sox2 and Nanog immunoenriched material to a slide from a separate whole genome design that has more complete coverage of the Oct4 promoter region. Where possible, we used the exact same labeled, purified material (both IP and whole cell extract control) that was used on the original 10-slide set. The results indicate that all three factors co-occupy the same area of the Oct4 upstream (FIG. 7).

### Oct4 and Sox2 Binding to UTF1 and FGF4

UTF1 and FGF4 have been identified as key targets of Oct4 and Sox2 in mouse ES cells (Nishimoto et al., 1999; Yuan et al., 1995). II is not known if UTF1 or FGF4 play similar roles or whether these can be considered model target genes in human ES cells. The binding of these factors to their respective genes occurs at the 3′ UTR and these sequences are not included in our current array design. The inclusion of the appropriate human sequences for FGF4 and UTF1 could serve as useful positive controls for our analysis, so we sought to determine whether these genes are also bound by Oct4 and Sox2 in human ES cells. We designed an array to contain the appropriate sequences. When possible, we used the exact same labeled, purified material (both IP and whole cell extract control) that was used on the original 10-slide set and hybridized labeled material from an Oct4 and Sox2 ChIP to this array. We found that Oct4 and Sox2 do occupy the 3′UTR of the co-activator UTF1, but that these factors are absent from FGF4 in human ES cells (FIG. 11). This is consistent with the variable expression data with regard to FGF4 in human ES cells.

### Distribution of Oct4, Sox2, and Nanog Binding Relative to Transcription Start Sites

We designed the arrays against the −8 kb to +2 kb region relative to each transcription start site because binding events in these regions are most likely to be connected with regulation of the associated gene. It is possible that a binding event controls a neighboring or even distal gene. Indeed, the further the binding event from the transcription start site of a gene, the more likely that event is associated with control of another gene.

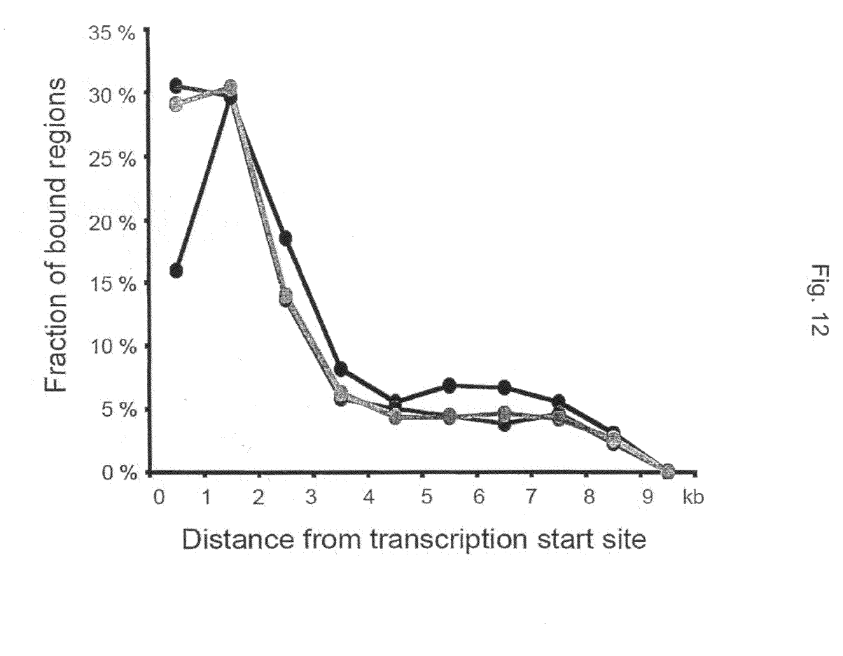

It was determined how often we find binding events in various portions of the −8 kb to +2 kb regions (FIG. 12). We found that 35-50% of the binding sites occurred within 1 kb of a transcriptions start site. We found that only a small portion (6%) of all the binding events we identified occur in the −8 kb to −7 kb region. We then measured the distance from the binding events that occur in the −8 kb to −7 kb region to the closest transcription start site. The transcription start site for an adjacent gene occurred within 8 kb in less than half of the cases (12, 31 and 37 binding events for Oct4, Sox2 and Nanog, respectively). For sites that are within 8 kb of multiple genes both genes were assigned as candidate targets. It would be difficult and perhaps inappropriate to assign one but not both proximal promoters as likely targets since it is known that transcription factor binding events can affect multiple adjacent promoters.

### Growth Conditions for Human Embryonic Stein Cells

Human embryonic stem (ES) cells were obtained from WiCell (Madison, Wis.; NIH Code WA09). Detailed protocol information on human ES cell growth conditions and culture reagents are available at http://www.mcb.harvard.edu/melton/hues. Briefly, passage 34 cells were grown in KO-DMEM medium supplemented with serum replacement, basic fibroblast growth factor (bFGF), recombinant human leukemia inhibitory factor (LIF) and a human plasma protein fraction. In order to minimize any MEF contribution in our analysis, H9 cells were cultured on a low density of irradiated murine embryonic fibroblasts (ICR MEFs) resulting in a ratio of approximately >8:1 H9 cell to MEF. The culture of H9 on low-density MEFs had no adverse affects on cell morphology, growth rate, or undifferentiated status as compared to cells grown under typical conditions. In addition, immunohistochemistry for pluripotency markers (e.g. Oct4, SSEA-3) indicated that H9 cells grown on a minimal feeder layer maintained the ability to generate derivates of ectoderm, mesoderm, and endoderm upon differentiation.

### Antibodies

The Nanog (AF1997 R&D Systems) and Sox2 (AF2018 R&D Systems) antibodies used in this study were immunoaffinity purified against the human protein and shown to recognize their target protein in Western blots and by immunocytochemistry (R&D Systems Minneapolis, Minn.). Multiple Oct4 antibodies directed against different portions of the protein were used for location analysis (AF1759 R&D Systems, sc-8628 Santa Cruz, sc-9081 Santa Cruz), some of which were immunoaffinity purified and have been shown to recognize their target protein in Western blots and by immunocytochemistry. Prior to conducting the experiments with Agilent arrays, we compared these three Oct4 antibodies by performing location analysis with self-printed promoter arrays and found that they performed similarly. ChIP experiments carried out with AF1759 and sc-8628 were hybridized to the Agilent 10-array sets. In addition, our immunofluorescence results indicated that a nuclear protein was detected only in undifferentiated ES cells with the Oct4 antibody (sc-9081) (FIG. 8; compare ES cell with MEF). E2F4 antibodies (sc-1082) were obtained from Santa Cruz Biotech and have been shown to specifically recognize previously reported E2F4 target genes (Table 3) (Ren et al., 2002; Weinsmann et al., 2002).

### Human ES Cell Controls

### Immunohistochemical Analysis of Pluripotency Markers

For analysis of pluripotency markers, cells were fixed in 4% paraformaldehyde for 30 minutes at room temperature and incubated overnight at 4° C. in blocking solution (5 ml Normal Donkey Solution: 195 ml PBS+0.1% Triton-X)(FIG. 8). After a brief wash in PBS, cells were then incubated with primary antibodies to Oct-3/4 (Santa Cruz sc-9081), SSEA-3 (MC-631; Solter and Knowles, 1979), SSEA-4 (MC-813-70; Solter and Knowles, 1979), Tra-1-60 (MAB4360; Chemicon International), and Tra-1-81 (MAB4381; Chemicon International) in blocking solution overnight at 4° C. Following incubation with primary antibody, cells were incubated with either rhodamine red or FITC-conjugated secondary antibody (Jackson Labs) for 2-5 hrs at 4° C. Nuclei were stained with 4′,6-diamidine-2-phenylidole dihydrochloride (DAPI). Epifluorescent images were obtained using a fluorescent microscope (Nikon TE300). Our analysis indicated that >80% of the H9 cells were strongly positive for all pluripotency markers. Alkaline phosphatase activity of human ES cells was analyzed using the Vector Red Alkaline Phosphatase Substrate Kit (Cat. No. SK-5100; Vector Laboratories) according to manufacturer's specifications and the reaction product was visualized using fluorescent microscopy.

### Teratoma Formation

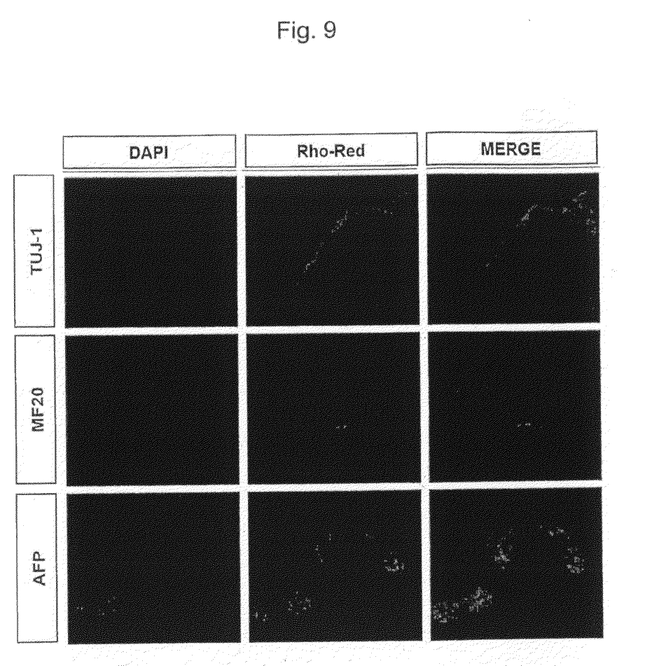

Teratomas were induced by injecting 2-5×10⁶ cells into the subcutaneous tissue above the rear haunch of 6 week old Nude Swiss (athymic, immunocompromised) mice. Eight to twelve weeks post-injection, teratomas were harvested and fixed overnight in 4% paraformaldehyde at 4° C. Samples were then immersed in 30% sucrose overnight before embedding the tissue in O.C.T freezing compound (Tissue-Tek). Cryosections were obtained and 10 μm sections were incubated with the appropriate antibodies as above and analyzed for the presence of the following differentiation markers by confocal microscopy (LSM 210): neuronal class II β-tubulin, Tuj1 (ectoderm; MMS-435P Covance); striated muscle-specific myosin, MF20 (mesoderm; kind gift from D. Fischman), and alphafetoprotein (endoderm; DAKO) (FIG. 9). 4′,6-diamidine-2-phenylidole dihydrochloride (DAPI) staining was used to identify nuclei. Antibody reactivity was detected for markers of all three germ layers confirming that the human embryonic stem cells used in our analysis had maintained differentiation potential.

### Embryoid Bodies (EB)

ES cells were harvested by enzymatic digestion and EBs were allowed to form by plating ˜1×10⁶ cells/well in suspension in 6-well non-adherent, low cluster dishes for 30 days. EBs were grown in the absence of leukemia inhibitory factor (LIF) and basic fibroblast growth factor (bFGF) in culture medium containing 2× serum replacement. EBs were then harvested, fixed for 30 minutes in 4% paraformaldehyde at room temperature, and placed in 30% sucrose overnight prior to embedding the tissue in O.C.T. freezing compound (Tissue-Tek). Cryosections were obtained as described for teratoma formation. Confocal images were obtained for all three germ layer markers again confirming that the H9 cells used in our analysis had maintained differentiation potential (data not shown; results similar to those shown in FIG. 9).

### Table 3:

TABLE 3
Oct4, Sox2, Nanog binding to Known Genes

| GENE | OCT4 | SOX2 | NANOG | E2F4 | Expressed |
|---|---|---|---|---|---|
| 13CDNA73 | — | — | 2003 | — | 0 |
| 182-FIP | — | 360 | — | — | 1 |
| 76P | — | — | — | 0 | 1 |
| 7h3 | — | — | — | 203 | 1 |
| AAMP | — | — | — | 0 | 1 |
| AASDH | 0 | 0 | 0 | — | 1 |
| AATF | — | — | — | 0 | 1 |
| ABC1 | — | — | — | 0 | 1 |
| ABCA5 | — | 4936 | 4936 | — | 0 |
| ABCB10 | — | 3818 | 3818 | — | 0 |
| ABCB6 | — | — | — | 0 | 1 |
| ABCB7 | — | 0 | 0 | — | 1 |
| ABCF2 | — | 31 | 31 | 31 | 1 |
| ABCG2 | — | — | 41 | — | 0 |
| ABHD11 | — | 4127 | 2246 | — | 1 |
| ABHD2 | — | — | 0 | — | 1 |
| ABHD8 | — | 5888 | 5888 | 1777 | 0 |
| ABLIM1 | — | 0 | — | — | 1 |
| ABTB2 | 820 | 820 | — | — | 1 |
| ACAD8 | — | — | — | 0 | 1 |
| ACADM | — | — | 799 | — | 1 |
| ACAT2 | — | — | 35 | — | 1 |
| ACCN1 | — | — | 675 | — | 0 |
| ACCN2 | — | — | 5968 | — | 0 |
| ACCN4 | 6895 | 6895 | 6895 | — | 0 |
| ACD | 5590 | — | — | 5590 | 1 |
| ACIN1 | — | 30 | — | — | 1 |
| ACO2 | — | 89 | 89 | — | 1 |
| ACOX1 | — | 140 | — | — | 1 |
| ACP2 | — | — | — | 3214 | 1 |
| ACSL4 | — | — | 871 | — | 1 |
| ACTN1 | — | — | — | 759 | 1 |
| ACTR1A | — | 0 | 0 | — | 1 |
| ACTR1B | — | — | 812 | 0 | 1 |
| ACYP1 | — | — | — | 0 | 1 |
| ACYP2 | — | — | — | 0 | 0 |
| ADAM28 | — | 0 | 0 | — | 0 |
| ADAMTS16 | 528 | 528 | 291 | — | 0 |
| ADAMTS19 | — | 538 | — | — | 0 |
| ADAMTS4 | — | — | 381 | — | 0 |
| ADAMTS5 | — | — | 2144 | — | 0 |
| ADAMTSL1 | 63 | 0 | 0 | — | 0 |
| ADAR | — | 707 | 707 | — | 1 |
| ADAT1 | — | — | — | 0 | 0 |
| ADD3 | 55 | 2683 | 2033 | — | 1 |
| ADFP | — | 1923 | 1489 | — | 1 |
| ADRA1A | 972 | 972 | 972 | — | 0 |
| ADRA2A | 369 | — | — | — | 0 |
| ADRBK2 | — | 42 | 559 | — | 1 |
| AEGP | — | — | — | 2779 | 0 |
| AF15Q14 | — | — | — | 0 | 1 |
| AGPAT4 | — | 1115 | — | — | 0 |
| AGPAT5 | — | — | — | 0 | 1 |
| AGTR2 | — | — | — | 25 | 0 |
| AHI1 | — | — | — | 0 | 1 |
| AHSA1 | — | — | 62 | — | 1 |
| AIP1 | — | 883 | — | — | 1 |
| AK3 | — | 983 | 708 | — | 1 |
| AK3L1 | — | — | 534 | — | 1 |
| AKT1S1 | — | 0 | — | — | 1 |
| ALCAM | — | 582 | 302 | — | 1 |
| ALDH16A1 | — | 966 | 966 | — | 0 |
| ALDH3A2 | — | — | — | 65 | 1 |
| ALDH6A1 | — | — | — | 266 | 1 |
| ALDH7A1 | — | 0 | — | — | 1 |
| ALDOA | — | — | — | 0 | 1 |
| ALG10 | — | — | — | 0 | 0 |
| ALG9 | — | — | 7413 | — | 1 |
| ALKBH | 0 | — | 0 | — | 1 |
| ALOX5 | — | — | — | 528 | 0 |
| ALOXE3 | 576 | — | — | — | 0 |
| ALPK3 | — | — | 1423 | — | 0 |
| ALPL | — | 625 | — | — | 1 |
| ALS2CR16 | — | — | 93 | 0 | 0 |
| AMIGO2 | 6018 | — | 5739 | — | 1 |
| AMOT | — | — | 310 | — | 0 |
| AMOTL1 | — | 3090 | 637 | — | 1 |
| ANAPC4 | — | — | 6536 | 7059 | 0 |
| ANGPT1 | — | — | 0 | — | 1 |
| ANKFY1 | — | — | — | 0 | 1 |
| ANKHD1 | 2659 | 0 | — | — | 1 |
| ANKMY2 | — | — | 2404 | — | 1 |
| ANKRD1 | 472 | 721 | 721 | — | 1 |
| ANKRD11 | — | — | — | 981 | 1 |
| ANKRD13 | — | — | — | 0 | 1 |
| ANKRD15 | 2272 | 1597 | 1597 | — | 1 |
| ANKS1 | — | — | — | 600 | 1 |
| ANLN | — | — | — | 0 | 1 |
| ANP32A | — | 2530 | 2530 | — | 1 |
| ANP32B | — | 511 | 511 | — | 1 |
| ANP32C | — | — | 6650 | 1057 | 0 |
| ANP32E | — | — | — | 0 | 1 |
| ANXA1 | — | — | 1668 | — | 1 |
| ANXA8 | — | — | 689 | — | 0 |
| AP1G1 | — | — | 0 | — | 1 |
| AP1S2 | — | — | 1967 | — | 1 |
| AP2A1 | — | 949 | 70 | — | 1 |
| AP2B1 | — | — | — | 0 | 1 |
| AP3B1 | — | 802 | 0 | — | 1 |
| AP3D1 | — | — | — | 0 | 1 |
| AP3S2 | — | — | — | 221 | 1 |
| APEX1 | — | 0 | — | — | 1 |
| APEX2 | — | — | 396 | — | 1 |
| APG4C | — | — | 0 | — | 1 |
| APH1A | — | 0 | 0 | — | 1 |
| APLP2 | — | — | 3917 | — | 1 |
| APOA2 | — | — | 1985 | — | 1 |
| APOB | — | — | 3042 | — | 1 |
| APOB48R | — | 2058 | 2058 | — | 0 |
| APOBEC3D | — | 0 | 0 | — | 0 |
| APOBEC3G | 6441 | 20 | 0 | — | 0 |
| APOM | — | — | 2875 | 2875 | 1 |
| APP | — | 1345 | — | — | 1 |
| AQP2 | — | 4546 | 4546 | — | 1 |
| AQP9 | — | — | 98 | — | 0 |
| ARCH | — | — | 0 | 89 | 0 |
| ARF3 | — | 7262 | 7660 | — | 1 |
| ARF4 | — | 0 | 0 | — | 1 |
| ARF4L | — | 0 | — | — | 1 |
| ARF6 | — | — | — | 0 | 1 |
| ARFGEF1 | — | 4758 | — | — | 1 |
| ARHGAP1 | — | 19 | 0 | — | 1 |
| ARHGAP11A | — | 0 | 0 | — | 1 |
| ARHGAP19 | — | — | 0 | 0 | 0 |
| ARHGEF17 | — | 1177 | — | — | 0 |
| ARID1A | — | — | — | 582 | 1 |
| ARID1B | 2966 | 2966 | 2686 | — | 1 |
| ARID4B | — | 0 | — | — | 1 |
| ARIH1 | — | 179 | 394 | — | 1 |
| ARL6 | — | 1024 | 1024 | — | 0 |
| ARL6IP6 | — | — | — | 0 | 1 |
| ARL8 | — | — | — | 304 | 1 |
| ARMC4 | — | — | 715 | — | 0 |
| ARMC6 | — | 803 | 803 | — | 1 |
| ARMCX1 | — | — | 0 | — | 1 |
| ARMCX5 | 0 | — | — | — | 1 |
| ARNTL2 | — | 325 | — | 99 | 0 |
| ARPC5 | 533 | — | — | — | 1 |
| ARPM1 | — | 2016 | 5759 | — | 0 |
| ARRDC3 | — | 1027 | — | — | 1 |
| ARSD | 2000 | 2000 | 546 | — | 0 |
| ARSF | — | — | 5346 | — | 0 |
| ARX | 2827 | — | 3039 | 2827 | 0 |
| ASAH1 | — | — | 0 | — | 1 |
| ASAM | — | — | 2816 | — | 1 |
| ASB1 | — | — | 271 | — | 1 |
| ASB18 | — | 7352 | — | — | 0 |
| ASCC2 | — | — | 3676 | — | 1 |
| ASCL2 | 6264 | — | 6589 | — | 0 |
| ASE-1 | — | — | — | 0 | 1 |
| ASF1B | — | — | — | 0 | 1 |
| ASH2L | — | — | — | 0 | 1 |
| ASK | — | — | — | 507 | 1 |
| ASNA1 | — | 54 | — | — | 1 |
| ASPM | — | — | — | 0 | 1 |
| ASXL1 | — | — | 615 | — | 1 |
| ATAD2 | 0 | 0 | 4 | 0 | 1 |
| ATAD3B | — | — | — | 0 | 0 |
| ATBF1 | 518 | 4127 | 787 | — | 0 |
| ATF2 | — | — | — | 4 | 1 |
| ATF3 | — | 1806 | — | — | 1 |
| ATF4 | — | 0 | 72 | 0 | 1 |
| ATF5 | — | — | — | 764 | 1 |
| ATF6 | — | 980 | — | — | 0 |
| ATF7IP | — | — | — | 0 | 1 |
| ATOH1 | — | 295 | — | — | 0 |
| ATOH8 | — | — | — | 57 | 0 |
| ATP11C | — | — | 518 | — | 1 |
| ATP5F1 | — | 0 | 0 | — | 1 |
| ATP6V1A | — | — | 1431 | — | 1 |
| ATP6V1D | 285 | — | — | 295 | 1 |
| ATP6V1G1 | 3693 | 3693 | 3693 | — | 1 |
| ATP7B | — | — | 786 | — | 0 |
| ATP8B3 | — | — | — | 0 | 0 |
| ATP9B | — | — | — | 0 | 1 |
| ATPAF2 | — | — | — | 0 | 1 |
| ATPIF1 | — | — | 0 | — | 1 |
| AUH | 461 | 461 | — | — | 1 |
| AUP1 | — | — | — | 0 | 1 |
| AVEN | — | 5 | — | — | 1 |
| AXIN2 | — | — | 0 | — | 1 |
| AZI1 | — | 123 | — | — | 1 |
| B2M | — | — | 0 | — | 1 |
| B3GALT4 | 5087 | 5087 | 5087 | 5221 | 1 |
| B4GALT6 | — | 1883 | 453 | 173 | 1 |
| BAG5 | 39 | — | 39 | 39 | 1 |
| BAMBI | 425 | 425 | 425 | — | 1 |
| BARHL2 | — | 3889 | — | — | 0 |
| BARX1 | — | — | — | 0 | 0 |
| BARX2 | — | — | 658 | — | 0 |
| BAT1 | — | — | — | 0 | 1 |
| BAT3 | — | — | 0 | 0 | 1 |
| BAT4 | — | — | — | 0 | 1 |
| BAT5 | — | 327 | — | — | 0 |
| BAZ1B | — | — | — | 131 | 1 |
| BBP | — | — | 0 | 0 | 1 |
| BC006122 | — | 0 | — | — | 0 |
| BC007862 | — | — | 0 | — | 1 |
| BC008141 | — | 6148 | — | — | 0 |
| BC008813 | — | — | 1686 | — | 0 |
| BC009415 | — | 422 | 702 | — | 1 |
| BC009550 | — | — | — | 567 | 0 |
| BC010732 | 662 | — | 903 | — | 0 |
| BC011742, | — | — | — | 0 | 0 |
| BC017255 | | | | | |
| BC012542 | — | — | 0 | 0 | 0 |
| BC013749 | — | — | — | 0 | 0 |
| BC014776 | — | — | 0 | — | 0 |
| BC014953 | — | — | 245 | — | 1 |
| BC016825 | — | 1198 | — | — | 1 |
| BC018206 | — | — | 37 | 37 | 1 |
| BC019341 | — | — | 274 | — | 1 |
| BC020923 | 235 | 235 | 235 | — | 0 |
| BC021127 | — | 1023 | 1023 | — | 1 |
| BC022357 | — | 0 | 5355 | 0 | 0 |
| BC022371 | — | 1687 | 1687 | — | 0 |
| BC023549 | — | — | 5159 | — | 0 |
| BC026345 | 16 | 0 | 0 | — | 0 |
| BC027178 | — | — | — | 435 | 1 |
| BC028301 | — | — | — | 0 | 0 |
| BC029609 | — | — | 0 | — | 0 |
| BC030245 | — | 7436 | 7436 | — | 1 |
| BC032347 | — | 0 | — | — | 0 |
| BC032958 | — | 672 | — | — | 0 |
| BC033154 | — | 26 | 26 | 0 | 0 |
| BC033643 | — | — | — | 0 | 0 |
| BC034752 | — | — | 2489 | — | 0 |
| BC036056 | — | — | — | 0 | 0 |
| BC039145 | — | 635 | 635 | — | 1 |
| BC040177 | — | — | 200 | — | 0 |
| BC040993 | — | — | 519 | — | 1 |
| BC044580 | — | 329 | 67 | 67 | 1 |
| BC045706 | — | — | 2242 | — | 0 |
| BC045834 | — | — | — | 0 | 0 |
| BC046199 | — | — | 268 | 268 | 1 |
| BC047471 | — | — | — | 0 | 0 |
| BC047718 | — | — | — | 0 | 0 |
| BC047782 | — | 284 | — | — | 0 |
| BC052341 | — | 0 | — | — | 1 |
| BC053996 | — | — | 2 | — | 0 |
| BC056682 | — | 2669 | 2669 | — | 1 |
| BC060847 | — | — | — | 598 | 0 |
| BC060851 | — | — | 5493 | — | 0 |
| BC060874 | — | — | 2688 | — | 0 |
| BC061909 | 82 | 82 | 82 | — | 1 |
| BC062632 | — | 1637 | — | — | 0 |
| BC063451 | — | — | 6294 | — | 0 |
| BC064390 | — | — | — | 0 | 1 |
| BC064584 | — | 0 | — | 0 | 1 |
| BC064948 | — | — | 74 | — | 1 |
| BC068520 | — | 124 | — | 373 | 0 |
| BC069363 | 3252 | 3532 | 3532 | — | 0 |
| BC071771 | — | 773 | — | — | 0 |
| BC075857 | — | — | 0 | 0 | 1 |
| BC080575 | — | — | — | 0 | 1 |
| BC080624 | 742 | — | 742 | — | 0 |
| BC-2 | — | — | — | 3655 | 1 |
| BCAR1 | — | — | 1828 | — | 0 |
| BCAS1 | — | — | 397 | — | 0 |
| BCAT1 | — | 595 | 595 | — | 1 |
| BCKDHA | — | 0 | 0 | 94 | 1 |
| BCL9 | — | 0 | 0 | — | 1 |
| BCL9L | — | — | 5597 | — | 1 |
| BCLAF1 | — | 1605 | 488 | — | 1 |
| BCS1L | — | — | — | 0 | 1 |
| BDH | 6964 | 6964 | 6964 | — | 0 |
| BFSP1 | — | — | 2622 | — | 0 |
| BG1 | — | 6075 | 6075 | — | 0 |
| BHLHB5 | 1095 | 815 | 0 | — | 0 |
| BIRC5 | — | — | — | 0 | 1 |
| BLCAP | 20 | — | 0 | — | 1 |
| BLM | — | — | — | 0 | 1 |
| BLOC1S1 | — | — | — | 0 | 1 |
| BLZF1 | — | 0 | 0 | — | 0 |
| BM039 | — | — | — | 0 | 1 |
| BMP2 | — | 4015 | 4015 | — | 1 |
| BMP5 | — | 0 | 0 | — | 0 |
| BMP7 | 2639 | 2639 | 2639 | — | 1 |
| BMPR1A | — | — | — | 826 | 1 |
| BMPR2 | — | 918 | 918 | — | 0 |
| BMSC-UbP | — | — | — | 0 | 1 |
| BMX | — | 148 | — | — | 0 |
| BNIP1 | — | — | 5128 | — | 1 |
| BRCA2 | — | — | — | 176 | 1 |
| BRCC2 | — | 0 | — | — | 0 |
| BRD8 | — | — | — | 0 | 1 |
| BRF1 | — | — | — | 0 | 0 |
| BRIP1 | — | 224 | — | 0 | 0 |
| BRMS1L | — | — | — | 0 | 0 |
| BSCL2 | — | — | 713 | — | 1 |
| BTBD6 | — | — | — | 0 | 1 |
| BTD | — | — | — | 49 | 1 |
| BTG1 | — | 0 | — | 0 | 1 |
| BTK | — | 4224 | — | — | 0 |
| BTRC | — | — | — | 186 | 1 |
| BUB1 | — | — | — | 0 | 1 |
| BUB1B | 5350 | 5350 | 5350 | 0 | 1 |
| BUB3 | 1189 | 0 | 0 | 0 | 1 |
| BZW2 | — | — | 1984 | — | 1 |
| C10orf117 | — | 0 | 0 | 0 | 1 |
| C10orf137 | — | — | — | 0 | 1 |
| C10orf22 | — | — | 7609 | — | 1 |
| C10orf26 | — | — | 274 | — | 1 |
| C10orf3 | — | — | 818 | 0 | 0 |
| C10orf48 | 483 | — | — | — | 0 |
| C10orf6 | — | — | — | 0 | 0 |
| C10orf7 | 0 | — | 0 | — | 1 |
| C10orf70 | — | 4002 | 1981 | — | 1 |
| C10orf78 | 0 | — | — | 0 | 0 |
| C10orf84 | — | 37 | — | — | 0 |
| C11orf1 | — | — | 0 | — | 1 |
| C11orf10 | — | — | — | 0 | 1 |
| C12orf14 | — | 0 | — | 0 | 1 |
| C12orf2 | 882 | 882 | 882 | — | 1 |
| C12orf8 | — | — | — | 30 | 1 |
| C13orf11 | — | — | — | 19 | 1 |
| C13orf17 | — | — | — | 0 | 1 |
| C13orf3 | — | — | — | 168 | 1 |
| C13orf7 | 185 | 0 | 0 | 0 | 1 |
| C14orf10 | — | 0 | 0 | 0 | 1 |
| C14orf105 | — | 0 | 0 | — | 0 |
| C14orf106 | — | — | — | 0 | 1 |
| C14orf108 | — | 0 | — | 0 | 1 |
| C14orf11 | — | 6973 | — | — | 1 |
| C14orf114 | — | — | 739 | — | 1 |
| C14orf115 | — | 0 | 0 | — | 1 |
| C14orf119 | — | 0 | — | — | 1 |
| C14orf122 | — | 45 | 57 | 0 | 1 |
| C14orf130 | — | — | — | 0 | 1 |
| C14orf133 | — | — | 0 | — | 1 |
| C14orf138 | — | 727 | 727 | — | 1 |
| C14orf140 | — | — | — | 4842 | 0 |
| C14orf142 | — | — | — | 0 | 0 |
| C14orf151 | 6968 | — | 6968 | 6968 | 1 |
| C14orf153 | 755 | — | 755 | 755 | 1 |
| C14orf156 | 0 | — | 0 | — | 1 |
| C14orf159 | — | 0 | — | — | 1 |
| C14orf31 | — | — | — | 3717 | 0 |
| C14orf80 | — | — | — | 111 | 1 |
| C14orf87 | — | — | — | 79 | 1 |
| C14orf9 | — | 12 | 12 | 231 | 1 |
| C14orf94 | — | — | — | 0 | 1 |
| C15orf20 | — | 0 | — | — | 1 |
| C15orf24 | — | — | 34 | — | 1 |
| C15orf25 | — | — | — | 0 | 1 |
| C15orf29 | 3817 | 3817 | 3817 | — | 1 |
| C16orf33 | — | — | — | 0 | 1 |
| C16orf48 | 0 | — | — | 0 | 0 |
| C16orf9 | — | — | — | 5106 | 1 |
| C17orf39 | — | — | — | 0 | 1 |
| C18orf19 | — | — | — | 0 | 1 |
| C18orf23 | — | — | 1214 | — | 0 |
| C18orf24 | — | — | — | 0 | 1 |
| C18orf37 | — | 0 | 0 | 0 | 1 |
| C18orf55 | — | — | — | 0 | 1 |
| C19orf14 | — | — | 209 | 0 | 1 |
| C19orf6 | — | — | 278 | — | 1 |
| C1orf16 | — | 1768 | — | — | 1 |
| C1orf21 | — | 2538 | 2538 | — | 1 |
| C1orf41 | — | — | — | 75 | 1 |
| C1orf8 | — | — | 0 | 0 | 1 |
| C1QA | 6596 | 6596 | — | — | 0 |
| C1QTNF6 | — | — | 78 | — | 0 |
| C1R | 1262 | — | — | — | 0 |
| C20orf12 | — | — | — | 0 | 0 |
| C20orf172 | — | — | — | 0 | 1 |
| C20orf175 | — | 4925 | 4925 | — | 0 |
| C20orf23 | — | 1220 | 1476 | — | 0 |
| C20orf30 | — | — | — | 5776 | 1 |
| C20orf42 | — | — | 0 | — | 1 |
| C20orf52 | — | — | 0 | — | 1 |
| C20orf72 | — | — | — | 0 | 1 |
| C20orf77 | — | — | — | 0 | 1 |
| C20orf86 | — | — | 555 | — | 0 |
| C20orf9 | — | — | 0 | — | 1 |
| C20orf96 | — | 0 | 0 | 0 | 1 |
| C21orf45 | — | — | — | 0 | 1 |
| C21orf49 | — | 0 | — | — | 0 |
| C21orf58 | — | — | — | 0 | 0 |
| C21orf59 | — | — | 0 | — | 1 |
| C21orf6 | — | — | 5132 | — | 0 |
| C21orf66 | — | 0 | — | — | 1 |
| C21orf88 | — | — | 931 | — | 0 |
| C22orf13 | — | 0 | — | — | 1 |
| C22orf18 | — | — | — | 75 | 1 |
| C2orf11 | 76 | — | — | — | 0 |
| C2orf13 | — | — | — | 0 | 0 |
| C2orf29 | — | — | — | 0 | 1 |
| C2orf7 | — | — | 294 | — | 0 |
| C3F | — | — | — | 213 | 0 |
| C3orf10 | — | — | 0 | 0 | 1 |
| C3orf19 | — | — | 0 | — | 1 |
| C4BPA | — | 2302 | — | — | 0 |
| C5orf4 | — | 7357 | 7357 | 7429 | 1 |
| C5R1 | — | 3582 | — | — | 0 |
| C6orf111 | 890 | 0 | 0 | — | 1 |
| C6orf117 | — | — | 201 | — | 1 |
| C6orf128 | 665 | — | — | — | 0 |
| C6orf130 | — | 179 | — | 0 | 1 |
| C6orf141 | — | 234 | 497 | — | 0 |
| C6orf152 | — | 7604 | 7328 | — | 0 |
| C6orf166 | — | 360 | 116 | — | 1 |
| C6orf167 | — | — | — | 0 | 1 |
| C6orf204 | — | 0 | 0 | — | 0 |
| C6orf21 | — | 3921 | — | — | 0 |
| C6orf32 | — | — | 2160 | — | 0 |
| C6orf47 | — | — | — | 4113 | 1 |
| C6orf48 | — | — | — | 0 | 1 |
| C6orf64 | — | — | 491 | — | 0 |
| C6orf72 | — | 179 | — | — | 0 |
| C6orf78 | — | — | 1194 | — | 0 |
| C7orf16 | 1124 | 1124 | 1124 | — | 0 |
| C7orf20 | — | — | — | 0 | 0 |
| C7orf27 | — | — | — | 0 | 1 |
| C7orf33 | 305 | 305 | 305 | — | 0 |
| C7orf35 | — | — | 0 | 27 | 1 |
| C9orf105 | — | — | — | 0 | 1 |
| C9orf156 | — | — | — | 0 | 1 |
| C9orf19 | — | — | 1118 | — | 1 |
| C9orf39 | — | — | — | 0 | 1 |
| C9orf40 | — | — | — | 0 | 1 |
| C9orf43 | — | 62 | — | 0 | 0 |
| C9orf5 | — | 0 | 0 | 0 | 1 |
| C9orf74 | 6029 | 6029 | 6029 | — | 1 |
| C9orf76 | — | 1555 | — | — | 1 |
| C9orf79 | — | — | 3336 | — | 0 |
| C9orf82 | — | — | 66 | — | 1 |
| C9orf93 | — | 0 | — | — | 0 |
| C9orf96 | — | — | — | 0 | 0 |
| C9orf97 | 126 | 5 | — | 126 | 1 |
| CA14 | — | 799 | 1058 | — | 0 |
| CA2 | 1710 | 1710 | 1710 | — | 1 |
| CA4 | 6876 | 6876 | 6876 | — | 1 |
| CA8 | — | 258 | 478 | — | 0 |
| CABLES1 | 1761 | 1761 | 1761 | — | 1 |
| CACNA1A | — | 412 | 412 | — | 1 |
| CACNA1G | 1780 | — | — | 1526 | 0 |
| CACNA2D1 | 953 | 953 | 678 | — | 1 |
| CACNG8 | — | — | 894 | — | 0 |
| CACYBP | — | — | — | 0 | 1 |
| CALB1 | — | — | 3389 | — | 1 |
| CALCA | — | — | 1436 | — | 0 |
| CALCRL | — | — | 0 | — | 0 |
| CALD1 | — | — | 0 | — | 1 |
| CALM2 | — | 1754 | 1754 | — | 1 |
| CALR | — | 0 | 0 | 0 | 1 |
| CAP1 | — | — | — | 95 | 1 |
| CAPON | 5812 | — | — | — | 0 |
| CAPZA2 | 4834 | 4834 | 4834 | — | 1 |
| CASP10 | — | 63 | — | — | 0 |
| CASP8AP2 | — | — | — | 0 | 1 |
| CASP9 | — | 1980 | 2213 | 0 | 1 |
| CATSPER2 | — | — | — | 0 | 0 |
| CAV1 | — | — | 901 | — | 1 |
| CBLN1 | — | — | — | 0 | 0 |
| CBWD1 | — | — | 0 | — | 0 |
| CBWD2 | — | — | 0 | — | 0 |
| CBX1 | — | — | — | 0 | 1 |
| CBX2 | — | 1145 | — | — | 0 |
| CBX3 | — | 336 | 260 | 336 | 1 |
| CBX5 | — | 0 | 0 | 0 | 1 |
| CBX8 | 7298 | 7298 | — | — | 0 |
| CCDC12 | — | — | 0 | — | 1 |
| CCDC6 | 1909 | — | 590 | — | 0 |
| CCHCR1 | — | — | — | 0 | 1 |
| CCL2 | 72 | 72 | 0 | — | 0 |
| CCND1 | — | 0 | — | — | 1 |
| CCNE2 | — | — | 815 | — | 0 |
| CCT5 | — | — | — | 13 | 1 |
| CCT6B | — | — | 0 | — | 1 |
| CCT7 | — | — | 526 | — | 1 |
| CD82 | 5611 | 5611 | 5830 | — | 0 |
| CD99L2 | 3550 | 3550 | 3550 | — | 0 |
| CDADC1 | — | — | — | 0 | 1 |
| CDC14A | — | — | — | 375 | 0 |
| CDC14B | 676 | 676 | 412 | 0 | 1 |
| CDCL6 | — | — | 3742 | — | 1 |
| CDC2 | — | 0 | 0 | 0 | 1 |
| CDC20 | — | — | — | 0 | 1 |
| CDC23 | — | — | — | 0 | 1 |
| CDC25A | — | — | — | 0 | 1 |
| CDC25C | — | — | — | 0 | 1 |
| CDC2L5 | — | — | — | 434 | 1 |
| CDC37 | — | — | — | 0 | 1 |
| CDC42BPA | — | 989 | — | — | 1 |
| CDC42EP4 | — | — | 730 | — | 1 |
| CDC45L | — | 0 | 0 | 0 | 1 |
| CDC7 | 379 | 118 | 118 | 0 | 1 |
| CDCA1 | — | — | — | 0 | 1 |
| CDCA2 | — | — | — | 0 | 1 |
| CDCA3 | — | — | — | 0 | 1 |
| CDCA5 | — | — | — | 0 | 1 |
| CDCA7 | — | — | — | 0 | 1 |
| CDCA8 | — | — | — | 0 | 1 |
| CDH1 | 3491 | — | 3491 | — | 1 |
| CDH2 | — | 744 | 1036 | — | 1 |
| CDH3 | — | 0 | — | — | 1 |
| CDH6 | — | 0 | — | — | 0 |
| CDH9 | — | 0 | 0 | — | 0 |
| CDK6 | — | 1491 | 1212 | — | 1 |
| CDK8 | — | — | — | 0 | 1 |
| CDKAL1 | — | 3884 | — | — | 0 |
| CDS2 | — | — | 422 | 0 | 1 |
| CDW92 | 1986 | 1986 | 1986 | 4421 | 1 |
| CDX1 | 23 | — | 23 | — | 0 |
| CDX2 | 1917 | — | 0 | — | 0 |
| CDX4 | — | 0 | 0 | — | 0 |
| CDYL | 1762 | 922 | 922 | — | 1 |
| CEBPZ | — | — | 0 | — | 1 |
| CEI | 801 | 1504 | 1504 | — | 0 |
| CENPA | — | — | — | 0 | 1 |
| CENTG2 | — | — | — | 0 | 1 |
| CEPT1 | — | — | 780 | — | 1 |
| CER1 | — | 0 | 0 | — | 1 |
| CERKL | 262 | — | 262 | 269 | 0 |
| CETN3 | — | 427 | 701 | — | 1 |
| CFC1 | — | — | 628 | — | 0 |
| CFDP1 | — | — | — | 0 | 1 |
| CFL1 | — | 0 | 23 | 0 | 1 |
| CGI-143 | — | — | — | 0 | 1 |
| CGI-37 | — | 0 | 0 | — | 1 |
| CGI-62 | — | — | 19 | — | 1 |
| CGI-94 | — | — | — | 0 | 1 |
| CHCHD3 | — | — | — | 0 | 1 |
| CHCHD5 | — | — | 0 | — | 1 |
| CHD2 | — | — | 0 | — | 1 |
| CHEK1 | — | — | — | 0 | 1 |
| CHEK2 | — | — | — | 515 | 1 |
| CHERP | — | — | — | 0 | 1 |
| CHES1 | — | 7046 | 1451 | — | 1 |
| CHKB | — | — | — | 0 | 1 |
| CHM | — | — | 0 | 0 | 0 |
| CHN2 | — | 4414 | 3229 | — | 0 |
| CHRAC1 | — | — | — | 409 | 0 |
| CHRFAM7A | — | — | — | 113 | 0 |
| CHRM5 | — | 6929 | — | — | 0 |
| CHRNA1 | 7216 | 7216 | 7216 | — | 0 |
| CHRNB3 | — | 1416 | — | — | 0 |
| CHRNE | — | 6037 | 5573 | — | 0 |
| CHST4 | — | — | 0 | — | 1 |
| CHST5 | 577 | — | — | 4205 | 1 |
| CIT | — | — | — | 0 | 1 |
| CITED2 | — | — | 815 | — | 1 |
| CKAP1 | — | — | — | 0 | 1 |
| CKS2 | — | — | 0 | 0 | 1 |
| CLDN1 | — | — | 3010 | — | 0 |
| CLDN6 | — | — | 3225 | 5849 | 1 |
| CLEC1B | — | 3626 | 3626 | — | 0 |
| CLEC3A | 521 | — | — | — | 0 |
| CLEC7A | 1067 | — | 1067 | — | 0 |
| CLIC1 | — | — | 2168 | 2168 | 1 |
| CLIC4 | — | 1042 | 593 | — | 1 |
| CLN3 | — | 0 | 0 | — | 1 |
| CLSPN | — | — | — | 0 | 0 |
| CLTA | — | — | — | 0 | 1 |
| CNAP1 | — | — | 0 | — | 1 |
| CNN2 | — | — | 4444 | — | 1 |
| CNN3 | — | — | 1388 | — | 1 |
| CNOT10 | — | — | — | 0 | 1 |
| CNOT6 | — | — | — | 333 | 1 |
| CNOT8 | — | 189 | 262 | 189 | 1 |
| CNTNAP3 | 1321 | — | 1041 | — | 1 |
| COG4 | — | — | — | 34 | 1 |
| COG7 | — | — | 0 | — | 1 |
| COL11A2 | — | — | — | 7758 | 1 |
| COL12A1 | 7204 | 6964 | 6964 | — | 1 |
| COL16A1 | — | — | — | 989 | 0 |
| COL21A1 | — | 978 | 497 | — | 0 |
| COL4A5 | — | — | 144 | — | 1 |
| COL4A6 | — | — | 570 | — | 1 |
| COL7A1 | — | — | 0 | — | 1 |
| COL8A1 | — | — | 4268 | — | 0 |
| COMMD3 | 1289 | 1289 | 980 | — | 1 |
| COMMD7 | 5773 | 5545 | 5545 | — | 1 |
| COPB | — | 0 | — | — | 1 |
| COPB2 | — | — | — | 1811 | 1 |
| COPE | — | — | 0 | — | 1 |
| COPS7A | — | 0 | — | — | 1 |
| COPS7B | — | — | — | 14 | 1 |
| CORO6 | — | — | — | 0 | 0 |
| COTL1 | — | — | 165 | — | 1 |
| COX6A1 | — | — | 0 | — | 1 |
| CP110 | — | — | 0 | 0 | 0 |
| CPA5 | — | — | 333 | — | 0 |
| CPB1 | 3251 | — | — | — | 0 |
| CPOX | — | — | — | 0 | 1 |
| CPS1 | 901 | 901 | 901 | — | 0 |
| CPT1A | 1261 | 1261 | 1261 | — | 1 |
| CPT2 | — | — | — | 0 | 1 |
| CREB3L4 | — | 0 | — | 3542 | 1 |
| CREG2 | — | 1645 | — | — | 0 |
| CRIM1 | — | 3444 | — | — | 1 |
| CRK7 | — | 564 | — | — | 1 |
| CRLF3 | — | — | — | 6935 | 1 |
| CRSP3 | — | — | 367 | — | 1 |
| CRSP6 | — | 0 | — | — | 1 |
| CRSP8 | — | — | — | 0 | 1 |
| CRYBA2 | — | — | 270 | — | 0 |
| CRYBA4 | — | — | 4419 | — | 0 |
| CRYBB1 | — | — | 483 | — | 0 |
| CRYZ | — | — | 475 | — | 1 |
| CS | — | — | 0 | — | 1 |
| CSAD | 1353 | 1073 | 1073 | — | 0 |
| CSGlcA-T | — | 4484 | 4959 | 4959 | 0 |
| CSMD3 | 64 | 344 | 64 | — | 0 |
| CSNK1E | — | 590 | 1544 | — | 1 |
| CSNK2B | — | — | — | 465 | 1 |
| CSPG2 | — | 321 | 34 | — | 1 |
| CSPG6 | — | — | — | 0 | 1 |
| CSTF1 | — | — | 0 | 0 | 1 |
| CSTF2T | — | — | — | 0 | 1 |
| CSTF3 | — | — | 12 | 12 | 1 |
| CTGF | 1520 | 1013 | 0 | — | 1 |
| CTH | — | 0 | — | — | 1 |
| CTMP | — | 283 | — | — | 0 |
| CTSL2 | — | — | 6151 | — | 1 |
| CTTNBP2 | — | — | 1688 | — | 0 |
| CTXN1 | — | — | — | 151 | 0 |
| CUGBP2 | — | — | 216 | — | 1 |
| CUL4B | — | — | — | 57 | 1 |
| CUZD1 | 0 | — | — | — | 0 |
| CXADR | — | — | 879 | — | 1 |
| CXCL5 | — | 0 | — | — | 1 |
| CXorf26 | 0 | — | 0 | — | 1 |
| CXorf41 | — | — | — | 0 | 0 |
| CYB5-M | — | — | 0 | 0 | 1 |
| CYB5R2 | — | 608 | — | 328 | 1 |
| CYBASC3 | — | — | — | 0 | 1 |
| CYLD | — | — | 0 | — | 1 |
| CYP1A1 | — | 923 | — | — | 0 |
| CYPLB1 | — | — | 130 | — | 1 |
| CYP24A1 | — | — | 557 | — | 0 |
| CYP26B1 | 692 | — | — | — | 0 |
| CYP2R1 | — | — | 0 | — | 1 |
| CYP2S1 | — | — | 2494 | — | 1 |
| CYR61 | — | 76 | 76 | 0 | 1 |
| CYYR1 | 988 | — | — | — | 0 |
| DACH1 | 2676 | 2676 | 2676 | — | 0 |
| DAND5 | — | — | — | 4624 | 0 |
| DARS | — | — | — | 0 | 1 |
| DATF1 | — | 310 | 4268 | — | 1 |
| DAZL | — | 5494 | — | — | 0 |
| DBCCR1L | 446 | 722 | 177 | — | 0 |
| DBR1 | 0 | — | — | 0 | 1 |
| DC13 | — | — | — | 50 | 1 |
| DCC1 | — | 601 | 601 | — | 1 |
| DCLRE1A | — | — | — | 0 | 0 |
| DCLRE1C | — | — | 397 | — | 1 |
| DCPS | — | — | — | 0 | 1 |
| DCX | — | — | 573 | — | 1 |
| DDX11 | — | — | — | 0 | 1 |
| DDX17 | — | 0 | 6440 | — | 1 |
| DDX18 | — | — | 1908 | — | 1 |
| DDX20 | — | — | 5668 | — | 1 |
| DDX21 | — | 0 | — | — | 1 |
| DDX23 | — | — | 0 | 0 | 1 |
| DDX31 | 612 | — | — | — | 1 |
| DDX39 | — | 0 | 0 | 0 | 1 |
| DDX41 | — | — | 0 | 0 | 1 |
| DDX49 | — | — | 49 | — | 1 |
| DDX5 | — | 31 | 31 | — | 1 |
| DDX50 | — | — | — | 312 | 1 |
| DEK | — | — | — | 30 | 1 |
| DEPDC1 | — | — | — | 0 | 1 |
| DEPDC2 | 0 | 127 | 127 | — | 0 |
| DERL2 | — | — | — | 105 | 1 |
| DFFA | — | — | — | 0 | 1 |
| DFNB31 | — | 33 | — | — | 0 |
| DGAT2L6 | — | 5186 | 5186 | — | 0 |
| DGKB | — | — | 4844 | — | 0 |
| DHCR7 | — | — | 1541 | — | 1 |
| DHDDS | 2337 | 2337 | 2337 | — | 1 |
| DHFRL1 | — | 0 | 0 | 0 | 0 |
| DHRS3 | 1109 | 1109 | 1109 | — | 1 |
| DHRS6 | — | 0 | — | — | 1 |
| DHX35 | — | — | — | 141 | 1 |
| DHX38 | — | 0 | 0 | — | 1 |
| DHX40 | — | — | — | 0 | 1 |
| DIAPH3 | — | 103 | — | 0 | 1 |
| DIRC2 | — | — | 244 | — | 1 |
| DKFZP434B168 | — | — | — | 0 | 1 |
| DKFZp434I1020 | — | — | — | 0 | 0 |
| DKFZp547D2210 | — | 6899 | — | — | 0 |
| DKFZp547G0215 | — | 103 | 103 | — | 0 |
| DKFZP564F0522 | — | 3778 | — | — | 1 |
| DKFZP564G2022 | — | — | 0 | — | 1 |
| DKFZP564K0822 | — | — | 946 | — | 1 |
| DKFZP564O0523 | — | — | 0 | — | 0 |
| DKFZP566D1346 | 0 | — | — | 0 | 0 |
| DKFZP586A0522 | — | — | 0 | — | 1 |
| DKFZp667B0210 | 1364 | 6447 | 5966 | — | 0 |
| DKFZp686I15217 | 3301 | — | — | — | 0 |
| DKFZp761A078 | — | — | — | 0 | 1 |
| DKK1 | 165 | 0 | 0 | — | 1 |
| DKKL1 | — | 0 | — | — | 0 |
| DLEU1 | — | — | — | 0 | 1 |
| DLEU2 | — | — | — | 0 | 0 |
| DLG7 | — | — | 0 | 0 | 1 |
| DLGAP1 | — | — | — | 7724 | 0 |
| DLL1 | 5366 | 7225 | — | — | 0 |
| DLX1 | 1203 | 1203 | 1203 | — | 0 |
| DLX2 | 2944 | — | — | — | 0 |
| DLX4 | 3011 | 5332 | 665 | — | 0 |
| DLX5 | 62 | 62 | 0 | — | 0 |
| DMD | — | — | — | 676 | 1 |
| DMRT1 | 995 | 715 | 715 | — | 0 |
| DMRT3 | — | — | 2390 | — | 0 |
| DMTF1 | — | — | — | 0 | 1 |
| DMXL1 | — | — | 439 | — | 1 |
| DNAJC8 | — | — | 2968 | — | 1 |
| DNAJC9 | 3494 | — | 3494 | 3494 | 1 |
| DNASE2 | — | — | — | 3404 | 0 |
| DNM2 | — | 77 | 77 | — | 1 |
| DOCK5 | — | — | 4470 | — | 0 |
| DOK3 | — | — | 6388 | 6388 | 0 |
| DPAGT1 | — | 0 | 0 | — | 1 |
| DPM1 | — | — | 0 | — | 1 |
| DPP3 | — | 0 | — | — | 1 |
| DPP9 | — | 168 | — | — | 0 |
| DPPA2 | — | 0 | 0 | — | 0 |
| DPPA4 | 0 | 0 | 0 | — | 1 |
| DPYSL2 | 383 | 137 | 383 | — | 1 |
| DPYSL3 | 1079 | 1079 | 1079 | — | 1 |
| DQX1 | — | — | — | 3074 | 0 |
| DRF1 | — | — | — | 0 | 0 |
| DSC3 | 0 | — | — | — | 0 |
| DSG2 | — | — | — | 253 | 1 |
| DTNA | 2067 | 2067 | 2067 | — | 1 |
| DTX2 | — | — | 3098 | — | 1 |
| DUSP12 | 483 | 235 | 483 | — | 1 |
| DUSP6 | 1365 | 83 | 0 | — | 1 |
| DVL2 | — | 5058 | 5058 | 5058 | 1 |
| DXS9879E | — | — | — | 0 | 1 |
| DYM | — | — | 4347 | — | 1 |
| E2F3 | — | 114 | 442 | 0 | 1 |
| EAF2 | — | — | — | 0 | 0 |
| EDA2R | — | — | 182 | — | 0 |
| EDD | 817 | 373 | 354 | 373 | 1 |
| EEF1B2 | — | — | — | 0 | 1 |
| EEF1E1 | — | — | 0 | 0 | 1 |
| EEF2 | — | 14 | 289 | — | 1 |
| EFTUD1 | 4271 | — | — | — | 1 |
| EGLN3 | — | 132 | 132 | — | 1 |
| EGR1 | — | — | — | 0 | 1 |
| EGR3 | — | 0 | — | — | 1 |
| EHBP1 | — | — | 247 | — | 1 |
| EHD4 | — | — | 0 | — | 1 |
| EIF1AY | 5364 | — | — | 5626 | 0 |
| eIF2A | — | — | 0 | — | 1 |
| EIF2AK3 | — | — | 0 | — | 1 |
| EIF2C2 | — | 452 | — | — | 1 |
| EIF2S1 | 0 | — | — | 0 | 1 |
| EIF3S12 | — | — | — | 0 | 1 |
| EIF3S5 | — | 0 | 0 | 0 | 1 |
| EIF3S7 | — | — | 0 | — | 1 |
| EIF3S8 | — | — | — | 0 | 1 |
| EIF3S9 | — | — | — | 374 | 1 |
| EIF4B | — | — | 383 | — | 1 |
| EIF4ENIF1 | — | — | 912 | — | 1 |
| EIF4G2 | — | 452 | 452 | — | 1 |
| ELAVL2 | 1270 | 1270 | — | — | 1 |
| ELL2 | — | — | 0 | — | 1 |
| ELMOD1 | — | 764 | — | — | 0 |
| EME1 | — | — | 0 | — | 0 |
| EMP2 | — | — | — | 0 | 1 |
| EMX2 | — | — | 360 | — | 0 |
| EN1 | 32 | 32 | 6863 | — | 0 |
| EN2 | 4817 | — | — | — | 0 |
| ENAH | — | — | — | 0 | 1 |
| ENC2 | — | — | — | 0 | 0 |
| ENDOGL1 | — | — | — | 0 | 0 |
| ENPEP | — | — | 2634 | — | 0 |
| ENPP2 | 0 | 0 | 0 | — | 1 |
| ENPP3 | — | — | 7867 | — | 0 |
| ENPP4 | — | — | — | 0 | 0 |
| ENSA | — | 0 | 0 | — | 1 |
| ENST00000215770 | — | — | 4483 | — | 0 |
| ENST00000217260 | — | — | 3184 | — | 0 |
| ENST00000217537 | — | 1048 | 1048 | — | 1 |
| ENST00000218188 | 5556 | — | — | — | 0 |
| ENST00000218328 | — | 1200 | — | — | 1 |
| ENST00000238256 | — | — | — | 0 | 1 |
| ENST00000244321 | — | — | — | 4746 | 1 |
| ENST00000246013 | — | 1465 | — | — | 0 |
| ENST00000246083 | 5210 | 4948 | 4948 | — | 0 |
| ENST00000250479 | — | — | 6733 | — | 0 |
| ENST00000252032 | — | 841 | 841 | — | 1 |
| ENST00000255500 | — | — | — | 343 | 1 |
| ENST00000258728 | — | — | 6576 | — | 0 |
| ENST00000259327 | — | 870 | — | — | 0 |
| ENST00000260257 | — | — | 0 | — | 0 |
| ENST00000263256 | — | — | — | 567 | 1 |
| ENST00000265140 | — | — | — | 0 | 0 |
| ENST00000268043 | — | 3926 | — | — | 0 |
| ENST00000269142 | — | — | 481 | — | 1 |
| ENST00000279028 | — | — | 4680 | — | 0 |
| ENST00000279873 | — | 0 | 0 | — | 1 |
| ENST00000285199 | — | — | — | 0 | 0 |
| ENST00000289348 | 945 | — | — | 781 | 0 |
| ENST00000290943 | — | — | 897 | — | 0 |
| ENST00000291011 | 110 | — | — | 3 | 0 |
| ENST00000291982 | 2790 | 2790 | 2790 | — | 0 |
| ENST00000293218 | — | 4573 | — | — | 0 |
| ENST00000296508 | 989 | 6693 | 6467 | — | 0 |
| ENST00000298125 | — | 317 | — | — | 0 |
| ENST00000298406 | 6875 | 6941 | 6628 | — | 1 |
| ENST00000298439 | — | — | — | 3215 | 0 |
| ENST00000300358 | — | — | — | 251 | 0 |
| ENST00000302155 | — | 4002 | 4002 | — | 1 |
| ENST00000303758 | — | — | 7347 | — | 0 |
| ENST00000304181 | 256 | — | 256 | — | 0 |
| ENST00000304908 | — | — | — | 583 | 0 |
| ENST00000304952 | 5191 | — | — | — | 0 |
| ENST00000305188 | — | — | — | 0 | 0 |
| ENST00000305321 | — | — | 0 | 0 | 0 |
| ENST00000305339 | — | — | — | 0 | 0 |
| ENST00000308142 | 4272 | 3993 | 4272 | — | 0 |
| ENST00000308618 | — | 4605 | 4605 | — | 0 |
| ENST00000309024 | — | 1507 | 1507 | — | 1 |
| ENST00000309467 | 1187 | 1187 | 1187 | — | 0 |
| ENST00000310496 | 701 | — | — | — | 1 |
| ENST00000313737 | — | 1396 | — | 1396 | 0 |
| ENST00000314890 | — | — | 127 | — | 0 |
| ENST00000316011 | — | 2792 | 2207 | — | 0 |
| ENST00000316333 | 4617 | — | 1908 | — | 0 |
| ENST00000317976 | — | — | — | 715 | 1 |
| ENST00000319884 | 1568 | 1568 | 1568 | — | 0 |
| ENST00000321662 | — | — | — | 157 | 0 |
| ENST00000321905 | — | — | 283 | 670 | 0 |
| ENST00000321991 | — | 7021 | — | 7298 | 0 |
| ENST00000322309 | — | — | 3612 | — | 0 |
| ENST00000322358 | — | 6736 | — | — | 0 |
| ENST00000322567 | — | — | 28 | — | 0 |
| ENST00000323051 | — | 1540 | — | — | 0 |
| ENST00000323426 | — | — | 6960 | 0 | 0 |
| ENST00000324434 | — | — | — | 0 | 0 |
| ENST00000325371 | — | — | — | 2574 | 0 |
| ENST00000326237 | — | — | — | 0 | 0 |
| ENST00000327804 | — | — | 2083 | — | 0 |
| ENST00000327898 | — | — | — | 0 | 0 |
| ENST00000327975 | 508 | 508 | — | — | 0 |
| ENST00000328281 | — | — | 2436 | — | 0 |
| ENST00000329118 | — | — | 5232 | — | 0 |
| ENST00000330469 | — | — | 376 | — | 0 |
| ENST00000330543 | — | — | 1773 | — | 0 |
| ENST00000330676 | — | 6688 | 6425 | — | 0 |
| ENST00000331014 | 865 | 865 | 586 | — | 0 |
| ENST00000331019 | — | — | — | 2612 | 0 |
| ENST00000332835 | — | — | 681 | — | 0 |
| ENST00000333380 | 6055 | 6055 | 6055 | — | 0 |
| ENST00000334280 | 2740 | — | — | — | 0 |
| ENST00000334440 | 1643 | 1643 | 1643 | — | 0 |
| ENST00000334534 | — | 0 | 0 | — | 0 |
| ENST00000334708 | — | 1190 | 773 | — | 0 |
| ENST00000335342, | 437 | 437 | — | — | 0 |
| ENST00000336377 | | | | | |
| ENST00000335498 | — | — | — | 0 | 0 |
| ENST00000335507 | — | — | — | 0 | 0 |
| ENST00000338461 | — | 375 | 95 | 375 | 0 |
| ENST00000339485 | — | 378 | — | 657 | 0 |
| ENST00000340225 | — | — | 0 | — | 0 |
| ENST00000340457 | — | — | — | 0 | 0 |
| ENST00000340967 | — | — | — | 338 | 0 |
| ENST00000341482 | — | — | 7811 | — | 0 |
| ENST00000343447 | 4004 | — | 3870 | — | 0 |
| ENST00000343804 | — | — | — | 5150 | 0 |
| ENST00000343904 | — | 4377 | 4377 | — | 0 |
| ENST00000345108 | — | — | — | 3898 | 0 |
| ENST00000354314 | — | — | — | 66 | 0 |
| ENST00000354576 | — | 3909 | — | — | 0 |
| ENST00000354929 | — | 2829 | — | — | 0 |
| ENST00000355830 | 6053 | 6053 | — | — | 0 |
| ENST00000355920 | — | 8000 | — | — | 0 |
| ENST00000356236 | — | — | — | 1155 | 0 |
| ENST00000356724 | — | 0 | 0 | — | 1 |
| ENST00000356869 | — | — | 2709 | — | 0 |
| ENST00000357217 | — | 2126 | 2126 | — | 0 |
| ENST00000357878 | — | 2750 | — | — | 0 |
| ENST00000358347 | — | — | 5191 | — | 0 |
| ENST00000358410 | — | — | — | 0 | 1 |
| ENST00000358688 | — | 7175 | 7175 | — | 0 |
| ENST00000359178 | — | — | 1688 | — | 0 |
| ENST00000359260 | — | — | 251 | — | 0 |
| ENST00000359554 | — | — | 5108 | — | 0 |
| ENST00000360566 | — | — | — | 0 | 0 |
| ENST00000360940 | — | 1189 | 1189 | 1189 | 0 |
| ENTPD1 | 81 | — | 0 | — | 1 |
| EOMES | 6354 | 6091 | 801 | — | 1 |
| EPHA1 | 1484 | 1484 | 1484 | — | 1 |
| EPHA4 | 4025 | 4025 | 4025 | — | 0 |
| EPHA5 | — | — | — | 6696 | 0 |
| EPHA7 | — | — | 330 | — | 0 |
| EPHB3 | — | — | 2681 | — | 0 |
| EPM2AIP1 | — | 66 | 0 | 66 | 1 |
| EPS15L1 | — | — | 845 | — | 1 |
| ERBB2 | — | 900 | 900 | — | 1 |
| ERCC1 | — | — | 3361 | — | 1 |
| ERH | — | — | 0 | — | 1 |
| ESM1 | — | — | 1390 | — | 0 |
| ESPL1 | — | — | — | 291 | 1 |
| EST1B | 92 | — | 92 | 0 | 1 |
| ESX1L | 2890 | 2233 | 2233 | — | 0 |
| ET | — | — | — | 0 | 0 |
| ETAA16 | — | — | — | 0 | 0 |
| ETHE1 | — | 6627 | — | — | 1 |
| ETNK1 | — | — | 319 | — | 1 |
| ETV3 | 1806 | — | 1806 | — | 0 |
| ETV5 | — | 1003 | 1003 | — | 0 |
| EVA1 | — | — | 23 | — | 0 |
| EVPL | — | — | 2168 | — | 0 |
| EVX1 | 1112 | — | 832 | — | 0 |
| EXO1 | — | — | — | 0 | 1 |
| EXOSC5 | — | 0 | 0 | 0 | 1 |
| EXOSC9 | 4360 | 4360 | 4360 | — | 1 |
| EXTL2 | — | — | 359 | — | 1 |
| F11R | — | — | 6951 | — | 1 |
| F25965 | — | — | 0 | — | 1 |
| F2R | — | — | — | 136 | 0 |
| FAM13C1 | — | 499 | 499 | — | 0 |
| FAM20C | — | — | 4110 | — | 1 |
| FAM33A | 0 | 0 | 0 | 0 | 1 |
| FAM35A | — | — | 1569 | — | 1 |
| FAM46B | — | — | — | 0 | 1 |
| FAM46D | — | — | — | 1064 | 0 |
| FAM53C | — | — | — | 5316 | 1 |
| FAM54A | — | — | 0 | 0 | 1 |
| FANCA | — | — | 6444 | — | 1 |
| FANCC | — | 211 | — | 0 | 1 |
| FANCD2 | — | — | — | 0 | 1 |
| FANCF | — | 0 | 0 | — | 1 |
| FANCL | — | — | 0 | 0 | 1 |
| FARSLA | — | 4553 | 4299 | 4553 | 1 |
| FAT | — | — | 708 | — | 1 |
| FBL | — | — | — | 197 | 1 |
| FBLN1 | — | — | 910 | — | 1 |
| FBXL11 | — | — | 2393 | — | 1 |
| FBXL14 | — | 6573 | 6573 | — | 1 |
| FBXL17 | — | — | 710 | — | 0 |
| FBXL19 | — | 0 | — | — | 1 |
| FBXO11 | — | 354 | — | — | 1 |
| FBXO15 | — | — | — | 333 | 0 |
| FBXO16 | — | 0 | — | — | 1 |
| FBXO31 | — | — | 4378 | — | 1 |
| FBXO40 | — | 0 | 0 | — | 0 |
| FBXW11 | 1792 | 1792 | 1792 | — | 1 |
| FCHSD2 | — | — | — | 338 | 0 |
| FDPS | — | — | 137 | — | 1 |
| FDXR | — | — | — | 0 | 1 |
| FEM1A | — | 19 | 19 | — | 1 |
| FEM1C | — | — | 4130 | — | 1 |
| FEN1 | — | — | — | 0 | 1 |
| FERD3L | 565 | 565 | 288 | — | 0 |
| FEZ1 | 858 | 858 | 1138 | — | 1 |
| FGF1 | 819 | 819 | 819 | — | 0 |
| FGF19 | — | 743 | 743 | — | 0 |
| FGF2 | 1255 | 1255 | 1053 | — | 1 |
| FGF20 | 0 | 0 | — | — | 0 |
| FGF3 | 0 | — | — | — | 0 |
| FGF8 | — | — | 3048 | — | 0 |
| FGFR1 | 5437 | 5437 | 5437 | — | 1 |
| FGFR2 | 870 | 870 | 870 | — | 1 |
| FGIF | — | — | 0 | 0 | 1 |
| FHIT | — | — | 650 | — | 1 |
| FHL3 | — | — | — | 7089 | 0 |
| FHOD1 | — | — | — | 0 | 1 |
| FIBL-6 | 196 | 0 | 0 | — | 0 |
| FIP1L1 | — | 0 | — | — | 1 |
| FKBP14 | — | 0 | 0 | — | 0 |
| FKBP1B | — | — | 1913 | — | 1 |
| FKBP3 | — | — | — | 0 | 1 |
| FKBP7 | — | 0 | 3641 | — | 0 |
| FKSG24 | — | — | 0 | 0 | 1 |
| FLI1 | 4606 | 5759 | 6026 | — | 0 |
| FLJ10006 | — | 297 | 16 | 0 | 1 |
| FLJ10036 | — | — | — | 0 | 1 |
| FLJ10156 | — | — | — | 0 | 1 |
| FLJ10233 | 7599 | — | — | 7599 | 1 |
| FLJ10287 | — | — | — | 76 | 0 |
| FLJ10357 | — | — | — | 0 | 1 |
| FLJ10374 | 0 | 0 | 0 | — | 1 |
| FLJ10514 | — | 94 | 0 | 0 | 1 |
| FLJ10618 | — | — | — | 54 | 1 |
| FLJ10634 | — | 0 | — | 0 | 0 |
| FLJ10652 | 978 | 978 | 978 | — | 1 |
| FLJ10661 | — | — | — | 0 | 0 |
| FLJ10769 | 1484 | 1484 | 1205 | — | 1 |
| FLJ10853 | — | — | — | 1484 | 1 |
| FLJ10884 | — | 24 | 0 | — | 1 |
| FLJ10891 | — | 0 | 0 | 0 | 1 |
| FLJ10901 | — | — | 519 | — | 0 |
| FLJ10922 | — | — | — | 155 | 0 |
| FLJ10970 | — | 2601 | — | — | 0 |
| FLJ10996 | — | 7012 | 7012 | — | 1 |
| FLJ11011 | — | 995 | 995 | — | 1 |
| FLJ11021 | — | 0 | 0 | 0 | 1 |
| FLJ11029 | 256 | 101 | 101 | 0 | 1 |
| FLJ11301 | 0 | 0 | — | — | 1 |
| FLJ11331 | — | 0 | 0 | 0 | 0 |
| FLJ11506 | — | — | — | 0 | 1 |
| FLJ12118 | — | — | 432 | — | 1 |
| FLJ12436 | — | — | — | 0 | 1 |
| FLJ12439 | — | 1953 | — | — | 1 |
| FLJ12476 | — | — | — | 0 | 0 |
| FLJ12505 | — | 0 | — | 0 | 1 |
| FLJ12529 | — | — | — | 0 | 1 |
| FLJ12572 | — | — | 1303 | 1582 | 0 |
| FLJ12666 | — | 0 | 0 | — | 1 |
| FLJ12735 | — | — | — | 0 | 1 |
| FLJ12770 | — | — | 0 | — | 1 |
| FLJ12788 | — | — | 0 | — | 1 |
| FLJ12806 | — | — | — | 4 | 1 |
| FLJ12886 | — | — | — | 0 | 1 |
| FLJ12973 | — | — | — | 0 | 1 |
| FLJ13096 | — | — | 597 | 7 | 0 |
| FLJ13197 | — | 2074 | 1795 | — | 0 |
| FLJ13236 | — | 3767 | — | — | 0 |
| FLJ13273 | — | — | 127 | — | 1 |
| FLJ13291 | — | — | — | 0 | 1 |
| FLJ13479 | — | 0 | — | 0 | 1 |
| FLJ13611 | — | — | — | 4419 | 1 |
| FLJ13912 | — | — | — | 0 | 1 |
| FLJ13941 | — | — | — | 0 | 0 |
| FLJ13984 | — | — | 390 | 0 | 1 |
| FLJ14001 | — | — | — | 0 | 1 |
| FLJ14281 | — | — | 2504 | 2783 | 1 |
| FLJ14346 | — | — | — | 0 | 1 |
| FLJ14397 | — | — | 0 | — | 0 |
| FLJ14451 | — | — | — | 0 | 1 |
| FLJ14640 | — | 95 | — | 0 | 1 |
| FLJ14816 | 0 | 0 | 0 | — | 0 |
| FLJ14936 | 367 | 0 | 367 | 0 | 1 |
| FLJ14981 | — | 169 | — | — | 1 |
| FLJ16008 | — | 539 | — | — | 0 |
| FLJ16517 | — | 0 | 0 | — | 0 |
| FLJ16641 | — | — | 143 | — | 0 |
| FLJ20010 | — | — | 0 | — | 0 |
| FLJ20097 | 0 | 0 | 0 | — | 0 |
| FLJ20130 | — | — | — | 78 | 0 |
| FLJ20156 | — | — | 121 | 0 | 1 |
| FLJ20211 | — | 7167 | 7167 | — | 1 |
| FLJ20225 | — | — | 1018 | — | 0 |
| FLJ20280 | — | — | — | 0 | 1 |
| FLJ20297 | — | — | — | 0 | 1 |
| FLJ20309 | — | 978 | — | — | 1 |
| FLJ20315 | — | 88 | 0 | — | 0 |
| FLJ20331 | — | — | — | 0 | 1 |
| FLJ20364 | — | — | — | 0 | 1 |
| FLJ20487 | — | — | — | 0 | 1 |
| FLJ20508 | — | — | — | 4 | 1 |
| FLJ20512 | — | 35 | 35 | — | 1 |
| FLJ20516 | — | 0 | — | 0 | 1 |
| FLJ20582 | — | 6314 | 6314 | — | 1 |
| FLJ20618 | — | — | — | 4956 | 1 |
| FLJ20641 | — | — | — | 0 | 1 |
| FLJ20643 | — | 0 | 0 | — | 1 |
| FLJ20758 | — | — | 0 | — | 1 |
| FLJ21062 | — | — | 743 | — | 0 |
| FLJ21128 | — | — | — | 0 | 1 |
| FLJ21616 | — | 0 | 0 | — | 1 |
| FLJ21816 | — | — | 107 | — | 1 |
| FLJ21908 | — | — | — | 0 | 1 |
| FLJ21945 | — | — | 0 | — | 1 |
| FLJ22104 | — | — | 0 | — | 1 |
| FLJ22313 | — | — | — | 0 | 1 |
| FLJ22390 | — | — | — | 315 | 0 |
| FLJ22471 | — | 483 | 483 | — | 0 |
| FLJ22573 | — | — | 5024 | — | 1 |
| FLJ22624 | — | — | — | 0 | 1 |
| FLJ22688 | — | 0 | 0 | 0 | 0 |
| FLJ22794 | — | 1359 | 2223 | — | 0 |
| FLJ22795 | — | — | 0 | — | 1 |
| FLJ22875 | — | 0 | 0 | 0 | 1 |
| FLJ22955 | — | 5297 | 135 | — | 1 |
| FLJ23221 | — | 3048 | 3048 | — | 1 |
| FLJ23235 | — | — | — | 68 | 0 |
| FLJ23263 | 0 | 0 | 0 | — | 0 |
| FLJ23447 | — | — | — | 544 | 0 |
| FLJ23451 | — | — | 36 | — | 0 |
| FLJ23518 | — | — | — | 122 | 0 |
| FLJ23861 | — | — | 708 | — | 0 |
| FLJ25333 | — | — | 1199 | — | 0 |
| FLJ25369 | 6216 | 6216 | 6216 | — | 0 |
| FLJ25416 | — | — | 0 | 0 | 1 |
| FLJ25791 | 1205 | 1205 | 1205 | — | 0 |
| FLJ25801 | — | 1717 | 1717 | — | 1 |
| FLJ30655 | — | — | — | 0 | 1 |
| FLJ30707 | — | 1189 | 1189 | — | 1 |
| FLJ30851 | — | — | — | 1037 | 0 |
| FLJ30990 | — | — | 0 | — | 0 |
| FLJ31153 | — | — | 31 | — | 1 |
| FLJ31438 | — | — | 0 | — | 0 |
| FLJ31846 | — | — | — | 282 | 0 |
| FLJ31952 | — | — | — | 0 | 0 |
| FLJ32001 | — | — | 0 | — | 0 |
| FLJ32063 | — | — | 4345 | — | 0 |
| FLJ32112 | — | — | 0 | 0 | 1 |
| FLJ32312 | 455 | 455 | — | — | 0 |
| FLJ32363 | — | — | — | 0 | 1 |
| FLJ32384 | — | 49 | 0 | — | 1 |
| FLJ32421 | — | — | — | 0 | 0 |
| FLJ32447 | 7153 | 5568 | 2626 | — | 0 |
| FLJ32499 | — | 76 | 0 | — | 0 |
| FLJ32675 | — | — | 205 | — | 0 |
| FLJ32734 | — | — | — | 1000 | 0 |
| FLJ32825 | — | 5356 | — | — | 0 |
| FLJ32949 | — | — | — | 0 | 0 |
| FLJ33167 | 6383 | 6383 | 4704 | — | 0 |
| FLJ33768 | 6696 | — | — | — | 0 |
| FLJ33817 | — | 395 | 132 | — | 1 |
| FLJ34047 | — | 6111 | 6111 | — | 0 |
| FLJ35382 | — | 3347 | — | — | 1 |
| FLJ35409 | 2741 | 7354 | 2963 | — | 0 |
| FLJ35867 | — | — | — | 1072 | 1 |
| FLJ35894 | — | 1610 | — | — | 0 |
| FLJ36754 | — | — | — | 0 | 1 |
| FLJ36812 | — | — | — | 0 | 0 |
| FLJ36980 | — | 59 | 59 | — | 0 |
| FLJ37118 | — | 4248 | — | — | 0 |
| FLJ38725 | — | — | — | 423 | 1 |
| FLJ38944 | — | — | — | 0 | 0 |
| FLJ39739 | — | — | 753 | — | 0 |
| FLJ39779 | 760 | 760 | 0 | — | 0 |
| FLJ40113 | — | — | 4909 | 5749 | 0 |
| FLJ40125 | — | 0 | — | — | 1 |
| FLJ40629 | — | — | — | 0 | 0 |
| FLJ40869 | — | — | — | 2 | 0 |
| FLJ41131 | — | 0 | 0 | 0 | 1 |
| FLJ42280 | 273 | — | — | — | 0 |
| FLJ43582 | 2689 | 1673 | 1673 | — | 0 |
| FLJ44186 | — | — | — | 0 | 0 |
| FLJ44313 | — | 718 | 718 | — | 0 |
| FLJ45187 | 2440 | 2440 | 2228 | — | 0 |
| FLJ45455 | — | — | 547 | — | 1 |
| FLJ45645 | — | — | 475 | — | 0 |
| FLJ45832 | 2335 | — | — | — | 0 |
| FLJ45983 | — | — | 806 | — | 0 |
| FLJ46082 | — | — | 3149 | 3149 | 0 |
| FLJ46347 | 1504 | 1504 | 1219 | — | 0 |
| FLJ90652 | — | — | — | 0 | 1 |
| FMO4 | — | 0 | 0 | — | 0 |
| FNDC3A | — | — | — | 0 | 1 |
| FOXA2 | 5 | 4792 | 4792 | — | 0 |
| FOXB1 | 2932 | 3755 | 521 | 7177 | 0 |
| FOXC1 | 2321 | — | 2321 | — | 0 |
| FOXD3 | 3922 | 1014 | 734 | 3922 | 0 |
| FOXG1B | — | — | 5816 | — | 0 |
| FOXJ1 | 1512 | — | 1512 | — | 0 |
| FOXJ2 | — | 6123 | 5852 | — | 1 |
| FOXL2 | — | 1916 | — | — | 0 |
| FOXM1 | — | — | — | 0 | 1 |
| FOXN4 | — | — | — | 0 | 0 |
| FOXO1A | 6723 | 5729 | 5527 | — | 1 |
| FOXO3A | — | — | 1412 | — | 1 |
| FOXP1 | — | 323 | — | — | 1 |
| FOXQ1 | — | — | — | 679 | 0 |
| FRAT2 | 2315 | 2086 | 2086 | — | 1 |
| FRS2 | — | 3108 | 2898 | — | 0 |
| FSTL5 | — | — | 646 | — | 0 |
| FTL | — | 288 | — | — | 1 |
| FTLL1 | 6122 | 6022 | 6022 | — | 0 |
| FUS | 6752 | 6752 | 790 | 223 | 1 |
| FUSIP1 | — | — | — | 0 | 1 |
| FXR1 | — | — | 283 | — | 1 |
| FXYD5 | — | 1909 | 1909 | — | 1 |
| FZD1 | — | 433 | — | 114 | 1 |
| FZD10 | 2446 | 2446 | 2446 | — | 1 |
| FZD2 | — | 213 | 213 | — | 1 |
| FZD3 | — | 3154 | — | — | 1 |
| FZD7 | — | 2244 | 2244 | — | 1 |
| FZD8 | — | — | 1895 | 407 | 1 |
| G22P1 | — | — | — | 0 | 1 |
| G3BP | — | 0 | — | — | 1 |
| GAB2 | — | — | — | 0 | 0 |
| GABARAPL2 | — | — | — | 5330 | 1 |
| GABRA5 | 1266 | — | — | — | 0 |
| GABRG3 | 1433 | — | 1154 | — | 0 |
| GABRR2 | 1644 | — | — | — | 0 |
| GAD1 | — | 3171 | 2925 | — | 0 |
| GAD2 | 1985 | 1985 | 1707 | — | 0 |
| GADD45A | — | — | — | 107 | 1 |
| GADD45G | 0 | 0 | — | — | 1 |
| GADD45GIP1 | — | — | — | 7745 | 1 |
| GALK2 | — | — | 0 | — | 1 |
| GALNT3 | 2574 | 2769 | 2574 | — | 0 |
| GALNT8 | 40 | 40 | 0 | — | 0 |
| GALNTL4 | — | — | 6218 | — | 0 |
| GANC | — | — | 779 | — | 1 |
| GAP43 | 0 | — | — | — | 1 |
| GART | — | — | 0 | — | 1 |
| GAS | — | 6590 | 6590 | — | 0 |
| GAS1 | 213 | — | — | — | 1 |
| GAS2L1 | — | — | 106 | — | 1 |
| GATA3 | — | — | 1291 | — | 0 |
| GATA4 | 3234 | — | — | — | 0 |
| GATA6 | 1227 | — | 1227 | — | 1 |
| GBA | — | — | — | 0 | 0 |
| GBF1 | — | 0 | 0 | — | 1 |
| GBX2 | 2867 | 6781 | 2867 | 2867 | 0 |
| GC20 | — | — | — | 0 | 1 |
| GCDH | — | — | — | 6053 | 1 |
| GCET2 | — | — | 3442 | — | 0 |
| GCM2 | — | 2318 | 4882 | — | 0 |
| GCNT2 | 0 | 0 | 0 | 6540 | 0 |
| GDAP1 | — | 0 | — | — | 1 |
| GDF3 | — | — | 637 | — | 1 |
| GEMIN7 | — | 235 | 0 | — | 0 |
| GFRA1 | 0 | — | — | 0 | 0 |
| GFRA2 | — | — | — | 1077 | 0 |
| GGA1 | — | 6565 | 6565 | — | 1 |
| GGPS1 | — | 0 | — | — | 1 |
| GIF | — | 460 | — | — | 0 |
| GIT2 | — | — | — | 2976 | 0 |
| GJA1 | 4186 | 163 | 0 | — | 1 |
| GK2 | — | 1870 | — | — | 0 |
| GLA | — | — | 0 | — | 1 |
| GLDC | 2024 | — | — | — | 1 |
| GLG1 | — | — | 178 | — | 1 |
| GLI3 | 5013 | 347 | 0 | — | 0 |
| GLUD1 | — | — | 1194 | — | 1 |
| Gm83 | — | — | 344 | — | 1 |
| GMNN | — | — | — | 41 | 1 |
| GNA13 | — | 4870 | 4870 | — | 1 |
| GNA14 | — | — | 446 | — | 0 |
| GNAI1 | — | 6514 | 6514 | — | 1 |
| GNG10 | 328 | 30 | 0 | — | 1 |
| GNG12 | — | — | 5680 | — | 1 |
| GOLGA4 | — | — | 851 | — | 1 |
| GOLGA6 | 3830 | 3541 | 3541 | — | 0 |
| GOLPH4 | — | — | 1264 | — | 1 |
| GORASP2 | — | — | 569 | — | 1 |
| GPAM | — | — | 1281 | — | 0 |
| GPC3 | 3381 | — | — | — | 1 |
| GPC4 | — | — | 877 | — | 1 |
| GPC6 | — | 2025 | 1459 | — | 1 |
| GPD2 | — | — | — | 0 | 1 |
| GPHN | — | — | — | 247 | 0 |
| GPR10 | 18 | — | — | — | 0 |
| GPR154 | 1117 | — | 1117 | — | 0 |
| GPR174 | — | — | — | 5787 | 0 |
| GPR31 | — | — | 5427 | — | 0 |
| GPRC6A | — | 760 | 479 | — | 0 |
| GPS1 | 510 | — | — | — | 1 |
| GRAP2 | 4652 | 4448 | 4448 | — | 0 |
| GREB1 | — | — | 564 | — | 0 |
| GRID2 | 606 | 829 | — | — | 1 |
| GRIM19 | — | — | 0 | — | 1 |
| GRIN3A | — | 609 | — | — | 0 |
| GRIPAP1 | — | — | 15 | — | 1 |
| GRK6 | — | 0 | — | — | 1 |
| GRM4 | — | 1222 | 1222 | — | 0 |
| GRPEL2 | — | — | 0 | — | 1 |
| GSC | 2223 | 96 | 96 | — | 0 |
| GSG2 | — | — | — | 0 | 0 |
| GSH1 | 1035 | — | — | — | 0 |
| GSH-2 | 5008 | 5008 | 399 | — | 0 |
| GSK3A | — | 0 | 0 | 0 | 1 |
| GSK3B | — | 1156 | — | — | 1 |
| GSPT2 | — | 358 | 83 | — | 1 |
| GSTT2 | — | — | 956 | — | 1 |
| GTF2E1 | — | — | 7882 | — | 1 |
| GTF2H2 | — | — | 0 | — | 1 |
| GTF3C4 | 245 | — | 6444 | — | 1 |
| GTF3C5 | — | — | — | 0 | 1 |
| GTL3 | — | 0 | — | 0 | 1 |
| GTPBP1 | 4129 | — | 0 | 0 | 1 |
| GTPBP3 | 0 | 0 | 0 | 0 | 1 |
| GTSE1 | — | — | — | 0 | 1 |
| GUCY2F | 3744 | — | — | — | 0 |
| GYPC | — | — | 681 | — | 1 |
| H17 | — | — | — | 0 | 1 |
| H2AFJ | 2247 | 738 | 738 | 0 | 1 |
| H2AFV | — | — | — | 0 | 1 |
| H2AFX | — | 6517 | 6240 | 0 | 1 |
| H2AFZ | — | — | 507 | 507 | 1 |
| H3F3B | — | 0 | — | — | 1 |
| HAND1 | 3387 | 6638 | 6358 | — | 0 |
| HAND2 | 1178 | 634 | 634 | — | 0 |
| HAS2 | 0 | 0 | 0 | 0 | 1 |
| HAT1 | — | — | — | 0 | 1 |
| HBG1 | 7177 | 7177 | 7177 | — | 0 |
| HBG2 | 2253 | 2253 | 2253 | — | 0 |
| HBP1 | — | 553 | 2898 | — | 1 |
| HBS1L | — | — | — | 0 | 1 |
| HBXAP | — | 0 | — | 0 | 1 |
| HCAP-G | — | — | — | 0 | 1 |
| HCFC1R1 | — | — | 1939 | 0 | 1 |
| HDAC11 | — | — | 6962 | — | 0 |
| HDAC2 | — | 912 | — | — | 1 |
| HDAC9 | — | 0 | — | — | 1 |
| HDCMA18P | — | 0 | 0 | 0 | 1 |
| HDGF2 | — | 398 | — | — | 1 |
| HDHD4 | — | — | — | 0 | 0 |
| HDLBP | — | — | — | 0 | 1 |
| HEAB | — | 7 | — | — | 1 |
| HECTD2 | — | 3525 | 3445 | — | 1 |
| HELLS | — | 491 | 491 | 0 | 1 |
| HEPH | — | 0 | — | — | 1 |
| HERC2 | — | — | — | 156 | 1 |
| HESX1 | 0 | 0 | 0 | — | 1 |
| HEY2 | — | — | 663 | — | 1 |
| HGD | — | — | 1084 | — | 0 |
| HHAT | — | 267 | 267 | — | 0 |
| HHEX | 1098 | 1098 | 1098 | — | 1 |
| HHLA3 | 57 | — | — | 0 | 1 |
| HIF1AN | — | 0 | — | — | 1 |
| HIRIP3 | — | — | — | 0 | 1 |
| HIS1 | — | 80 | 80 | 0 | 1 |
| HIST1H1A | — | 1225 | 1484 | 3241 | 0 |
| HIST1H1B | 3205 | 1862 | 1862 | 1786 | 0 |
| HIST1H1D | 0 | 0 | 0 | 5251 | 0 |
| HIST1H1E | — | — | 1290 | — | 0 |
| HIST1H2AB | — | — | — | 1110 | 0 |
| HIST1H2AE | — | 0 | 896 | 0 | 0 |
| HIST1H2AG | — | — | — | 0 | 0 |
| HIST1H2AH | — | 7506 | — | 0 | 0 |
| HIST1H2AI | — | 0 | — | 0 | 0 |
| HIST1H2AJ | — | 0 | — | 0 | 0 |
| HIST1H2AK | — | — | — | 0 | 0 |
| HIST1H2AL | — | 0 | 0 | 0 | 0 |
| HIST1H2AM | 0 | 0 | 0 | 0 | 0 |
| HIST1H2BB | — | 225 | — | — | 0 |
| HIST1H2BD | — | — | 0 | — | 1 |
| HIST1H2BE | 0 | 0 | 0 | — | 0 |
| HIST1H2BF | 0 | 0 | 0 | 0 | 0 |
| HIST1H2BG | — | 0 | 1144 | 0 | 0 |
| HIST1H2BH | — | 0 | 0 | 0 | 0 |
| HIST1H2BI | 510 | — | — | 1216 | 0 |
| HIST1H2BJ | — | 6068 | — | 0 | 0 |
| HIST1H2BK | — | — | — | 0 | 0 |
| HIST1H2BL | — | 0 | — | 0 | 0 |
| HIST1H2BM | — | 45 | — | 0 | 0 |
| HIST1H2BN | 5912 | — | — | 0 | 0 |
| HIST1H2BO | 240 | 0 | 240 | 0 | 0 |
| HIST1H3A | — | 0 | 889 | 540 | 0 |
| HIST1H3B | — | — | — | 0 | 0 |
| HIST1H3C | — | 1221 | — | — | 0 |
| HIST1H3D | 253 | 0 | 0 | 0 | 0 |
| HIST1H3E | — | 0 | 0 | 6090 | 0 |
| HIST1H3F | — | 624 | 80 | 0 | 0 |
| HIST1H3G | 1670 | — | — | 0 | 0 |
| HIST1H3H | — | 1340 | — | 1235 | 0 |
| HIST1H3I | 755 | 755 | 755 | 718 | 0 |
| HIST1H3J | 1928 | 0 | 0 | 39 | 0 |
| HIST1H4A | — | 0 | 0 | 0 | 0 |
| HIST1H4B | — | — | — | 4330 | 0 |
| HIST1H4C | — | 0 | 0 | 0 | 1 |
| HIST1H4D | — | 201 | 482 | 7756 | 0 |
| HIST1H4E | 4658 | 0 | 0 | 0 | 0 |
| HIST1H4F | 4905 | 4905 | 5179 | 0 | 0 |
| HIST1H4G | — | 4254 | 3710 | 3337 | 0 |
| HIST1H4I | — | 0 | — | 0 | 0 |
| HIST1H4K | 0 | — | — | 0 | 0 |
| HIST1H4L | 1950 | 0 | 0 | 0 | 0 |
| HIST2H2AA | — | 0 | 0 | 0 | 1 |
| HIST2H2AB | 0 | 0 | — | 0 | 0 |
| HIST2H2AC | 704 | 0 | — | 164 | 0 |
| HIST2H2BE | 996 | 0 | — | 456 | 1 |
| HIST2H3C | — | 0 | 0 | 0 | 0 |
| HIST2H4 | 0 | 0 | 0 | 0 | 0 |
| HIST3H2A | — | 320 | 320 | 0 | 1 |
| HIST3H2BB | — | 73 | 73 | 0 | 0 |
| HIST4H4 | 744 | 1803 | 744 | 3072 | 0 |
| HLA-DQA1 | — | — | 621 | — | 0 |
| HLXB9 | — | 3252 | 3252 | — | 0 |
| HMG20A | 0 | 0 | 0 | — | 1 |
| HMGB2 | 0 | — | — | 0 | 1 |
| HMGB3 | — | — | — | 0 | 1 |
| HMGN3 | — | — | — | 0 | 1 |
| HMGN4 | — | — | 1202 | — | 1 |
| HMOX1 | — | 3899 | — | — | 1 |
| HN1 | 447 | 0 | 0 | — | 1 |
| HNRPA1 | — | 0 | 0 | 0 | 1 |
| HNRPA2B1 | — | 116 | 186 | 0 | 1 |
| HNRPAB | — | — | — | 0 | 1 |
| HNRPC | — | 46 | — | 46 | 1 |
| HNRPD | — | — | — | 521 | 1 |
| HNRPH2 | — | — | 59 | — | 1 |
| HNRPH3 | — | — | — | 94 | 1 |
| HNRPK | — | 1748 | — | — | 1 |
| HNRPL | — | 240 | — | — | 1 |
| HNRPLL | — | — | — | 165 | 1 |
| HNRPR | — | — | — | 50 | 1 |
| HNRPU | — | — | — | 0 | 1 |
| HNRPUL1 | — | 0 | 0 | 0 | 1 |
| HOP | 5942 | 5942 | 5942 | — | 0 |
| HOXA1 | — | 3287 | 645 | — | 0 |
| HOXA13 | 5810 | — | — | — | 0 |
| HOXA3 | 2297 | — | — | — | 0 |
| HOXA4 | 637 | — | — | — | 0 |
| HOXA6 | 7473 | — | — | — | 0 |
| HOXA7 | 221 | — | — | — | 0 |
| HOXA9 | 1232 | — | — | — | 0 |
| HOXB1 | 0 | 0 | 0 | — | 0 |
| HOXB13 | — | 4347 | 1201 | — | 0 |
| HOXB3 | 6846 | 124 | 124 | — | 0 |
| HOXB4 | 2952 | — | — | — | 0 |
| HOXB5 | 2758 | — | 2117 | — | 1 |
| HOXB6 | 210 | — | 1940 | — | 0 |
| HOXB9 | — | — | 5435 | — | 0 |
| HOXC12 | 867 | — | — | — | 0 |
| HOXC4 | 1313 | 1313 | 476 | — | 0 |
| HOXD1 | — | — | 615 | — | 0 |
| HOXD11 | 2737 | — | 3015 | — | 0 |
| HOXD12 | — | — | 5258 | — | 0 |
| HOXD13 | — | 3642 | 1198 | — | 0 |
| HPCL2 | — | — | — | 297 | 1 |
| HPRP8BP | — | — | — | 16 | 1 |
| HPSE2 | — | 0 | 0 | — | 0 |
| HRB2 | — | — | 0 | — | 1 |
| HRBL | — | — | — | 0 | 0 |
| HRG | — | — | 1731 | — | 0 |
| HRMT1L6 | — | — | — | 0 | 1 |
| HSC20 | 5032 | 5032 | 5032 | 291 | 0 |
| HSD17B12 | — | 0 | 0 | 0 | 1 |
| HSD17B8 | — | — | — | 4185 | 1 |
| HSFY2 | — | — | — | 5318 | 0 |
| HSPA4 | — | — | 21 | — | 1 |
| HSPA5 | — | — | 0 | — | 1 |
| HSPA8 | — | — | — | 320 | 1 |
| HSPBAP1 | — | — | 251 | — | 1 |
| HSPC016 | — | — | — | 17 | 1 |
| HSPC111 | — | — | 0 | — | 1 |
| HSPC129 | — | — | — | 0 | 1 |
| HSPC138 | — | 0 | — | — | 1 |
| HSPC150 | — | 0 | 0 | 0 | 1 |
| HSPC171 | — | — | 2181 | 0 | 1 |
| HSPC196 | — | — | — | 262 | 1 |
| HSPC268 | — | 0 | 821 | 523 | 0 |
| HSPCA | 2026 | — | 2026 | — | 1 |
| HSPCB | — | 0 | — | — | 1 |
| hSyn | 5009 | 83 | 2742 | 83 | 1 |
| HTF9C | — | — | — | 57 | 0 |
| HTLF | — | — | — | 140 | 0 |
| HTPAP | — | 1156 | — | — | 1 |
| HTR2B | — | — | 301 | — | 0 |
| HTR2C | 2228 | — | — | — | 0 |
| HUMMLC2B | — | 3750 | 3750 | 3896 | 0 |
| HYPE | 2791 | 2791 | — | — | 1 |
| HYPK | — | — | — | 3763 | 1 |
| ICMT | 3422 | 3221 | 3422 | — | 1 |
| ID1 | — | 820 | 820 | — | 1 |
| ID2 | 694 | — | 3709 | — | 1 |
| IDH3A | — | — | 1345 | — | 1 |
| IDH3G | — | 0 | — | — | 1 |
| IER2 | — | — | — | 638 | 1 |
| IER3IP1 | — | — | — | 0 | 1 |
| IER5L | 526 | 526 | 526 | — | 1 |
| IFI16 | 2580 | 2053 | 2053 | — | 1 |
| IFIH1 | 2572 | 2572 | 2572 | — | 0 |
| IFRG28 | — | 7476 | 7476 | — | 0 |
| IGFBP2 | — | 584 | 584 | — | 1 |
| IGFBP3 | — | — | 2381 | — | 1 |
| IK | — | 0 | — | — | 1 |
| IL12A | — | 6556 | — | — | 0 |
| IL1RAPL1 | 688 | — | 688 | — | 1 |
| IL22RA2 | — | — | 1192 | — | 0 |
| IL23A | — | — | — | 4655 | 0 |
| IL2RG | 6495 | 6350 | 6350 | — | 0 |
| IL4I1 | — | — | — | 448 | 0 |
| ILF2 | — | 0 | 0 | — | 1 |
| ILF3 | — | 0 | 0 | 0 | 1 |
| IMAA | — | 698 | 698 | — | 1 |
| IMP-1 | — | 1470 | 1751 | — | 0 |
| IMP-3 | — | 0 | 0 | 0 | 1 |
| ING1 | — | — | 7076 | — | 1 |
| ING4 | — | 0 | 0 | — | 1 |
| INHA | 5730 | 6328 | 5730 | — | 0 |
| INHBA | — | 1031 | — | — | 1 |
| INHBC | — | — | — | 3263 | 1 |
| INM01 | — | 542 | 542 | — | 1 |
| INPP4A | — | — | 1243 | — | 1 |
| INSM1 | 1959 | 1959 | 1959 | — | 0 |
| IPF1 | 1909 | 2189 | 2189 | — | 0 |
| IPLA2(GAMMA) | — | — | — | 0 | 1 |
| IPMK | — | — | 790 | — | 0 |
| IPO8 | — | — | — | 0 | 1 |
| IQCB1 | — | — | — | 0 | 1 |
| IQCE | — | — | — | 2311 | 0 |
| IQGAP3 | — | — | — | 0 | 1 |
| IQSEC2 | — | 1278 | 1053 | — | 0 |
| IRX2 | 81 | 844 | 844 | — | 1 |
| IRX5 | — | — | — | 201 | 0 |
| ISL1 | 358 | 3363 | 358 | — | 0 |
| ISL2 | 3 | — | — | — | 0 |
| ISOC2 | — | — | 3106 | — | 1 |
| ITGA7 | — | — | — | 7729 | 1 |
| ITGB1 | — | — | 3392 | — | 1 |
| ITGB3BP | — | — | — | 0 | 1 |
| ITIH5L | 5775 | 5775 | — | — | 0 |
| JARID2 | 385 | 64 | 2426 | — | 1 |
| JMJD1A | 218 | 0 | 0 | — | 1 |
| JRKL | — | — | — | 873 | 0 |
| JUB | — | 167 | 167 | — | 1 |
| JUN | — | — | 0 | 0 | 1 |
| JUND | 0 | — | — | — | 1 |
| JUP | 7078 | 8 | 7078 | — | 1 |
| K-ALPHA-1 | — | — | — | 0 | 1 |
| KATNAL2 | — | 20 | — | — | 0 |
| KATNB1 | — | — | 1492 | — | 1 |
| KAZALD1 | — | — | — | 29 | 0 |
| KBTBD10 | — | 1495 | 1495 | — | 0 |
| KBTBD6 | — | — | — | 0 | 1 |
| KBTBD7 | — | — | — | 0 | 1 |
| KCNA1 | — | — | — | 1469 | 0 |
| KCNH4 | — | — | — | 0 | 0 |
| KCNH5 | 1269 | 1009 | — | — | 0 |
| KCNH8 | — | 686 | 966 | 966 | 0 |
| KCNJ1 | — | 3181 | 3181 | — | 0 |
| KCNJ4 | — | — | 6522 | — | 0 |
| KCNJ6 | — | 0 | — | — | 0 |
| KCNK1 | — | — | — | 0 | 1 |
| KCNMB2 | — | 0 | 0 | — | 0 |
| KCNMB4 | 5054 | 5054 | — | — | 1 |
| KCNN2 | 1644 | 0 | 1380 | — | 1 |
| KCNN3 | 3255 | 2995 | — | — | 0 |
| KCNQ1DN | — | 1425 | 1425 | — | 0 |
| KCTD13 | — | — | — | 70 | 0 |
| KCTD15 | — | — | 1068 | — | 1 |
| KCTD2 | — | — | 4900 | — | 1 |
| KCTD9 | — | — | — | 382 | 1 |
| KDELR3 | — | — | 6235 | — | 1 |
| KDR | 6593 | 6316 | 6593 | — | 1 |
| KENAE | — | — | — | 2482 | 0 |
| KHDRBS1 | — | — | 5417 | — | 1 |
| KHSRP | — | — | 218 | — | 1 |
| KIAA0056 | — | — | — | 0 | 1 |
| KIAA0063 | 849 | — | 5207 | 4927 | 1 |
| KIAA0101 | 1216 | — | 1216 | 0 | 1 |
| KIAA0117 | — | — | — | 54 | 1 |
| KIAA0152 | — | — | — | 97 | 1 |
| KIAA0174 | 0 | — | — | 0 | 1 |
| KIAA0241 | — | — | 5044 | 5004 | 1 |
| KIAA0247 | — | 372 | — | — | 1 |
| KIAA0251 | — | — | — | 0 | 1 |
| KIAA0406 | — | — | — | 8 | 1 |
| KIAA0495 | — | 0 | — | — | 1 |
| KIAA0586 | — | 300 | — | — | 0 |
| KIAA0652 | — | — | 0 | — | 1 |
| KIAA0701 | — | — | — | 0 | 1 |
| KIAA0738 | — | 4534 | 4534 | — | 1 |
| KIAA0773 | — | — | 6277 | — | 1 |
| KIAA0895 | — | — | — | 0 | 1 |
| KIAA0962 | — | 41 | 41 | 1992 | 1 |
| KIAA1002 | — | — | — | 0 | 1 |
| KIAA1143 | 0 | 0 | 0 | 0 | 1 |
| KIAA1164 | — | 0 | 0 | 0 | 1 |
| KIAA1166 | — | 0 | — | — | 1 |
| KIAA1217 | — | — | 919 | — | 1 |
| KIAA1229 | — | 0 | 0 | — | 1 |
| KIAA1279 | 0 | 0 | — | 0 | 1 |
| KIAA1280 | — | — | 3611 | — | 1 |
| KIAA1344 | — | — | — | 109 | 1 |
| KIAA1468 | — | 200 | — | — | 0 |
| KIAA1505 | — | 0 | 0 | 0 | 0 |
| KIAA1529 | 0 | — | — | — | 0 |
| KIAA1573 | — | 1499 | 1499 | — | 1 |
| KIAA1586 | — | — | — | 0 | 1 |
| KIAA1623 | 2243 | 2243 | 1938 | 2243 | 1 |
| KIAA1683 | 7039 | — | — | — | 0 |
| KIAA1715 | — | — | — | 0 | 0 |
| KIAA1754 | — | 169 | 0 | — | 0 |
| KIAA1764 | — | — | — | 0 | 1 |
| KIAA1799 | — | — | — | 27 | 0 |
| KIAA1853 | 0 | 0 | — | — | 0 |
| KIAA1919 | 4548 | 4548 | 4548 | — | 0 |
| KIAA1972 | — | — | 6990 | — | 1 |
| KIF11 | — | 1349 | — | 0 | 1 |
| KIF13A | — | 532 | 812 | 0 | 0 |
| KIF15 | 0 | 0 | 0 | 0 | 1 |
| KIF18A | — | — | — | 0 | 1 |
| KIF20A | — | — | — | 0 | 1 |
| KIF22 | — | — | — | 0 | 1 |
| KIF2C | — | 0 | — | 0 | 1 |
| KIF9 | — | — | 1031 | — | 1 |
| KITLG | 301 | 790 | 790 | 301 | 0 |
| KLF15 | — | — | 5075 | — | 0 |
| KLF5 | 3447 | 3447 | 3447 | — | 1 |
| KLF6 | — | — | 809 | — | 0 |
| KLF7 | 1333 | — | 274 | — | 1 |
| KLHDC1 | — | — | — | 4794 | 0 |
| KLHL10 | — | — | — | 2111 | 0 |
| KLHL13 | — | 0 | 0 | — | 0 |
| KLHL14 | 283 | — | — | — | 0 |
| KLHL18 | — | — | 591 | — | 1 |
| KLHL4 | 15 | — | 15 | — | 1 |
| KLHL5 | 0 | 0 | 0 | — | 1 |
| KLRA1 | 6640 | — | — | — | 0 |
| KNTC1 | — | 0 | 0 | 0 | 1 |
| KPNA3 | — | 511 | 511 | 6 | 1 |
| KRT12 | — | 6651 | 6651 | — | 0 |
| KRT18 | — | — | 2845 | — | 1 |
| KRTAP13-3 | — | 547 | 178 | — | 0 |
| KRTAP13-4 | — | 3188 | 3188 | — | 0 |
| KRTAP19-6 | 428 | — | — | — | 0 |
| KRTAP3-1 | 608 | — | 0 | — | 0 |
| LAMA4 | 612 | 340 | 108 | — | 1 |
| LARGE | 2392 | 2392 | 2392 | — | 1 |
| LARS | — | — | 0 | — | 1 |
| LASP1 | — | 1697 | — | — | 1 |
| LAT1-3TM | — | 693 | 693 | — | 1 |
| LBP | 3564 | 3564 | 3564 | — | 0 |
| LBX1 | 32 | 32 | 32 | — | 0 |
| LCMT2 | — | — | 0 | 0 | 1 |
| LDHA | — | — | — | 0 | 1 |
| LDOC1 | — | — | — | 4281 | 1 |
| LECT1 | — | — | 850 | — | 1 |
| LEF1 | 608 | — | 2505 | — | 0 |
| LEFTY1 | — | 653 | 653 | — | 1 |
| LEFTY2 | 4499 | 4107 | 4107 | — | 1 |
| LENG9 | — | 138 | — | — | 0 |
| LEPROTL1 | — | — | 7505 | — | 1 |
| LETM2 | — | 22 | 22 | — | 0 |
| LGALS13 | 5514 | — | — | — | 0 |
| LGI1 | 454 | 454 | 454 | — | 0 |
| LHFPL1 | — | 0 | — | — | 0 |
| LHPP | 7128 | 7128 | 7128 | — | 1 |
| LHX1 | 1125 | — | 1125 | — | 0 |
| LHX2 | 563 | 5417 | 5417 | — | 0 |
| LHX4 | — | — | 739 | — | 0 |
| LHX5 | 6103 | 6103 | 6358 | — | 0 |
| LHX8 | — | 1442 | — | — | 0 |
| LIAS | — | 133 | — | — | 0 |
| LIM2 | — | 6157 | 6157 | 6157 | 0 |
| LIPI | — | — | — | 2721 | 0 |
| LMCD1 | — | — | 1608 | — | 1 |
| LMNB1 | — | — | — | 0 | 1 |
| LMO3 | — | 2425 | 2425 | — | 0 |
| LMO6 | — | 4553 | — | — | 0 |
| LOC112937 | — | — | 2789 | — | 0 |
| LOC113828 | — | — | 0 | — | 0 |
| LOC115509 | — | — | 0 | 0 | 0 |
| LOC116238 | — | — | 1328 | — | 1 |
| LOC124402 | — | — | 1072 | 0 | 1 |
| LOC124491 | 7164 | 7164 | 7164 | 0 | 1 |
| LOC126147 | — | — | 1294 | — | 0 |
| LOC127253 | — | — | 370 | — | 1 |
| LOC130951 | — | — | 0 | — | 0 |
| LOC132321 | — | — | — | 0 | 0 |
| LOC134145 | — | — | — | 0 | 0 |
| LOC137392 | — | — | 238 | — | 1 |
| LOC148898 | — | — | 1391 | — | 1 |
| LOC151963 | — | — | 531 | — | 0 |
| LOC153364 | 1906 | 1906 | 1906 | — | 0 |
| LOC153684 | — | — | 1370 | — | 0 |
| LOC162427 | — | 0 | 0 | 0 | 1 |
| LOC169355 | 0 | 0 | 0 | — | 0 |
| LOC196394 | — | — | 3647 | — | 0 |
| LOC200008 | — | — | 0 | 0 | 0 |
| LOC201895 | — | 320 | — | — | 0 |
| LOC220074 | — | — | — | 0 | 0 |
| LOC220869 | — | — | 0 | 3108 | 0 |
| LOC221711 | — | 1915 | 0 | — | 0 |
| LOC283337 | 5066 | 5066 | 5066 | — | 0 |
| LOC283377 | — | — | — | 0 | 0 |
| LOC283932 | — | 0 | — | — | 0 |
| LOC338761 | — | 5411 | — | — | 0 |
| LOC339448 | — | 193 | — | 202 | 1 |
| LOC349136 | 5584 | 535 | 5584 | — | 0 |
| LOC375035 | — | 0 | — | 0 | 0 |
| LOC387700 | — | 658 | 658 | — | 0 |
| LOC388152 | — | — | 0 | — | 0 |
| LOC388818 | — | 3037 | — | — | 0 |
| LOC401137 | — | — | 4610 | — | 0 |
| LOC403313 | — | 21 | 283 | — | 0 |
| LOC440138 | — | — | 0 | — | 0 |
| LOC440590 | 6014 | 6014 | 5667 | — | 0 |
| LOC51057 | — | — | 85 | — | 0 |
| LOC51066 | — | — | 2713 | — | 0 |
| LOC51333 | — | — | — | 6679 | 0 |
| LOC55831 | — | — | — | 0 | 1 |
| LOC55924 | — | — | 1603 | — | 0 |
| LOC55954 | — | 46 | 0 | 46 | 0 |
| LOC57117 | — | — | 13 | — | 1 |
| LOC63920 | — | — | — | 0 | 0 |
| LOC89894 | — | — | — | 0 | 1 |
| LOC90799 | — | 218 | 218 | — | 1 |
| LOC91661 | — | 0 | — | 0 | 0 |
| LOC91689 | — | — | 0 | — | 1 |
| LOH11CR2A | — | 0 | 0 | — | 1 |
| LOXL2 | — | 0 | — | — | 1 |
| LPHN1 | — | 789 | — | 789 | 1 |
| LRAT | 84 | 0 | 0 | — | 1 |
| LRFN3 | 4254 | 4254 | 4254 | — | 1 |
| LRFN5 | — | — | 7113 | — | 0 |
| LRIG3 | — | — | 1504 | — | 1 |
| LRP2 | — | 1955 | 1955 | — | 1 |
| LRP3 | — | — | 7221 | — | 1 |
| LRRC2 | 7863 | 6303 | 445 | — | 0 |
| LRRC28 | — | — | 93 | 93 | 0 |
| LRRC29 | — | — | 1889 | 0 | 0 |
| LRRN1 | 1485 | 1485 | 959 | — | 1 |
| LRRN6A | 1174 | 1174 | 1174 | — | 1 |
| LRRTM3 | 0 | 0 | 0 | — | 0 |
| LSM3 | — | 0 | 0 | 0 | 1 |
| LSM4 | — | 0 | — | 0 | 1 |
| LSM5 | — | — | 0 | 0 | 1 |
| LTBR | — | 6954 | 6954 | — | 0 |
| LUC7L | — | — | — | 0 | 1 |
| LUC7L2 | — | — | — | 0 | 1 |
| LUZP4 | — | — | — | 5655 | 0 |
| LY6G5B | — | — | — | 5536 | 0 |
| LY96 | 5412 | 5412 | 5412 | — | 0 |
| LYPDC1 | — | 1264 | 704 | — | 1 |
| LYPLA3 | — | — | — | 0 | 1 |
| LZTS2 | — | 2554 | — | — | 0 |
| M11S1 | — | 0 | — | — | 1 |
| M96 | — | 0 | — | 0 | 1 |
| MAB21L1 | 261 | 507 | 261 | — | 0 |
| MAC30 | — | — | — | 0 | 1 |
| MAD1L1 | 1868 | — | — | — | 0 |
| MAD2L1 | — | — | — | 0 | 1 |
| MADP-1 | — | 5265 | 5265 | — | 1 |
| MAGEB3 | — | 1451 | 1192 | — | 0 |
| MAGED2 | 3169 | 3169 | — | — | 1 |
| Magmas | — | — | — | 0 | 1 |
| MAK | — | — | — | 0 | 0 |
| MAK3 | — | — | 609 | — | 1 |
| MAMDC2 | — | — | 0 | — | 0 |
| MAN2C1 | 529 | 529 | 529 | — | 1 |
| MAP17 | — | 2809 | — | — | 0 |
| MAP1A | — | — | — | 6246 | 0 |
| MAP1LC3B | — | — | 2986 | — | 1 |
| MAP3K11 | — | 959 | 959 | — | 1 |
| MAP3K12 | — | 449 | 321 | 1056 | 1 |
| MAP3K14 | — | — | — | 0 | 0 |
| MAP3K3 | 13 | — | — | 13 | 1 |
| MAP4K1 | — | — | — | 669 | 1 |
| MAP4K3 | — | — | — | 0 | 1 |
| MAPBPIP | — | 0 | — | — | 1 |
| MAPK14 | — | — | 1469 | — | 1 |
| MAPK8 | — | — | 1639 | — | 1 |
| MAPRE2 | — | 294 | — | — | 1 |
| MAPT | — | — | — | 1318 | 0 |
| MARCH7 | — | — | 0 | 0 | 1 |
| MARVELD2 | — | — | 861 | — | 1 |
| MAST1 | — | 1701 | — | — | 1 |
| MASTL | — | — | — | 0 | 1 |
| MAT2B | — | 612 | 0 | 612 | 1 |
| MAWBP | — | — | — | 926 | 0 |
| MBC2 | — | — | — | 0 | 1 |
| MBNL3 | — | — | — | 5409 | 0 |
| MCC | 691 | — | 691 | — | 1 |
| MCFP | — | — | — | 701 | 0 |
| MCM10 | — | — | — | 0 | 1 |
| MCM3 | — | — | — | 0 | 1 |
| MCM4 | — | — | — | 0 | 1 |
| MCM5 | — | — | — | 185 | 1 |
| MCM6 | — | — | — | 0 | 1 |
| MCTS1 | — | 0 | 0 | — | 1 |
| MDC1 | — | 4443 | — | 1665 | 1 |
| MDH1 | — | — | 0 | — | 1 |
| MDM2 | — | — | 0 | 95 | 0 |
| MDS1 | — | — | 1685 | — | 0 |
| ME3 | 6644 | 6371 | 6371 | — | 0 |
| MED12 | 282 | 282 | 282 | — | 1 |
| MED25 | — | 4307 | 4562 | 4586 | 1 |
| MEF2A | — | — | 210 | — | 1 |
| MEIS1 | 595 | 848 | 0 | — | 0 |
| MEIS2 | 7229 | — | — | — | 1 |
| MEP50 | — | 0 | 0 | — | 1 |
| METT5D1 | — | — | — | 0 | 0 |
| METTL2 | — | — | — | 0 | 0 |
| MFAP1 | — | — | — | 1351 | 1 |
| MFTC | — | — | 49 | — | 0 |
| MGAT3 | — | 2942 | — | — | 0 |
| MGC10485 | — | — | — | 347 | 1 |
| MGC13053 | — | — | — | 554 | 0 |
| MGC13102 | 0 | — | 0 | 0 | 0 |
| MGC13125 | — | — | — | 0 | 1 |
| MGC13170 | — | — | — | 0 | 1 |
| MGC13204 | — | — | — | 0 | 1 |
| MGC14151 | — | 0 | 0 | — | 1 |
| MGC14161 | — | — | — | 0 | 0 |
| MGC14289 | — | 1938 | 1938 | — | 0 |
| MGC14376 | — | 719 | 456 | — | 0 |
| MGC14798 | 1583 | 1583 | 1583 | — | 1 |
| MGC15407 | — | — | 0 | — | 1 |
| MGC15668 | — | — | — | 0 | 1 |
| MGC16279 | — | — | — | 304 | 0 |
| MGC17986 | — | — | — | 0 | 0 |
| MGC20398 | — | — | 0 | — | 0 |
| MGC20781 | — | — | — | 397 | 1 |
| MGC21654 | — | — | — | 0 | 0 |
| MGC2198 | — | — | 6 | — | 1 |
| MGC23908 | — | 43 | — | 0 | 1 |
| MGC2560 | — | — | 0 | — | 0 |
| MGC2655 | — | — | 2029 | 0 | 0 |
| MGC26717 | 6916 | — | 6692 | — | 0 |
| MGC2714 | — | — | 585 | — | 1 |
| MGC2731 | — | — | — | 0 | 0 |
| MGC27466 | — | — | 0 | 0 | 1 |
| MGC2747 | — | — | 0 | — | 1 |
| MGC2752 | — | — | — | 1525 | 0 |
| MGC2803 | — | 1912 | — | — | 1 |
| MGC2941 | — | 158 | 158 | 158 | 1 |
| MGC29649 | — | — | 2022 | — | 0 |
| MGC29814 | — | 8 | 8 | — | 1 |
| MGC29891 | — | — | — | 0 | 0 |
| MGC3040 | — | 3300 | 2851 | — | 1 |
| MGC32020 | — | 0 | — | 0 | 0 |
| MGC3248 | — | — | 308 | — | 1 |
| MGC33367 | — | — | 3765 | — | 0 |
| MGC33371 | — | 0 | — | 0 | 1 |
| MGC34713 | — | — | — | 0 | 0 |
| MGC34799 | — | 0 | 0 | — | 0 |
| MGC34830 | 2568 | 2568 | 2568 | — | 0 |
| MGC35048 | — | — | 5648 | — | 1 |
| MGC35212 | — | 5923 | — | — | 0 |
| MGC35402 | — | 0 | 0 | 0 | 0 |
| MGC35440 | — | — | 1350 | — | 0 |
| MGC3794 | — | — | — | 0 | 1 |
| MGC39518 | — | — | 147 | — | 0 |
| MGC39545 | 0 | 0 | 124 | — | 0 |
| MGC40168 | 1584 | 1383 | 1584 | — | 1 |
| MGC40179 | — | — | 7219 | 7219 | 0 |
| MGC4170 | 1711 | 1711 | 1711 | — | 1 |
| MGC4308 | — | — | — | 22 | 1 |
| MGC43122 | — | — | — | 133 | 0 |
| MGC45800 | — | — | 198 | — | 0 |
| MGC4645 | — | — | 710 | — | 0 |
| MGC47869 | — | 0 | 0 | 0 | 1 |
| MGC4796 | — | — | 119 | — | 0 |
| MGC48625 | — | 16 | — | — | 1 |
| MGC50844 | — | — | — | 91 | 1 |
| MGC51082 | — | 0 | — | — | 1 |
| MGC5178 | — | — | 0 | 0 | 1 |
| MGC5309 | — | — | — | 0 | 1 |
| MGC5391 | — | — | 96 | — | 1 |
| MGC54289 | — | — | 513 | — | 1 |
| MGC5508 | — | 7257 | 7257 | 7257 | 1 |
| MGC70924 | — | — | — | 38 | 0 |
| MGC8721 | — | — | 4454 | — | 1 |
| MGC88374 | — | 0 | — | — | 0 |
| MGC88387 | 0 | — | — | — | 1 |
| MGC8902 | — | — | — | 4483 | 0 |
| MGC8974 | — | — | — | 0 | 1 |
| MGC90512 | — | — | — | 3730 | 0 |
| MGEA5 | 109 | 0 | 0 | 0 | 1 |
| MICA | — | — | 2909 | — | 1 |
| MICB | — | — | 3204 | — | 1 |
| MIG-6 | — | 1215 | — | — | 1 |
| MIR16 | — | — | 1081 | 1081 | 1 |
| MIS12 | — | — | — | 0 | 1 |
| MKI67 | — | — | — | 0 | 1 |
| MKKS | — | 0 | 0 | — | 1 |
| MKRN1 | 4760 | — | 4760 | — | 1 |
| MLH1 | — | 0 | 0 | 0 | 1 |
| MLH3 | — | — | — | 0 | 0 |
| MLL | — | — | — | 179 | 1 |
| MLLT10 | 4775 | 5016 | 4775 | — | 1 |
| MLR2 | — | — | 833 | — | 0 |
| MMAB | — | — | — | 0 | 1 |
| MMD | — | — | — | 0 | 1 |
| MMP10 | — | 0 | 0 | — | 0 |
| MMP16 | 0 | 0 | — | — | 0 |
| MMP2 | 393 | — | 257 | — | 1 |
| MMP9 | — | — | 3594 | — | 1 |
| MNS1 | — | — | — | 0 | 1 |
| MOBK1B | — | — | — | 9 | 1 |
| MOBKL2B | — | — | 707 | — | 1 |
| MOBP | — | 0 | 0 | — | 1 |
| MOCS3 | — | — | 0 | — | 0 |
| MORF4L1 | — | 335 | 599 | 63 | 1 |
| MORF4L2 | — | 382 | 0 | — | 1 |
| MOSPD3 | — | 17 | — | — | 1 |
| MOV10 | — | — | 2095 | — | 1 |
| MPDZ | — | — | 875 | — | 1 |
| MPHOSPH1 | — | 0 | — | 0 | 1 |
| MPHOSPH6 | — | — | — | 0 | 1 |
| MPP4 | — | 641 | 641 | — | 0 |
| MPP6 | — | — | 5517 | — | 1 |
| MR-1 | — | — | — | 0 | 1 |
| MRE11A | — | — | 0 | 0 | 1 |
| MRO | 231 | 231 | — | — | 0 |
| MRP63 | — | — | — | 29 | 1 |
| MRPL13 | 0 | — | — | 0 | 1 |
| MRPL15 | — | 217 | — | — | 1 |
| MRPL17 | — | — | — | 0 | 1 |
| MRPL18 | — | — | — | 322 | 1 |
| MRPL27 | — | — | 0 | — | 1 |
| MRPL34 | — | — | — | 0 | 1 |
| MRPL37 | — | — | 0 | 0 | 1 |
| MRPL39 | — | — | — | 0 | 1 |
| MRPL43 | — | 6858 | — | — | 1 |
| MRPL47 | — | 0 | — | — | 1 |
| MRPL51 | — | — | 497 | — | 0 |
| MRPS11 | — | 4721 | — | — | 1 |
| MRPS16 | 0 | — | 0 | 0 | 0 |
| MRPS18B | — | 197 | — | — | 1 |
| MRPS23 | — | 0 | — | — | 1 |
| MRPS24 | — | — | — | 0 | 1 |
| MRPS25 | — | — | — | 0 | 1 |
| MRPS27 | — | 0 | — | 0 | 1 |
| MRPS31 | — | 0 | — | 0 | 1 |
| MS4A4A | — | — | 4387 | — | 0 |
| MSC | 152 | 397 | 397 | — | 1 |
| MSH2 | — | — | 0 | 0 | 1 |
| MSH5 | — | — | 0 | 0 | 0 |
| MSH6 | — | — | — | 0 | 1 |
| MSL3L1 | — | 1003 | 1003 | — | 1 |
| MST1 | — | — | — | 848 | 0 |
| MTAP | — | — | — | 0 | 1 |
| MTBP | 0 | — | — | 0 | 0 |
| MTHFD1 | — | — | — | 0 | 1 |
| MTHFD2 | — | — | — | 0 | 1 |
| MTM1 | 1589 | 1589 | 1589 | — | 1 |
| MTMR1 | — | — | 4015 | — | 1 |
| MTR | — | — | — | 0 | 1 |
| MTRF1L | — | — | — | 8 | 1 |
| MTSS1 | — | 1858 | — | — | 1 |
| MUC15 | — | — | 6875 | — | 0 |
| MUS81 | — | 1666 | 1666 | 1666 | 1 |
| MUTYH | — | — | — | 0 | 1 |
| MVK | — | — | — | 0 | 1 |
| MXD1 | — | — | — | 0 | 0 |
| MXD3 | — | — | — | 526 | 1 |
| MYC | — | — | — | 0 | 1 |
| MYEF2 | 140 | — | 2 | — | 1 |
| MYF5 | 1749 | 1749 | 967 | — | 0 |
| MYF6 | — | — | 433 | — | 0 |
| MYH2 | — | — | 227 | — | 0 |
| MYNN | — | 415 | — | — | 1 |
| MYO3A | — | — | 460 | — | 1 |
| MYO9A | — | 512 | — | — | 1 |
| MYOD1 | — | — | 6906 | 714 | 0 |
| MYST3 | 1255 | 984 | 1255 | — | 1 |
| NAALAD2 | 340 | 96 | 96 | — | 1 |
| NADSYN1 | — | — | 2637 | — | 1 |
| NAG | — | 11 | — | — | 1 |
| NAGPA | — | — | — | 0 | 0 |
| NANOG | 0 | 0 | 0 | — | 1 |
| NAP1L2 | — | — | 0 | — | 1 |
| NAPA | — | — | — | 0 | 1 |
| NASP | — | — | — | 0 | 1 |
| NBR1 | — | 0 | 0 | — | 1 |
| NCBP1 | 434 | 313 | — | 434 | 1 |
| NCDN | — | 2392 | — | — | 1 |
| NCOA1 | 6590 | 6434 | — | — | 0 |
| NCOR1 | — | — | 1301 | — | 1 |
| NDNL2 | — | — | 624 | — | 1 |
| NDUFA11 | — | 0 | — | 0 | 1 |
| NDUFA2 | — | 0 | — | — | 1 |
| NDUFB3 | — | — | 6 | — | 1 |
| NDUFB5 | — | 0 | — | — | 1 |
| NDUFB8 | — | 5679 | — | — | 1 |
| NDUFS1 | — | — | — | 0 | 1 |
| NDUFS2 | — | — | 121 | — | 1 |
| NDUFV3 | — | — | — | 6671 | 1 |
| NEBL | 1470 | 948 | 667 | — | 1 |
| NEDD4L | — | 834 | 1114 | — | 1 |
| NEDD9 | — | 59 | 0 | — | 0 |
| NEF3 | 1344 | — | — | — | 1 |
| NEFL | 347 | — | — | — | 1 |
| NEGR1 | — | — | 1343 | — | 0 |
| NEIL3 | — | — | — | 0 | 1 |
| NEK2 | — | — | — | 0 | 1 |
| NEK7 | — | — | 0 | — | 0 |
| NEK8 | — | — | 299 | — | 0 |
| NELL1 | 546 | 546 | — | — | 0 |
| NES | — | — | — | 0 | 1 |
| NEUROD1 | — | — | 0 | — | 0 |
| NEUROG1 | 7228 | 6988 | 6988 | — | 0 |
| NEUROG2 | 1093 | 5371 | — | — | 0 |
| NFAT5 | — | — | 462 | — | 1 |
| NFATC2IP | — | — | — | 0 | 1 |
| NFE2 | — | 219 | — | — | 0 |
| NFE2L3 | 1162 | 1162 | 1162 | — | 1 |
| NFIA | 4553 | 4553 | 4273 | — | 0 |
| NFIX | 6658 | 3798 | 3798 | — | 0 |
| NFKBIL1 | — | — | — | 5362 | 0 |
| NFKBIZ | — | — | 6553 | — | 0 |
| NFS1 | — | — | 0 | — | 1 |
| NFYA | — | 0 | — | 0 | 0 |
| NHLRC2 | — | — | — | 0 | 1 |
| NID67 | 960 | 960 | 960 | — | 1 |
| NIF3L1 | — | — | 0 | — | 1 |
| NIFUN | — | — | 0 | 0 | 1 |
| NIN | — | — | — | 0 | 1 |
| NIP30 | — | — | 4675 | — | 1 |
| NIT1 | — | 6073 | — | — | 1 |
| NKIRAS1 | — | 0 | — | — | 1 |
| NKTR | — | 5713 | 5562 | — | 1 |
| NKX2-2 | 215 | 441 | 441 | — | 0 |
| NKX2-3 | 1468 | 7568 | 2570 | 2783 | 0 |
| NKX2-5 | 7489 | — | — | — | 0 |
| NKX6-1 | 4003 | — | — | — | 0 |
| NM_001005287 | — | — | 5823 | — | 0 |
| NM_012294 | — | — | 3753 | — | 0 |
| NM_020642 | — | — | — | 0 | 1 |
| NM_021000 | — | 6539 | 6539 | — | 0 |
| NM_207512 | — | — | 297 | — | 0 |
| NME7 | — | 66 | 0 | — | 1 |
| NMT1 | — | — | 0 | — | 1 |
| NMU | — | 787 | 787 | — | 1 |
| NODAL | — | — | 427 | — | 1 |
| NOL4 | — | 424 | — | — | 0 |
| NOL6 | — | 0 | 0 | — | 1 |
| NOLA3 | — | — | — | 0 | 1 |
| NOLC1 | — | — | 114 | 0 | 1 |
| NOP5/NOP58 | — | — | — | 0 | 1 |
| NOPE | — | — | 148 | — | 1 |
| NOTCH1 | — | 353 | — | — | 1 |
| NP | — | 7417 | 7417 | 7417 | 1 |
| N-PAC | — | — | — | 569 | 1 |
| NPAS2 | — | — | 1184 | — | 1 |
| NPAS3 | 3427 | 2538 | 3427 | — | 0 |
| NPL4 | 1689 | — | — | — | 1 |
| NPM1 | — | — | — | 0 | 1 |
| NPR1 | — | 7196 | 7196 | — | 0 |
| NPY2R | — | 5401 | — | — | 0 |
| NR0B2 | — | — | — | 7513 | 0 |
| NR1H3 | — | — | — | 4588 | 1 |
| NR2E1 | 5018 | 5273 | 2445 | 807 | 0 |
| NR2F1 | 6505 | — | — | — | 0 |
| NR2F2 | 3811 | — | — | — | 1 |
| NR3C2 | — | — | 838 | — | 0 |
| NR4A2 | 5653 | 5900 | 2738 | — | 0 |
| NR4A3 | 862 | 601 | — | — | 0 |
| NR6A1 | 1211 | 68 | 1211 | 68 | 0 |
| NRL | — | 0 | 0 | — | 0 |
| NRN1 | 0 | — | — | — | 0 |
| NS3TP2 | 141 | 0 | 0 | — | 0 |
| NSE2 | 1155 | — | — | — | 1 |
| NSUN3 | — | 0 | 0 | 0 | 1 |
| NT5C2 | — | — | 3629 | — | 1 |
| NT5C3 | — | — | — | 215 | 1 |
| NT5E | 1075 | — | — | — | 0 |
| NTAN1 | — | — | — | 0 | 1 |
| NUBP2 | — | — | — | 126 | 1 |
| NUCKS | 92 | 0 | 92 | 0 | 1 |
| NUDC | — | — | — | 0 | 1 |
| NUDT4 | — | 3507 | — | — | 1 |
| NUDT5 | 0 | — | 0 | — | 1 |
| NUMA1 | — | — | — | 0 | 1 |
| NUP107 | — | — | — | 0 | 1 |
| NUP155 | 56 | — | — | 0 | 0 |
| NUP160 | — | 0 | — | — | 1 |
| NUP214 | — | — | — | 0 | 1 |
| NUP37 | — | — | — | 1304 | 1 |
| NUP54 | — | — | 58 | — | 1 |
| NUPL1 | — | — | — | 0 | 1 |
| NUSAP1 | — | 7 | 0 | 0 | 1 |
| NUT | — | — | — | 1293 | 0 |
| NUTF2 | — | — | — | 0 | 1 |
| NVL | — | — | 0 | — | 1 |
| NYD-SP17 | — | — | 0 | — | 0 |
| OACT2 | — | — | 687 | — | 1 |
| OAZ2 | — | 1181 | 183 | — | 1 |
| ODAG | — | — | 809 | — | 1 |
| ODF1 | — | — | 6836 | — | 0 |
| OFCC1 | — | 3991 | — | — | 0 |
| OGT | — | 0 | — | — | 1 |
| OIP5 | — | 0 | 0 | 0 | 1 |
| OLFM2 | 0 | 381 | — | — | 0 |
| OLFML2A | 7071 | 4979 | 7071 | 5525 | 0 |
| OLFML3 | 1972 | 161 | 1972 | — | 1 |
| OLIG3 | 1636 | 0 | 0 | — | 0 |
| ONECUT1 | 2238 | 2238 | 1687 | — | 0 |
| ONECUT2 | 1810 | — | 1810 | — | 0 |
| OPA3 | — | — | — | 0 | 0 |
| OR10K2 | — | — | 1309 | — | 0 |
| OR13C4 | — | — | 5130 | — | 0 |
| OR2B3 | — | 5972 | 5972 | — | 0 |
| OR2M2 | 5796 | — | — | — | 0 |
| OR3A2 | — | 1707 | — | — | 0 |
| OR3A4 | — | 4285 | — | — | 0 |
| OR4C16 | — | — | — | 2348 | 0 |
| OR51I2 | — | 386 | 386 | — | 0 |
| OR51M1 | — | — | 670 | — | 0 |
| OR52A5 | 4364 | — | 4364 | — | 0 |
| OR52N5 | — | — | — | 5006 | 0 |
| OR5A2 | — | — | 3460 | — | 0 |
| OR5AR1 | 3412 | 3112 | 3112 | — | 0 |
| ORC1L | 191 | 0 | 191 | 0 | 1 |
| ORC3L | — | — | — | 0 | 1 |
| ORC6L | — | 0 | 81 | 0 | 1 |
| OS-9 | — | — | 0 | — | 1 |
| OSBPL1A | — | 326 | 326 | — | 1 |
| OSGEP | — | 3 | 6450 | 6669 | 1 |
| OSR1 | 4188 | — | 1763 | — | 1 |
| OSR2 | 282 | 562 | 842 | — | 0 |
| OSRF | — | — | — | 30 | 0 |
| OTP | 630 | 2597 | 2597 | — | 0 |
| OTUB1 | — | — | 7547 | — | 1 |
| OTX1 | 1174 | 4310 | 1174 | 5873 | 0 |
| OXA1L | — | 0 | 0 | — | 1 |
| OXSR1 | — | — | — | 260 | 0 |
| P15RS | — | — | 5778 | — | 1 |
| P4HA1 | — | — | 1434 | — | 1 |
| PA2G4 | — | — | — | 0 | 1 |
| PAFAH1B3 | — | — | — | 0 | 1 |
| PAG | 5055 | — | — | — | 0 |
| PAH | — | — | 0 | — | 0 |
| PAI-RBP1 | — | — | — | 0 | 1 |
| PAK1 | 5592 | 5592 | — | — | 1 |
| PAPOLA | — | — | — | 0 | 1 |
| PAPSS2 | — | — | 7560 | — | 1 |
| PARD3 | 1390 | — | — | — | 1 |
| PARG | 0 | 0 | 0 | 0 | 1 |
| PARP11 | — | — | — | 0 | 1 |
| PARP2 | — | — | — | 0 | 1 |
| PARP4 | — | — | — | 0 | 0 |
| PARP8 | — | — | 0 | — | 1 |
| PASK | — | — | — | 542 | 1 |
| PAX2 | 5824 | 3932 | — | — | 0 |
| PAX3 | — | — | 1862 | — | 0 |
| PAX6 | 118 | 118 | 118 | — | 0 |
| PAX7 | 272 | — | — | — | 0 |
| PBK | — | — | — | 0 | 1 |
| PBOV1 | 4554 | — | — | — | 0 |
| PBX1 | — | — | 850 | — | 1 |
| PCBP1 | — | 1500 | 1500 | — | 1 |
| PCDH12 | — | 2891 | — | — | 0 |
| PCDH17 | — | 6714 | 1514 | — | 0 |
| PCDH8 | — | 2861 | 2861 | — | 0 |
| PCDHB1 | — | — | 0 | — | 0 |
| PCDHB3 | — | — | — | 2575 | 0 |
| PCDHGA1 | — | — | 656 | — | 0 |
| PCF11 | — | 0 | — | 0 | 1 |
| PCGF4 | 3063 | 3063 | 5821 | — | 0 |
| PCIA1 | — | 27 | 27 | 3852 | 1 |
| PCNA | — | — | 839 | 0 | 1 |
| PCNT2 | — | — | — | 0 | 1 |
| PCSK5 | — | 176 | — | — | 1 |
| PCTK1 | — | 0 | 0 | — | 1 |
| PCTK2 | 1176 | 1176 | 617 | — | 1 |
| PCYT1B | — | 0 | 0 | — | 0 |
| PDCL | 0 | 0 | 0 | — | 1 |
| PDE10A | 4511 | 2902 | 4511 | — | 0 |
| PDE6D | — | — | — | 4864 | 1 |
| PDF | — | 0 | 0 | — | 1 |
| PDHA2 | — | — | 1214 | — | 0 |
| PDHB | — | — | 723 | — | 1 |
| PDZK8 | — | — | — | 0 | 1 |
| PDZRN4 | — | 1586 | — | — | 0 |
| PEA15 | — | — | 1097 | — | 1 |
| PER2 | — | 140 | — | — | 1 |
| PERLD1 | — | 982 | 982 | — | 1 |
| PERP | — | — | 729 | — | 1 |
| PEX1 | — | — | 93 | — | 1 |
| PEX13 | 116 | 116 | — | — | 0 |
| PEX14 | — | — | — | 2050 | 1 |
| PEX5L | — | 290 | 290 | — | 0 |
| PF6 | — | — | 0 | 0 | 0 |
| PFKFB1 | — | — | 5596 | — | 0 |
| PFKFB4 | — | — | — | 697 | 0 |
| PFN4 | — | — | — | 0 | 0 |
| Pfs2 | — | — | — | 0 | 1 |
| PFTK1 | 819 | 819 | 819 | — | 1 |
| PGBD4 | — | — | 0 | — | 0 |
| PGEA1 | 26 | 26 | — | — | 1 |
| PGM2L1 | — | 0 | — | — | 1 |
| PHAX | — | 5161 | — | — | 1 |
| PHF17 | 0 | 0 | 0 | — | 1 |
| PHF21A | 4885 | — | — | — | 0 |
| PHF21B | — | — | 4785 | — | 0 |
| PHF3 | — | — | — | 0 | 1 |
| PHF5A | — | 0 | 0 | — | 0 |
| PHF8 | 3634 | 3634 | 3634 | — | 1 |
| PHGDH | 0 | — | 1640 | 0 | 1 |
| PHPT1 | — | — | — | 0 | 1 |
| PHTF2 | — | — | 127 | 75 | 1 |
| PHYHIP | — | — | 7050 | — | 0 |
| PIAS1 | — | — | — | 0 | 1 |
| PICALM | — | — | 496 | — | 1 |
| PIG8 | — | 320 | — | 0 | 1 |
| PIGC | — | — | — | 0 | 1 |
| PIGL | — | — | 0 | — | 1 |
| PIGN | — | 0 | — | — | 0 |
| PIK3R2 | — | — | 549 | — | 1 |
| PIK3R3 | — | 0 | — | 0 | 1 |
| PIM1 | — | — | — | 30 | 1 |
| PIM2 | — | — | 1426 | — | 1 |
| PIN4 | — | 0 | — | — | 1 |
| PIP5K1C | 0 | 0 | — | — | 1 |
| PIPOX | 0 | 0 | 0 | — | 1 |
| PITX2 | — | 5312 | 5312 | — | 1 |
| PITX3 | — | 3726 | 3726 | — | 0 |
| PKD1 | — | — | — | 0 | 1 |
| PKD1-like | — | 1949 | — | — | 1 |
| PKIB | — | — | 0 | — | 1 |
| PKIG | — | — | 6464 | — | 1 |
| PKLR | — | — | 7131 | — | 0 |
| PKMYT1 | — | — | 1247 | — | 1 |
| PKN2 | — | — | — | 337 | 1 |
| PLA2G4A | 73 | — | — | — | 0 |
| PLAA | 0 | — | — | 0 | 1 |
| PLEK | — | 2730 | — | — | 0 |
| PLEKHA3 | — | 1449 | 1459 | — | 1 |
| PLEKHA4 | — | 3599 | — | — | 0 |
| PLEKHA8 | — | 1283 | 236 | — | 0 |
| PLEKHA9 | — | — | — | 0 | 0 |
| PLEKHF1 | — | — | 1117 | — | 0 |
| PLEKHG3 | — | 2248 | 2248 | — | 1 |
| PLEKHJ1 | — | — | — | 163 | 1 |
| PLK1 | — | — | — | 0 | 1 |
| PLSCR1 | — | — | — | 0 | 1 |
| PLXNA2 | — | — | — | 0 | 0 |
| PNAS-4 | — | — | 276 | — | 1 |
| PNKP | — | — | — | 0 | 1 |
| PNMA1 | — | 6558 | 6255 | — | 1 |
| PNN | — | — | — | 0 | 1 |
| PODXL | — | — | 1208 | — | 1 |
| POLA2 | — | — | — | 0 | 1 |
| POLD3 | — | — | — | 0 | 1 |
| POLDIP3 | — | 0 | — | — | 1 |
| POLE | — | — | — | 0 | 1 |
| POLE2 | — | — | — | 0 | 1 |
| POLE3 | — | 0 | — | 0 | 1 |
| POLL | — | — | 0 | — | 1 |
| POLR1A | — | — | 0 | — | 1 |
| POLR2I | — | — | — | 0 | 1 |
| POLR2J2 | — | — | 0 | 0 | 1 |
| POLR3D | — | — | 5201 | — | 1 |
| POLR3E | — | 35 | — | — | 1 |
| POLR3F | — | — | — | 80 | 1 |
| POLR3G | 1766 | 1766 | 1766 | — | 1 |
| POLR3K | — | — | — | 0 | 1 |
| POM121 | — | 0 | — | — | 1 |
| PORIMIN | — | 732 | 476 | — | 1 |
| POU2F1 | — | 237 | 237 | 237 | 1 |
| POU2F3 | 2974 | — | — | — | 0 |
| POU3F2 | 1541 | — | 1323 | — | 0 |
| POU3F4 | 1451 | 1047 | — | — | 0 |
| POU4F1 | — | — | 3578 | — | 1 |
| POU5F1 | 1103 | 1103 | 1103 | ND | 1 |
| PPAP2A | 1109 | 1109 | 1109 | — | 1 |
| PPARGC1A | — | — | 0 | — | 0 |
| PPFIBP1 | — | — | 1045 | — | 1 |
| PPIA | — | — | — | 0 | 1 |
| PPIL1 | — | — | — | 0 | 0 |
| PPIL3 | — | — | 0 | — | 1 |
| PPIL4 | — | — | — | 149 | 1 |
| PPIL5 | — | — | — | 0 | 1 |
| PPM1B | — | 323 | — | 602 | 1 |
| PPM1D | — | — | — | 0 | 1 |
| PPP1R10 | — | 0 | — | — | 1 |
| PPP1R11 | — | 1322 | — | — | 1 |
| PPP1R12B | — | 5814 | 5814 | 5814 | 0 |
| PPP1R13L | — | — | — | 0 | 0 |
| PPP1R14D | — | 2259 | 2259 | — | 0 |
| PPP1R15A | — | 0 | — | — | 1 |
| PPP1R16B | — | — | 542 | — | 1 |
| PPP1R2 | — | 5762 | 4770 | — | 1 |
| PPP1R7 | — | — | — | 0 | 1 |
| PPP2R1A | — | 182 | 0 | — | 1 |
| PPP2R1B | 4651 | 4651 | 579 | — | 1 |
| PPP2R3A | 944 | 944 | 944 | — | 1 |
| PPP2R3B | — | — | — | 1233 | 0 |
| PPP2R5A | — | — | — | 220 | 1 |
| PPP2R5C | — | 0 | 107 | — | 1 |
| PPP6C | — | — | — | 0 | 1 |
| PRAC | 534 | 534 | 286 | 791 | 0 |
| PRC1 | — | — | — | 0 | 1 |
| PRCC | — | 0 | — | — | 1 |
| PRCP | — | — | 981 | 981 | 1 |
| PRDM13 | 2428 | — | 0 | — | 0 |
| PRDM14 | 3558 | 3558 | 71 | — | 1 |
| PRDX1 | — | 2566 | — | — | 1 |
| PREPL | — | — | 0 | — | 1 |
| PREX1 | — | — | 1521 | — | 0 |
| PRKAR1A | — | 0 | — | — | 1 |
| PRKCDBP | 5250 | 5250 | 957 | — | 1 |
| PRKDC | — | — | — | 159 | 1 |
| PRKRIP1 | 0 | 0 | — | — | 1 |
| PRMT7 | — | — | — | 0 | 1 |
| PRNP | — | 5813 | 6093 | — | 1 |
| PRO0149 | — | — | 239 | — | 1 |
| PRO1853 | — | — | 6 | — | 1 |
| PRO1855 | — | — | — | 0 | 1 |
| PROX1 | 3930 | 3930 | 3930 | — | 0 |
| PRP19 | — | 84 | 0 | 0 | 1 |
| PRPF31 | 0 | — | 0 | — | 1 |
| PRPF39 | — | — | 7058 | — | 1 |
| PRPS1 | 1126 | — | — | — | 1 |
| PRPSAP1 | — | 0 | — | — | 1 |
| PRRG1 | — | — | — | 1286 | 1 |
| PRSS25 | — | — | — | 0 | 1 |
| PRSS8 | — | 968 | 968 | — | 1 |
| PS1D | — | — | — | 0 | 1 |
| PSEN2 | — | 7187 | 7187 | — | 1 |
| PSENEN | — | — | 1856 | — | 1 |
| PSMA1 | — | — | 248 | — | 1 |
| PSMA3 | 4565 | — | — | — | 1 |
| PSMAL | — | — | 484 | — | 0 |
| PSMB1 | — | 337 | 586 | — | 1 |
| PSMB4 | — | — | 0 | — | 1 |
| PSMB5 | — | — | 0 | — | 1 |
| PSMC2 | — | 0 | 0 | — | 1 |
| PSMD14 | — | — | 0 | — | 1 |
| PSMD4 | — | — | — | 0 | 1 |
| PSMD9 | — | — | 0 | — | 1 |
| PTBP1 | — | — | — | 0 | 1 |
| PTCD2 | — | 0 | — | 0 | 0 |
| PTCH | — | 823 | 823 | — | 0 |
| PTD015 | — | 0 | — | 225 | 1 |
| PTE1 | — | 653 | 653 | — | 1 |
| PTF1A | 387 | 809 | 5273 | — | 0 |
| PTGER2 | — | — | 66 | — | 0 |
| PTGS2 | — | — | 1521 | — | 0 |
| PTHLH | 242 | 717 | 717 | — | 0 |
| PTN | — | 1325 | 1325 | — | 1 |
| PTPN1 | — | — | 5668 | — | 1 |
| PTPN2 | 993 | 993 | 993 | — | 1 |
| PTPN23 | — | — | — | 0 | 1 |
| PTPN3 | — | 5846 | — | — | 1 |
| PTPRJ | — | — | 5725 | — | 0 |
| PTRF | 7027 | — | — | — | 0 |
| PUM1 | — | 0 | — | — | 1 |
| PVRL1 | — | 631 | — | — | 0 |
| PWP1 | — | 410 | 410 | — | 1 |
| PXMP2 | — | — | — | 232 | 1 |
| PXMP3 | — | — | 0 | 283 | 1 |
| PYC1 | — | 4429 | — | — | 0 |
| PYGM | — | 1619 | 1619 | — | 0 |
| QARS | — | — | 0 | — | 1 |
| QPCTL | — | — | — | 334 | 0 |
| QTRT1 | — | — | — | 5317 | 0 |
| R3HDM | — | — | 397 | 397 | 1 |
| RAB14 | — | — | — | 69 | 1 |
| RAB15 | — | 1936 | 1936 | — | 1 |
| RAB17 | — | 3361 | 3361 | — | 1 |
| RAB25 | — | 68 | 68 | — | 1 |
| RAB30 | — | 0 | 0 | — | 0 |
| RAB36 | — | — | — | 3175 | 1 |
| RAB38 | — | — | 3897 | — | 1 |
| RAB3D | — | — | 6669 | — | 0 |
| RAB3-GAP150 | — | 1369 | 1369 | — | 1 |
| RAB4B | — | 172 | — | — | 1 |
| RAB5A | 771 | 771 | 496 | — | 1 |
| RAB5B | — | — | 0 | — | 1 |
| RAB5C | — | — | — | 0 | 1 |
| RAB8A | — | — | — | 254 | 1 |
| RABL3 | — | — | 0 | — | 1 |
| RABL4 | — | — | 0 | — | 1 |
| RABL5 | — | — | — | 0 | 1 |
| RACGAP1 | — | — | — | 26 | 1 |
| RAD17 | — | — | — | 395 | 1 |
| RAD18 | — | — | 1166 | — | 0 |
| RAD23A | — | 7072 | 7072 | 7072 | 1 |
| RAD51 | — | — | — | 0 | 1 |
| RAD51C | 53 | — | — | 0 | 1 |
| RAD54B | 171 | 0 | 0 | 0 | 1 |
| RAD54L | — | — | — | 0 | 1 |
| RAD9B | — | — | — | 0 | 0 |
| RAE1 | — | — | — | 0 | 1 |
| RAI1 | — | — | 784 | — | 1 |
| RaLP | — | 0 | 0 | — | 0 |
| RAM2 | — | — | — | 0 | 1 |
| RAMP | — | — | — | 0 | 1 |
| RANBP1 | — | — | — | 0 | 1 |
| RANBP10 | — | 0 | 0 | — | 1 |
| RANBP3 | — | — | — | 0 | 1 |
| RANGAP1 | — | — | 0 | — | 1 |
| RAP1A | — | — | 0 | — | 1 |
| RAP1GDS1 | — | — | 951 | — | 0 |
| RAP80 | — | 3513 | 3513 | — | 1 |
| RARB | — | — | 822 | — | 1 |
| RARSL | — | — | — | 0 | 1 |
| RASA1 | — | 4248 | 4248 | — | 1 |
| RASGRF2 | 3221 | 2747 | 2747 | — | 1 |
| RASIP1 | — | 5721 | 5998 | — | 0 |
| RASL11B | — | 1277 | 997 | — | 1 |
| RAVER1 | — | — | — | 0 | 0 |
| RAX | — | 2559 | 2278 | — | 0 |
| RBAK | — | 0 | — | — | 0 |
| RBBP4 | — | — | 547 | 0 | 1 |
| RBBP5 | 0 | — | — | — | 0 |
| RBBP9 | — | 0 | — | — | 1 |
| RBED1 | — | — | — | 0 | 0 |
| RBL1 | — | — | — | 0 | 1 |
| RBM11 | — | — | — | 6316 | 0 |
| RBM14 | — | — | 0 | 0 | 1 |
| RBM22 | 7421 | 0 | 7421 | 0 | 1 |
| RBM23 | — | 0 | — | 0 | 1 |
| RBM24 | — | — | 1631 | — | 0 |
| RBM4 | — | — | 0 | 0 | 1 |
| RBM7 | — | — | 0 | — | 1 |
| RBM9 | — | — | 0 | — | 1 |
| RBP1 | — | — | 936 | — | 1 |
| RBPMS | — | 1776 | 1010 | — | 1 |
| RC74 | — | 0 | 0 | — | 0 |
| RCOR3 | — | 1756 | — | — | 1 |
| RDH10 | 5882 | 776 | — | — | 1 |
| RDH11 | — | 556 | — | — | 1 |
| RDH5 | — | — | — | 3533 | 0 |
| RER1 | — | — | — | 0 | 1 |
| REST | 1573 | 0 | 0 | — | 1 |
| RetSat | — | — | — | 103 | 1 |
| RFC1 | — | — | — | 0 | 1 |
| RFC2 | — | — | — | 0 | 1 |
| RFC3 | — | — | — | 0 | 1 |
| RFC4 | — | — | — | 0 | 1 |
| RFX1 | — | 113 | 113 | 113 | 1 |
| RFX4 | 0 | 0 | — | — | 0 |
| RGC32 | — | 1255 | — | — | 0 |
| RGL1 | 1479 | — | — | — | 0 |
| RGS10 | — | 1047 | 1047 | — | 1 |
| RGS20 | — | — | 2722 | — | 0 |
| RGS4 | — | 5279 | 5279 | — | 0 |
| RGS9 | — | — | 4277 | — | 0 |
| RGSL2 | — | 0 | 0 | — | 0 |
| RHBDL4 | — | — | 1136 | — | 0 |
| RHD | 5944 | 0 | 0 | — | 0 |
| RIBC1 | — | — | — | 0 | 0 |
| RIF1 | 52 | 331 | 52 | 0 | 1 |
| RIPK1 | — | 856 | 856 | — | 1 |
| RKHD2 | — | 886 | — | — | 1 |
| RNASE4 | — | — | — | 0 | 0 |
| RNASE6 | — | 806 | 50 | — | 0 |
| RNF121 | — | — | 0 | — | 1 |
| RNF123 | — | — | — | 0 | 1 |
| RNF127 | 2917 | 2643 | 2643 | — | 0 |
| RNF14 | — | 4384 | — | — | 1 |
| RNF144 | — | 1478 | 1758 | — | 0 |
| RNF17 | — | — | 2164 | — | 0 |
| RNF2 | — | — | 1038 | — | 1 |
| RNF24 | 1595 | 1595 | 1595 | 0 | 1 |
| RNF25 | — | — | 0 | — | 1 |
| RNF31 | — | 5388 | 5388 | 5388 | 1 |
| RNF34 | — | — | — | 0 | 1 |
| RNF41 | — | — | — | 2155 | 1 |
| RNMT | — | — | — | 0 | 1 |
| RNPC2 | — | 0 | 0 | — | 1 |
| RNPS1 | — | — | — | 0 | 1 |
| RNUT1 | — | — | 0 | — | 1 |
| ROR1 | 595 | 595 | 595 | — | 1 |
| RORB | 3033 | 3033 | 2374 | — | 0 |
| RP11-529I10.4 | — | — | 0 | — | 1 |
| RP6-213H19.1 | — | — | — | 5 | 0 |
| RPL15 | — | 0 | — | — | 1 |
| RPL17 | — | — | 115 | — | 1 |
| RPL21 | — | — | 185 | — | 1 |
| RPL30 | — | 0 | — | — | 1 |
| RPL32 | 0 | — | 105 | — | 1 |
| RPL36A | — | 0 | — | — | 1 |
| RPL4 | — | — | — | 251 | 1 |
| RPL41 | — | — | — | 1566 | 1 |
| RPL7 | — | 2243 | — | — | 1 |
| RPL9 | — | 0 | — | — | 1 |
| RPLP1 | — | 0 | 0 | — | 1 |
| RPP25 | — | — | 1733 | — | 1 |
| RPP30 | — | — | — | 236 | 1 |
| RPRM | — | 761 | 837 | — | 0 |
| RPS13 | — | — | 0 | 0 | 1 |
| RPS18 | 22 | 22 | 22 | 156 | 1 |
| RPS2 | — | — | — | 7064 | 1 |
| RPS23 | — | — | — | 0 | 1 |
| RPS26 | — | 0 | 0 | — | 1 |
| RPS27A | — | — | 0 | — | 1 |
| RPS29 | — | 0 | — | — | 1 |
| RPS3A | 557 | 1451 | 277 | — | 1 |
| RPS6KA6 | — | — | — | 1381 | 0 |
| RQCD1 | — | 0 | 0 | 0 | 0 |
| RRAGD | — | 782 | — | — | 0 |
| RRAS | — | 0 | 0 | 171 | 0 |
| RRM1 | — | — | — | 0 | 1 |
| RRM2 | — | — | — | 0 | 1 |
| RRN3 | — | 0 | 0 | 0 | 1 |
| RRS1 | — | 7303 | 6250 | — | 1 |
| RSBN1L | — | — | — | 0 | 1 |
| RTBDN | — | 673 | — | — | 0 |
| RTDR1 | — | — | — | 0 | 0 |
| RTN2 | — | 2496 | — | — | 1 |
| RTTN | — | — | — | 0 | 1 |
| RUNX1T1 | — | — | 7 | — | 1 |
| RXRB | — | — | — | 199 | 1 |
| RXRG | — | 446 | 446 | — | 0 |
| RYR3 | — | — | 5343 | — | 1 |
| S100A11 | — | — | 6739 | — | 1 |
| S100PBPR | — | — | — | 196 | 1 |
| SAE1 | — | — | — | 0 | 1 |
| SALL1 | 4010 | 4010 | 2451 | — | 1 |
| SALL2 | — | — | 6347 | — | 1 |
| SALL3 | — | — | 4498 | — | 1 |
| SAMSN1 | — | — | — | 0 | 0 |
| SAP30 | — | 923 | — | — | 1 |
| SART3 | — | — | 0 | 0 | 1 |
| SASH1 | — | 0 | 0 | — | 1 |
| SAT | — | 0 | — | — | 1 |
| SAT2 | — | — | 0 | — | 1 |
| SATB2 | — | — | 4840 | — | 0 |
| SAV1 | — | 1089 | 1089 | 0 | 1 |
| SB145 | — | 0 | 0 | — | 0 |
| SB153 | — | — | — | 0 | 0 |
| SBP1 | — | 7276 | 7276 | — | 1 |
| SC5DL | — | 0 | 79 | — | 1 |
| SCAND2 | — | — | 0 | — | 0 |
| SCG3 | — | 0 | — | — | 1 |
| SCGB3A2 | — | — | 2882 | — | 1 |
| SCML1 | — | — | 79 | 0 | 0 |
| SCN3A | — | 22 | — | — | 0 |
| SCNM1 | — | 0 | 36 | — | 1 |
| SCNN1A | — | 1156 | 1372 | — | 1 |
| SDC4 | — | — | 1015 | — | 1 |
| SDCCAG1 | — | 0 | 0 | — | 1 |
| SDCCAG10 | — | — | — | 23 | 1 |
| SDCCAG33 | — | 0 | 117 | — | 0 |
| SDCCAG8 | — | 112 | — | — | 1 |
| SDHD | — | 0 | — | — | 1 |
| SEC10L1 | — | 0 | — | 0 | 1 |
| SEC11L1 | — | — | — | 0 | 1 |
| SEC22L1 | — | — | 619 | — | 1 |
| SEC22L3 | — | — | 388 | — | 1 |
| SEC8L1 | — | — | 0 | — | 1 |
| SECISBP2 | — | — | 7195 | 6913 | 1 |
| SEMA3A | 5973 | 1715 | 1444 | — | 0 |
| SEMA3C | — | — | 370 | — | 1 |
| SEMA4F | — | — | 6217 | — | 1 |
| SEMA6A | — | 1561 | — | — | 1 |
| SENP2 | — | 3451 | 3451 | — | 1 |
| SENP8 | — | 181 | — | — | 0 |
| SEPP1 | — | — | 228 | — | 1 |
| SEPT2 | — | — | — | 0 | 1 |
| SEPT6 | — | 3164 | 3164 | — | 1 |
| SERF2 | — | — | — | 0 | 1 |
| SERP1 | — | — | 0 | — | 1 |
| SERPINA1 | — | 5829 | 5829 | — | 1 |
| SERPINB3 | — | — | 23 | — | 0 |
| SERPINB4 | — | 23 | — | — | 0 |
| SERPIND1 | — | — | — | 2186 | 0 |
| SERPINH1 | — | — | 1002 | — | 1 |
| SESN3 | 1385 | 3952 | 1385 | — | 0 |
| SESTD1 | — | — | 3502 | — | 0 |
| SET | 0 | 0 | 70 | — | 1 |
| SF3A2 | — | — | — | 0 | 1 |
| SF3B1 | — | — | 0 | — | 1 |
| SF3B3 | — | — | — | 0 | 1 |
| SF3B5 | — | 0 | 0 | 0 | 1 |
| SFI1 | — | — | 4920 | — | 1 |
| SFPQ | — | 0 | — | 0 | 1 |
| SFRP1 | 1025 | 1025 | 1025 | — | 1 |
| SFRP2 | 1197 | 1197 | 1197 | — | 1 |
| SFRS11 | — | — | — | 0 | 1 |
| SFRS14 | — | 632 | 632 | — | 1 |
| SFRS15 | — | 813 | 813 | — | 1 |
| SFRS2 | — | — | — | 0 | 1 |
| SFRS3 | — | — | — | 301 | 1 |
| SFRS4 | 4338 | 4338 | 3904 | — | 1 |
| SFRS7 | — | 0 | — | 0 | 1 |
| SFXN1 | 651 | 651 | — | — | 1 |
| SGK | — | — | 3348 | — | 1 |
| SGKL | — | 404 | 162 | — | 1 |
| SGNE1 | — | — | 0 | — | 1 |
| SGOL1 | — | — | — | 0 | 1 |
| SGOL2 | — | — | — | 0 | 1 |
| SGTA | — | 0 | — | — | 1 |
| SH3GL3 | — | 0 | — | — | 1 |
| SH3RF2 | — | — | 0 | — | 0 |
| SHBG | — | — | 0 | — | 0 |
| SHC3 | 0 | 0 | 0 | — | 0 |
| SHCBP1 | — | — | — | 0 | 1 |
| SHQ1 | — | — | — | 0 | 1 |
| SHREW1 | — | 3599 | 3599 | — | 0 |
| ShrmL | 239 | 4 | 4 | — | 0 |
| SIL | — | — | — | 0 | 1 |
| SILV | — | — | 6807 | — | 1 |
| SIM1 | 2141 | — | 976 | — | 0 |
| SIM2 | — | — | 3384 | — | 0 |
| SIMP | — | — | — | 800 | 1 |
| SIN3B | — | — | 3663 | — | 1 |
| SIVA | — | — | — | 0 | 1 |
| SIX1 | 1202 | — | 5287 | — | 0 |
| SIX2 | — | 62 | — | — | 0 |
| SIX3 | — | 3506 | 132 | — | 0 |
| SIX4 | — | — | — | 306 | 0 |
| SIX5 | — | — | — | 77 | 0 |
| SIX6 | 788 | — | — | — | 0 |
| SKIL | 2445 | 2445 | 1181 | — | 1 |
| SLAC2-B | — | 0 | 0 | 0 | 1 |
| SLC10A2 | — | 0 | 0 | — | 0 |
| SLC12A4 | — | — | — | 0 | 0 |
| SLC12A6 | — | — | — | 5398 | 1 |
| SLC13A1 | — | — | 5493 | — | 0 |
| SLC13A4 | — | — | 0 | — | 0 |
| SLC15A2 | — | 0 | 0 | — | 0 |
| SLC16A6 | — | 2513 | — | — | 0 |
| SLC17A6 | — | 164 | — | — | 0 |
| SLC1A1 | — | — | 796 | — | 1 |
| SLC1A5 | — | — | — | 0 | 1 |
| SLC24A2 | 1272 | 1272 | 991 | — | 0 |
| SLC24A3 | 4546 | 4546 | 3676 | — | 0 |
| SLC25A18 | — | 219 | — | — | 0 |
| SLC26A8 | — | — | 1469 | — | 0 |
| SLC2A12 | — | 3146 | — | — | 0 |
| SLC2A4 | — | — | — | 0 | 0 |
| SLC30A1 | 1999 | 2264 | 1999 | — | 0 |
| SLC30A7 | — | — | 436 | — | 1 |
| SLC31A1 | — | — | — | 0 | 1 |
| SLC36A4 | — | — | 27 | — | 1 |
| SLC38A1 | — | — | — | 992 | 1 |
| SLC38A2 | — | 483 | — | — | 1 |
| SLC39A1 | — | 0 | — | 0 | 1 |
| SLC39A5 | — | — | — | 6132 | 0 |
| SLC39A7 | — | — | — | 431 | 1 |
| SLC39A9 | — | — | 0 | — | 1 |
| SLC3A2 | — | 0 | — | 0 | 1 |
| SLC40A1 | — | — | 494 | — | 1 |
| SLC4A1AP | 143 | — | 0 | — | 1 |
| SLC7A11 | — | 0 | 0 | — | 1 |
| SLC7A5 | — | 673 | 673 | — | 1 |
| SLC9A2 | — | 653 | 653 | — | 0 |
| SLC9A5 | — | — | — | 1035 | 0 |
| SLC9A9 | — | 350 | — | — | 0 |
| SLCO4A1 | — | — | — | 120 | 1 |
| SLCO4C1 | — | — | 1066 | — | 0 |
| SLCO5A1 | — | 1357 | — | — | 0 |
| SLITRK5 | — | 1086 | 1591 | — | 0 |
| SLITRK6 | — | — | 0 | — | 0 |
| SMAD3 | — | 1225 | 1225 | — | 1 |
| SMAD7 | — | — | — | 1843 | 1 |
| SMARCA3 | — | 20 | 0 | — | 1 |
| SMARCA5 | — | — | — | 6145 | 1 |
| SMARCAD1 | 0 | 0 | 0 | 0 | 1 |
| SMARCB1 | — | — | — | 175 | 1 |
| SMC1L1 | — | — | — | 0 | 1 |
| SMC4L1 | — | — | — | 0 | 1 |
| SMC6L1 | — | — | — | 101 | 1 |
| SMPDL3A | — | — | 2860 | — | 0 |
| SNAPC1 | — | 0 | — | — | 1 |
| SNAPC3 | — | 0 | — | — | 1 |
| SNAPC5 | — | — | — | 7333 | 1 |
| SNRP70 | — | — | 0 | 0 | 1 |
| SNRPA | — | 0 | 0 | 0 | 1 |
| SNRPA1 | — | — | — | 0 | 1 |
| SNRPB | — | — | — | 0 | 1 |
| SNRPD1 | — | — | — | 0 | 1 |
| SNRPD2 | — | — | — | 0 | 1 |
| SNRPD3 | — | 0 | — | — | 1 |
| SNRPE | — | — | 0 | 0 | 1 |
| SNRPG | — | — | — | 0 | 1 |
| SNRPN | 0 | 0 | 0 | — | 1 |
| SNX1 | — | 1574 | 1790 | 1854 | 1 |
| SNX11 | — | — | — | 6218 | 1 |
| SNX13 | — | — | — | 0 | 1 |
| SNX5 | 5904 | — | — | 0 | 1 |
| SNX7 | — | — | — | 44 | 1 |
| SOCS3 | — | 4326 | 4260 | — | 0 |
| SOCS4 | — | — | 0 | 0 | 0 |
| SON | — | — | 0 | — | 1 |
| SORBS1 | 1507 | — | 1210 | — | 1 |
| SORT1 | — | 1377 | — | — | 1 |
| SOX17 | 1337 | — | — | — | 1 |
| SOX2 | 3469 | 1115 | 2975 | — | 1 |
| SOX5 | 0 | 0 | 0 | — | 0 |
| SP2 | — | 163 | 0 | 0 | 1 |
| SP4 | — | 1148 | — | 0 | 0 |
| SP8 | 408 | — | — | — | 0 |
| SPACA1 | — | — | 1671 | — | 0 |
| SPAG5 | — | — | — | 0 | 1 |
| SPAG6 | 6453 | 6453 | 290 | — | 0 |
| SPAG9 | 0 | 0 | 0 | 0 | 1 |
| SPARC | — | — | 1369 | — | 1 |
| SPATA11 | — | — | 0 | 0 | 1 |
| SPATA16 | — | 1014 | 1014 | — | 0 |
| SPBC24 | — | — | — | 0 | 0 |
| SPBC25 | — | — | — | 0 | 1 |
| SPCS2 | — | 96 | — | 0 | 1 |
| SPG20 | — | — | 300 | — | 1 |
| SPIC | 966 | 696 | 696 | — | 0 |
| SPIN-2 | — | — | — | 1166 | 0 |
| SPIRE2 | — | — | 4861 | — | 1 |
| SPPL2A | — | — | — | 0 | 0 |
| SPRED1 | 453 | 198 | 453 | 0 | 1 |
| SPRY1 | — | — | 0 | — | 1 |
| SR-A1 | — | 1317 | 1317 | 1317 | 1 |
| SRISNF2L | — | — | 6214 | — | 0 |
| SRPR | — | — | — | 0 | 1 |
| SRPX2 | — | — | 7288 | — | 0 |
| SRRM1 | — | — | — | 291 | 1 |
| SS18L2 | — | — | 7724 | — | 1 |
| SSA2 | — | — | — | 325 | 1 |
| SSB3 | — | — | — | 0 | 1 |
| SSBP2 | 4074 | — | 2272 | — | 1 |
| SSBP3 | — | 1261 | 1261 | — | 1 |
| SSH2 | — | — | 781 | — | 0 |
| SSR3 | — | — | — | 1152 | 1 |
| SSR4 | — | 0 | — | — | 1 |
| SSTR1 | 4884 | 4884 | — | — | 0 |
| SSX2IP | — | — | — | 0 | 1 |
| SSX3 | — | 2943 | — | — | 0 |
| ST3GAL2 | — | 1366 | 1366 | — | 1 |
| ST5 | — | — | — | 80 | 1 |
| ST6GAL2 | 3159 | 3159 | 2918 | — | 0 |
| ST8SIA4 | 153 | — | 153 | — | 0 |
| STAF65(gamma) | 0 | — | 0 | — | 1 |
| STAM | — | — | — | 0 | 1 |
| STAP2 | — | 39 | — | — | 1 |
| STARD10 | — | 202 | — | — | 0 |
| STAT1 | — | — | — | 5568 | 1 |
| STAT3 | 403 | 403 | 403 | — | 1 |
| STC1 | — | 1251 | 1251 | — | 1 |
| STCH | — | 0 | — | — | 1 |
| STEAP2 | 1447 | 1447 | 1447 | — | 0 |
| STK11IP | — | — | 0 | — | 1 |
| STK36 | — | — | 0 | — | 1 |
| STK4 | — | — | — | 0 | 1 |
| STK6 | — | — | 0 | 0 | 1 |
| STMN2 | 0 | — | — | — | 1 |
| STRA6 | — | 0 | 0 | — | 0 |
| STRN | — | — | 508 | — | 1 |
| STX10 | — | — | — | 1316 | 1 |
| STXBP2 | — | 7241 | 7241 | — | 1 |
| STYX | — | — | — | 0 | 0 |
| SUCLG1 | — | — | — | 132 | 1 |
| SUFU | — | 90 | 602 | — | 1 |
| SULF1 | 5926 | 0 | 0 | — | 1 |
| SULT1C2 | 2003 | — | — | — | 0 |
| SUMO1 | — | — | 0 | 0 | 1 |
| SUPT16H | — | — | — | 0 | 1 |
| SUPT4H1 | — | — | 776 | — | 1 |
| SURF4 | — | — | — | 0 | 1 |
| SUSD1 | — | — | 5667 | — | 0 |
| SYNGR4 | — | — | — | 0 | 0 |
| SYNPR | 253 | 253 | 253 | — | 0 |
| SYT10 | — | — | — | 0 | 0 |
| T | — | — | 4668 | 808 | 0 |
| T1A-2 | — | 0 | 0 | — | 1 |
| TAF11 | — | — | — | 0 | 1 |
| TAF12 | 5475 | 5475 | 5475 | — | 1 |
| TAF15 | — | 63 | — | — | 1 |
| TAL1 | 380 | 380 | 380 | — | 1 |
| TALDO1 | 2902 | 2902 | 2902 | — | 1 |
| TAOK2 | — | — | — | 0 | 1 |
| TARBP2 | — | 1882 | 1754 | 0 | 1 |
| TAS2R7 | — | — | 0 | — | 0 |
| TBC1D10B | — | 910 | 839 | 910 | 1 |
| TBC1D15 | — | — | — | 0 | 1 |
| TBC1D17 | — | 115 | — | — | 1 |
| TBC1D22B | 530 | — | — | — | 1 |
| TBCC | — | — | 0 | — | 1 |
| TBCE | — | — | — | 56 | 1 |
| TBK1 | — | — | 815 | — | 1 |
| TBL1X | — | — | 0 | — | 1 |
| TBL1XR1 | 1687 | 1687 | 1407 | — | 1 |
| TBL3 | — | — | — | 0 | 1 |
| TBN | — | — | 4204 | — | 0 |
| TBP | — | 176 | 176 | — | 1 |
| TBX20 | 6993 | — | 2783 | — | 0 |
| TBX3 | 754 | — | — | — | 0 |
| TBX5 | 71 | 2795 | 2795 | — | 0 |
| TBX6 | — | — | — | 367 | 1 |
| TCEA1 | — | — | — | 0 | 1 |
| TCEAL8 | — | — | 0 | — | 1 |
| TCERG1 | — | — | 36 | — | 1 |
| TCF12 | 6934 | — | — | — | 1 |
| TCF19 | — | — | — | 0 | 0 |
| TCF20 | 2271 | 2271 | 2271 | — | 1 |
| TCF21 | 47 | — | — | — | 0 |
| TCF3 | — | — | — | 2857 | 1 |
| TCF4 | 710 | — | — | — | 1 |
| TCF7L1 | 1308 | 1308 | 1046 | 1308 | 1 |
| TCF7L2 | — | 0 | 0 | — | 1 |
| TCF8 | — | — | — | 1599 | 0 |
| TCOF1 | — | — | — | 97 | 1 |
| TCP1 | — | — | — | 0 | 1 |
| TCP11 | — | — | 6410 | — | 0 |
| TDE1 | — | — | 2465 | — | 1 |
| TDGF1 | 0 | 0 | 0 | — | 1 |
| TDO2 | — | — | 2805 | — | 0 |
| TDRD5 | 5499 | — | — | — | 0 |
| TEAD2 | — | 931 | — | — | 1 |
| TERF1 | — | — | 5907 | 0 | 1 |
| TESK2 | — | 0 | 0 | 0 | 0 |
| TEX14 | 599 | — | — | 319 | 0 |
| TFAM | — | — | — | 24 | 1 |
| TFAP2C | 4090 | 4090 | 4090 | — | 0 |
| TFAP2D | 1025 | — | — | — | 0 |
| TFB2M | — | — | 0 | — | 1 |
| TFCP2L3 | 864 | 864 | 864 | — | 1 |
| TFPT | 0 | — | 0 | — | 1 |
| TGIF2 | 1418 | 1418 | — | — | 1 |
| THAP1 | — | — | 0 | — | 1 |
| THAP10 | — | — | 0 | 1 | 0 |
| THAP8 | — | — | 38 | 0 | 1 |
| THBS1 | — | — | 974 | 0 | 0 |
| THBS2 | 593 | 593 | 593 | — | 1 |
| THOP1 | — | 1531 | — | — | 1 |
| THRAP6 | — | — | — | 0 | 1 |
| THRB | — | — | 1248 | — | 0 |
| THUMPD3 | — | — | — | 0 | 1 |
| THY28 | — | — | — | 0 | 1 |
| TIA1 | — | 0 | 0 | — | 1 |
| TIAL1 | — | 0 | 0 | 0 | 1 |
| TIAM1 | — | — | 6104 | 245 | 1 |
| TIF1 | 1134 | 1062 | 819 | 819 | 1 |
| TIGD7 | — | — | — | 3942 | 0 |
| TIMELESS | — | — | — | 0 | 1 |
| TIMM23 | 0 | 0 | 0 | 0 | 1 |
| TIMM8B | — | 0 | — | — | 1 |
| TIMM9 | — | 822 | — | — | 1 |
| TIMP4 | — | 0 | 0 | — | 1 |
| TIP120A | — | — | 77 | — | 1 |
| TITF1 | 0 | 0 | 0 | — | 0 |
| TJP3 | 7534 | 7024 | — | — | 1 |
| TK1 | — | — | — | 0 | 1 |
| TLE1 | — | 263 | — | 1180 | 1 |
| TLE2 | — | 6432 | 6432 | 6432 | 1 |
| TLE3 | 377 | 106 | 0 | — | 1 |
| TLK1 | — | — | — | 0 | 1 |
| TLL2 | — | — | — | 321 | 0 |
| TLP19 | — | 0 | — | 29 | 1 |
| TLX1 | 6891 | — | 6891 | — | 0 |
| TM4SF2 | — | — | — | 6230 | 0 |
| TM4SF6 | — | — | 0 | — | 1 |
| TMC1 | — | 2524 | 2524 | — | 0 |
| TMEM16A | — | — | 4873 | — | 0 |
| TMEM23 | 1449 | 1449 | 1659 | — | 1 |
| TMEM24 | — | 4874 | 4874 | — | 0 |
| TMEM30A | 0 | — | — | — | 1 |
| TMEM33 | — | — | — | 5830 | 1 |
| TMEM38A | — | — | 715 | — | 0 |
| TMEM43 | — | — | 2557 | — | 1 |
| TMP21 | — | — | 0 | 212 | 1 |
| TMSB10 | — | 1256 | 1256 | — | 1 |
| TNC | 176 | 0 | 0 | — | 1 |
| TNFAIP2 | — | 3588 | — | — | 1 |
| TNFAIP6 | — | 520 | 520 | — | 0 |
| TNFRSF12A | — | — | 1052 | — | 1 |
| TNFSF5IP1 | — | 439 | — | — | 1 |
| TNPO1 | — | — | 883 | — | 1 |
| TNRC6A | 304 | 304 | 304 | — | 1 |
| TOE1 | — | — | — | 0 | 0 |
| TOMM34 | — | — | — | 5648 | 1 |
| TOP2A | 4894 | 4894 | 4894 | — | 1 |
| TOPBP1 | — | — | — | 0 | 1 |
| TORC2 | — | — | — | 4736 | 0 |
| TP53BP1 | — | — | — | 216 | 1 |
| TPCN2 | — | — | 4280 | — | 0 |
| TPM3 | — | 3067 | — | 0 | 1 |
| TPX2 | — | — | 0 | 0 | 1 |
| TRA1 | — | 278 | — | 0 | 1 |
| TRA2A | 1162 | 1162 | — | — | 1 |
| TRAF7 | — | — | 32 | — | 1 |
| TRAM1L1 | — | 478 | — | — | 0 |
| TRAPPC6B | — | — | — | 4544 | 1 |
| TREX1 | — | — | — | 6532 | 0 |
| TRIM16 | — | 2426 | 2426 | — | 1 |
| TRIM22 | 0 | 0 | 0 | — | 1 |
| TRIM37 | — | — | — | 0 | 1 |
| TRIM39 | — | — | 1570 | — | 0 |
| TRIM41 | — | — | — | 146 | 1 |
| TRIM5 | 4316 | 4316 | 4316 | — | 0 |
| TRIM54 | — | — | 4 | — | 0 |
| TRIM6 | — | — | 868 | — | 0 |
| TRIP10 | — | — | 1514 | — | 1 |
| TRIP11 | — | — | — | 0 | 1 |
| TRIP4 | 4503 | — | 4123 | 5747 | 1 |
| TRIT1 | — | 3167 | — | — | 1 |
| TROAP | — | — | — | 0 | 1 |
| TRPC4AP | — | 1763 | 1362 | — | 1 |
| TRPM3 | — | 289 | 289 | — | 0 |
| TRPS1 | 451 | — | — | — | 1 |
| TSC22D1 | 2529 | 2529 | 2311 | — | 1 |
| TSC22D2 | — | — | 4601 | — | 1 |
| TSGA14 | — | 9 | — | — | 1 |
| TSKS | — | 1494 | 3290 | — | 0 |
| TSNAX | — | — | — | 0 | 1 |
| TSNAXIP1 | — | 0 | 0 | — | 0 |
| TSPYL4 | — | — | — | 848 | 1 |
| TSSC1 | — | — | 74 | — | 1 |
| TSSK6 | — | — | 0 | — | 0 |
| TTC12 | — | 0 | — | 0 | 0 |
| TTC13 | — | — | 5453 | — | 0 |
| TTC15 | — | — | 1248 | — | 0 |
| TTC21B | — | — | — | 74 | 0 |
| TTF1 | — | — | 0 | 0 | 1 |
| TTF2 | — | 0 | 0 | 0 | 1 |
| TUBB | — | 0 | — | 2381 | 1 |
| TUBG1 | — | 115 | 0 | 0 | 1 |
| TUBGCP5 | — | — | — | 0 | 1 |
| TWIST1 | — | — | 768 | — | 0 |
| TXN2 | 14 | — | — | — | 1 |
| TXNDC | — | — | — | 58 | 1 |
| TXNDC11 | — | — | — | 0 | 1 |
| TXNDC5 | — | — | 151 | — | 1 |
| TXNL4B | — | 0 | 0 | — | 0 |
| TXNRD1 | — | 361 | 145 | — | 1 |
| TYMS | — | — | — | 166 | 1 |
| U2AF1L3 | — | — | 2069 | — | 0 |
| UBA2 | — | — | — | 0 | 1 |
| UBAP1 | — | — | — | 0 | 1 |
| UBAP2 | — | — | 949 | 125 | 1 |
| UBC | — | 2177 | 2177 | 51 | 1 |
| UBE2C | — | 0 | — | 0 | 1 |
| UBE2D3 | 1926 | 1926 | 0 | — | 1 |
| UBE2M | — | — | — | 0 | 1 |
| UBE2S | — | — | 175 | 175 | 1 |
| UBE4B | — | — | — | 0 | 1 |
| UBN1 | — | — | — | 0 | 1 |
| UBOX5 | — | 0 | — | — | 0 |
| UBP1 | — | 0 | 0 | 0 | 1 |
| UBQLN4 | — | 421 | 5998 | — | 1 |
| UBR1 | — | 1383 | — | — | 1 |
| UCHL5 | — | — | — | 0 | 1 |
| UCK2 | — | — | — | 1092 | 1 |
| UCRC | — | 102 | 0 | 0 | 1 |
| UFD1L | — | 0 | 0 | 481 | 1 |
| Ufm1 | 2566 | 2566 | 2566 | — | 1 |
| UGP2 | — | — | — | 0 | 1 |
| UGT2B10 | — | 302 | — | — | 0 |
| UGT8 | — | 702 | — | — | 1 |
| UMPS | — | — | — | 0 | 1 |
| UNC5C | — | — | — | 6201 | 0 |
| UNC5D | — | — | 856 | — | 1 |
| UNG | — | — | — | 0 | 1 |
| UNQ1940 | 4985 | 4985 | 4985 | — | 0 |
| UNQ9370 | 5283 | — | 5071 | — | 0 |
| UNQ9372 | — | — | 5526 | — | 0 |
| UQCRC1 | — | — | — | 0 | 1 |
| USF1 | — | — | 0 | — | 0 |
| USH3A | — | — | 1118 | — | 0 |
| USP1 | — | — | — | 0 | 1 |
| USP10 | — | — | 1382 | — | 1 |
| USP16 | — | — | 0 | — | 1 |
| USP25 | — | 2119 | — | — | 1 |
| USP3 | — | — | 437 | — | 1 |
| USP37 | — | 266 | 0 | 0 | 0 |
| USP44 | 0 | 0 | 0 | — | 1 |
| USP49 | — | 404 | — | — | 1 |
| USP5 | — | — | — | 362 | 1 |
| USP52 | — | — | — | 0 | 1 |
| USP7 | 1641 | 1641 | 1641 | — | 1 |
| UTP14A | — | — | 0 | 423 | 1 |
| UTS2 | — | — | 0 | — | 0 |
| UXS1 | — | — | 1086 | — | 1 |
| VAPB | — | — | 79 | — | 1 |
| VASP | — | 7384 | — | — | 1 |
| VAT1 | — | 5111 | — | — | 1 |
| VCL | — | — | — | 0 | 1 |
| VDP | — | 1 | — | — | 1 |
| VIM | — | 852 | 583 | — | 1 |
| VLDLR | — | — | 7402 | — | 1 |
| VMD2L3 | — | — | 112 | — | 0 |
| VNIR2 | — | 2965 | — | — | 0 |
| VPS18 | — | — | — | 0 | 1 |
| VPS29 | — | — | — | 6 | 1 |
| VPS35 | — | 384 | 109 | 0 | 1 |
| VPS52 | 0 | 0 | 0 | 0 | 1 |
| VRK2 | — | — | 7152 | — | 1 |
| VRK3 | — | — | 87 | 495 | 1 |
| VSX1 | — | — | 1131 | — | 0 |
| VTI1A | 0 | — | — | — | 0 |
| WARS | — | — | 0 | — | 1 |
| WBP11 | — | 0 | 0 | 0 | 1 |
| WDFY1 | — | — | — | 452 | 1 |
| WDFY2 | — | 18 | — | — | 1 |
| WDFY3 | 3702 | — | — | — | 1 |
| WDHD1 | — | — | 234 | 0 | 1 |
| WDR20 | 1820 | — | 1820 | — | 1 |
| WDR23 | — | 0 | 0 | — | 1 |
| WDR34 | — | — | — | 0 | 1 |
| WDR36 | 0 | 0 | 0 | 0 | 1 |
| WDR4 | — | — | — | 6110 | 1 |
| WDR49 | 271 | 37 | 0 | — | 0 |
| WDR50 | — | — | — | 0 | 1 |
| WDR56 | — | — | — | 0 | 1 |
| WDR6 | — | — | 0 | 0 | 1 |
| WDTC1 | — | — | 609 | — | 1 |
| WEE1 | 3570 | — | 3570 | — | 1 |
| WFDC6 | — | — | 354 | — | 0 |
| WHSC1L1 | — | 3670 | 3516 | — | 1 |
| WIT-1 | 1635 | — | 1635 | 1635 | 0 |
| WNT3 | — | — | 2842 | — | 0 |
| WT1 | 207 | — | 207 | 92 | 0 |
| WWTR1 | — | 4142 | 4142 | — | 0 |
| XAB1 | — | — | 5809 | — | 1 |
| XAB2 | — | 0 | 0 | — | 1 |
| XAGE2 | 6588 | — | — | — | 0 |
| XCL1 | 4934 | 4934 | 4934 | — | 0 |
| XK | — | 20 | 20 | — | 0 |
| XLKD1 | — | — | 199 | — | 0 |
| XPC | — | 184 | 184 | 184 | 0 |
| XPO1 | — | — | — | 188 | 1 |
| XRCC1 | — | — | — | 0 | 1 |
| XRCC5 | — | — | — | 0 | 1 |
| XTP7 | 0 | — | — | — | 0 |
| YAF2 | 1290 | 1865 | 1290 | — | 0 |
| YAP1 | — | — | 1395 | — | 1 |
| YARS | — | — | — | 0 | 1 |
| YME1L1 | — | — | — | 964 | 1 |
| YPEL4 | — | 0 | — | — | 0 |
| YWHAE | — | — | — | 0 | 1 |
| YWHAQ | — | — | — | 0 | 1 |
| ZA20D1 | — | — | 1025 | — | 1 |
| ZA20D3 | — | — | 587 | — | 1 |
| ZADH2 | — | 1315 | 1315 | — | 0 |
| ZBED3 | — | 304 | — | — | 0 |
| ZBTB1 | — | — | — | 478 | 1 |
| ZCCHC12 | — | — | 0 | — | 0 |
| ZCCHC14 | — | 1314 | — | 96 | 1 |
| ZCCHC3 | — | 6739 | 6739 | 6739 | 1 |
| ZCCHC7 | — | — | — | 0 | 1 |
| ZCWCC1 | — | — | — | 6756 | 1 |
| ZDHHC14 | — | 5240 | 5240 | — | 0 |
| ZDHHC6 | 0 | — | — | — | 1 |
| ZF | — | — | — | 0 | 1 |
| ZFHX1B | 2058 | 2332 | 2332 | — | 1 |
| ZFOC1 | — | 997 | 997 | — | 1 |
| ZFP160L | — | 2001 | — | — | 0 |
| ZFP29 | — | 0 | — | — | 1 |
| ZFP36L1 | 2497 | 2497 | 1733 | — | 1 |
| ZFP42 | — | — | 2279 | — | 1 |
| ZFP95 | 4206 | — | — | — | 1 |
| ZFPL1 | — | — | — | 0 | 1 |
| ZFPM1 | — | — | — | 0 | 0 |
| ZFYVE19 | — | 68 | — | 68 | 1 |
| ZHX2 | 608 | — | — | — | 1 |
| ZIC1 | 1135 | 0 | 1135 | — | 1 |
| ZIC2 | 7510 | 2715 | 7268 | 0 | 1 |
| ZIC3 | 1607 | 1607 | 1607 | — | 1 |
| ZIC4 | 990 | 711 | 711 | — | 0 |
| ZIC5 | 2143 | 2143 | 1834 | 6588 | 0 |
| ZMPSTE24 | 0 | — | — | — | 1 |
| ZMYND12 | — | — | — | 6270 | 0 |
| ZNF140 | — | 0 | — | — | 1 |
| ZNF142 | — | — | — | 0 | 1 |
| ZNF174 | — | — | 0 | 0 | 1 |
| ZNF175 | — | 0 | — | — | 0 |
| ZNF180 | — | 0 | — | 0 | 0 |
| ZNF184 | — | 6253 | — | — | 0 |
| ZNF185 | — | — | 1652 | — | 1 |
| ZNF193 | — | 0 | — | — | 0 |
| ZNF202 | — | — | 0 | — | 1 |
| ZNF206 | — | — | 5302 | — | 1 |
| ZNF217 | — | 1130 | 850 | — | 1 |
| ZNF222 | — | 1023 | — | — | 0 |
| ZNF224 | — | 0 | 0 | 0 | 0 |
| ZNF225 | — | 0 | 0 | — | 0 |
| ZNF226 | — | — | 0 | — | 1 |
| ZNF227 | — | 213 | — | — | 0 |
| ZNF228 | — | — | 0 | — | 1 |
| ZNF281 | — | 191 | 191 | — | 1 |
| ZNF286 | — | 91 | 91 | — | 1 |
| ZNF300 | — | 0 | 0 | 0 | 1 |
| ZNF305 | — | — | 0 | — | 0 |
| ZNF312 | 843 | 1112 | 0 | — | 0 |
| ZNF326 | — | — | — | 131 | 1 |
| ZNF331 | — | 130 | 0 | 0 | 1 |
| ZNF335 | — | 0 | — | 0 | 1 |
| ZNF342 | — | 357 | 357 | — | 0 |
| ZNF367 | — | — | 1866 | 0 | 0 |
| ZNF393 | — | 701 | 413 | — | 0 |
| ZNF394 | 0 | — | — | — | 0 |
| ZNF398 | — | — | 2215 | — | 1 |
| ZNF408 | — | 0 | 0 | — | 0 |
| ZNF416 | — | 4618 | 4618 | — | 0 |
| ZNF417 | — | — | — | 0 | 0 |
| ZNF42 | — | — | — | 0 | 1 |
| ZNF434 | — | — | 0 | 0 | 1 |
| ZNF436 | — | — | 1184 | — | 0 |
| ZNF46 | — | — | — | 596 | 0 |
| ZNF473 | — | — | 80 | 0 | 1 |
| ZNF484 | — | — | 0 | — | 0 |
| ZNF503 | — | — | 714 | 151 | 0 |
| ZNF521 | — | — | 0 | — | 1 |
| ZNF547 | — | 0 | 0 | — | 0 |
| ZNF548 | — | 0 | — | — | 0 |
| ZNF551 | — | — | 0 | — | 1 |
| ZNF554 | — | — | — | 0 | 0 |
| ZNF565 | — | — | — | 241 | 0 |
| ZNF566 | — | 0 | 0 | 0 | 0 |
| ZNF569 | 0 | 0 | — | — | 0 |
| ZNF570 | 1128 | 1378 | — | — | 0 |
| ZNF575 | — | 695 | — | — | 0 |
| ZNF582 | 0 | — | — | 0 | 0 |
| ZNF584 | — | — | 0 | 7124 | 0 |
| ZNF589 | — | — | — | 0 | 1 |
| ZNF600 | — | — | — | 0 | 0 |
| ZNF613 | — | 206 | — | 486 | 0 |
| ZNF624 | — | — | — | 0 | 0 |
| ZNF646 | — | 0 | — | 0 | 1 |
| ZNF75A | — | — | — | 0 | 0 |
| ZNF79 | 4645 | — | — | — | 0 |
| ZNF83 | — | — | — | 0 | 1 |
| ZNF9 | — | — | 472 | — | 1 |
| ZNFN1A3 | 897 | — | — | — | 0 |
| ZNRF2 | — | — | 2455 | — | 1 |
| ZP4 | — | — | 3245 | — | 0 |
| ZPBP2 | 4943 | — | — | — | 0 |
| ZRANB3 | — | — | 0 | 0 | 0 |
| ZSWIM3 | — | 426 | 426 | — | 0 |
| ZWINT | — | — | — | 0 | 1 |

# = distance from nearest transcription start site
1 = expressed
— = not bound

### Example 6

### Mapping PRC2 Subunit Genome Occupancy in ES Cells

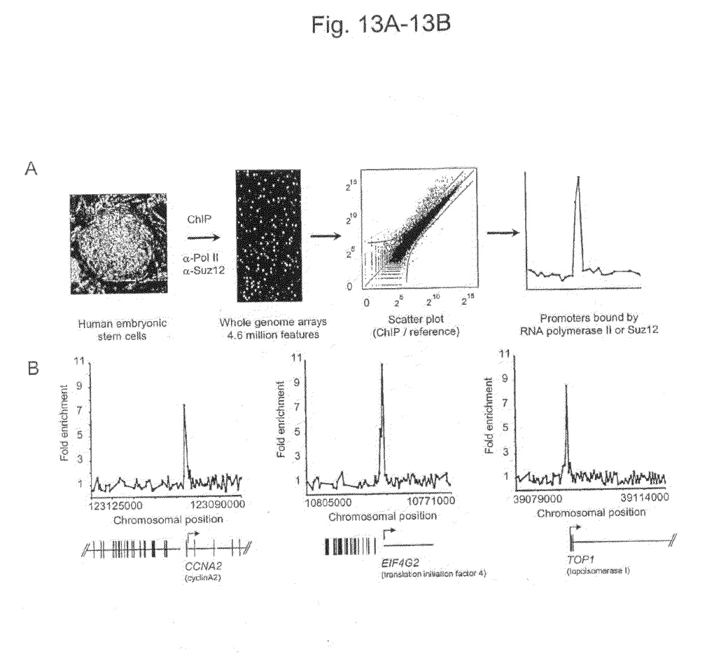

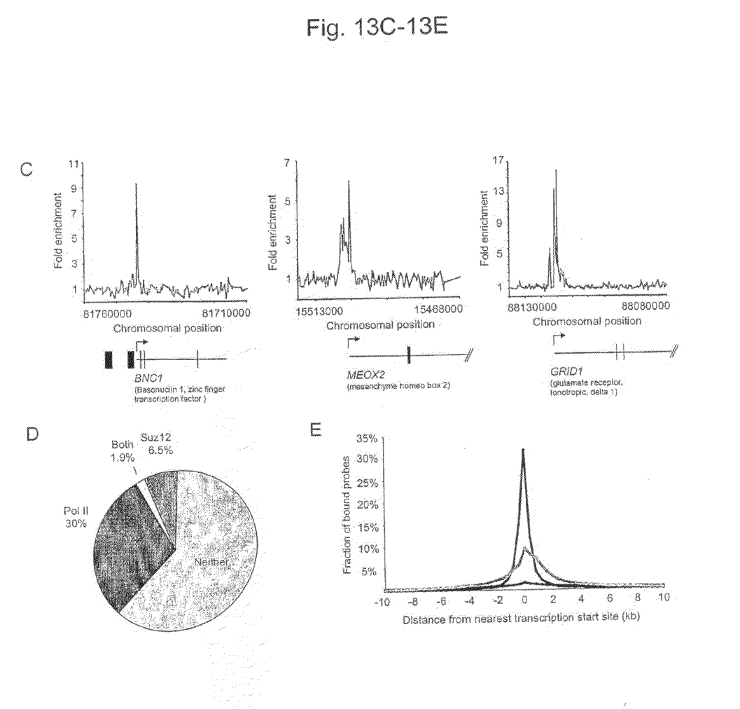

Applicants mapped the location of both RNA polymerase II and the Suz12 subunit of PRC2 genome-wide in human ES cells (FIG. 13). The initiating form of RNA polymerase II was mapped to test the accuracy of the method and provide a reference for comparison with sites occupied by PRC2. The Suz12 subunit of PRC2 is critical for the function of the complex and was selected for these genome-wide experiments. Human ES cells (H9, NIH code WA09) were analyzed by immunohistochemistry for characteristic stem cell markers, tested for their ability to generate cell types from all three germ layers upon differentiation into embryoid bodies and shown to form teratomas in immunocompromised mice.

DNA sequences bound by RNA polymerase II were identified in replicate chromatin-immunoprecipitation (ChIP) experiments using DNA microarrays that contain over 4.6 million unique 60-mer oligonucleotide probes spanning the entire non-repeat portion of the human genome (FIG. 13). To obtain a probabilistic assessment of binding events, an algorithm was implemented that incorporates information from multiple probes representing contiguous regions of the genome and threshold criteria were established to identify a dataset with minimal false positives and false negatives. RNA polymerase II was associated with the promoters of 7,106 of the approximately 22,500 annotated human genes, indicating that one-third of protein-coding genes are prepared to be transcribed in ES cells. Three lines of evidence suggest this dataset is high quality. Most of the RNA polymerase II sites (87%) occurred at promoters of known or predicted genes. Transcripts were detected for 88% of the genes bound by RNA polymerase II in previous expression experiments in ES cells. Finally, independent analysis using gene-specific PCR indicated that the frequency of false positives was approximately 4% and the frequency of false negatives was approximately 30% in this dataset. A detailed analysis of the RNA polymerase II dataset, including binding to miRNA genes, was also done.

The sites occupied by Suz12 were then mapped throughout the entire non-repeat genome in H9 ES cells using the same approach described for RNA polymerase II (FIG. 13C). Suz12 was associated with the promoters of 1,893 of the approximately 22,500 annotated human genes, indicating that −8% of protein-coding genes are occupied by Suz12 in ES cells. Independent site-specific analysis indicated that the frequency of false positives was approximately 3% and the frequency of false negatives was approximately 27% in this dataset.

Comparison of the genes occupied by Suz12 with those occupied by RNA polymerase II revealed that the two sets were largely exclusive (FIG. 13D). There were, however, genes where Suz12 and RNA polymerase II co-occupied promoters. At these genes, PRC complexes may fail to block assembly of the preinitiation complex (Dellino et al., 2004) consistent with the observation that Polycomb group proteins can associate with components of the general transcription apparatus (Breiling et al., 2001; Saurin et al., 2001).

The vast majority of Suz12 bound sites were found at gene promoters (FIG. 13E). Ninety-five percent of the Suz12 bound regions were found within 1 kb of known or predicted transcription start sites. This suggests that Suz12 functions in human ES cells primarily at promoters rather than at distal regulatory elements. It is interesting that 90% of all Suz12 bound regions are within 1 kb of CpG islands, given the recent discovery of a mechanistic link between Polycomb group (PcG) proteins and DNA methyltransferases (Vire et al., 2006).

### Example 7

### Global Transcriptional Repression by PRC2

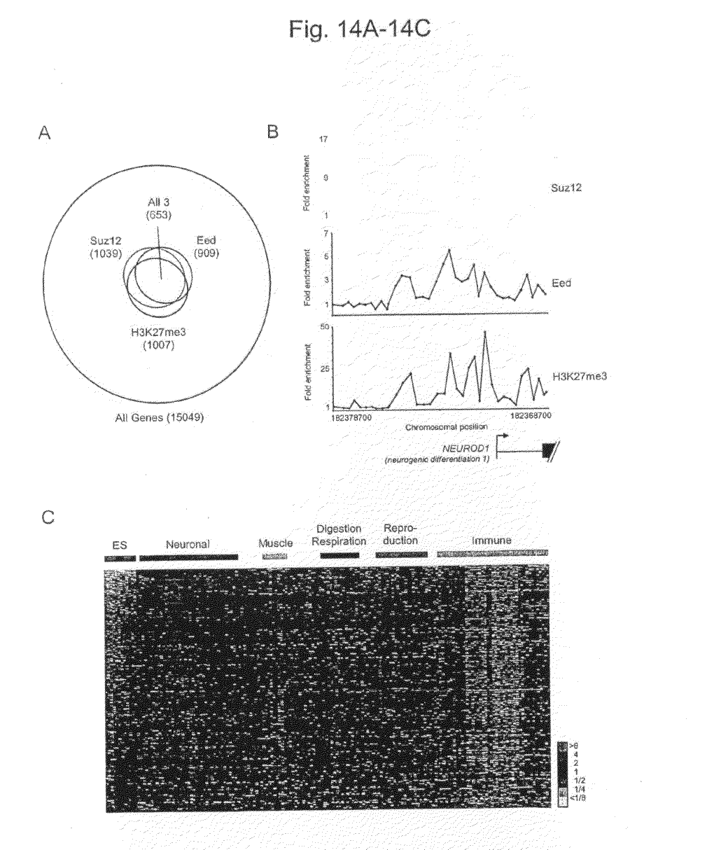

PRC2 is composed of three core subunits, Suz12, Eed and Ezh2, and has been shown to mediate histone H3K27 methylation at specific genes in vivo. To confirm that Suz12 is associated with active PRC2 at target genes, Applicants used chromatin immunoprecipitation with antibodies against Eed and the trimethylated histone H3 lysine-27 (H3K27me3) mark and analyzed the results with promoter microarrays. Applicants found that Eed and the histone H3K27me3 mark co-occurred with Suz12 at most genes using a high-confidence binding threshold (FIG. 14). The false negative rates of thresholded data can lead to an underestimate of the similarity between different datasets. Plotting raw enrichment ratios for genes associated with Suz12, Eed or H3K27me3 demonstrates that Suz12 binding represents PRC2 binding at almost all target genes.

Genetic and biochemical studies at selected genes indicate that PRC2-mediated H3K27 methylation represses gene expression, but it has not been established if it acts as a repressor genome-wide. If genes occupied by Suz12 are repressed by PRC2, then transcripts from these genes should generally be present at lower levels in ES cells than in differentiated cell types. To test this prediction, Applicants compared the expression levels of PRC2-occupied genes in four different ES cell lines with the expression level of these genes in 79 differentiated human cell and tissue types (Sato et al., 2003; Abeyta et al., 2004; Su et al., 2004). Applicants found that PRC2 occupied genes were generally underexpressed in ES cells relative to other cell types (FIG. 14C). A small fraction of the genes occupied by PRC2 were relatively overexpressed in ES cells (FIG. 14C); these tended to show less extensive Suz12 occupancy and were more likely to be co-occupied by RNA polymerase II These results are consistent with the model that PRC2-mediated histone H3K27 methylation promotes gene silencing at the majority of its target genes throughout the genome in ES cells.

### Example 8

### Key Developmental Regulators are Targets of PRC2

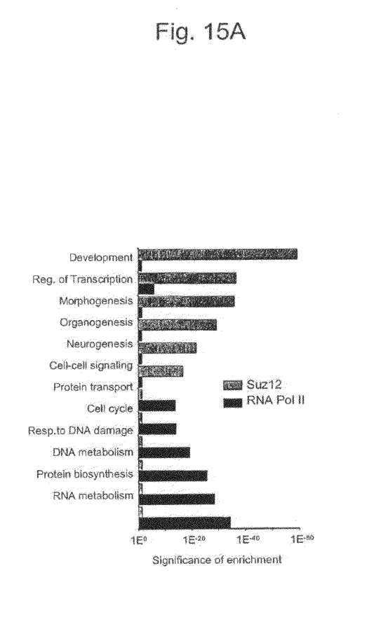

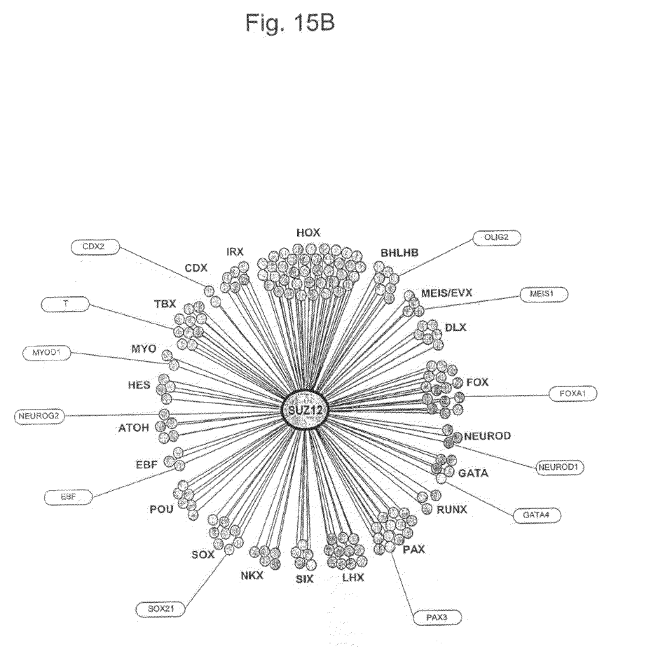

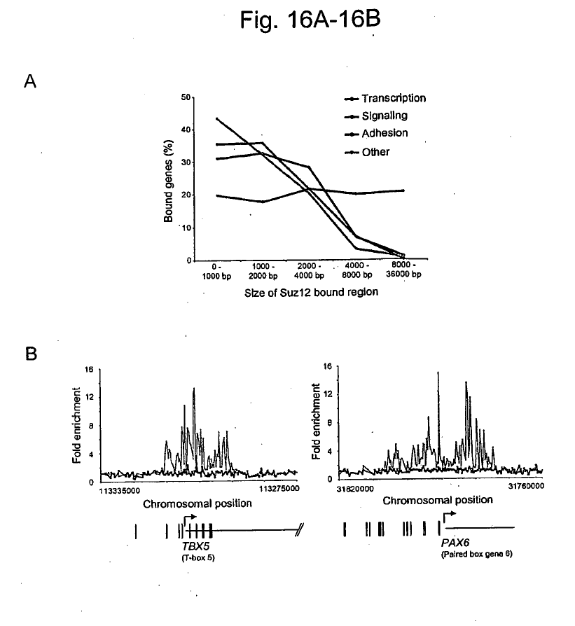

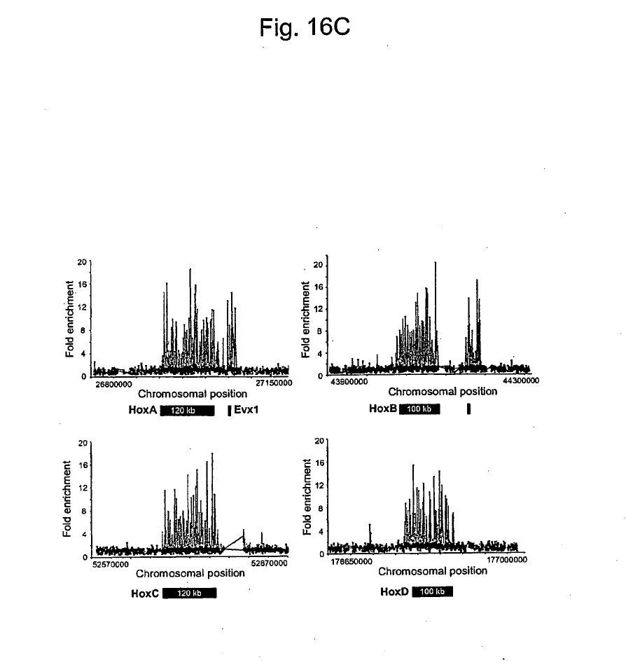

Examination of the targets of Suz12 revealed that they were remarkably enriched for genes that control development and transcription (FIG. 15) and that Suz12 tended to occupy large domains at these genes (FIG. 16). Although only 8% of all annotated genes were occupied by Suz12, −50% of those encoding transcription factors associated with developmental processes were occupied by Suz12. By comparison, RNA polymerase II preferentially occupied genes involved in a broad spectrum of cell proliferation functions such as nucleic acid metabolism, protein synthesis and cell cycle (FIG. 15A and examples in FIG. 13B).

It was striking that Suz12 occupied many families of genes that control development and transcription (FIG. 15B). These included 39 of 40 of the homeotic genes found in the Hox clusters and the majority of homeodomain genes. Suz12-bound homeodomain genes included almost all members of the DLX, IRX, LHX and PAX gene families, which regulate early developmental steps in neurogenesis, hematopoiesis, axial patterning, tissue patterning, organogenesis and cell-fate specification. Suz12 also occupied promoters for large subsets of the FOX, SOX and TBX gene families. The forkhead family of FOX genes is involved in axial patterning and tissue development from all three germ layers (Lehmann et al., 2003). Mutations in members of the SOX gene family alter cell-fate specification and differentiation and are linked to several developmental diseases (Schepers et al., 2002). The TBX family of genes regulates a wide variety of developmental processes such as gastrulation, early pattern formation, organogenesis and limb formation (Showell et al., 2004). Thus, the genes preferentially bound by Suz12 have functions that, when expressed, promote differentiation. This is likely to explain, at least in part, why PRC2 is essential for early development and ES cell pluripotency.

A remarkable feature of PRC2 binding at most genes encoding developmental regulators was the extensive span over which the regulator occupied the locus (FIG. 16). For the majority (72%) of bound sites across the genome, Suz12 occupied a small region of the promoter similar in size to regions bound by RNA polymerase II (FIG. 13). For the remaining bound regions, Suz12 occupancy encompassed large domains spanning 2-35 kb and extending from the promoter into the gene. A large portion of genes encoding developmental regulators (72%) exhibited these extended regions of Suz12 binding. In some cases, binding encompassed multiple contiguous genes. For instance, Suz12 binding extended ˜100 kb across the entire HoxA, HoxB, HoxC and HoxD clusters but did not bind to adjacent genomic sequences, yielding a highly defined spatial pattern (FIG. 16B). In contrast, clusters of unrelated genes, such as the interleukin 1-beta cluster, were not similarly bound by Suz12. Thus, genes encoding developmental regulators showed an unusual tendency to be occupied by PRC2 over much or all of their transcribed regions.

### Example 9

### PRC2 and Highly Conserved Elements

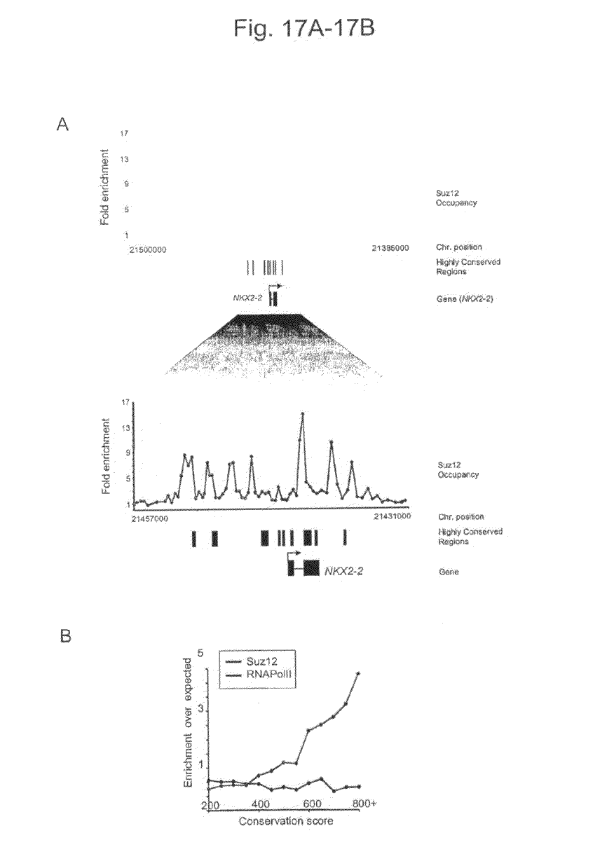

Previous studies have noted that many highly conserved non-coding elements of vertebrate genomes are associated with genes encoding developmental regulators (Bejerano et al., 2004; Siepel et al., 2005; Woolfe et al., 2005). Given Suz12's strong association with this class of genes, Applicants investigated the possibility that Suz12 bound regions are associated with these highly conserved elements. Inspection of individual genes suggested that Suz12 occupancy was associated with regions of sequence conservation (FIG. 17A). Eight percent of the approximately 1,400 highly conserved non-coding DNA elements described by Woolfe and colleagues (Woolfe et al., 2005) were found to be associated with the Suz12-bound developmental regulators (p-value 10-14). Using entries from the PhastCons database of conserved elements (Siepel et al., 2005), Applicants found that Suz12 occupancy of highly conserved elements was highly significant (using highly conserved elements with a LoD conservation score of 100 or better, the p-value for significances was less than 10-85). Since PRC2 has not been shown to directly bind DNA sequences, Applicants expect that specific DNA-binding proteins occupy the highly conserved DNA sequences and may associate with PRC2, which spreads and occupies adjacent chromatin. Thus, the peaks of Suz12 occupancy might not be expected to precisely colocate with the highly conserved elements, even if these elements are associated with PRC2 recruitment.

Remarkably, the degree of the association between Suz12 binding and conserved sequences increases when considering sequences with an increasing degree of conservation (FIG. 17B). By comparison, RNA polymerase II showed no such enrichment. These results suggest that the subset of highly conserved non-coding elements at genes encoding developmental regulators may be associated with PcG mediated silencing of these regulators.

### Example 10

### Signaling Genes are Among PRC2 Targets

The targets of Suz12 were also enriched for genes that encode components of signaling pathways (FIG. 15A). There is evidence that transforming growth factor-beta (TGF□), bone morphogenic protein (BMP), wingless-type MMTV integration site (Wnt), and fibroblast growth factor (FGF) signaling pathways, which are required for gastrulation and lineage differentiation in the embryo, are also essential for self-renewal and differentiation of ES cells in culture (Loebel et al., 2003; Molofsky et al., 2004). Suz12 generally occupied the promoters of multiple components of these pathways, but it occupied larger domains within a group of signaling genes that contained highly conserved elements. This group contained members of the Wnt family (WNT1, WNT2, WNT6) as well as components of the TGF□ superfamily (BMP2, GDF6). Recent studies have shown that Wnt signaling plays a role in pluripotency and self-renewal in both mouse and human ES cells (Sato et al., 2004) and our results suggest that it is important to maintain specific family members in a repressed state in ES cells.

### Example 11

### Activation of PRC2 Target Genes During Differentiation

PRC2 is associated with an important set of developmental regulators that must be silent in ES cells but activated during differentiation. This observation suggests that PRC2 ultimately functions to repress occupied genes in ES cells and that these genes may be especially poised for transcriptional activation during ES cell differentiation. Applicants reasoned that if this model is correct, genes bound by Suz12 should be preferentially activated upon ES cell differentiation or in cells that lack Suz12. Furthermore, in differentiated cells, Suz12 might continue to be observed at silent genes but must be removed from genes whose expression is essential for that cell type.

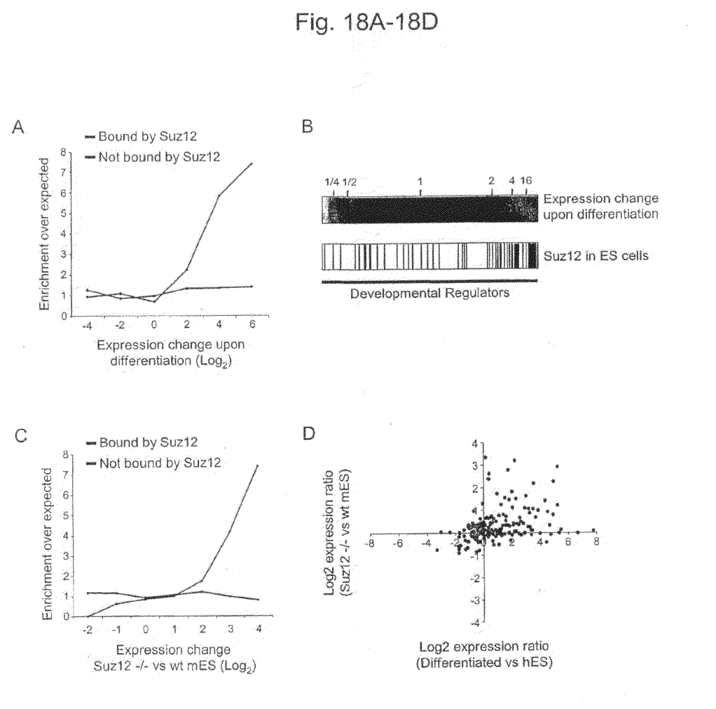

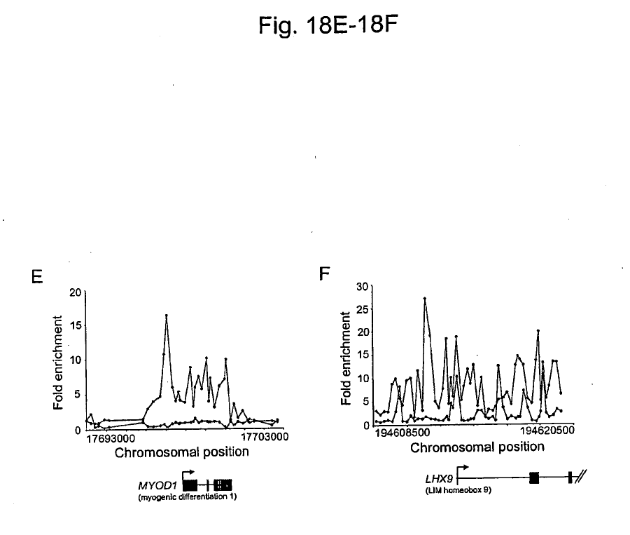

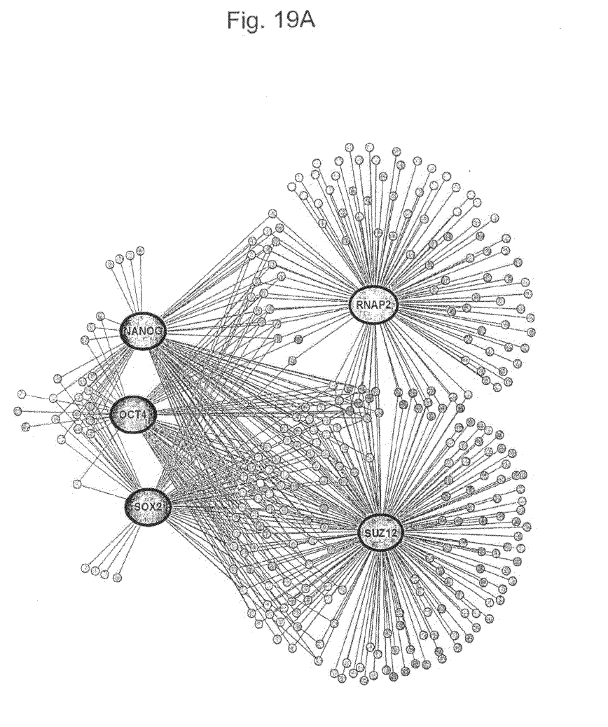

Applicants first examined gene expression in ES cells stimulated to undergo differentiation (Sato et al., 2003). Applicants found that genes occupied by Suz12 were more likely to be activated during ES cell differentiation than genes that were not occupied by Suz12 (FIG. 18A), indicating that Suz12-occupied genes show preferential activation during differentiation under these conditions. Thirty-six percent of genes bound by Suz12 showed greater than 2-fold increases in expression during ES cell differentiation whereas only 16% of genes not bound by Suz12 showed such an increase. This effect was particularly striking at the set of developmental regulators (FIG. 18B). Suz12 occupied most (83%) of the developmental regulators that were induced more than 10-fold during ES cell differentiation.

Applicants next examined the expression of Suz12 target genes in Suz12-deficient cell lines derived from homozygous mutant blastocysts. Applicants reasoned that genes bound by Suz12 in human ES cells have orthologs in mice that should be upregulated in Suz12-deficient mouse cells, although Applicants expected the overlap in these sets of genes to be imperfect because of potential differences between human and mouse ES cells, the possible repression of PRC2 target genes by additional mechanisms, and pleiotropic effects of the Suz12 knockout on genes downstream of Suz12-target genes. Differences in gene expression between Suz12 homozygous mutant cells and wild-type ES cells were measured using gene expression microarrays and the human Suz12 binding data mapped to orthologous mouse genes using HomoloGene (www.ncbi.nlm.nih.gov/HomoloGene). Applicants found that a significant portion of mouse genes whose counterparts were bound by Suz12 in human ES cells were upregulated in Suz12-deficient mouse cells (70 of 346 genes, p=6×10-4). Orthologs of genes occupied by Suz12 in human ES cells were more likely to be activated and less likely to be repressed in Suz12-deficient mouse cells than orthologs of genes not occupied by Suz12 (FIG. 18C). Furthermore, Applicants found that the Suz12 target genes that were induced upon human ES cell differentiation were generally also induced upon loss of Suz12 in mouse cells (FIG. 18D). Genes that were activated during ES cell differentiation and in Suz12-deficient cells included the transcriptional regulators GATA2, GATA3, GATA6, HAND1, MEIS2 and SOX17, the signaling proteins WNT5A, DKK1 (dickkopf homolog 1), DKK2, EFNA1 (ephrin A1), EFNB1, EPHA4 (ephrin receptor A4) and EPHB3 and the cell cycle inhibitor CDKN1A. These data indicate that Suz12 is necessary to fully repress the genes that are occupied by PRC2 in wild-type ES cells.

If PRC2 functions to repress genes in ES cells that are activated during differentiation, then in differentiated tissues Suz12 occupancy should be diminished at genes encoding developmental regulators that have a role in specifying the identity of that tissue, similar to results seen with Ezh2 at specific genes in mouse (Caretti et al., 2004). To test this, Applicants designed an array focused on the promoters of developmental regulators and used ChIP-Chip to investigate Suz12 occupancy at these promoters in primary differentiated muscle cells. The results demonstrated that genes encoding key regulators of muscle differentiation, including MyoD, displayed greatly diminished Suz12 occupancy when compared to ES cells (FIG. 18E). MyoD is a master regulator for muscle differentiation (Tapscott, 2005), and the gene encoding this transcription factor displayed no significant Suz12 occupancy when compared to the levels of Suz12 occupancy observed in ES cells. Genes encoding other transcriptional regulators that play a central role in muscle development, such as Pax3 and Pax7 (Brand-Saberi, 2005), showed reduced levels of Suz12 occupancy in muscle cells relative to ES cells. In contrast, other developmental regulators important for differentiation of non-muscle tissues remained occupied by Suz12 in differentiated muscle cells (FIG. 18F). These data support a model where PRC2 binding in ES cells represses key developmental regulators that are later expressed during differentiation.

### Example 12

### Targets of PRC2 are Shared with Key ES Cell Regulators

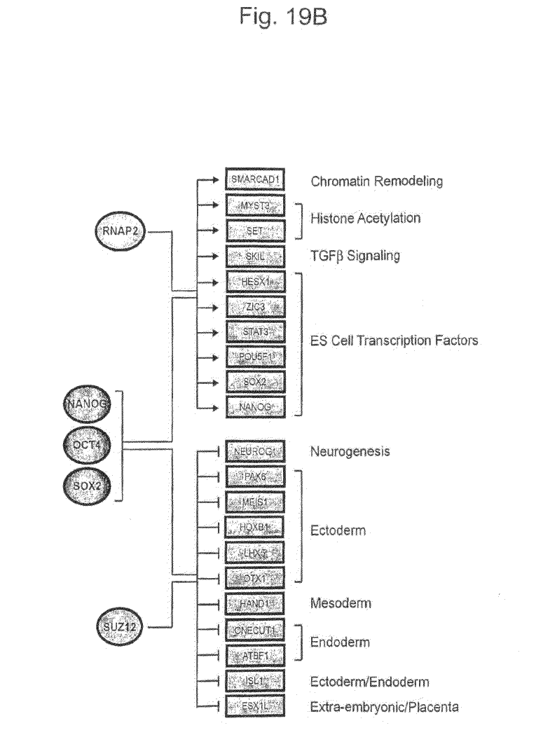

The transcription factors Oct4, Sox2 and Nanog have essential roles in early development and are required for the propagation of undifferentiated ES cells in culture (Nichols et al., 1998; Avilion et al., 2003; Chambers et al., 2003; Mitsui et al., 2003). Applicants recently reported that these transcription factors occupied promoters for many important developmental regulators in human ES cells (Boyer et al., 2005). This led Applicants to compare the set of genes encoding developmental regulators and occupied by Oct4, Sox2 and Nanog with those occupied by PRC2 (FIG. 19). Applicants found that each of the three DNA-binding transcription factors occupied approximately one-third of the PRC2-occupied genes that encode developmental transcription factors (FIG. 19A). Remarkably, Applicants found that the subset of genes encoding developmental regulators that were occupied by Oct4, Sox2 and Nanog and repressed in the regulatory circuitry highlighted in Boyer et al. were almost all occupied by PRC2 (FIG. 19B). These included transcription factors known to be important for differentiation into extra-embryonic, endodermal, mesodermal, and ectodermal lineages (e.g., ESX1L, ONECUT1, HAND1, HOXB1). As expected, active genes encoding ES cell transcription factors (e.g., ZIC3, STAT3, OCT4, NANOG) were occupied by Oct4, Sox2, Nanog and RNA polymerase II, but not PRC2 (FIG. 19B).

The observation that Oct4, Sox2 and Nanog are bound to a significant subset of developmental genes occupied by PRC2 supports a link between repression of developmental regulators and stem cell pluripotency. Like PRC2, Oct4 and Nanog have been shown to be important for early development and ES cell identity. It is possible, therefore, that inappropriate regulation of developmental regulators that are common targets of Oct4, Nanog and PRC2 contributes to the inability to establish ES cell lines in OCT4, NANOG and EZH2 mutants (Nichols et al., 1998; O'Carroll et al., 2001; Chambers et al., 2003; Mitsui et al., 2003).

Experimental procedures for examples 6-12 were carried out as follows:

### Cells and Cell Culture

Human H9 ES cells (WiCell, Madison, Wis.) were cultured as described (Boyer et al., 2005). Primary human skeletal muscle cells were obtained from Cell Applications (San Diego, Calif.) and expanded and differentiated into myotubes according to the supplier's protocols. Suz12−/− mouse cell lines were derived from blastocysts from crosses between heterozygous Suz12 mutant animals.

### Chromatin Immunoprecipitation and DNA Microarray Analysis

ChIP was combined with DNA microarray analysis as described (Boyer et al., 2005). The antibodies used here were specific for hypophosphorylated RNA polymerase II (8WG16) (Thompson et al., 1989), Suz12 (Upstate, 07-379), Eed (Hamer et al., 2002), H3K27me3 (Abcam, AB6002), and total histone H3 (Abcam, AB1791). The design of the oligo-based arrays were manufactured by Agilent Technologies. A whole-chip error model was used to calculate confidence values from the enrichment ratio and the signal intensity of each probe (probe p-value) and of each set of three neighboring probes (probe-set p-value). Probe-sets with significant probe-set p-values (p<0.001) and significant individual probe p-values were judged to be bound. Bound regions were assigned to genes if they were within 1 kb of the transcription start site from one of five genomic databases; RefSeq, MGC, Ensembl, UCSC Known Gene or H-Inv. All microarray data is available at ArrayExpress under the accession designation E-WMIT-7.

### Gene Expression Analysis

Gene expression data was collated from H1 ES cells (Sato et al., 2003), H9, HSF1 and HSF6 ES cells (Abeyta et al., 2004), and 79 differentiated human cell and tissue types (Su et al., 2004). Replicate gene expression data was obtained for wild-type mouse ES cells and Suz12-deficient cells using Agilent Mouse Development arrays.

### REFERENCES

Abeyta, M. J., Clark, A. T., Rodriguez, R. T., Bodnar, M. S., Pera, R. A., and Firpo, M. T. (2004). Unique gene expression signatures of independently derived human embryonic stem cell lines. Hum Mol Genet 13, 601-608.
Akasaka, T., van Lohuizen, M., van der Lugt, N., Mizutani-Koseki, Y., Kanno, M., Taniguchi, M., Vidal, M., Alkema, M., Berns, A., and Koseki, H. (2001). Mice doubly deficient for the Polycomb Group genes Mel18 and Bmi1 reveal synergy and requirement for maintenance but not initiation of Hox gene expression. Development 128, 1587-1597.
Ambrosetti, D. C., Basilico, C., and Dailey, L. (1997). Synergistic activation of the fibroblast growth factor 4 enhancer by Sox2 and Oct-3 depends on protein-protein interactions facilitated by a specific spatial arrangement of factor binding sites. Mol Cell Biol 17, 6321-6329.
Avilion, A. A., Nicolis, S. K., Pevny, L. H., Perez, L., Vivian, N., and Lovell-Badge, R. (2003). Multipotent cell lineages in early mouse development depend on SOX2 function. Genes Dev 17, 126-140.
Ballas, N., Grunseich, C. Lu, D. D., Speh, J. C., and Mandel, G. (2005). REST and its corepressors mediate plasticity of neuronal gene chromatin throughout neurogenesis. Cell 121, 645-657.
Bartel, D. P. (2004). MicroRNAs: genomics, biogenesis, mechanism, and function. Cell 116, 281-297.
Bejerano, G., Pheasant, M., Makunin, I., Stephen, S., Kent, W. J., Mattick, J. S., and Haussler, D. (2004). Ultraconserved elements in the human genome. Science 304, 1321-1325.
Bender, M., Turner, F. R., and Kaufman, T. C. (1987). A development genetic analysis of the gene regulator of postbithorax in Drosophila melanogaster. Dev Biol 119, 418-432.
Birve, A., Sengupta, A. K., Beuchle, D., Larsson, J., Kennison, J. A., Rasmuson-Lestander, A., and Muller, J. (2001). Su(z)12, a novel Drosophila Polycomb group gene that is conserved in vertebrates and plants. Development 128, 3371-3379.
Bortvin, A., Eggan, K., Skaletsky, H., Akutsu, H., Berry, D. L., Yanagimachi, R., Page, D. C., and Jaenisch, R. (2003). Incomplete reactivation of Oct4-related genes in mouse embryos cloned from somatic nuclei. Development 130 1673-1680.
Botquin, V., Hess, H., Fuhrmann, G., Anastassiadis, C., Gross, M. K., Vriend, G., and Scholer, H. R. (1998). New POU dimer configuration mediates antagonistic control of an osteopontin preimplantation enhancer by Oct-4 and Sox-2. Genes Dev 12, 2073-2090.
Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Bozdech, Z., Zhu, J., Joachimiak, M. P., Cohen, F. E., Pulliam, B., and DeRisi, J. L. (2003). Expression profiling of the schizont and trophozoite stages of Plasmodium falciparum with a long-oligonucleotide microarray. Genome Biol 4, R9.
Brand-Saberi, B. (2005). Genetic and epigenetic control of skeletal muscle development. Ann Anat 187, 199-207.
Brandenberger, R., Khrebtukova, I., Thies. R. S., Miura, T., Jingli, C., Puri, R., Vasicek, T., Lebkowski, J., and Rao, M. (2004). MPSS profiling of human embryonic stem cells. BMC Dev Biol. 4, 10.
Breiling, A., Turner, B. M., Bianchi, M. E., and Orlando, V. (2001). General transcription factors bind promoters repressed by Polycomb group proteins. Nature 412, 651-655.
Cao, R., Wang, L., Wang, H., Xia, L., Erdjument-Bromage, H., Tempst, P., Jones, R. S., and Zhang, Y. (2002). Role of histone H3 lysine 27 methylation in Polycomb-group silencing. Science 298, 1039-1043.
Cao, R., and Zhang, Y. (2004). SUZ12 is required for both the histone methyltransferase activity and the silencing function of the EED-EZH2 complex. Mol Cell 15, 57-67.
Caretti, G., Di Padova, M., Micales, B., Lyons, G. E., and Sartorelli, V. (2004). The Polycomb Ezh2 methyltransferase regulates muscle gene expression and skeletal muscle differentiation. Genes Dev 18, 2627-2638.
Catena, R., Tiveron, C., Ronchi, A., Porta, S., Ferri, A., Tatangelo, L., Cavallaro, M., Favaro, R., Ottolenghi, S., Reinbold, R., et al. (2004). Conserved POU binding DNA sites in the Sox2 upstream enhancer regulate gene expression in embryonic and neural stem cells. J Biol Chem 279, 41846-41857.
Chambers, I. (2004). The molecular basis of pluripotency in mouse embryonic stem cells. Cloning Stem Cells 6, 386-391.
Chambers, I., Colby, D., Robertson, M., Nichols, J., Lee, S., Tweedie, S., and Smith, A. (2003). Functional expression cloning of Nanog, a pluripotency sustaining factor in embryonic stem cells. Cell 113, 643-655.
Czermin, B., Melfi, R., McCabe, D., Seitz, V., Imhof, A., and Pirrotta, V. (2002). Drosophila enhancer of Zeste/ESC complexes have a histone H3 methyltransferase activity that marks chromosomal Polycomb sites. Cell 111, 185-196.
Dailey, L., and Basilico, C. (2001). Coevolution of HMG domains and homeodomains and the generation of transcriptional regulation by Sox/POU complexes. J Cell Physiol 186, 315-328.
Dellino, G. I., Schwartz, Y. B., Farkas, G., McCabe, D., Elgin, S. C., and Pirrotta, V. (2004). Polycomb silencing blocks transcription initiation. Mol Cell 13, 887-893.
Denell, R. E., and Frederick, R. D. (1983). Homoeosis in Drosophila: a description of the Polycomb lethal syndrome. Dev Biol 97, 34-47.
Duncan, I. (1986). Control of bithorax complex functions by the segmentation gene fushi tarazu of D. melanogaster. Cell 47, 297-309.
Faust, C., Lawson, K. A., Schork, N. J., Thiel, B., and Magnuson, T. (1998). The Polycomb-group gene eed is required for normal morphogenetic movements during gastrulation in the mouse embryo. Development 125, 4495-4506.
Francis, N. J., Saurin, A. J., Shao, Z., and Kingston, R. E. (2001). Reconstitution of a functional core polycomb repressive complex. Mol Cell 8, 545-556.
Franke, A., DeCamillis, M., Zink, D., Cheng, N., Brock, H. W., and Paro, R. (1992). Polycomb and polyhomeotic are constituents of a multimeric protein complex in chromatin of Drosophila melanogaster. Embo J 11, 2941-2950.
Gerhard, D. S., Wagner, L., Feingold, E. A., Shenmen, C. M., Grouse, L. H., Schuler, G., Klein, S. L., Old, S., Rasooly, R., Good, P., et al. (2004). The status, quality, and expansion of the NIH full-length cDNA project: the Mammalian Gene Collection (MGC). Genome Res 14, 2121-2127.
Guenther, M. G., Jenner, R. G., Chevalier, B., Nakamura, T., Croce, C. M., Canaani, E., and Young, R. A. (2005). Global and Hox-specific roles for the MLL1 methyltransferase. Proc Natl Acad Sci USA 102, 8603-8608.
Hamer, K. M., Sewalt, R. G., den Blaauwen, J. L., Hendrix, T., Satijn, D. P., and Otte, A. P. (2002). A panel of monoclonal antibodies against human polycomb group proteins. Hybrid Hybridomics 21, 245-252.
Harbison, C. T., Gordon, D. B., Lee, T. I., Rinaldi, N. J., Macisaac, K. D., Danford, T. W., Hannett, N. M., Tagne, J. B., Reynolds, D. B., Yoo, J., Jennings, E. G., Zeitlinger, J., Pokholok, D. K., Kellis, M., Rolfe, P. A., Takusagawa, K. T., Lander, E. S., Gifford, D. K., Fraenkel, E., Young, R. A. (2004). Transcriptional Regulatory Code of a Eukaryotic Genome. Nature 431, 99-104.
Hart, A. H., Hartley, L., Ibrahim, M., and Robb, L. (2004). Identification, cloning and expression analysis of the pluripotency promoting Nanog genes in mouse and human. Dev Dyn 230, 187-198.
Hay, D. C., Sutherland, L., Clark, J., and Burdon, T. (2004). Oct4 knockdown induces similar patterns of endoderm and trophoblast differentiation markers in human and mouse embryonic stem cells. Stem Cells 22, 225-235.
He, J. Tegen, S. B., Krawitz, A. R., Martin, G. S., and Luo, K. (2003). The transforming activity of Ski and SnoN is dependent on their ability to repress the activity of Smad proteins. J Biol Chem. 278 30540-30547.
Hodgson, J. W., Argiropoulos, B., and Brock, H. W. (2001). Site-specific recognition of a 70-base-pair element containing d(GA)(n) repeats mediates bithoraxoid polycomb group response element-dependent silencing. Mol Cell Biol 21, 4528-4543.
Hombria, J. C., and Lovegrove, B. (2003). Beyond homeosis—HOX function in morphogenesis and organogenesis. Differentiation 71, 461-476.
Horard, B., Tatout, C., Poux, S., and Pirrotta, V. (2000). Structure of a polycomb response element and in vitro binding of polycomb group complexes containing GAGA factor. Mol Cell Biol 20, 3187-3197.
Hubbard, T., Andrews, D., Caccamo, M., Cameron, G., Chen, Y., Clamp, M., Clarke, L., Coates, G., Cox, T., Cunningham, F., et al. (2005). Ensembl 2005. Nucleic Acids Res
33 Database Issue, D447-453.
Hughes, T. R., Marton, M. J., Jones, A. R., Roberts, C. J., Stoughton, R., Armour, C. D., Bennett, H. A., Coffey, E., Dai, H., He, Y. D., et al. (2000). Functional discovery via a compendium of expression profiles. Cell 102, 109-126.
James, D., Levine, A. J., Besser, D., and Hemmati-Brivanlou, A. (2005). TGFbeta/activin/nodal signaling is necessary for the maintenance of pluripotency in human embryonic stem cells. Development 132, 1273-1282.
Kanellopoulou, C., Muljo, S. A., Kung, A. L., Ganesan, S., Drapkin, R., Jenuwein, T., Livingston, D. M., and Rajewsky, K. (2005). Dicer-deficient mouse embryonic stem cells are defective in differentiation and centromeric silencing. Genes Dev 19, 489-501.
Kennison, J. A. (2004). Introduction to Trx-G and Pc-G genes. Methods Enzymol 377, 61-70.
Kirmizis, A., Bartley, S. M., Kuzmichev, A., Margueron, R., Reinberg, D., Green, R., and Farnham, P. J. (2004). Silencing of human polycomb target genes is associated with methylation of histone H3 Lys 27. Genes Dev 18, 1592-1605.
Kuroda, T., Tada, M., Kubota, H., Kimura, H., Hatano, S. Y., Suemori, H., Nakatsuji, N., and Tada, T. (2005). Octamer and Sox elements are required for transcriptional cis regulation of Nanog gene expression. Mol Cell Biol 25, 2475-2485.
Kuzmichev, A., Jenuwein, T., Tempst, P., and Reinberg, D. (2004). Different EZH2-containing complexes target methylation of histone H1 or nucleosomal histone H3. Mol Cell 14, 183-193.
Kuzmichev, A., Margueron, R., Vaquero, A., Preissner, T. S., Scher, M., Kirmizis, A., Ouyang, X., Brockdorff, N., Abate-Shen, C., Farnham, P., and Reinberg, D. (2005). Composition and histone substrates of polycomb repressive group complexes change during cellular differentiation. Proc Natl Acad Sci USA 102, 1859-1864.
Kuzmichev, A., Nishioka, K., Erdjument-Bromage, H., Tempst, P., and Reinberg, D. (2002). Histone methyltransferase activity associated with a human multiprotein complex containing the Enhancer of Zeste protein. Genes Dev 16, 2893-2905.
Lee, J. H., Hart, S. R., and Skalnik, D. G. (2004). Histone deacetylase activity is required for embryonic stem cell differentiation. Genesis 38, 32-38.
Lee, T. I., Rinaldi, N. J., Robert, F., Odom, D. T., Bar-Joseph, Z., Gerber, G. K., Hannett, N. M., Harbison, C. T., Thompson, C. M., Simon, I., et al. (2002). Transcriptional regulatory networks in Saccharomyces cerevisiae. Science 298, 799-804.
Lehmann, O. J., Sowden, J. C., Carlsson, P., Jordan, T., and Bhattacharya, S. S. (2003). Fox's in development and disease. Trends Genet 19, 339-344.
Levine, S. S., King, I. F., and Kingston, R. E. (2004). Division of labor in polycomb group repression. Trends Biochem Sci 29, 478-485.
Levine, S. S., Weiss, A., Erdjument-Bromage, H., Shao, Z., Tempst, P., and Kingston, R. E. (2002); The core of the polycomb repressive complex is compositionally and functionally conserved in flies and humans. Mol Cell Biol 22, 6070-6078.
Lewis, E. B. (1978). A gene complex controlling segmentation in Drosophila. Nature 276, 565-570.
Lewis, B. P., Burge, C. B., and Bartel, D. P. (2005). Conserved seed pairing, often flanked by adenosines, indicates that thousands of human genes are microRNA targets. Cell 120, 15-20.
Loebel, D. A., Watson, C. M., De Young, R. A., and Tam, P. P. (2003). Lineage choice and differentiation in mouse embryos and embryonic stem cells. Dev Biol 264, 1-14.
Lund, A. H., and van Lohuizen, M. (2004). Polycomb complexes and silencing mechanisms. Curr Opin Cell Biol 16, 239-246.
Ma, H. W., Kumar, B., Ditges, U., Gunzer, F., Buer, J., and Zeng, A. P. (2004). An extended transcriptional regulatory network of Escherichia coli and analysis of its hierarchical structure and network motifs. Nucleic Acids Res 32, 6643-6649.
Mangan, S., and Alon, U. (2003). Structure and function of the feed-forward loop network motif. Proc Natl Acad Sci USA 100, 11980-11985.
Mangan, S., Zaslaver, A., and Alon, U. (2003). The coherent feedforward loop serves as a sign-sensitive delay element in transcription networks. J Mol Biol 334, 197-204.
Matin, M. M., Walsh, J. R., Gokhale, P. J., Draper, J. S., Bahrami, A. R., Morton, L, Moore, H. D., and Andrews, P. W. (2004). Specific knockdown of Oct4 and beta2-microglobulin expression by RNA interference in human embryonic stem cells and embryonic carcinoma cells. Stem Cells 22, 659-668.
Mayhall, E. A., Paffett-Lugassy, N., and Zon, L. I. (2004). The clinical potential of stem cells. Curr Opin Cell Biol 16, 713-720.
McAdams, H. H., and Arkin, A. (1997). Stochastic mechanisms in gene expression. Proc Natl Acad Sci USA 94, 814-819.
Milo, R., Shen-Orr, S., Itzkovitz, S., Kashtan, N., Chklovskii, D., and Alon, U. (2002). Network motifs: simple building blocks of complex networks. Science 298, 824-827.
Mitsui, K., Tokuzawa, Y., Itoh, H., Segawa, K., Murakami, M., Takahashi, K., Maruyama, M., Maeda, M., and Yamanaka, S. (2003). The homeoprotein Nanog is required for maintenance of pluripotency in mouse epiblast and ES cells. Cell 113, 631-642.
Molofsky, A. V., Pardal, R., and Morrison, S. J. (2004). Diverse mechanisms regulate stem cell self-renewal. Curr Opin Cell Biol 16, 700-707.
Mulholland, N. M., King, I. F., and Kingston, R. E. (2003). Regulation of Polycomb group complexes by the sequence-specific DNA binding proteins Zeste and GAGA. Genes Dev 17, 2741-2746.
Muller, I., Hart, C. M., Francis, N. J., Vargas, M. L., Sengupta, A., Wild, B., Miller, E. L., O'Connor, M. B., Kingston, R. E., and Simon, J. A. (2002). Histone methyltransferase activity of a Drosophila Polycomb group repressor complex. Cell 111, 197-208.
Natarajan, K., Meyer, M. R., Jackson, B. M., Slade, D., Roberts, C., Hinnebusch, A. G., Marton, M. J. (2001). Transcriptional profiling shows that Gcn4p is a master regulator of gene expression during amino acid starvation in yeast. Mol Cell Biol 21 4347-4368.
Nichols, J., Zevnik, B., Anastassiadis, K., Niwa, H., Klewe-Nebenius, D., Chambers, I., Scholer, H., and Smith, A. (1998). Formation of pluripotent stem cells in the mammalian embryo depends on the POU transcription factor Oct4. Cell 95, 379-391.
Nishimoto, M., Fukushima, A., Okuda, A., and Muramatsu, M. (1999). The gene for the embryonic stem cell coactivator UTF1 carries a regulatory element which selectively interacts with a complex composed of Oct-3/4 and Sox-2. Mol Cell Biol 19, 5453-5465.
Niwa, H. (2001). Molecular mechanism to maintain stem cell renewal of ES cells. Cell Struct Funct 26, 137-148.
Niwa, H., Miyazaki, J., and Smith, A. G. (2000). Quantitative expression of Oct-3/4 defines differentiation, dedifferentiation or self-renewal of ES cells. Nat Genet 24, 372-376.
O'Carroll, D., Erhardt, S., Pagani, M., Barton, S. C., Surani, M. A., and Jenuwein, T. (2001). The polycomb-group gene Ezh2 is required for early mouse development. Mol Cell Biol 21, 4330-4336.
Odom, D. T., Zizlsperger, N., Gordon, D. B., Bell, G. W., Rinaldi, N. J., Murray, H. L., Volkert, T. L., Schreiber, J., Rolfe, P. A., Gifford, D. K., et al. (2004). Control of pancreas and liver gene expression by HNF transcription factors. Science 303, 1378-1381.
Okumura-Nakanishi, S., Saito, M., Niwa, H., and Ishikawa, F. (2005). Oct-3/4 and Sox2 regulate Oct-3/4 gene in embryonic stem cells. J Biol Chem 280, 5307-5317.
Orlando, V., and Paro, R. (1995). Chromatin multiprotein complexes involved in the maintenance of transcription patterns. Curr Opin Genet Dev 5, 174-179.
Pasini, D., Bracken, A. P., Jensen, M. R., Denchi, E. L., and Helin, K. (2004). Suz12 is essential for mouse development and for EZH2 histone methyltransferase activity. Embo J 23, 4061-4071.
Pasquinelli, A. E., Hunter, S., and Bracht, J. (2005). MicroRNAs: a developing story. Curr Opin Genet Dev 15, 200-205.
Pera, M. F., and Trounson, A. O. (2004). Human embryonic stem cells: prospects for development. Development 131, 5515-5525.
Pesce, M., and Scholer, H. R. (2001). Oct-4: gatekeeper in the beginnings of mammalian development. Stem Cells 19, 271-278.
Pirrotta, V. (1998). Polycombing the genome: PcG, trxG, and chromatin silencing. Cell 93, 333-336.
Pruitt, K. D., Tatusova, T., and Maglott, D. R. (2005). NCBI Reference Sequence (RefSeq): a curated non-redundant sequence database of genomes, transcripts and proteins. Nucleic Acids Res 33 Database Issue, D501-504.
Remenyi, A., Scholer, H. R., and Wilmanns, M. (2004). Combinatorial control of gene expression. Nat Struct Mol Biol 11, 812-815.
Ren, B., Cam, H., Takahashi, Y., Volkert, T., Terragni, J., Young, R. A., and Dynlacht, B. D. (2002). E2F integrates cell cycle progression with DNA repair, replication, and G(2)/M checkpoints. Genes Dev. 16 245-256.
Resendis-Antonio, O., Freyre-Gonzalez, J. A., Menchaca-Mendez, R., Gutierrez-Rios, R. M., Martinez-Antonio, A., Avila-Sanchez, C., and Collado-Vides, J. (2005). Modular analysis of the transcriptional regulatory network of E. coli. Trends Genet 21, 16-20.
Reubinoff, B. E., Pera, M. F., Fong, C. Y., Trounson, A., and Bongso, A. (2000). Embryonic stem cell lines from human blastocysts: somatic differentiation in vitro. Nat Biotechnol 18, 399-404.
Ringrose, L., and Paro, R. (2004). Epigenetic regulation of cellular memory by the Polycomb and Trithorax group proteins. Annu Rev Genet 38, 413-443.
Rodda, D. J., Chew, J-L., Lim, L-H., Loh, Y-H., Wang, B., Ng, H-H., and Robson, P. (2005). Transcriptional Regulation of Nanog by Oct4 and Sox2. J Biol Chem, In press.
Rosenfeld, N., Elowitz, M. B., and Alon, U. (2002). Negative autoregulation speeds the response times of transcription networks. J Mol Biol 323, 785-793.
Sato, N., Meijer, L., Skaltsounis, L., Greengard, P., and Brivanlou, A. H. (2004). Maintenance of pluripotency in human and mouse embryonic stem cells through activation of Wnt signaling by a pharmacological GSK-3-specific inhibitor. Nat Med 10, 55-63.
Sato, N., Sanjuan, I. M., Heke, M., Uchida, M., Naef, F., and Brivanlou, A. H. (2003). Molecular signature of human embryonic stem cells and its comparison with the mouse. Dev Biol 260, 404-413.
Saurin, A. J., Shao, Z., Erdjument-Bromage, H., Tempst, P., and Kingston, R. E. (2001). A Drosophila Polycomb group complex includes Zeste and dTAFII proteins. Nature 412, 655-660.
Schepers, G. E., Teasdale, R. D., and Koopman, P. (2002). Twenty pairs of sox: extent, homology, and nomenclature of the mouse and human sox transcription factor gene families. Dev Cell 3, 167-170.
Scholer, H. R., Dressler, G. R., Balling, R., Rohdewohld, H., and Gruss, P. (1990). Oct-4: a germine-specific transcription factor mapping to the mouse t-complex. Embo J 9, 2185-2195.
Shao, Z., Raible, F., Mollaaghababa, R., Guyon, J. R., Wu, C. T., Bender, W., and Kingston, R. E. (1999). Stabilization of chromatin structure by PRC1, a Polycomb complex. Cell 98, 37-46.
Shen-Orr, S. S., Milo, R., Mangan, S., and Alon, U. (2002). Network motifs in the transcriptional regulation network of Escherichia coli. Nat Genet 31, 64-68.
Showell, C., Binder, O., and Conlon, F. L. (2004). T-box genes in early embryogenesis. Dev Dyn 229, 201-218.
Siepel, A., Bejerano, G., Pedersen, J. S., Hinrichs, A. S., Hou, M., Rosenbloom, K., Clawson, H., Spieth, J., Hillier, L. W., Richards, S., et al. (2005). Evolutionarily conserved elements in vertebrate, insect, worm, and yeast genomes. Genome Res 15, 1034-1050.
Simon, J., Chiang, A., and Bender, W. (1992). Ten different Polycomb group genes are required for spatial control of the abdA and AbdB homeotic products. Development 114, 493-505.
Solter, D., and Knowles, B. B. (1979). Developmental stage-specific antigens during mouse embryogenesis. Curr Top Dev Biol 13 Pt 1, 139-165.
Strutt, H., Cavalli, G., and Paro, R. (1997). Co-localization of Polycomb protein and GAGA factor on regulatory elements responsible for the maintenance of homeotic gene expression. Embo J 16, 3621-3632.
Su, A. I., Wiltshire, T., Batalov, S., Lapp, H., Ching, K. A., Block, D., Zhang, J., Soden, R., Hayakawa, M., Kreiman, G., Cooke. M. P., Walker, J. R., and Hogenesch, J. B. (2004). A gene atlas of the mouse and human protein-encoding transcriptomes. Proc Natl Acad Sci USA 101, 6062-6067.
Tapscott, S. J. (2005). The circuitry of a master switch: Myod and the regulation of skeletal muscle gene transcription. Development 132, 2685-2695.
Thieffry, D., Salgado, H., Huerta, A. M., and Collado-Vides, J. (1998). Prediction of transcriptional regulatory sites in the complete genome sequence of Escherichia coli K-12. Bioinformatics 14, 391-400.
Thompson, N. E., Steinberg, T. H., Aronson, D. B., and Burgess, R. R. (1989). Inhibition of in vivo and in vitro transcription by monoclonal antibodies prepared against wheat germ RNA polymerase II that react with the heptapeptide repeat of eukaryotic RNA polymerase II. J Biol Chem 264, 11511-11520.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Tie, F., Furuyama, T., Prasad-Sinha, J., Jane, E., and Harte, P. J. (2001). The Drosophila Polycomb Group proteins ESC and E(Z) are present in a complex containing the histone-binding protein p55 and the histone deacetylase RPD3. Development 128, 275-286.
van der Lugt, N. M., Alkema, M., Berns, A., and Deschamps, J. (1996). The Polycomb-group homolog Bmi-1 is a regulator of murine Hox gene expression. Mech Dev 58, 153-164.
van Lohuizen, M. (1998). Functional analysis of mouse Polycomb group genes. Cell Mol Life Sci 54, 71-79.
Vire, E., Brenner, C., Deplus, R., Blanchon, L., Fraga, M., Didelot, C., Morey, L., Van Eynde, A., Bernard, D., Vanderwinden, J. M., Bollen, M., Esteller, M., Di Croce, L., de Launoit, Y., Fuks, F. (2006). The Polycomb group protein EZH2 directly controls DNA methylation. Nature 439, 871-874.
Wang, J., Mager, J., Schnedier, E., and Magnuson, T. (2002). The mouse PcG gene eed is required for Hox gene repression and extraembryonic development. Mamm Genome 13, 493-503.
Wei, C. L., Miura, T., Robson, P., Lim, S. K., Xu, X. Q., Lee, M. Y., Gupta, S., Stanton, L., Luo, Y., Schmitt, J., Thies, S., Wang, W., Khrebtukova, I., Zhou, D., Liu, E. T., Ruan, Y. J., Rao, M., and Lim B. (2005). Transcriptome profiling of human and murine ESCs identifies divergent paths required to maintain the stem cell state. Stem Cells 23, 166-185.
Weinmann, A. S., Yan, P. S. Oberley, M. J. Huang, T. H., Farnham, P. J. (2002). Isolating human transcription factor targets by coupling chromatin immunoprecipitation and CpG island microarray analysis. Genes Dev. 16 235-244.
Woolfe, A., Goodson, M., Goode, D. K., Snell, P., McEwen, G. K., Vavouri, T., Smith, S. F., North, P., Callaway, H., Kelly, K., et al. (2005). Highly conserved non-coding sequences are associated with vertebrate development. PLoS Biol 3, e7.
Yuan, H., Corbi, N., Basilico, C., and Dailey, L. (1995). Developmental-specific activity of the FGF4 enhancer requires the synergistic action of Sox2 and Oct-3. Genes Dev 9, 2635-2645.
Zaehres, H., Lensch, M. W., Daheron, L., Stewart, S. A., Itskovitz-Eldor, J. and Daley, G. Q. (2005). High-Efficiency RNA Interference in Human Embryonic Stem Cells. Stem Cells 23, 299-305.

1. A method for identifying regions of a genome to which a protein of interest binds, the method comprising the steps of:
(a) producing a mixture comprising DNA fragments to which the protein of interest is bound;
(b) isolating one or more DNA fragments to which the protein of interest is bound from the mixture produced in step (a); and
(c) identifying regions of the genome which are complementary to the DNA fragments isolated in step (b),
thereby identifying regions of the genome to which the protein of interest binds.

2. The method of claim 1, wherein the mixture in step (a) is from a stem cell.

3. The method of claim 2, wherein the stem cell is an embryonic stem cell.

4. The method of claim 2, wherein said stem cell is derived from mice, rats, rabbits, cows, pigs, humans, or non-human primates.

5. The method of claim 2, where the cell maintains the potential to differentiate into endoderm, mesoderm, and ectoderm tissues.

6. The method of claim 1 or claim 2, comprising, between Steps (b) and (c), generating a probe from the one, or more of the isolated DNA fragments.

7. The method of claim 6, wherein step (c) comprises combining the probe with one or more sets of distinct oligonucleotide features bound to a surface of a solid support, wherein the distinct oligonucleotide features are each complementary to a region of the genome, under conditions in which specific hybridization between the probe and the oligonucleotide features can occur, and detecting said hybridization, wherein hybridization between the labeled probe and a oligonucleotide feature relative to a suitable control indicates that the protein of interest is bound to the region of the genome to which the sequence of the oligonucleotide feature is complementary.

8. The method of claim 7, wherein each set of distinct oligonucleotide features is complementary to a promoter region of a gene.

9. The method of claim 8, wherein the promoter region of the gene comprises from about 8 kb upstream to about 2 kb downstream of a transcriptional start site.

10. The method of claim 8, wherein the distinct oligonucleotide features are complementary to adjacent regions in the genome that are spaced from 10 bp to 5000 bp of each other.

11. The method of claim 10, wherein the oligonucleotide features comprise DNA or RNA or modified forms thereof.

12. The method of claim 11 wherein the modified forms of DNA are PNA or LNA molecules.

13. The method according to claim 8, wherein said oligonucleotide features bound to a surface of a solid support includes sequences representative of promoter regions distributed across at least a portion of a genome.

14. The method of claim 7, wherein the oligonucleotide features are complementary to at least 50% of the promoter regions in the genome.

15. The method according to claim 7, wherein the solid support is a planar substrate.

16. The method according to claim 7, wherein said planar substrate is glass.

17. The method of claim 7, wherein the sets of distinct oligonucleotide features bound to a solid surface comprise an array.

18. The method of claim 17, wherein the array is a tiled array

19. A method for identifying the differentiation potential of a test cell, comprising
(i) identifying regions of the genome of the test cell to which a protein binds, according to the method of claim 1,
(ii) comparing the regions identified in (i) to one or more reference set of bound regions, each reference set of bound regions indicative of a differentiation potential; and
wherein a match between the regions identified in (i) and one of the reference set of bound regions indicates that the test cell has the differentiation potential corresponding to the reference set of bound regions.

20. The method of claim 19, wherein the protein is a DNA-binding protein.

21. The method of claim 19, wherein the protein is a transcriptional repressor or a transcriptional repressor.

22. The method of claim 19, wherein the protein is Oct4, Sox2 or Nanog.

23. The method of claim 19, wherein the protein is RNA polymerase II.

24. The method of claim 19, wherein the protein is a component of the Polycomb Repression Complex 2 (PRC2).

25. The method of claim 24, wherein the protein is a SUZ12.

26. The method of claim 19, wherein the protein is a histone.

27. The method of claim 19, wherein the test cell is an stem cell.

28. The method of claim 27, wherein the test cell is an embryonic stem cell.

29. The method of claim 28, wherein the embryonic stem cell is an human embryonic stem cell.

30. The method of claim 27, wherein the stem cell is a cancer stem cell.

31. The method of claim 19, wherein the test cell maintains the potential to differentiate into at least one tissue selected from of endoderm, mesoderm, and ectoderm tissue.

32. The method of claim 31, where the test cell maintains the potential to differentiate into endoderm, mesoderm, and ectoderm tissues.

33-42. (canceled)

43. A method for identifying an agent that promotes the differentiation of a stem cell towards a given lineage, comprising
(a) providing a culture comprising the stem cell;
(b) contacting said culture with the agent;
(c) identifying regions of the genome of the stem cell to which a protein binds according to the method of claim 1; and
(d) comparing the regions identified in (c) to one or more reference sets of bound regions indicative of cell differentiating towards the given lineage, wherein a match between the regions identified in (c) and one of the reference sets of bound regions indicates that the agent promotes the differentiation of the stem cell towards the given lineage.

44. The method of claim 43, wherein the protein is Oct4, Sox2 or Nanog.

45. The method of claim 43, wherein the protein is RNA polymerase II or SUZ12.

46. The method of claim 43, wherein the agent is selected from nucleic acids, peptides, polypeptides, small organic molecules, antibodies, ribozymes, antisense oligonucleotides, and RNAi constructs.

47. The method of claim 43, wherein the cell is an embryonic stem cell.

48. The method of claim 43, wherein the stem cell is an human embryonic stem cell.

49. The method of claim 43, wherein the stem cell is a cancer stem cell.

50. The method of claim 43, wherein the stem cell maintains the potential to differentiate into at least one tissue selected from of endoderm, mesoderm, and ectoderm tissue.

51. The method of claim 43, where the stem cell maintains the potential to differentiate into endoderm, mesoderm, and ectoderm tissues.

52. The method of claim 43, where the stem cell maintains the potential to differentiate into a neuronal cell type.

53. The method of claim 43, wherein said stem cell is derived from mice, rats, rabbits, cows, pigs, humans, or non-human primates.

54. The method of claim 43, wherein the stem cell is an human embryonic stem cell H9.

55. A method of obtaining a set of nucleotide array probes tiling the promoter region of a gene, the method comprising:
(i) selecting a transcriptional start site for the gene;
(ii) identifying masked and unmasked subsequences within a contiguous window of genomic DNA sequence that comprises the transcriptional start site;
(iii) for each unmasked subsequence greater than 20 nucleotides in length, generate a subset of preliminary probes, the preliminary probe having
(a) a length of between 40 and 80 nucleotides; and
(b) a sequence identical to a portion of the unmasked subsequence,
and
(iv) from the preliminary probes of step (iii), selecting a set of nucleotide array probes tiling the promoter region of the gene, wherein the array probes that are selected
(a) have one or more desirable hybridization properties; and
(b) tile the promoter region with an average density of about from 100 to about 1000 bp.

56. The method of claim 55, wherein the transcriptional start site for the gene is selected from a group of transcriptional start sites, each resulting in a transcript, wherein the start site selected results in the longest transcript.

57. The method of claim 55, wherein the window of genomic DNA sequence spans from about 8 kb upstream of the start site to about 2 kb downstream.

58. The method of claim 55, wherein each probe in the subset has a length of between 50 and 70 nucleotides.

59. The method of claim 55, wherein the desirable properties for the array probes are selected from (a) a GC nucleotide content of between 30 and 100 percent; (b) low self-binding; (c) low complexity; and (d) high uniqueness.

60. The method of claim 55, wherein the array probes in the subgroup tile the promoter region with a density of about from 200 bp to about 300 bp.

61. The method of claim 55, further comprising the step of:
(v) selecting additional preliminary probes for the set of nucleotide array probes, wherein the additional preliminary probes:
(a) have less desirable hybridization properties than the preliminary probes selected in step (iv); and
(b) are flanked by two nucleotide array probes, wherein the distance between the two flanking probes along the genome is greater than the average distance between adjacent nucleotide array probes of step (iv).

62. The method of claim 61, wherein the distance along the genome between the two flanking probes along the genome is greater than the average distance between adjacent nucleotide array probes of step (iv) by at least 100 bp.

63. The method of claim 55, comprising the step of:
(v) synthesizing a plurality of nucleic acid probes, each probe having the nucleotide sequence of a nucleotide array probe.

64. The method of claim 63, comprising the step of:
(vi) immobilizing the plurality of probes on a glass surface.

65. The method of claim 55, wherein the gene is a mammalian gene.

66. The method of claim 65, wherein the gene is a human gene.

67. The method of claim 55, further comprising synthesizing at least one of the nucleotide array probes.

68. The method of claim 67, further comprising immobilizing the nucleotide array probe on a microarray.

69. A method of obtaining a set of nucleotide array probes tiling the promoter region of a set of genes from a genome or a portion thereof, the method comprising identifying, for each gene, a set of nucleotide array probes tiling the promoter region of the gene according to the method of claim 55.

70-73. (canceled)
